(19) United States
(12) Patent Application Publication
(10) Pub. No.: US 2026/0337150 A1
(43) Pub. Date: Oct. 29, 2026

(54) NEW CLASS OF LIPIDS FOR DELIVERING ACTIVE INGREDIENTS INTO CELLS

(71) Applicant: OZ BIOSCIENCES, MARSEILLE CEDEX 09 (FR)

(72) Inventors: Olivier ZELPHATI, ROQUEFORT LA BEDOULE (FR); Florent POULHES, MARSEILLE (FR)

(21) Appl. No.: 19/485,648

(22) PCT Filed: Jun. 21, 2024
(86) PCT No.: PCT/EP2024/067547

§ 371 Date: Nov. 18, 2025

(30) Foreign Application Priority Data
Jun. 22, 2023 (EP) 23305999.7

(51) Int. Cl. C07C 237/22; A61K 47/18; A61K 47/22; C07C 231/00; C07D 207/06; C07D 233/61

(52) U.S. Cl. CPC C07C 237/22; A61K 47/18; A61K 47/22; C07C 231/00; C07D 207/06; C07D 233/61

(57) ABSTRACT

The present invention concerns a lipid of formula (I), a composition comprising said lipid of formula (I), a process for manufacturing said lipid of formula (I) and/or said composition, the lipid of formula (I) and/or the composition for its use as a medicament, the use of said lipid of formula (I) and/or of said composition as a vector for delivering an active ingredient to subject(s), organ(s), cell(s) and/or tissue(s). a method for transfecting cells with a nucleic acid and/or a protein and/or a peptide and/or a polysaccharide and/or a lipid and/or a small organic or inorganic molecule and/or any type of bioactive molecule using said lipid of formula (I) and/or said composition, transfected cells obtained by said method, and a kit comprising said lipid of formula (I) and/or said composition.

### FIELD OF THE INVENTION

The present invention concerns a lipid of formula (I), a composition comprising said lipid of formula (I), a process for manufacturing said lipid of formula (I) and/or said composition, the lipid of formula (I) and/or the composition for its use as a medicament, the use of said lipid of formula (I) and/or of said composition as a vector for delivering an active ingredient to subject(s), organ(s), cell(s) and/or tissue(s). a method for transfecting cells with a nucleic acid and/or a protein and/or a peptide and/or a polysaccharide and/or a lipid and/or a small organic or inorganic molecule and/or any type of bioactive molecule using said lipid of formula (I) and/or said composition, transfected cells obtained by said method, and a kit comprising said lipid of formula (I) and/or said composition.

### BACKGROUND AND PRIOR ART

Gene therapy, including nucleic acid-mediated therapy, represents today one of the most promising opportunities for curing diseases otherwise impossible to fight by traditional means. These innovative protocols imply the delivery in cellulo of biologically active ingredients in a safe way, using a broad variety of delivery vehicles. The intracellular delivery of biologically active agents into cells, organs and/or organisms finds applications in a large variety of field ranging from biology to medicine. In biology, the introduction of nucleic acids (transfection) such as DNA or RNA can be used in particular for studying the regulation of the expression of genes, clarifying, silencing or editing their functions as well as overexpressing them. In the same way, the intracellular transport of peptides, proteins, polysaccharides, lipids, small organic and inorganic molecules and any other biologically active molecule renders possible to study the fundamental biological mechanisms and to develop therapeutics. Nucleic acids therapeutics are susceptible to degradation by nucleases and cannot penetrate the cells due to their size and charge. It is also the case with various proteins, peptides or small molecules. Accordingly, development of drug delivery systems to target cells by clinically translatable systems is critical to provide opportunities to tackle a series of life-threatening diseases.

Among the large number of techniques used for introducing nucleic acids into cells, lipids and particularly cationic lipids (positively charged at physiological pH) in the form of systems which are organized or not (such as liposomes, uni-lamellar or multi-lamellar vesicles, hexagonal phases and/or micelles), and/or a mixture of lipids are probably the most widely used. They form complexes with nucleic acids such as lipoplexes or lipopolyplexes or lipid nanoparticles (LNPs) and allow the delivery of nucleic acids, proteins and peptides to pass through the cell membranes. Positively charged lipid formulation forms complexes with negatively charged nucleic acid. The resulting positively charged complexes can then bind to the negatively charged cell membrane. The exact mechanism of how nucleic acid/chemical complexes pass through cell membrane is unclear but it is believed that endocytosis is involved in the process of cellular uptake. The efficiency of chemical methods is largely dependent on factors such as pH, nucleic acid/reagent ratio, salt content or temperature. As a consequence, it is clearly difficult to predict how a chemical reagent will behave in transfection, especially in an in vivo application.

Among all the main known cationic lipid structures, we can cite in a non-exhaustive manner and by way of examples: monovalent cationic lipids in the form of quaternary ammonium salts, such as DOTMA (N-[1-(2,3-dioleyloxy) propyl]-N, N, N-trimethylammonium chloride), DOTAP (I, 2-dioleoy-3-tri-methylarnmonium propane) or DDAB (dioctadecyldimethylammonium bromide), monovalent cationic lipids in the form of pyridinium salts such as SAINT-2 (N-methyl-4-(dioleyl)methylpyridinium chloride), multivalent cationic lipids in the form of lipospermines such as DOGS (5-carboxyspermylglycine-dio-ctadecylamine), and DOSPA (2,3-dioleyloxy-N-[2 (sperminecarboxamido)ethyl]-N, N-dimethyl-propanammonium trifluoroacetate), multivalent cationic lipids in the form of lipopolylysines and cholesterol cationic derivatives such as DC-Chol (3-[N—(N′—N′,-dimethylaminomethane)-carbamoyl]cholesterol).

The use of cationic lipids (formulated or not with a neutral or helper lipid) for transfection has numerous advantages as they are not immunogenic, unlike viral agents, are simple to use, make it possible to deliver nucleic acids without a size limit, have high affinity with any kind of nucleic acids and can be produced in large quantities. Furthermore, lipofection (delivery of nucleic acids by lipid-based formulation) mediated by formulated cationic lipids is the most used method in research laboratories for transfecting cells in vitro due to its ease of use, its efficacy in the presence or absence of serum for a large variety of cell types, in particular adherent cells, and its versatility for the delivery of nucleic acids. These assets justify the large number of research program dealing with the development of both in vitro and in vivo cationic lipids as transfection vectors. However, these synthetic vectors are poorly or not at all effective for the transfection of certain cell types, in particular cells in suspension such as lymphocytes, stem cells, primary cells (non-dividing) etc. The reduced efficacy of the cationic lipids in transfecting certain cells is explained by the poor ability of lipoplexes to attach to the surface of these cells.

In this way, various approaches using specific ligands such as transferrin, epidermal growth factor (EGP), monoclonal antibodies or peptides, coupled or attached with a cationic polymer or a co-lipid formulated with cationic lipids, have been successfully tested. However, quiescent cells are also very difficult to transfect using lipofection, as the absence of means for targeting the nucleus, such as nuclear localization sequences prevent the complexes present in the cytosol to pass through the pores of the nuclear membrane and thus to be transcribed. Several approaches using ternary complexes (lipoplexes: cationic lipids and nucleic acids plus a nuclear targeting element) based on histones, peptide nuclear localization sequences, have been studied in order to get round this problem. However, the addition of these targeting elements to the cationic lipids makes the formulation and preparation of the complexes difficult and their use is thus very limited. Furthermore, and most importantly, the in vivo efficacy of lipofection remains too low, limited by the enzymatic degradation of the complexes, their pharmacology and by the presence of transfection inhibitors in body fluids and mucus, such as proteins and polysaccharides, which strongly inhibit transfection. The main parameters affecting lipoplex transfection efficacy have been widely studied, suggesting that the best levels of in vivo gene expression were obtained using high lipid/nucleic acid ratios which lead to significant toxicity. Thus, the in vivo cytotoxicity of the cationic lipids remains the major drawbacks of this strategy, mainly because of poor biodegradability of the cationic lipids which are not naturally present in cells as well as the use of permanent positive charge that is well-known for being detrimental to cell's survival.

Various efforts have been made to address those issues, such as the synthesis of lipids having a bond sensitive to the reducing medium in the fatty chains in order to facilitate their metabolism, or the incorporation of a vinyl ether group in the spacer arm of a cationic lipid. Vinyl group being sensitive to acidic pH, it allowed a rapid cleavage of the lipid in the acid medium of the endosomes, leading to a destabilization of the structure of the lipoplexes embedded in the endosome's membrane, allowing thus early release of the DNA in the cytosol. Similarly, a family of cationic lipids provided with a spacer arm containing a linear or cyclic orthoester group stable at physiological pH, that hydrolysed at acid pH has been described, while there are numerous examples of cationic lipids containing ester groups such as DMT-M (Gly) and DO™ (Gly)-tetraesters.

In the same way, to tackle biodegradability issue, lack of targeting and to add an additional biocompatibility feature, amino acid-based lipids with cleavable linkers have been designed and developed (WO 2009/106713 A3). These molecules displayed significant progress in transfection efficiency, safety and extended transfection applications to a variety of biomolecules, due to their innovative design. However, the presence of permanent positive charge(s) at physiological pH, minimizes their in vivo efficiency despite a structure-related better biodegradability in intracellular medium.

As recent reports demonstrated that the combined presence of permanent positive charge and of non-degradable backbones were the two main obstacles for safe and efficient in vivo gene delivery, new generations of lipids have been developed to reduce toxicity of permanently charged cationic lipid without compromising transfection efficacy. Based on the same structural model (a polar nitrogen-based head, a linker region, and two or multiple nonpolar hydrocarbon tails), they present an optimal pKa value of 6.2-7.0, which is designed to be positively charged at acidic pH to allow efficient encapsulation of nucleic acids such as DNAs or RNAs into lipid nanoparticles (LNPs) and effective DNA or RNA intracytoplasmic release upon protonation under acidic condition (endosomal pathway). On the other hand, they are neutral under physiologic conditions (pH=7.0 to 7.4) which prevent nonspecific interactions with fluids components and tissues, higher biocompatibility level and significant reduction of toxicity in comparison to the permanently charged cationic lipids. Lipids with an appropriate pKa would change their electrostatic charge to ensure proper vector formation, optimal in vivo circulation, and efficient cytosolic release of the therapeutic cargo. Specifically, such ionizable lipids should be designed to be positively charged at acidic pH during the production of lipid nanoparticles (LNPs) so that the electrostatic interactions with the Nucleic Acids (NAs) could be maximized (high NA condensation=high loading efficiency). Then, the lipids should turn into almost neutral under physiological conditions to prevent rapid sequestration by immune cells during systemic circulation. Also, the ionizable lipids should become positive again upon exposure to the typical acidic environment of the endosomes, thus, destabilizing the endosomal membrane and promoting the cytosolic delivery of the genetic cargo. Finally, in the cytosol, the neutral pH would favour the release of the NAs from the ionizable lipids, thus, enabling their free interaction with the cell machinery. The ionizable lipids is thus the critical component of the nanoparticle formulation since it is responsible to protect nucleic acids from degradation and to release the NAs into cells.

Ionizable lipids have been associated with specific lipidic components into supramolecular architectures known as lipid nanoparticles (LNP) or stable nucleic acid lipid particles (SNALP) or lipid polycations-DNA (LPD) or solid lipid nanoparticles (SLN) or nanostructured lipid carriers (NLC) when encapsulating a biomolecule such as a nucleic acid. These components provide the LNPs with additional features that potentialize the positive effect carried by ionizable components such as neutral phospholipids or sterol derivatives. Furthermore, when addressing in vivo injection, polyethylene glycol (PEG) modified-lipids (PEGylation) have been included in the LNPs formulation to increase its circulation stability, reduce nonspecific detrimental interactions, reduce their aggregation, and thus improve nucleic acid delivery efficiency. The versatile nature of lipids and their capacity to be easily modulated and modified make them ideal candidates for the transport of long and fragile nucleic acids such as DNA, mRNA or saRNA. In recent years, the number of research studies oriented towards the design and synthesis of such LNPs and especially on the design and synthesis of ionizable lipids has grown exponentially. Some recent examples may be found in comprehensive reviews (An ionizable lipid toolbox for RNA delivery, Han et al., Nature Communications 12, 7233 (2021); Lipid nanoparticles for mRNA delivery, Hou et al., Nature reviews materials 6, pages 1078-1094 (2021)). The molecules described in these different studies highlighted the fact that ionizable lipids used in LNPs can be of different structures that may impact positively nucleic acid delivery, especially in vivo. Accordingly in recent years several ionizable lipid structure have been described:
unsaturated ionizable lipids (such as DLin-MC3-DMA, A6, OF-02, A18-Iso5-2DC18),
multi-tail ionizable lipids (such as SM102, ALC-0315, A9, Lipid 2,2 (8,8); 98N12-5, C12-200, cKK-E12 and 9A1P9),
ionizable polymer-lipids (such as 7C1 and G0-C14),
acid-sensitive ionizable lipids (such as L319, 304O3, OF-Deg-Lin and 306-O12B)
branched-tail ionizable lipids (such as 306Oi10 and FTT5).

To date, formulation in lipid nanoparticles (LNPs) represents the most advanced delivery platform for gene therapy, vaccines and reliable candidates to treat manifold diseases. LNPs were recently FDA approved for the treatment of amyloidosis disease by delivery of siRNA, and more recently for the widely distributed SARS-COV-2 vaccines, based on mRNA-LNPs. Interestingly, ionizable lipids efficient for siRNA (such as DLin-MC3-DMA) did not share the same structural patterns than those used for efficient mRNA delivery (such as SM102 and ALC-0315). This demonstrates the need to develop a reliable platform for the synthesis of a wide range of ionizable lipids able to provide components perfectly tuned to the requirements of each possible application. One universal LNPs based synthetic platform will be more convenient, cost effective and somehow safest for nucleic acids therapeutics. Furthermore, and despite the current LNPs breakthrough, there are still some concerns about the inherent toxicity of some of these nanocarriers. Notably, the biodistribution and cellular uptake of LNPs which are affected by their surface composition as well as by their colloidal stability. Consequently, new generation of tunable lipids with higher biodegradability & biocompatibility, better capacity to form small and polydisperse nanoparticles, diverse and/or broader pKa range in order to target different tissues, diseases or harsh biological environment and higher compatibility towards multiple nucleic acids or other types of molecules such as proteins, antibodies, peptides is needed.

The present inventors have surprisingly found a new class of lipids, and in particular a new class of tunable lipids, which can be used for delivering active agents into cells without having the above-mentioned drawbacks.

In particular, the present inventors have surprisingly found a new versatile family of lipids, preferably tunable lipids, which are compatible with the presence of serum and are non-toxic, allowing an effective in vitro, ex vivo and in vivo delivery of all types of biomolecules such as nucleic acids, peptides, proteins, polysaccharides, and lipids into living cells.

In particular, the present inventors have surprisingly found new class of lipids that may comprise a wide variety of amine-based end-groups allowing a fine tuning of reversible positive charges thereby conferring to the new class of lipids a net pKa value consistent with the formulation of LNPs associated to efficient delivery of biomolecules into cells. The innovative structure of these new lipids, preferably new tunable lipids, combines:
The amphiphilic properties of the lipids in order to form organized structures (such as liposomes, lipid nanoparticles and micelles) allowing the transport and vectorization of active molecules towards their target, their properties of forming non-covalent complexes with negatively charged nucleic acids, and their properties of destabilizing the cell membranes.
The properties of the head group of the lipid, preferably of the tunable head group of the lipid, advantageously constituted by a chemical moiety centred on a Nitrogen atom, itself part of a chemical group chosen to become protonated only at acidic pH while remaining neutral otherwise. Moreover, sequences of these groups, preferably of these tunable groups, interact with several types of biomolecules including nucleic acids, peptides and proteins and are completely non-toxic.
The ability to degrade rapidly in intracellular environment due to the presence of a spacer arm between the lipophilic part and the head, preferably the tunable head, provided with a functional group incorporating a bond sensitive to its environment (such as pH, oxidation-reduction and/or enzymes) which can be cleaved in cytosolic medium and/or ester bond in the hydrophobic chain. The compounds originating from the resultant degradation are natural molecules (fatty acids. amino acids-based products) which are easily metabolized by the cell. The biodegradable character of these molecules makes them non-cytotoxic as a result.

The present invention thus offers an innovative solution to produce delivery system such as lipid nanoparticles or liposomes or lipid-based particles using a brand-new series of lipidic molecules able to efficiently complex, protect and deliver a wide array of various active ingredients due to their carefully chosen molecular design. The present invention describes with care an association of a lipophilic area mixing in adequate proportions several hydrophobic moieties such as alkyl chains, unsaturations, and optionally bioreducible groups that offer to these molecules an increased biocompatible and/or biodegradable character. This strategic choice is completed by the incorporation of a linker pattern that plays the dual role of maintaining a right distance between hydrophobic and hydrophilic parts and of contributing to the biodegradability of the whole structure. Finally, the polar head is constituted by a chemical moiety centred on a Nitrogen atom, itself part of a chemical group chosen to become protonated only at acidic pH (i.e., pH=3.5-6.9) while remaining not charged at higher pH.

Special care has been put to the incorporation of biodegradable or bioreducible moiety into the lipidic chemical structure. Indeed, such moiety provides the molecule a faster metabolization and its removal from the body following delivery of the active pharmaceutical ingredient to a target area.

Additionally, compounds claimed in this invention are all parts of a pharmaceutical formulation, meaning that their efficiency can rely on the choice of the other components of the formulation, their association (self-assembly) using a carefully experimental procedure including for example micro-fluidic processing, high pressure flow or extrusion systems or any other controlled formulations procedure.

### SUMMARY OF THE INVENTION

The present invention relates to a lipid of formula (I), or a stereoisomer thereof, or a pharmaceutically acceptable salt thereof:

R-Sp-Zₛ (I)

where:
R comprises one or more branched or linear, unsaturated or saturated, optionally fluorinated, alkyl chains, comprising 6 to 48 carbon atoms, preferably between 10 and 36 carbon atoms, optionally comprising one or more heteroatoms other than fluorine; or one or more cyclic or polycyclic groups known to be lipophilic, optionally comprising one or more heteroatoms, such as a steroid group optionally comprising one or more heteroatoms, a polyaromatic group optionally comprising one or more heteroatoms, or an alkaloid derivative group optionally comprising one or more heteroatoms; or a natural or synthetic lipid optionally comprising one or more heteroatoms; or a combination thereof;
Sp comprises one or more branched or linear, unsaturated or saturated, optionally fluorinated alkyl chains, comprising 6 to 24 carbon atoms, preferably between 10 and 18 carbon atoms, optionally comprising one or more heteroatoms and/or bioreducible bonds; and
Zₛ comprises one or more branched or linear, unsaturated or saturated, optionally fluorinated alkyl chains, comprising 2 to 36 carbon atoms, preferably comprising 2 to 24 carbon atoms, optionally comprising one or more heteroatoms other than fluorine, and/or bioreducible bonds, covalently linked to one or more linear or cyclic nitrogen-based chemical moieties;
   characterized in that:
the lipid does not include any positive charge at pH ranging from 7.0 to 7.4 whereas it displays one or several positive charges when the pH is below 7.0, preferably ranges from 2.0 and 7.0, more preferably ranges from 4.0 and 7.0 and even more preferably ranges from 4.5 and 7.0;
R corresponds to formula (II):

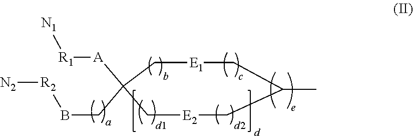

where:
N₁ and N₂, identical or different represent a linear, branched saturated or unsaturated, hydrocarbon group comprising from 1 to 8 carbon atoms, preferably from 1 to 4, used to terminate carbon chains, preferably N₁ and N₂, identical or different, are either a methyl, ethyl, n-propyl, i-propyl, n-butyl, s-butyl, i-butyl or t-butyl group;
R₁ and R₂, identical or different, represent a linear, branched and/or cyclic, saturated or unsaturated, hydrocarbon group comprising from 6 to 24 carbon atoms, preferably from 10 to 18 and optionally comprising one or more heteroatoms;
A and B, identical, represent a O—C(O), C(O) NH—, —NHCO—, O—C(O)—O, NH—C(O)—NH, NH—C(O)—O, O—C(O)—NH, —S—C(O)—S—, —O—C(S)—S—, —S—C(O)—O, —NH— or —S— group;
a is an integer comprised between 1 and 4, preferably a is an integer equal to 1, 2 or 3;
b is an integer comprised between 0 and 6, preferably b is an integer equal to 0 or 1;
E₁ and E₂, identical or different, represent a —C(O)O—, —C(O)—NH—, —NH—, —O— or —S— group;
c is an integer comprised between 0 and 2;
d is an integer equal to 0 or 1;
d₁ is an integer comprised between 0 and 6, preferably d₁ is an integer equal to 0 or 1;
d₂ is an integer comprised between 0 and 6, preferably d₂ is an integer comprised between 0 and 2; and
e is an integer comprised between 0 and 6, preferably between 0 and 2, and more preferably between 0 and 1;
Sp corresponds to the general formula (III):

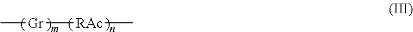

where:
Gr represents a linear or branched hydrocarbon group, comprising from 1 to 15 carbon atoms, preferably from 1 to 8, and optionally comprising one or more heteroatoms;
m is an integer equal to 0 or 1;
RAc represents an amino acid radical; and
n is an integer equal to 0 or 1; and
Zs corresponds to formula (IV):

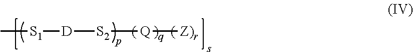

where:
S₁ and S₂, identical or different, represent a linear or branched hydrocarbon group comprising from 1 to 15 carbon atoms, preferably from 1 to 6 carbon atoms, optionally comprising one or more heteroatoms preferably chosen from nitrogen, oxygen, sulfur, bromine, iodine, chlorine and fluorine, more preferably from nitrogen, sulphur, bromine, iodine, chlorine and fluorine;
D represents one or more vinyl ether groups, one or more acylhydrazone groups, one or more carbonate bond, a disulfide bond, an ester bond or carbonate bond or one or more photosensitive groups;
p is an integer equal to 0 or 1;
Q is a branched hydrocarbon group comprising from 1 to 20 carbon atoms, preferably from 1 to 12 carbon atoms, and one or more heteroatoms, and which can optionally be covalently coupled with the S₂ or RAc or Gr or R group on the one hand, and to at least two Q and/or Z groups on the other hand;
q is an integer comprised between 0 and 8, preferably between 0 and 3;
Z is a nitrogen (N) centred terminal ionizable moiety, preferably Z represents a tertiary aliphatic amine such as N,N-dialkylamine, or a cyclic amine moiety such as N-substituted Pyrrolidine, N-substituted piperidine, N-substituted morpholine, N-substituted piperazine or N-substituted pyridine; where, Z does not include any positive charge at pH ranging from 7.0 to 7.4, whereas it displays one or several positive charges when the pH ranges from 2.0 and 7.0, preferably from 4.0 and 7.0 and more preferably from 4.5 and 7.0.
r is an integer comprised between 1 and 16, preferably between 1 and 8, it being preferably understood that if q is equal to 1 then r is at least equal to 2 and that if r is greater than 1, then the Z groups can be identical or different; and
s is an integer equal to 1 or 2, the following compounds being excluded from general formula (I):

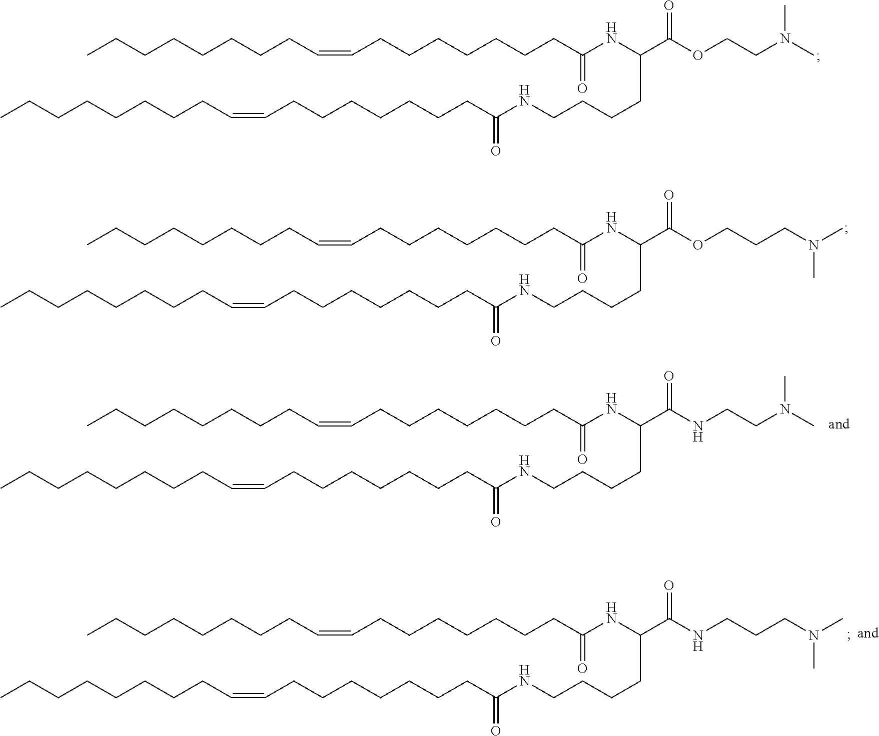

in formula (III):
Gr is absent or Gr serves as a spacer arm and corresponds to the molecular pattern noted as —W₄—Y₄—W₄—,
   where Y₄ represents a bridging alkylene group comprising from 1 to 8 carbon atoms, preferentially from 1 to 4, whilst W₄ represents a C(O)—O, C(O)—NH, C(O)—S, S(O), S(O)—O, C(S)—O, —O— or —NH— group.
group; or
Gr corresponds to the formula —W₄—Y₄—W₄—,
   where Y₄ has the same meaning as previously defined and is connected by a carbonate or carbamate bond to the lipophilic region R on the one hand, and either to the RAc radical, or directly to the Si or Q or Z group on the other hand; and
   in formula (III) the amino acid radical RAc is chosen from aspartic acid, glutamic acid, isoleucine, leucine, lysine, ornithine, glycine and phenylalanine, and more preferably from aspartic acid, glutamic acid, glycine, ornithine and lysine.

The present invention also relates to a composition comprising the lipid of formula (I) as previously defined.

The present invention also relates to a process for manufacturing the lipid of formula (I) as previously defined and/or the composition as previously defined.

The present invention also relates to a lipid of formula (I) as previously defined and/or composition as previously defined for its use as a medicament.

The present invention also relates to the use of the lipid of formula (I) as previously defined and/or of the composition as previously defined as a vector for delivering an active ingredient to subject(s), organ(s), cell(s) and/or tissue(s).

The present invention also relates a method for transfecting cells with a nucleic acid and/or a protein and/or a peptide and/or a polysaccharide and/or a lipid and/or a small organic or inorganic molecule and/or any type of bioactive molecule using the lipid of formula (I) as previously defined and/or the composition as previously defined.

The present invention also relates to transfected cells obtained by the method as previously defined.

The present invention also relates a kit comprising the lipid of formula (I) as previously defined and/or the composition as previously defined further comprising a nucleic acid and/or a protein and/or a peptide and/or a polysaccharide and/or a lipid and/or a small organic or inorganic molecule and/or any type of bioactive molecule, and optionally instructions of use.

### DEFINITIONS

By “lipid” it is preferably understood an organic amphiphilic molecule that includes in its structure an apolar hydrophobic carbonated region and a polar hydrophilic region, linked together by a chemical spacer. By “tunable lipid” it is preferably understood a lipid as previously defined having an hydrophilic area that may include one or several positive charges depending on the pH of its direct environment, enabling the possibility for the user to tune its chemical properties by choosing the right pH. Preferably, it does not present any permanent positive charges at physiological pH (7.0-7.4) while it will become cationic as the pH of its physiological environment decreases. Preferably, its amphiphilic nature makes it generally insoluble in water and soluble in some extent in organic solvent. Preferably, the term “tunable lipid” also implies that such lipid, when processed properly may form self-assembled structures in aqueous environment.

By “biodegradable lipid” it is preferably understood an organic amphiphilic molecule that includes in its structure an apolar hydrophobic carbonated region and a polar hydrophilic region, linked together by a chemical spacer, and that organic amphiphilic molecule chemical and physical characteristics may undergo deterioration and may completely degrade under biological conditions when exposed to microorganisms, aerobic, and anaerobic processes. In addition, the compounds or chemical entities resulting of the degradation of this molecule are biocompatible.

By “biodegradable bond” or “biodegradable group of atoms”, it is preferably understood that the designed chemical bond or group of atoms may be degradated, i.e. broken, in biological environment, under biological conditions when exposed to microorganisms, aerobic, and anaerobic processes. In addition, the compounds or chemical entities resulting of the degradation of this bond or groups of atoms are preferably biocompatible.

By “biocompatible” it is preferably understood, that the molecule, or group of atoms, or chemical entity could be in contact with a living system without provoking any adverse effect.

By “hydrolysable bond” or “hydrolysable group of atoms”, it is preferably understood that the designed chemical bond or group of atoms may be degradated, i.e. broken, in biological environment, by the action of water molecules. This action might be triggered or accelerated by specific pH conditions.

By “bioreducible bond” or “bioreducible group of atoms”, it is preferably understood that the designed chemical bond or group of atoms may be reduced, i.e. broken, in biological environment, following an oxydo-reduction pathway.

Examples of bioreducible bonds or bioreducible group of atoms include, but are not limited to, disulphide bond, ester bond, vinyl ether bond and/or acyl-hydrazone bond.

By “photosensitive bond” or “photosensitive group of atoms”, it is preferably understood that the designed chemical bond or group of atoms may be reduced, i.e. broken, by the application of light of define wavelength.

Examples of photosensitive bonds or photosensitive group of atoms include, but are not limited to, diazo, nitrobenzyl and/or phenacyl groups.

In the present invention, by “lipophilic group of atom” it is preferably understood that the group of atom contains apolar carbonated region reducing the potency of this group of atoms to get dissolved in an aqueous environment. It is widely understood that lipophilic could be substituted by the term hydrophobic without changing its meaning. Thus, in the present invention the terms “lipophilic” and “hydrophobic” may be used interchangeably.

In the present invention, by “hydrophilic group of atom”, it is preferably understood that the group of atom contains polar regions, usually centered on heteroatoms such as oxygen, nitrogen, sulfur and/or phosphorous, increasing the potency of this group of atoms to get dissolved in water. It is widely understood that hydrophilic could be substituted by the term lipophobic without changing its meaning. Thus, in the present invention the terms “hydrophilic” and “lipophobic” may be used interchangeably.

By “terminal ionizable moiety”, it is preferably understood that Z is a chemical group that has the potency to attract or liberate a proton by change of the pH of the medium. In the present invention, by ionizable it is preferably understood that the global pKa of Z confers to this group the possibility to attract one or several protons available in the medium to gain one or several positive charges when the pH is decreasing. As a consequence, it is preferably considered that Z can attract one or several protons when the pH of the medium goes below the value of the physiological pH, i.e. when pH<7.4. Preferably, it is considered that Z can attract one or several protons available in the medium to gain one or several positive charges when the pH is comprised between 2 and 7.4, preferably between 4 and 7.4 and more preferably between 4.5 and 7.

Examples of terminal ionizable groups include, but are not limited to, N,N-dialkyl aliphatic tertiary amines, incorporating or not heteroatoms such as oxygen, sulphur, nitrogen and/or phosphorous, halogens, or combination thereof.

Preferably, in the present invention, the terminal ionizable moiety Z does not bear any charges at physiological pH due to its pKa value. Preferably, the terminal ionizable moiety Z does not bear any charges at physiological pH, while Z can attract one or several protons when the pH of the medium goes below the value of the physiological pH, i.e. when pH<7.4, to gain one or several positive charges. Preferably, the terminal ionizable moiety Z does not bear any charges at physiological pH, while Z can attract one or several protons available in the medium to gain one or several positive charges when the pH is comprised between 4 and 7.4, preferably between 4.5 and 7.

By terminal position of ionisable moiety, it is preferably understood that pKa of the terminal ionisable moiety is directly responsible of the global pKa of the molecules defined by the formula (I). As a consequence, pKa of the terminal ionisable moiety is directly responsible of the global charges of the molecules defined by the formula (I). Thus, as the terminal ionizable moiety Z preferably does not bear any charges at physiological pH due to its pKa value, the molecules defined by the formula (I) preferably do not bear any charges at physiological pH.

Preferably, as Z can attract one or several protons available in the medium to gain one or several positive charges when the pH is comprised between 4 and 7.4, the molecules defined by the formula (I) can gain one or several positive charges when the pH is comprised between 4 and 7.4. More preferably, as Z can attract one or several protons available in the medium to gain one or several positive charges when the pH is comprised between 4.5 and 7, the molecules defined by the formula (I) can gain one or several positive charges when the pH is comprised between 4.5 and 7.

By “pH of the medium” it is preferably understood the pH of the medium comprising the compound of the present invention.

By “physiological pH” it is preferably understood a pH ranging from 7.0 to 7.4.

In the present invention, unless otherwise indicated, by “heteroatom” it is preferably understood an atom chosen from nitrogen, oxygen, sulfur, phosphorus and halogens.

In the present invention, unless otherwise indicated, by “halogen” it is preferably understood bromine, iodine, chlorine and fluorine.

By “fluorinated” it is preferably understood comprising one or more fluorine atoms.

By “comprising one or more heteroatoms other than fluorine” it is preferably understood comprising one or more atoms chosen from nitrogen, oxygen, sulfur, phosphorus and halogens other than fluorine.

By “hydrocarbon group” is preferably understood any group comprising one or more carbon atoms, optionally bonded to one or more hydrogen atoms.

By “one or more” it is preferably understood one, two, three, four, five, six, seven, eight, nine or ten, more preferably one, two, three, four or five and even more preferably one, two or three.

By “transfection” it is preferably understood the process to introduce native, wild type or recombinant genetic material or proteins into the target cell via synthetic or non-viral delivery systems. The nature of the genetic material introduced, and the mechanisms involved in this step are dependent on the type of nucleic acids, proteins or small molecules used. For example, the genetic materials introduced can encode a large variety of gene of interest such as reporter genes, enzymes, endonucleases, nucleases or recombinase for genome editing, short hairpin RNA, antisense RNA for gene silencing, mRNA, tRNA, receptors such as engineering antigen receptor (including, but not limited to, T cell receptor, chimeric antigen receptor and chimeric cytokine receptor), growth factors, hormone, cell surface proteins, secreted proteins, signalling proteins or any polypeptides, and/or proteins or nucleic acids with therapeutic interest.

By “biologically active molecule”, “active molecule”, “biologically active agent” and/or “active ingredient”, it is preferably understood any molecule or macromolecule having a specific activity in cells and used in numerous fields ranging from cell biology to medicine. Examples of such molecule or macromolecule include, but are not limited to, nucleic acid(s), polysaccharide(s), lipid(s), polynucleotide(s), morpholino oligonucleotide(s), aptamer(s), protein(s), peptide(s), polypeptide(s), peptoid(s), small organic or inorganic molecule(s), drug(s) or any compounds of pharmaceutical interest, antigen(s), and mixtures thereof.

By “small organic or inorganic molecule(s)” it is preferably understood a molecule of known structure and molecular weight, that is not of polymeric nature, and that has preferentially a molecular weight below 1500 g/mol.

Drug(s) or any compounds of pharmaceutical interest may be any molecule or compound, including a salt or derivative thereof, capable of exerting a desired effect such as a biological, physiological, and/or cosmetic effect, on a cell, tissue, tumor, organ, or subject. Examples of drug(s) or any compounds of pharmaceutical interest include, but are not limited to, oncology drugs (including chemotherapy drugs, hormonal therapeutic agents, immunotherapeutic agents, and/or radiotherapeutic agents), lipid-lowering agents, anti-viral drugs, anti-inflammatory compounds, antidepressants, stimulants, analgesics, antibiotics, birth control medication, antipyretics, vasodilators, anti-angiogenics, cytovascular agents, signal transduction inhibitors, cardiovascular drugs such as anti-arrhythmic agents, hormones, vasoconstrictors, and/or steroids. More particularly, examples of drug(s) or any compounds of pharmaceutical interest include, but are not limited to, folate or sodium valproate, antibodies, antigens, lymphokines, interleukins, necrosis and apoptosis factors, interferons, growth factors, tissue plasminogen activators, factor VIIIc, erythropoietin, insulin, calcitocin, thymidine kinase, and combination thereof.

Drug(s) or any compounds of pharmaceutical interest may be therapeutically active themselves or may be prodrugs, which become active upon further modification.

Examples of nucleic acids, include but are not limited to, deoxyribonucleic acid (DNA) such as a gene or plasmid DNA (pDNA), linear coding DNA, artificial chromosomes, antisense oligonucleotides (ASOs) or a ribonucleotide acid (RNA) such as messenger RNAs (mRNAs), small interfering RNAs (siRNA), self-amplifying RNAs (saRNAs), circular RNA (cirRNA), microRNAs (miRNAs), single-guide RNAs (sgRNAs)-mediated CRISPR-Cas9 system, double-stranded RNA (dsRNA), short hairpin RNA (shRNAs) ribozymes, or a modified nucleic acid such as a peptide nucleic acid (PNA), locked nucleic acid (LNA), morpholino oligonucleotides and/or aptamers. Nucleic acids may be natural or artificial. Nucleic acids may be of animal, human, plant, bacterial or viral origin. Nucleic acids can be used to express, alter, downregulate or silence the translation (that is the expression) of a gene of interest. Their function as a therapeutic agent can be a gene or mRNA coding for a polypeptide and/or a protein of interest in a host cell or can be an antisense function controlling the expression of a gene, its transcription to RNA or its translation to protein. They can also act as a ribozyme or interfering RNA (such as sRNA, shRNA or miRNA) with the expression of a gene. They can also edit a gene by correcting or adding a mutation (such as CRISPR Cas9).

Non-limiting examples of genes of interest include, but are not limited to, genes associated with metabolic diseases and disorders (such as liver diseases and disorders), genes associated with cell proliferation, tumorigenesis, and/or cell transformation (including a cell proliferative disorder such as cancer), angiogenic genes, receptor ligand genes, immunomodulator genes (such as those associated with inflammatory and autoimmune responses), genes associated with viral infection and survival, and/or genes associated with neurodegenerative disorders.

The nucleic acid that is present in a lipid-nucleic acid particle can be in any form. The nucleic acid can, for example, be single-stranded DNA or RNA, or double-stranded DNA or RNA, or DNA-RNA hybrids. Non-limiting examples of double-stranded RNA include siRNA and miRNA.

Non-limiting examples of single-stranded RNA include mRNA. Non-limiting examples of double-stranded DNA include plasmid and linearized DNA. The nucleic acid may optionally comprise one or more modified nucleotides including, but not limited to, 2′—O-methyl nucleotides, 2′-deoxy nucleotides, 2′-O-(2-methoxyethyl) nucleotides, locked nucleic acid (LNA) nucleotides, 5-C-methyl nucleotides, 4′-thio nucleotides, -amino nucleotides, 2′-C-allyl nucleotides, and mixtures thereof.

For use in the fields of gene therapy, vaccines and immunotherapy, the nucleic acid may advantageously be DNA and RNA and preferably comprises an expression cassette constituted by one or more sequences of DNA or RNA encoding the polypeptide of interest under the control of one or more promoters and a transcriptional terminator, which are active in the target cells.

By “polypeptide” it is preferably understood any amino acid chain irrespective of its size. Thus, this term may cover peptides and proteins.

By “target cell” it is preferably understood any cell that can be targeted for transfection.

By “cell” is preferably understood a single and/or isolated cell, or a cell that is part of a multicellular entity such as a tissue, an organism or a cell culture. The cell can be a primary cell or established cell lines or a stem cell or a progenitor cell.

The term “primary cell” as used herein is well known in the art and may refer to a cell that has been isolated from a tissue and has been established for growth in vitro.

The cell can be an eukaryotic cell such as for example, but not limited to, endothelial, epithelial, fibroblastic, hepatic, hematopoietic (including but not limited to lymphocytes and/or monocytes macrophages natural killer dendritic cells), muscle, nerve cells (an in particular all types of neurons including but not limited to cortical, hippocampal, sensory neurons, motor neurons, dorsal route ganglion, oligodendrocytes, cerebellar granule, neural stem cells and basket cells, Betz cells, medium spiny neurons, Purkinje cells, pyramidal cells, Renshaw cells, granule cells or anterior horn cells and glial cells astrocytes), stem cells, embryonic stem cells, hematopoietic stem cells, germ cells, a tumor cells, a lymphoid cell lineage and/or somatic cells.

An eukaryotic cell as used herein, can refer to any cell of a multi-cellular eukaryotic organism, including cells from animals like vertebrates. Preferably, said target cell can be a mammalian cell. The term “mammalian cell” as used herein, is well known in the art and may refer to any cell belonging to or derived from an animal that is grouped into the class of Mammalia.

The cells can be prokaryotic (such as bacteria), plant cells, insect, yeast, or parasite cells.

The cells can be differentiated or totipotent or omnipotent or multipotent or oligopotent or unipotent or pluripotent. The cells can be embryonic stem cells or induced pluripotent stem cells (iPS) or differentiated from embryonic or iPS. The cells to be transfected can be non-permissive cells, hard-to-infect cells, or easy to infect cells. Tumor cells and cell lines as known in the art can be for example, but not limited to, lymphoma cell lines (such as for example Jurkat, CEM, H9, Daudi, SUP-M2 and KARPAS-299), dendritic cell lines (such as DC2.4 and Mutu), natural killer cell lines (such as NK-92 and KHYG-1), epithelial cell lines (such as NIH-3T3, COS, CHO and HEK293), pancreatic tumor cells (such as PANC-1), a stem cell line (KG1a), breast cancer cells (such as MCF-7, T74D and MDA-MB361), a neuroblastoma (such as SH-SY5Y and N2a), a fibrosarcoma (such as HT1080), an astrocytoma (such as 1321N1), and/or endothelial cells (such as HUVEC, HMEC and HCAEC).

By “signalling molecules and molecules that may be involved in the signalling pathways inside of the target cells” it is preferably understood activators or inhibitors of the protein kinase C or phospholipase signalling pathway, such as activators or inhibitors of the cellular metabolism pathways (such as for example AMPK signalling, insulin receptor signalling and glutamine metabolism pathway), activators or inhibitors of the TLR-pathway (such as Toll-Like Receptor), of the PRRs-pathway (Pattern Recognition Receptors), of the PAMPs-pathway (Pathogen-Associated Molecular Patterns), of the DAMPs-pathway (Damage-Associated Molecular Patterns), AhR ligands (aryl hydrocarbon receptor ligands), the NOD-Like receptors (NLRs), the RIG-I-Like receptors (RLRs), cytosolic DNA sensors (CDS), the C-type lectin receptors (CLRs), activators or inhibitors of inflammasomes or inflammation or autophagy pathway, activators or inhibitors of the JAK/STAT pathway, activators or inhibitors of the ubiquitin and ubiquitin-like/proteasome pathway, activators or inhibitors of the PI3K/AKT pathway, activators or inhibitors of the tyrosine kinase, activators or inhibitors of the MAP kinase pathways, activators or inhibitors of the GPCR, activators or inhibitors of the STING pathways, calcium, cAMP signalling pathway, activators or inhibitors of the cell cycle, checkpoint control and DNA repair mechanisms, activators or inhibitors of the apoptosis pathway, regulators of the reactive oxygen species (such as ROS and NOS), activators or inhibitors of immune checkpoints, regulators of protein synthesis and RNA degradation process, regulators of keys elements having a role at the chromatin or DNA level (such as activators or inhibitors of protein acetylation, histone lysine methylation, DNA methylation and nuclear receptor signalling), regulators of microtubule and actin dynamics pathways.

By “lipid of formula (I)” it is preferably understood the lipid of the present invention.

Preferably, the terms “conjugated” and “coupled” used herein have the same meaning.

### DETAILED DESCRIPTION

For the purpose of this application, unless otherwise stated, the ranges of values indicated are inclusive.

A first object of the present invention relates to a lipid, preferably a tunable biodegradable lipid of formula (I), or a stereoisomer thereof, or a pharmaceutically acceptable salt thereof:

R-Sp-Zₛ (I)

### Wherein:

R represents a lipophilic region, preferably R comprises one or more branched or linear, unsaturated or saturated, optionally fluorinated, alkyl chains, comprising 6 to 48 carbon atoms or between 6 to 24 carbon atoms, preferably between 10 and 36 carbon atoms, more preferably between 10 to 18 carbon atoms, optionally comprising one or more heteroatoms other than fluorine; or one or more cyclic or polycyclic groups known to be lipophilic, optionally comprising one or more heteroatoms, such as a steroid group optionally comprising one or more heteroatoms, a polyaromatic group optionally comprising one or more heteroatoms, or an alkaloid derivative group optionally comprising one or more heteroatoms; or a natural or synthetic lipid optionally comprising one or more heteroatoms; or a combination thereof.
Sp represents a spacer region used to separate the lipophilic region from the hydrophilic polar region, preferably hydrophilic tunable polar region, more preferably Sp comprises one or more branched or linear, unsaturated or saturated, optionally fluorinated alkyl chains, comprising 6 to 24 carbon atoms, preferably between 10 and 18 carbon atoms, optionally comprising one or more heteroatoms and/or bioreducible bonds; and
Zₛ represents a hydrophilic polar region, preferably a hydrophilic tunable polar region, used to complex and efficiently deliver biologically active component into cells. More preferably, Zₛ comprises one or more branched or linear, unsaturated or saturated, optionally fluorinated alkyl chains, comprising 2 to 36 carbon atoms, preferably comprising 2 to 24 carbon atoms, optionally comprising one or more heteroatoms other than fluorine, and/or bioreducible bonds, covalently linked to one or more linear or cyclic nitrogen-based chemical moieties, that preferably does not bear charges at physiological pH but become positively charged if the pH of the medium dropped below the pKa value of the entity. This polar region might incorporate bioreducible molecular patterns enabling a faster release of the biologically active compound and enhanced biocompatibility of the whole molecule.

Preferably, due to the definition of Zₛ, the lipids of the present invention, preferably the tunable lipids of the present invention, do not include any positive charge at physiological pH (7.0-7.4), which implies that the pKa of the molecules covered by the formula (I) is always lower than 7.0.

Preferably, due to the definition of Zₛ, the lipids of the present invention, preferably the tunable lipids of the present invention, do not include any positive charge at physiological pH (7.0-7.4), whereas it displays one or several positive charges when the pH of the medium falls below the pKa of the molecules covered by the formula (I), which is always lower than 7.0. Preferably, due to the definition of Zₛ, the lipids of the present invention, preferably the tunable lipids of the present invention, do not include any positive charge at physiological pH (7.0-7.4), whereas it displays one or several positive charges when the pH of the medium ranges from 2.0 and 7.0, preferably from 4.0 and 7.0 and more preferably between 4.5 and 7.0.

In other words, preferably, Zₛ cannot include primary aliphatic amine groups, succinate groups, and/or carboxylic groups in combination with tertiary amine groups (but Zₛ could include carboxylic groups and no tertiary amine groups and Zₛ could include tertiary amine groups and no carboxylic groups).

Examples of nitrogen-based chemical moieties include, but are not limited to, amines, amides, oximes, hydroxylamines, carbamates, isocyanates, isothiocyanates or cyanhydrins, either aliphatic or aromatic, or combination thereof.

Examples of cyclic or polycyclic groups known to be lipophilic, optionally comprising one or more heteroatoms, include, but are not limited to, cycloalkane, cycloalkenes, cycloalkynes and/or steroid groups.

Examples of steroid group include, but are not limited to, cholesterol and its derivatives, ergosterol, estradiol, stigmasterol, testosterone, progesterone and/or androsterone.

Examples of cycloalkane, include, but are not limited to, cyclopentane, cyclohexane, cyclooctane, cyclodecane, bicyclohexane and/or tricyclohexane.

Examples of cycloalkene, include, but are not limited to, cyclopropene, cyclobutene, cyclopentene, cyclohexene, cycloheptene, 1,3-cyclohexadiene, 1,4-cyclohexadiene, 1,5-Cyclooctadiene, cis-cyclooctene and/or trans-cyclooctene.

Examples of cycloalkynes, include, but are not limited to, cyclododecyne, cyclodecyne, cyclononyne and/or cyclooctyne.

By “cholesterol derivative”, it is preferably understood, cholesterol, bile acids, Vitamin D₂, Progestins, glucocorticoids, estrogens, androgens, phytosterols and/or oxysterols.

Examples of polyaromatic group include, but are not limited to, naphthalene derivatives, dansyl derivatives and/or anthracene derivatives.

By “naphtalene derivative” it is preferably understood 1-Naphtoic acid, 1-Naphtol, 1-Nitronaphtalen and/or 1-halogenonaphtalene.

By “dansyl derivatives” it is preferably understood 5-(dimethylamino)naphthalene-1-sulfone.

By “anthracene derivative” it is preferably understood anthrancene, mono-hydroxyanthracene, and/or poly-hydroxy-anthracene.

Examples of alkaloid derivative include, but are not limited to, hygrine, retronecine, indicine, N-acetylloline, atropine, morphine, mescaline, adrenaline, putrescine, spermine, theobromine, cathinone, sedamine, connine, matrine, swainsonine, pipecolic acid, iophorecine, amurensine, berberine, glaucine, imerubine, muscimol, serotonine, quinine, ergotamine, colchicine, solanidine, cyclopamine, batrachotoxin and/or isoquinoline.

Examples of natural or synthetic lipid include, but are not limited to, phosphatidylcholine or its derivatives, phosphatidylethanolamine or its derivatives, phosphatidylinositol or its derivatives, sphingomyelin or its derivatives, phosphatic acid or its derivatives, lyso-phosphatidylcholine or its derivatives, lyso-phosphatidylethanolamine or its derivatives, glycero-3-phosphocholine or its derivatives, and/or glycerol or its derivatives.

By “bioreducible bonds” it is preferably understood disulphide bond, ester bond, vinyl ether bond and/or acyl-hydrazone bond.

By “bioreducible molecular patterns” it is preferably understood a biocompatible or bioinspired groups of atoms that can undergo degradation or deterioration under biological conditions when exposed to microorganisms, aerobic, and anaerobic processes, increasing as a consequence biocompatibility of the whole molecule. Examples of bioreducible molecular patterns include, but are not limited to, bifunctional ribose groups, ethylene oxide groups and/or amino acids groups.

Preferably, the following compounds are excluded from general formula (I):

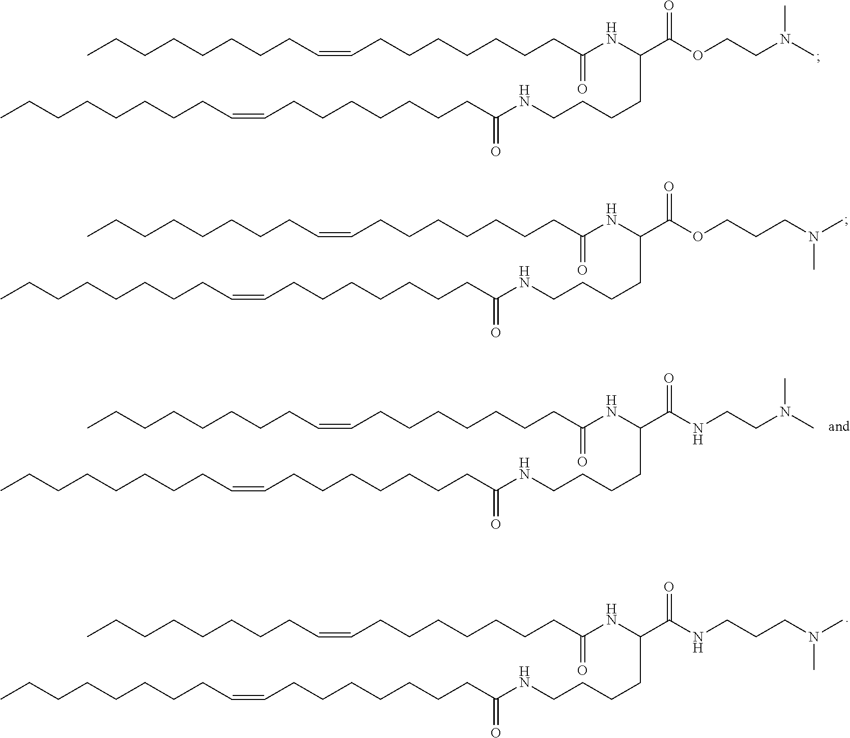

Preferably, in formula (I) as previously defined R corresponds to formula (II):

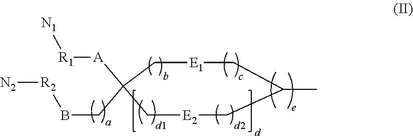

wherein:
N₁ and N₂, identical or different represent a linear, branched saturated or unsaturated, hydro-carbon group comprising from 1 to 8 carbon atoms, preferably from 1 to 4, used to terminate carbon chains, preferably N₁ and N₂, identical or different, are either a methyl, ethyl, n-propyl, i-propyl, n-butyl, s-butyl, i-butyl or t-butyl group;
R₁ and R₂, identical or different, represent a linear, branched and/or cyclic, saturated or unsaturated, hydrocarbon group comprising from 6 to 24 carbon atoms, preferably from 10 to 18 and optionally comprising one or more heteroatoms;
A and B, identical, represent a O—C(O), C(O) NH—, —NHCO—, O—C(O)—O, NH—C(O)—NH, NH—C(O)—O, O—C(O)—NH, —S—C(O)—S—, —O—C(S)—S—, —S—C(O)—O, —NH— or —S— group, or A and B, identical or different, represent a O—C(O), C(O) NH—, —NHCO—, —NH— or —O— group, preferably A and B, identical represent a O—C(O), C(O) NH—, —NHCO—, O—C(O)—O, NH—C(O)—NH, NH—C(O)—O, O—C(O)—NH, —S—C(O)—S—, —O—C(S)—S—, —S—C(O)—O, —NH— or —S— group;
a is an integer comprised between 1 and 4, preferably a is an integer equal to 1, 2 or 3;
b is an integer comprised between 0 and 6, preferably b is an integer equal to 0 or 1;
E₁ and E₂, identical or different, represent a —C(O)O—, —C(O)—NH—, —NH—, —O— or —S— group;
c is an integer comprised between 0 and 2;
d is an integer equal to 0 or 1;
d₁ is an integer comprised between 0 and 6, preferably d₁ is an integer equal to 0 or 1;
d₂ is an integer comprised between 0 and 6, preferably d₂ is an integer comprised between 0 and 2;
e is an integer comprised between 0 and 6, preferably between 0 and 2, and more preferably between 0 and 1.

Preferably, in formula (II) as previously defined, when a is an integer equal to 4, the following compounds are excluded:

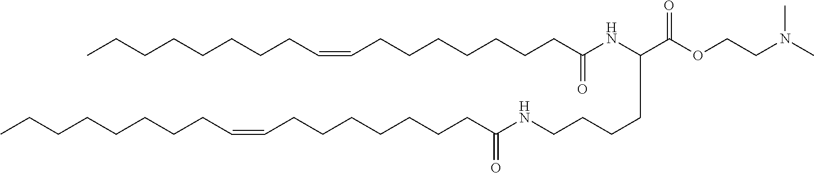 [SMILES: CCCCCCCC/C=C\CCCCCCCC(=O)NCCCCC(NC(=O)CCCCCCC/C=C\CCCCCCCC)C(=O)OCCN(C)C]

wherein according to formula I Sp is absent and Zs is CH₂—CH₂—N(CH₃)₂ and wherein according to formula II N₁=N₂=CH₃, R₁=R₂=C₁₇H₃₀, A and B identical representing —C(O)NH—, a=4, E₁ representing a —C(O)O— group E₂ being absent and b=c=e=d1=d2=0;

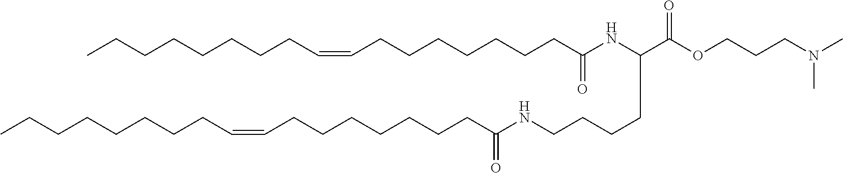 [SMILES: CCCCCCCC/C=C\CCCCCCCC(=O)NCCCCC(NC(=O)CCCCCCC/C=C\CCCCCCCC)C(=O)OCCCN(C)C]

wherein according to formula I Sp is absent and Zs is CH₂—CH₂—CH₂—N(CH₃)₂ and wherein according to formula II N₁=N₂=CH₃, R₁=R₂=C₁₇H₃₀, A and B identical representing —C(O)NH-a=4, E₁ representing a —C(O)O— group E₂ being absent and b=c=e=d1=d2=0;

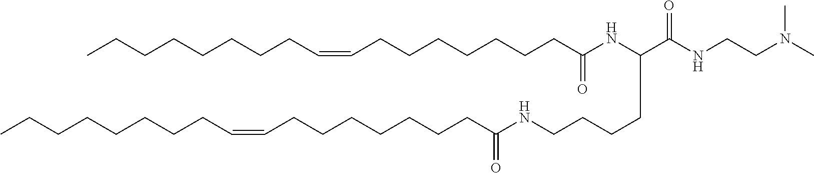 [SMILES: CCCCCCCC/C=C\CCCCCCCC(=O)NCCCCC(NC(=O)CCCCCCC/C=C\CCCCCCCC)C(=O)NCCN(C)C]

wherein according to formula I Sp is absent and Zs is CH₂—CH₂—N(CH₃)₂ and wherein according to formula II N₁=N₂=CH₃, R₁=R₂=C₁₇H₃₀, A and B identical representing —C(O)NH—, a=4, E₁ representing a —C(O)NH— group E₂ being absent and b=c=e=d1=d2=0;

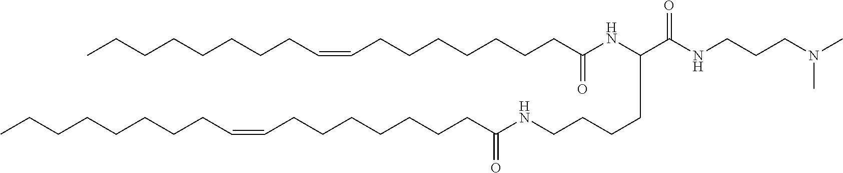 [SMILES: CCCCCCCC/C=C\CCCCCCCC(=O)NCCCCC(NC(=O)CCCCCCC/C=C\CCCCCCCC)C(=O)NCCCN(C)C]

wherein according to formula I Sp is absent and Zs is CH₂—CH₂—CH₂—N(CH₃)₂ and wherein according to formula II N₁=N₂=CH₃, R₁=R₂=C₁₇H₃₀, A and B identical representing —C(O)NH—, a=4, E₁ representing a —C(O)NH— group E₂ being absent and b=c=e=d1=d2=0.

Preferably, in the above formula (II), it is likely understood that if either or both R₁ and R₂ are branched, each alkyl chain of the branched molecular pattern would be terminated by a N₁ and/or N₂ moiety as previously defined.

Preferably, in the above formula (II), it is likely understood that if d equals 0, d₁ and d₂ would automatically equals 0 and E₂ would be absent.

Preferably, in formula (I) as previously defined Sp corresponds to the general formula (III):

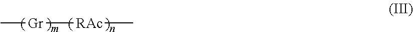

wherein:
Gr represents a linear or branched hydrocarbon group, comprising from 1 to 15 carbon atoms, preferably from 1 to 8, and optionally comprising one or more heteroatoms;
m is an integer equal to 0 or 1;
RAc represents an amino acid radical; and
n is an integer equal to 0 or 1.

By “amino acid radical” it is preferably understood, the group of atoms that consists of this amino acid when the latter is covalently bonded, on the one hand to Gr or R group, and on the other hand to one or more Zₛ groups.

Thus, in formula (III) as previously defined Gr and RAc can be absent when m and/or n is equal to 0.

Preferably, in formula (I) as previously defined Zₛ corresponds to formula (IV):

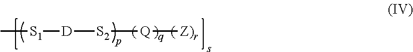

wherein:
S₁ and S₂, identical or different, represent a linear or branched hydrocarbon group comprising from 1 to 15 carbon atoms, preferably from 1 to 6 carbon atoms, optionally comprising one or more heteroatoms preferably chosen from nitrogen, oxygen, sulfur, bromine, iodine, chlorine and fluorine, more preferably from nitrogen, sulphur, bromine, iodine, chlorine and fluorine;
D represents a functional group which can incorporate at least one bond which is sensitive to its environment, which is stable in extracellular medium and rapidly cleaved in the intracellular medium as it is sensitive to stimuli such as pH reduction (for example vinyl ether, or carbonate bond, or acylhydrazone groups sensitive to acid medium) or a change in the oxidation-reduction potential (for example a disulfide bond, cleaved in reducing medium), to enzymes (for example an ester bond, cleaved by endogenous esterases); or also to light radiation (bearing photosensitive groups for example); preferably D represents one or more vinyl ether groups, one or more acylhydrazone groups, one or more carbonate bond, a disulfide bond, an ester bond or one or more photosensitive groups, preferably one or more vinyl ether groups, one or more acylhydrazone groups, a disulfide bond, an ester bond or one or more photosensitive groups;
p is an integer equal to 0 or 1;
Q is a branched hydrocarbon group comprising from 1 to 20 carbon atoms, preferably from 1 to 12 carbon atoms, and one or more heteroatoms, and which can optionally be covalently coupled with the S₂ or RAc or Gr or R group on the one hand, and to at least two Q and/or Z groups on the other hand;
q is an integer comprised between 0 and 8, preferably between 0 and 3;
Z is a nitrogen (N) centred terminal ionizable moiety preferably incorporating chemical functions bearing no charges at physiological pH that can go positively charged if the pH of the medium dropped below the pKa value of the entity, more preferably incorporating chemical functions bearing no positive charges at physiological pH (7.0-7.4); more preferably Z does not include any positive charge at physiological pH (7.0-7.4) whereas it displays one or several positive charges when the pH of the medium ranges from 2.0 and 7.0, preferably from 4.0 and 7.0 and more preferably from 4.5 and 7.0.

More preferably Z represents a tertiary aliphatic amine such as N,N-dialkylamine, or a cyclic amine moiety such as N-substituted Pyrrolidine, N-substituted piperidine, N-substituted morpholine, N-substituted piperazine or N-substituted pyridine. Substituents include, but are not limited to, alkyl groups such as one or more methyl, ethyl, n-propyl, i-propyl, n-butyl, s-butyl, i-butyl or t-butyl group(s) and/or aromatic rings based on benzene or naphthalene groups; where said substituents may include one or more heteroatoms;
r is an integer comprised between 1 and 16, preferably between 1 and 8, it being preferably understood that if q is equal to 1 then r is at least equal to 2 and that if r is greater than 1, then the Z groups can be identical or different; and
s is an integer equal to 1 or 2.

Examples of photosensitive groups include, but are not limited to, diazo, nitrobenzyl and/or phenacyl groups.

By “amino acid radical” is preferably understood, the group of atoms that consists of this amino acid when the latter is covalently bonded, on the one hand to the Gr or R group, and on the other hand to one or more S₁ or Q or Z groups.

Preferably, in formula (IV) as previously defined Z can be described according to formula (V):

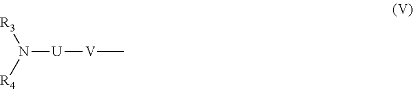

wherein: (V)
R₃ and R₄ are either the same or different and selected from C1-C₆ alkyl, C₂-C₆ alkenyl, or C₂-C₆ alkynyl, optionally substituted by one or more methyl, ethyl, n-propyl, i-propyl, n-butyl, s-butyl, i-butyl or t-butyl group(s), or R₃ and R₄ may join to form an optionally substituted by one or more methyl, ethyl, n-propyl, i-propyl, s-butyl, i-butyl or t-butyl group(s) heterocyclic ring such as pyrrol, pyrrolidine, pyridine, piperazine, morpholine, piperidine or indoline optionally substituted by one or more methyl, ethyl, n-propyl, i-propyl, s-butyl, i-butyl or t-butyl group(s).
N is a Nitrogen;
V is O, S, N(R₅), C(O), C(O)O, OC(O), C(O)N(R₅), N(R₅)C(O), OC(O)N(R₅), N(R₅)C(O)O, C(O)S, C(S)O, S(O), S(O)(O), C(S), or an optionally substituted heterocyclic ring such as a pyrrole, pyrrolidine, pyridine, piperazine, morpholine, piperidine or indoline, optionally substituted by one or more methyl, ethyl, n-propyl, i-propyl, n-butyl, s-butyl, i-butyl or t-butyl group(s), wherein R₅ is either an hydrogen (H) or a C₁-C₁₀ alkyl, C₂-C₁₀ alkenyl, or C₂-C₁₀ alkynyl, optionally substituted by one or more methyl, ethyl, n-propyl, i-propyl, n-butyl, s-butyl, i-butyl or t-butyl group(s); preferably R₅ is selected from the group consisting of hydrogen (H), a C₁ alkyl (methyl) group, and a C₂, C₃, C₄, C₅, C₆, C₇, C₈, C₉, and C₁₀ alkyl, alkenyl, and alkynyl group, incorporating or not one or more heteroatoms, such as —O, —NH, —S and mixtures of thereof;
U is either absent or is a C1-C₁₂ alkyl, C₂-C₁₂ alkenyl, or C₂-C₁₂ alkynyl, optionally substituted by one or more methyl, ethyl, n-propyl, i-propyl, n-butyl, s-butyl, i-butyl or t-butyl group(s) and optionally substituted by one or more heteroatoms or group of heteroatoms preferably chosen from nitrogen, oxygen, sulfur, bromine, iodine, chlorine, fluorine, phosphorous, or combination thereof.

Preferably, R₃ and R₄ are each independently a C1-C₂ alkyl, C1-C₃ alkyl, C1-C₄ alkyl, C1-C₅ alkyl, C₂-C₃ alkyl, C₂-C₄ alkyl, C₂-C₅ alkyl, C₂-C₆ alkyl, C₃-C₄ alkyl, C₃-C₅ alkyl, C₃-C₆ alkyl, C₄-C₅ alkyl, C₄-C₆ alkyl, C₅-C₆ alkyl, C₂-C₃ alkenyl, C₂-C₄ alkenyl, C₂-C₅ alkenyl, C₂-C₆ alkenyl, C₃-C₄ alkenyl, C₃-C₅ alkenyl, C₃-C₆ alkenyl, C₄-C₅ alkenyl, C₄-C₆ alkenyl, C₅-C₆ alkenyl, C₂-C₃ alkynyl, C₂-C₄ alkynyl, C₂-C₆ alkynyl, C₂-C₆ alkynyl, C₃-C₄ alkynyl, C₃-C₆ alkynyl, C₃-C₆ alkynyl, C₄-C₅ alkynyl, C₄-C₆ alkynyl, or C₅-C₆ alkynyl, optionally substituted by one or more methyl, ethyl, n-propyl, i-propyl, n-butyl, s-butyl, i-butyl or t-butyl group(s).

Preferably, R₃ and R₄ are both methyl groups, either ethyl groups, or a combination of one methyl group and one ethyl group.

Preferably, R₃ and R₄ are joined to form an optionally substituted heterocyclic ring comprising 1, 2, 3, 4, 5, 6, or more carbon atoms and 1, 2, 3, 4, or more heteroatoms such as nitrogen (N), oxygen (O), sulfur (S), and mixtures thereof. The optionally substituted heterocyclic ring may comprise:
from 2 to 10 carbon atoms and from 1 to 3 heteroatoms such as nitrogen (N), oxygen (O), and/or sulfur (S); or
an imidazole, triazole (such as 1,2,3-triazole, 1,2,4-triazole), pyrazole, thiazole, pyrrole, furan, oxazole, isoxazole, oxazoline, oxazolidine, oxadiazole, tetrazole, pyrrole, pyrrolidine, pyridine, piperazine, morpholine, piperidine or indoline, optionally substituted by alkyl groups such as one or more methyl, ethyl, n-propyl, i-propyl, n-butyl, s-butyl, i-butyl or t-butyl group(s), and/or by aromatic rings based on benzene or naphthalene groups, that may include one or more heteroatoms, or
5 carbon atoms and 1 nitrogen atom, wherein the heterocyclic ring can be substituted with a substituent such as a hydroxyl (—OH) group at the ortho, meta, and/or para positions; or
an imidazole group, optionally substituted by alkyl groups such as one or more methyl, ethyl, n-propyl, i-propyl, n-butyl, s-butyl, i-butyl or t-butyl group(s), and/or by aromatic rings based on benzene or naphthalene groups, that may include one or more heteroatoms.

In the above mentioned formula (II), when R₁ and R₂ are different, their structure can be described according to formulae (VI a) and (VI b):

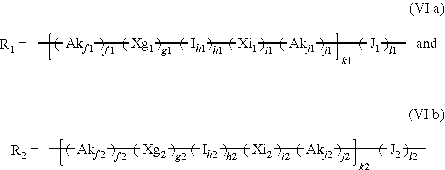

wherein:
Ak_{f1}, Akⱼ₁, Ak_{f2}, Akⱼ₂ identical or different, represent a linear or branched hydrocarbon group comprising from 1 to 22 carbon atoms, preferably from 1 to 12 carbon atoms, optionally comprising one or more heteroatoms;
f1 and f2 are integers equal to 0 or 1;
j1 and j2 are integers equal to 0 or 1;
Xg₁, Xi₁, Xg₂ and Xi₂, identical or different can be O, S, N(R,), C(O), C(O)O, OC(O), C(O)N(R,), N(R₇)C(O), OC(O)N(R,), N(R₇)C(O)O, C(O)S, C(S)O, S(O), S(O)(O), C(S), wherein R₇ is either an hydrogen (H) or a C₁-C₁₀ alkyl, C₂-C₁₀ alkenyl, or C₂-C₁₀ alkynyl, optionally substituted by one or more methyl, ethyl, n-propyl, i-propyl, n-butyl, s-butyl, i-butyl or t-butyl group(s); more preferably R₇ is selected from the group consisting of hydrogen (H), a C₁ alkyl (methyl) group, and a C₂, C₃, C₄, C₅, C₆, C₇, C₈, C₉, and C₁₀ alkyl, alkenyl, and alkynyl group, optionally comprising one or more heteroatoms, such as —O—, NH—, S and mixtures of thereof. Preferably, it is likely understood that if Xg₁, Xi₁, Xg₂ and Xi₂ happened to be different in nature while being independently represented by N(R₇), C(O)N(R₇), N(R₇)C(O), OC(O)N(R₇), N(R₇)C(O)O, then R₇ might be of several different nature among the possibilities depicted above within the same molecule;
g1 and g2 are integers equal to 0 or 1;
i1 and i2 are integers equal to 0 or 1;
Iₕ₁ and Iₕ₂, identical or different, both represent a linear or branched non-saturated hydrocarbon chains comprising from 2 to 16 carbon atoms, preferably from 2 to 8 carbon atoms and at least one unsaturation, more preferably Iₕ₁ and Iₕ₂, identical or different are independently

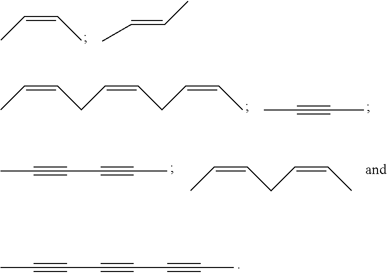

h1 and h2 are integers equal to 0 or 1;
J₁ and J₂ are branched hydrocarbon groups comprising from 1 to 20 carbon atoms, preferably from 1 to 12, and/or one or more heteroatoms; J₁ can optionally be covalently coupled with either Ak_{f1}, Akⱼ₁, or Xg₁, Xi₁, or Iₕ₁ group on the one hand, and to A on the other hand, whereas J₂ can optionally be covalently coupled with either Ak_{f}2, Akf₂ Xg₂ and Xi₂, or Iₕ₂ on the one hand, and to B on the other hand;
I₁ and I₂ are integers equal to 0 or 1; it being preferably understood that if I₁ is equal to 0, then either Ak_{f1}, Akⱼ₁, or Xg₁, Xi₁, or Iₕ₁ group is covalently attached to A. Similarly, if l2 is equal to 0, then either Ak_{f2} Akⱼ₂ Xg₂ and Xi₂, or Iₕ₂ group is covalently attached to B;
k1 and k2 are integers comprised between 1 or 3, it being preferably understood that if l1 is equal to 1 then k1 is at least equal to 2. Similarly, if l2 is equal to 1 then k2 is at least equal to 2.

In the above mentioned formula (IV), when R₁ and R₂ are identical, their structure can be described according to formula (VII):

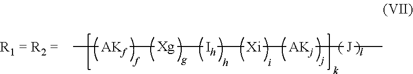

wherein:
Ak_{f} and Akⱼ, identical or different, represent a linear or branched hydrocarbon group comprising from 1 to 22 carbon atoms, preferably from 1 to 12 carbon atoms, optionally comprising one or more heteroatoms;
f is an integer equal to 0 or 1;
j is an integer equal to 0 or 1;
Xg and Xi, identical or different can be O, S, N(R₇), C(O), C(O)O, OC(O), C(O)N(R₇), N(R₇)C(O), OC(O)N(R₇), N(R₇)C(O)O, C(O)S, C(S)O, S(O), S(O)(O), C(S), wherein R₇ is either an hydrogen (H) or a C₁-C₁₀ alkyl, C₂-C₁₀ alkenyl, or C₂-C₁₀ alkynyl, optionally substituted by one or more methyl, ethyl, n-propyl, i-propyl, n-butyl, s-butyl, i-butyl or t-butyl group(s); more preferably R₇ is selected from the group consisting of hydrogen (H), a C1 alkyl (methyl) group, and a C₂, C₃, C₄, C₅, C₆, C₇, C₈, C₉, and C₁₀ alkyl, alkenyl, and alkynyl group, optionally comprising one or more heteroatoms, such as —O—, NH—, S and mixtures of thereof;
g is an integer equal to 0 or 1;
i is an integer equal to 0 or 1;
Iₕ represent a linear or branched non-saturated hydrocarbon chain comprising from 2 to 16 carbon atoms, preferably from 2 to 8 carbon atoms and at least one unsaturation, more preferably Iₕ is selected from:

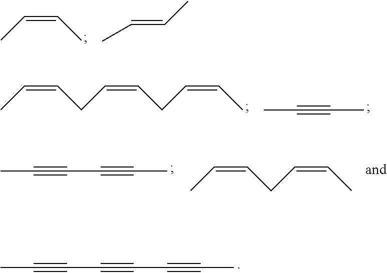

h is an integer equal to 0 or 1;
J is a branched hydrocarbon groups comprising from 1 to 20 carbon atoms, preferably from 1 to 12, and/or one or more heteroatoms. J can optionally be covalently coupled with the Akⱼ or Xi or Iₕ or Xg or Ak_{f} group on the one hand, and to A or B on the other hand;
I is an integer equal to 0 or 1; it being preferably understood that if I is equal to 0, then either Akⱼ or Xi or Iₕ or Xg or Ak_{f} is covalently attached to A on the one hand and to B on the other hand;
k is an integer comprised between 1 or 3; it being preferably understood that if I is equal to 1 then k is at least equal to 2.

Preferably, in formula (I) as previously defined R incorporates an amino-acid acting as “biocompatible template”.

Examples of amino-acid acting as “biocompatible template” include, but are not limited to, glutamic acid, aspartic acid and/or ornithine.

By “biocompatible template” it is preferably understood a multivalent amino-acid acting as a basis for the structural backbone of these new lipids, preferably new tunable lipids, such as glutamic acid, aspartic acid, lysine and ornithine that each present three chemical functions able to react with a broad variety of components following the basic principles of the peptide coupling chemistry. In the case of glutamic acid and aspartic acid, two carboxylic functions and one primary amine are free to be used for coupling, while in the case of lysine and ornithine, it is two amine functions for a carboxylic acid function that are available.

More particularly, by “biocompatible template” it is preferably understood a multivalent amino-acid acting as a basis for the structural backbone of these new lipids, understanding that those amino-acids could belong to the L or D series.

Examples of biocompatible template may also include groups of atoms defined in R that do not belong to the amino-acid library but constitutes biosynthetic precursors of amino-acids. Examples of such groups of atoms defined in R that do not belong to the amino-acid library but constitutes biosynthetic precursors of amino-acids include, but are not limited to, derivatives of a-ketoglutaric acid, L-Malic acid, D-Malic acid, oxaloacetic acid or combination thereof.

Preferably, in formula (I) as previously defined R is selected from:

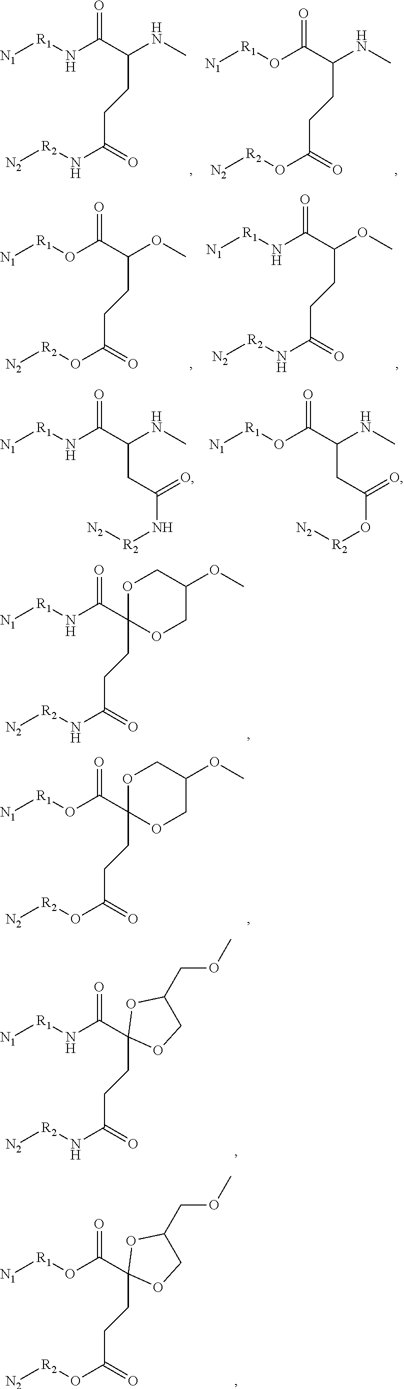

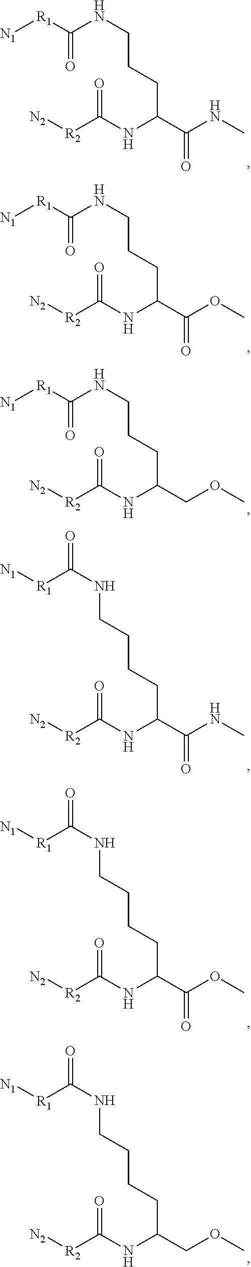

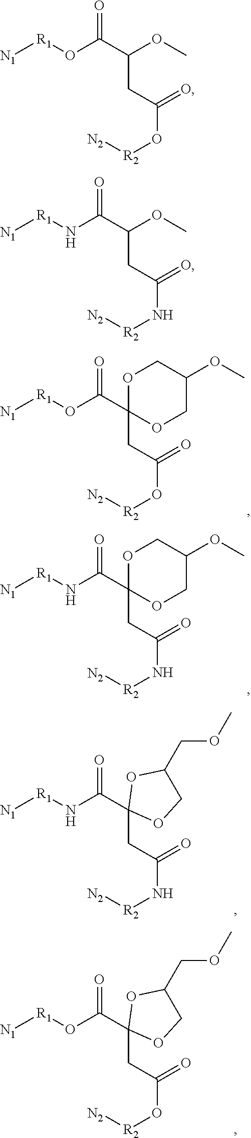

### (Formula VIII)

wherein, R₁, R₂, N₁ and N₂ are as previously defined.

More preferably, in formula (I) as previously defined R is selected from:

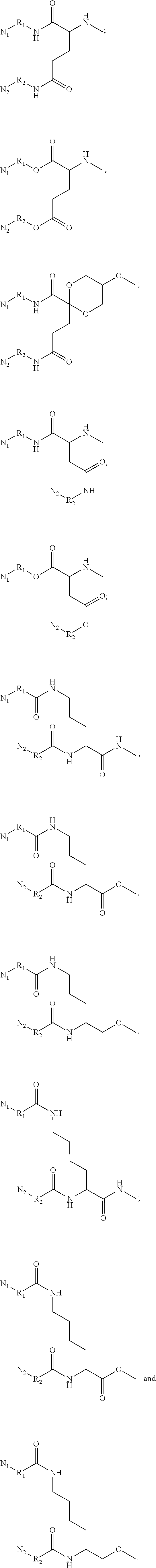

### (Formula VIII)

wherein, R₁, R₂, N₁ and N₂ are as previously defined.

Preferably, in formulae (VI a) and (VI b) as previously defined, J₁ and J₂, identical or different correspond to formula —CO—Y₁—NH—Y₂—N—[Y₃—NH]₂— or —NH—Y₂—N—[Y₃—NH]₂—, wherein Y₁, Y₂ and Y₃, identical or different, represent a bridging alkylene group comprising from 1 to 8 carbon atoms, preferentially from 1 to 4.

Preferably, Y₁ represents a methylene, whilst Y₂ and Y₃ represent a bridging alkylene group comprising from 1 to 4 carbon atoms and more preferably 2 carbon atoms.

Preferably, J₁ and J₂ therefore possess three functional groups making it possible for J₁ to form a covalent bond optionally with at least two iterations of either Ak_{f1}, Akⱼ₁, or Xg₁, Xi₁, or Iₕ₁ group on the one hand, and with A on the other hand, whereas J₂ can optionally be covalently coupled with at least two iterations of either Ak_{f2}, Akⱼ₂ Xg₂ and Xi₂, or Iₕ₂ on the one hand, and with B on the other hand.

Preferably, in the case where R₁ and R₂ are identical, then J₁ and J₂ are identical and can be named J, which defined the same way as J₁ and J₂. Thus, in this case, J possesses three functional groups making it possible for J to form a covalent bond optionally with at least two iterations of Ak_{f}, Akⱼ, or Xg, Xi₁ or Iₕ group on the one hand, and with A&B on the other hand.

In a particular embodiment, in formula (III) as previously defined, Gr can be absent or Gr can serve as a spacer arm and thus corresponds to the molecular pattern noted as —W₄—Y₄—W₄—,

wherein Y₄ represents a bridging alkylene group comprising from 1 to 8 carbon atoms, preferentially from 1 to 4, whilst W₄ represents a —O—, —CO— or —NH— group.

In a particular embodiment, in formula (III) as previously defined, Gr corresponds to the formula CO—Y₄—CO,

wherein Y₄ has the same meaning as previously defined and is connected by an amide bond to the lipophilic region R on the one hand, and either to the RAc radical, or directly to the S₁ or Q or Z group on the other hand.

Preferably, in formula (III) as previously defined, Gr can be absent or Gr can serve as a spacer arm and thus corresponds to the molecular pattern noted as —W₄—Y₄—W₄—,

wherein Y₄ represents a bridging alkylene group comprising from 1 to 8 carbon atoms, preferentially from 1 to 4, whilst W₄ represents a C(O)—O, C(O)—NH, C(O)—S, S(O), S(O)—O, C(S)—O, —O— or —NH— group.

Even more preferably, in formula (III) as previously defined, Gr corresponds to the formula —W₄—Y₄—W₄—,

wherein Y₄ has the same meaning as previously defined and is connected by a carbonate or carbamate bond to the lipophilic region R on the one hand, and either to the RAc radical, or directly to the S₁ or Q or Z group on the other hand.

Preferably, the amino acid, the radical of which is denoted RAc in formula (III) as previously defined, is chosen from aspartic acid, glutamic acid, alanine, arginine, asparagine, cysteine, glutamine, glycine, histidine, isoleucine, leucine, lysine, methionine, ornithine, phenylalanine, praline, serine, threonine, tyrosine, tryptophan and valine.

Preferably, in formula (III) as previously defined, the amino acid, the radical of which being denoted RAc is chosen from aspartic acid, glutamic acid, isoleucine, leucine, lysine, ornithine, glycine and phenylalanine, and more preferably from aspartic acid, glutamic acid, glycine, ornithine and lysine, preferably from aspartic acid, glutamic acid, isoleucine, leucine, lysine and phenylalanine, and more preferably from aspartic acid, glutamic acid, and lysine. However, this amino acid can also be chosen from rarer amino acids such as for example, ρ-alanine, γ-aminobutyric acid, a-aminoadipic acid, hydroxyproline, hydroxylysine, phenylserine, α,β-diaminopimelic acid, ornithine and any other modified amino acids, any amino acid being suitable since it comprises, by definition, two functional groups, one being carboxylic acid, the other amine, allowing it to bind covalently, on the one hand to the spacer arm Gr or to R and, on the other hand, to at least one S₁ or Q or Z group. Preferably, the choice of the amino acid depends in particular on the value that it is desired to give to s in formula (IV), to the extent that it must comprise at least three functional groups to allow s to be equal to 2, whilst it is sufficient for it to comprise only two functional groups to have s equal to 1. Preferably, it is preferred that RAc is an amino acid radical belonging to the L series. However, it is also possible that RAc is an amino acid radical of the D series.

Preferably, in formula (IV), S₁ can correspond to the molecular pattern noted as: —W₅—Y₅— and S₂ can correspond to the pattern noted as: —Y₆—W₆— in which Y₅ and Y₅, identical or different, represent a bridging alkylene group comprising from 1 to 8 carbon atoms, preferentially from 1 to 4, whilst W₅ and W₆, identical or different, represent a —CO—, —NH-or-O— group. In this case, preferably, Si forms a covalent bond with an RAc, Gr or R group on the one hand, and with the D group on the other hand, whilst S₂ forms a covalent bond with a D group on the one hand, and with a Q or Z group on the other hand.

Preferably, in formula (IV) as previously defined, D represents an ester (—CO—O—), carbonate (—O—C(O)—O—), disulfide (—S—S—), vinyl ether (—O—C═C—), acylhydrazone (CO—NR—N═CR′R″) group, preferably an ester (—CO—O—), disulfide (—S—S—), vinyl ether (—O—C═C—), acylhydrazone (CO—NR—N═CR′R″) group, the ester and disulfide groups being particularly preferred. In this case, preferably, these groups form a covalent bond with the S₁ group on the one hand and the S₂ group on the other hand.

Preferably, Q corresponds to the molecular pattern noted as —CO—Y₇—NH—Y₈—N—[Y₉—NH]₂— or as —NH—Y₈—N—[Y₉—NH]₂—, in which Y₇, Y₈ and Y₉, identical or different, represent a bridging alkylene group comprising from 1 to 8 carbon atoms, preferentially from 1 to 4. Additionally, Y₇ preferably represents a methylene, whilst Y₈ and Y₉ preferably represent a bridging alkylene group comprising from 1 to 4 carbon atoms and, better still, 2 carbon atoms. In this case, preferably, Q therefore possesses three functional groups making it possible for it to form a covalent bond with optionally S₂ or RAc or Gr or R on the one hand, and at least two Q and/or Z groups on the other hand.

Preferably, in formula (IV) as previously defined, Z represents ionizable molecular pattern able to form a covalent bond optionally with Q or S₂ or RAc or Gr or R on the one hand, and one or two basic reactive functions (such as for example amine and alcohol) making it possible to form a covalent bond optionally with one or two other Z groups on the other hand.

By “ionizable molecular pattern” it is preferably understood a pattern of chemical functions incorporating at least one nitrogen atom and bearing no charges at physiological pH (pH=7.0-7.4), that can go positively charged if the pH of the medium dropped below the pKa value of the entity.

Preferably, by “ionizable molecular pattern” it is preferably understood a pattern of chemical functions incorporating at least one nitrogen atom and bearing no charges at physiological pH (pH=7.0-7.4), that can display one or several positive charges when the pH of the medium ranges from 2.0 and 7.0, preferably from 4.0 and 7.0 and more preferably from 4.5 and 7.0.

More preferably, the compounds of formula (I) are selected from:

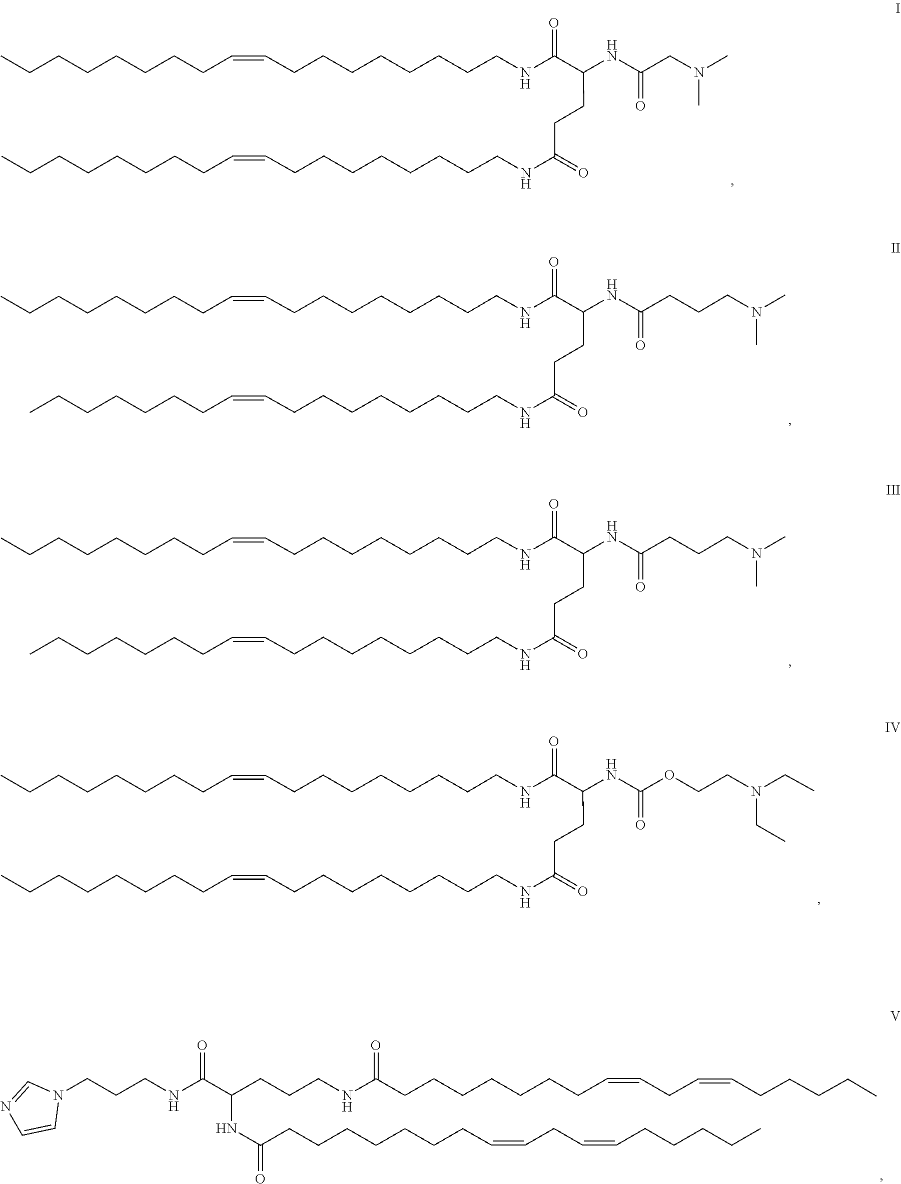

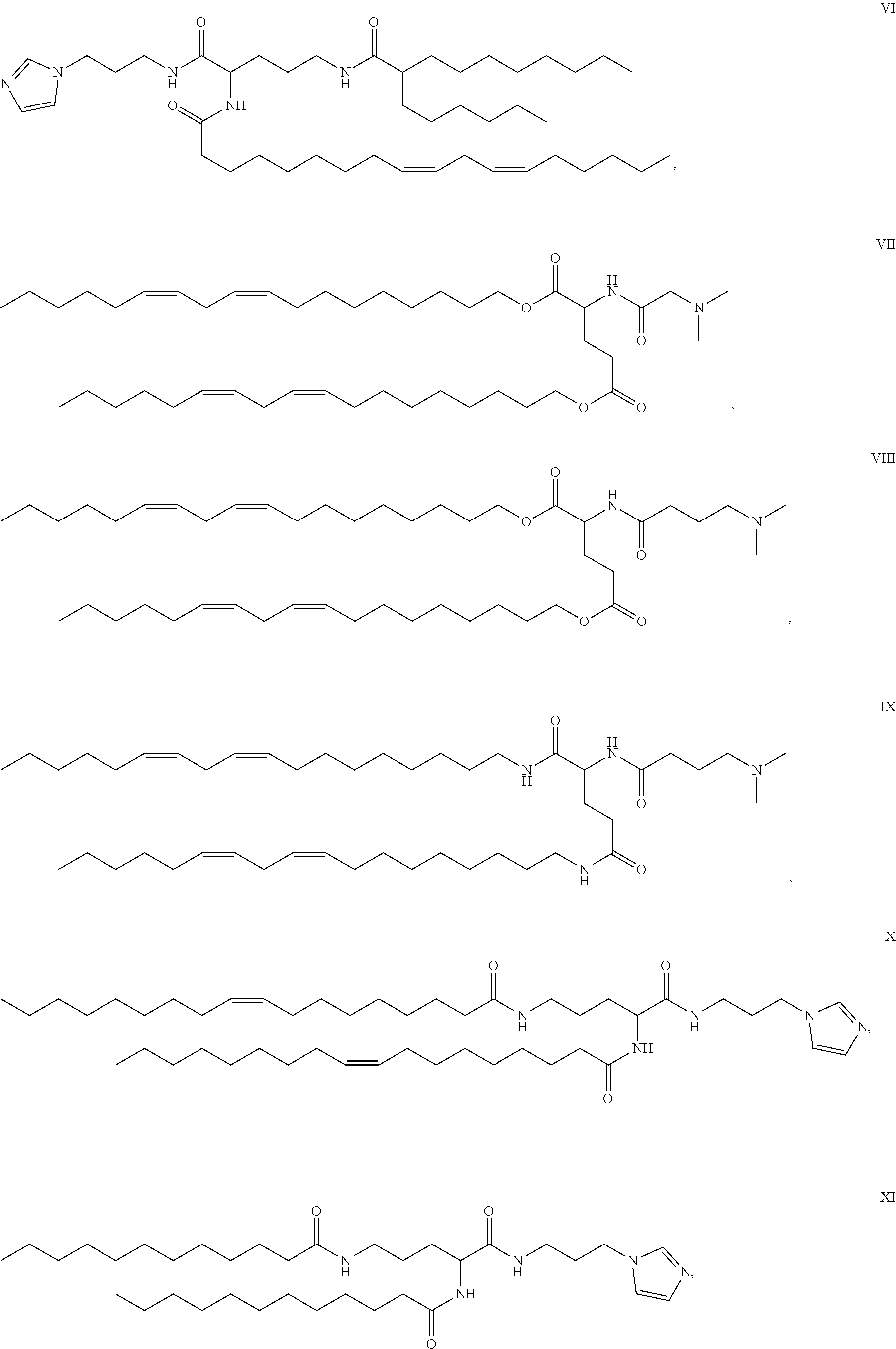

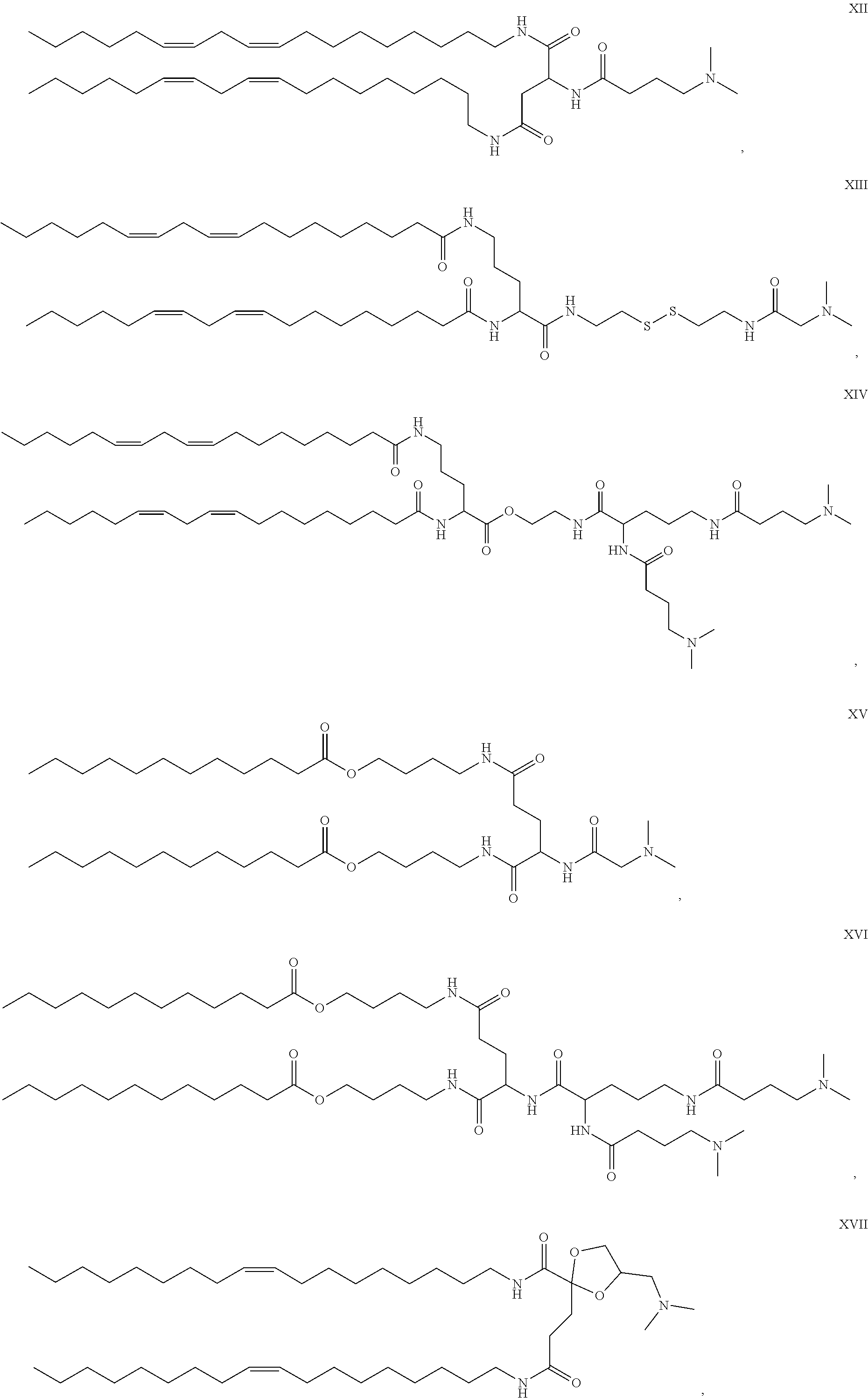

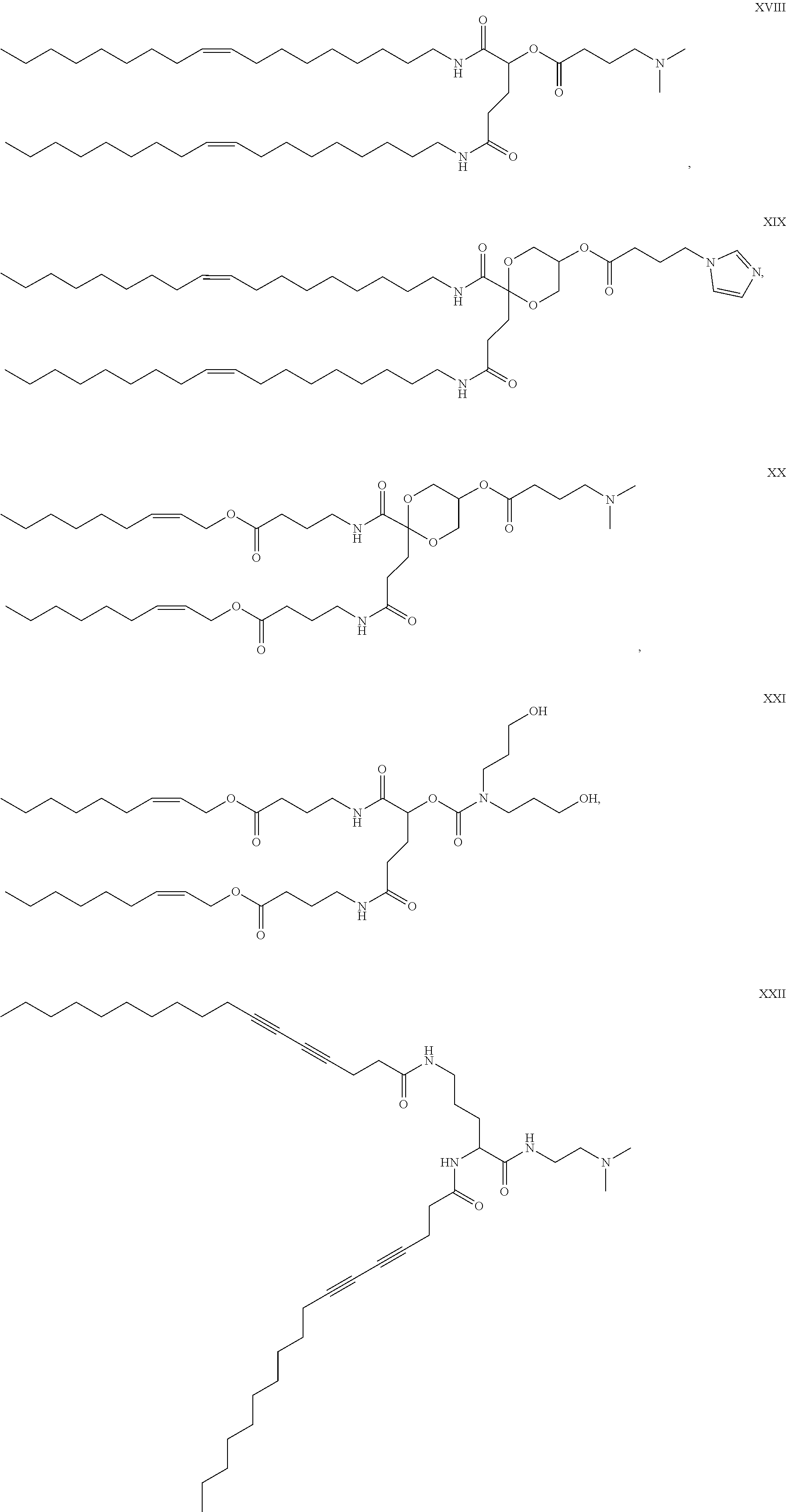

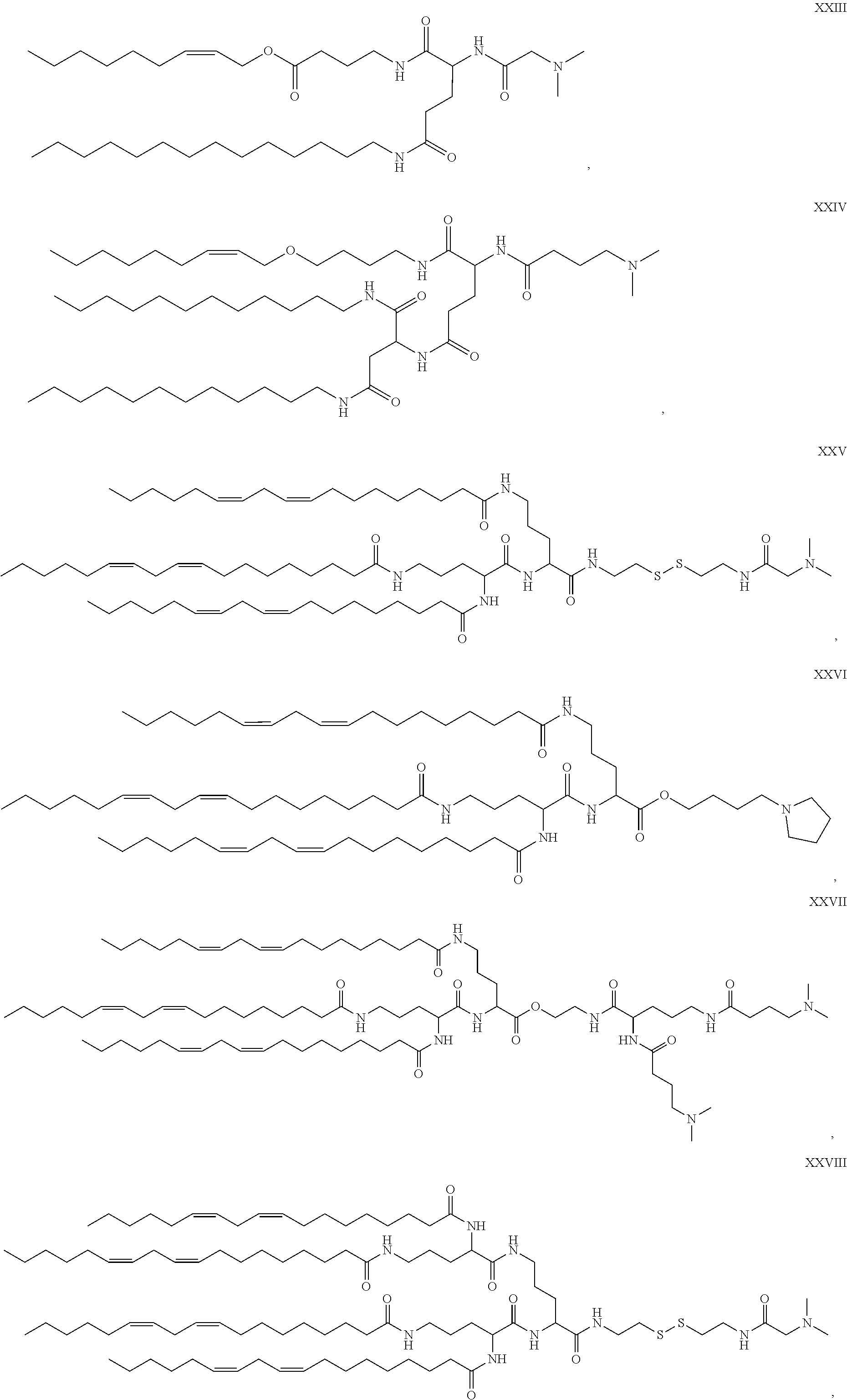

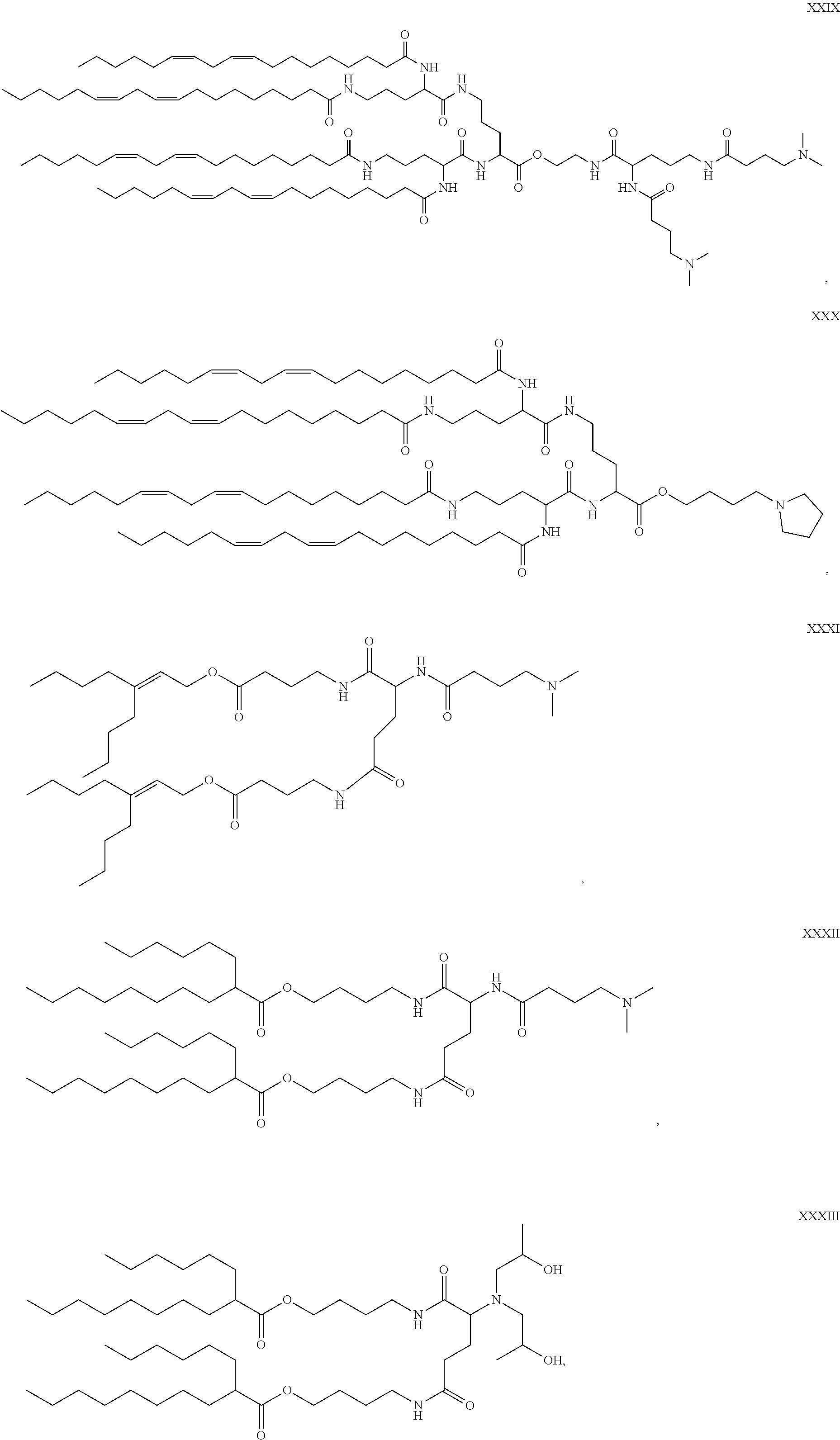

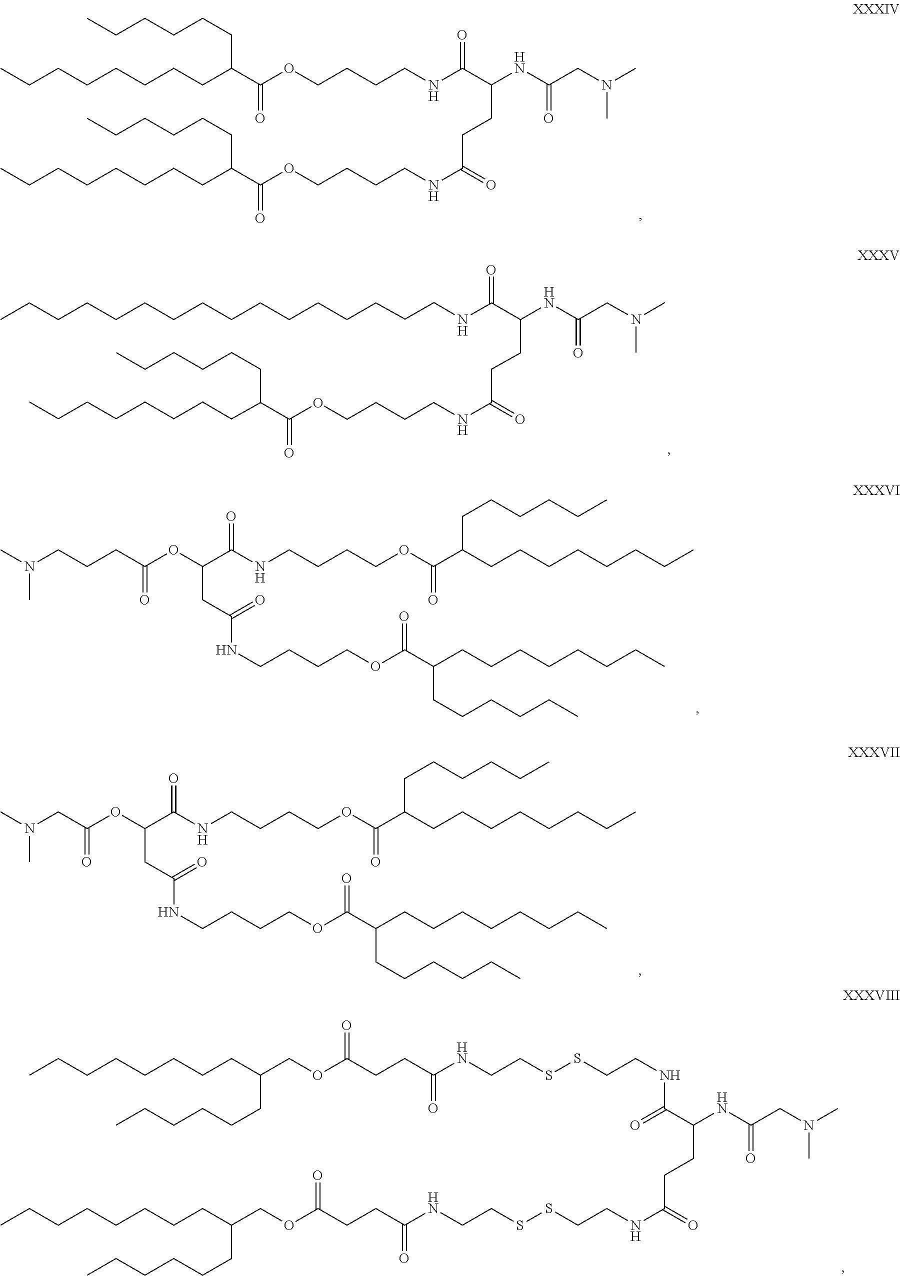

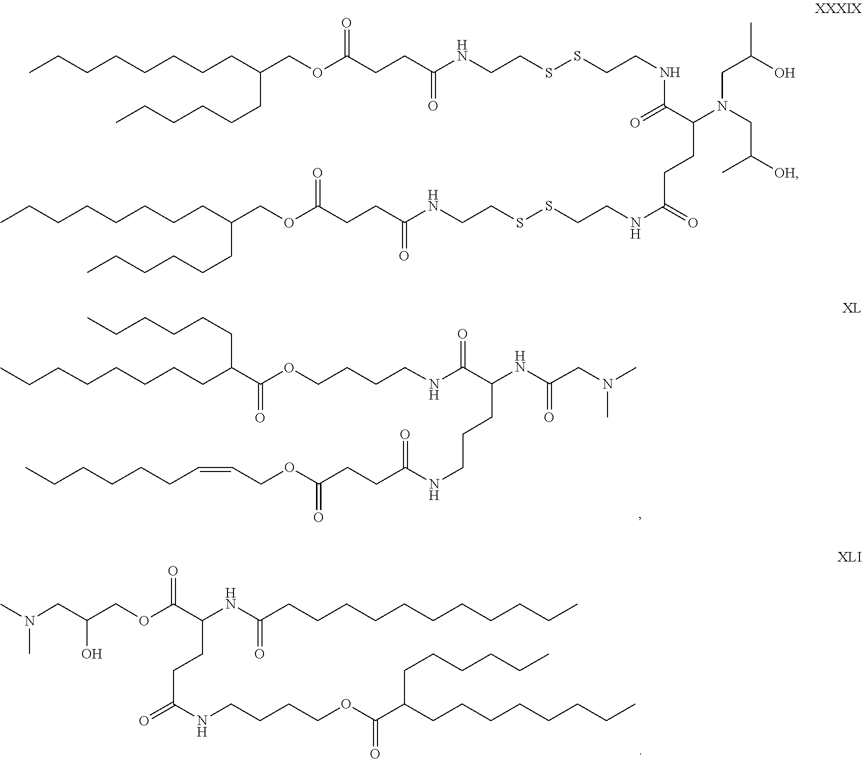

The present invention also relates to the following compounds:

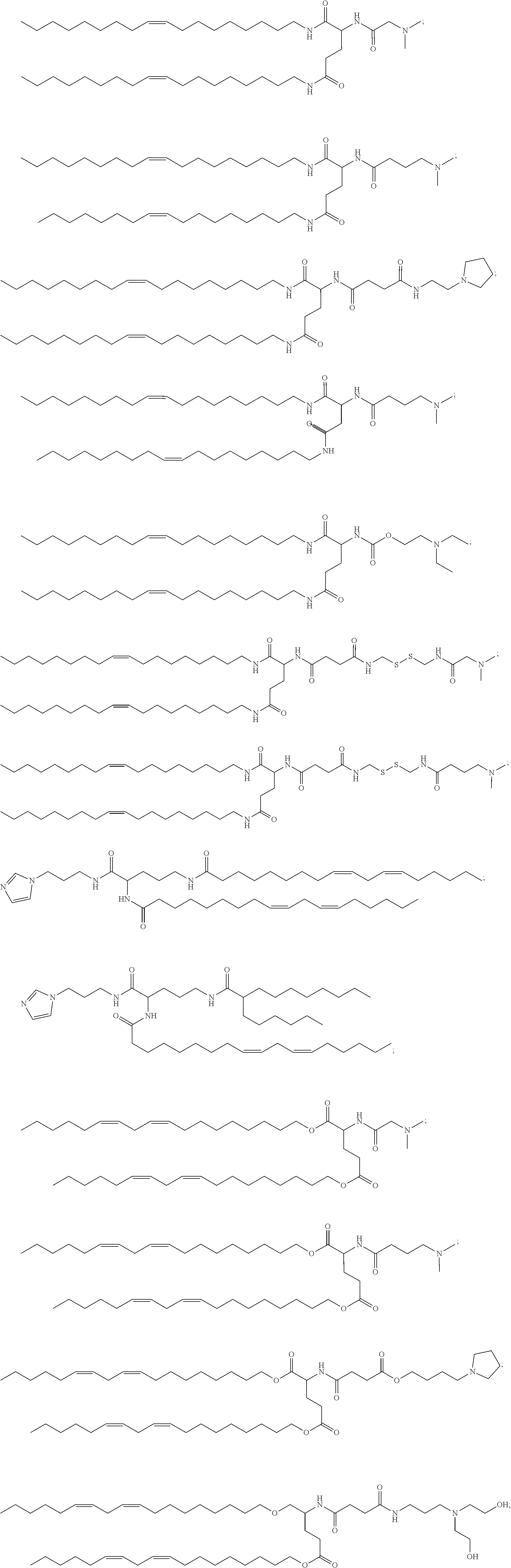

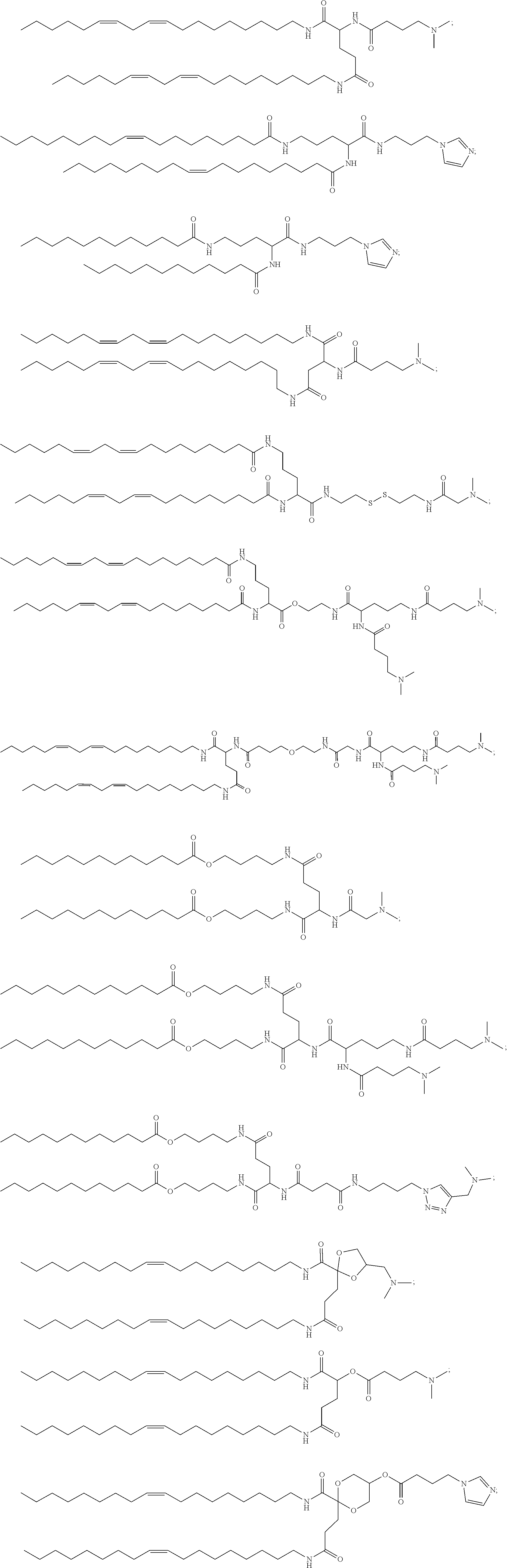

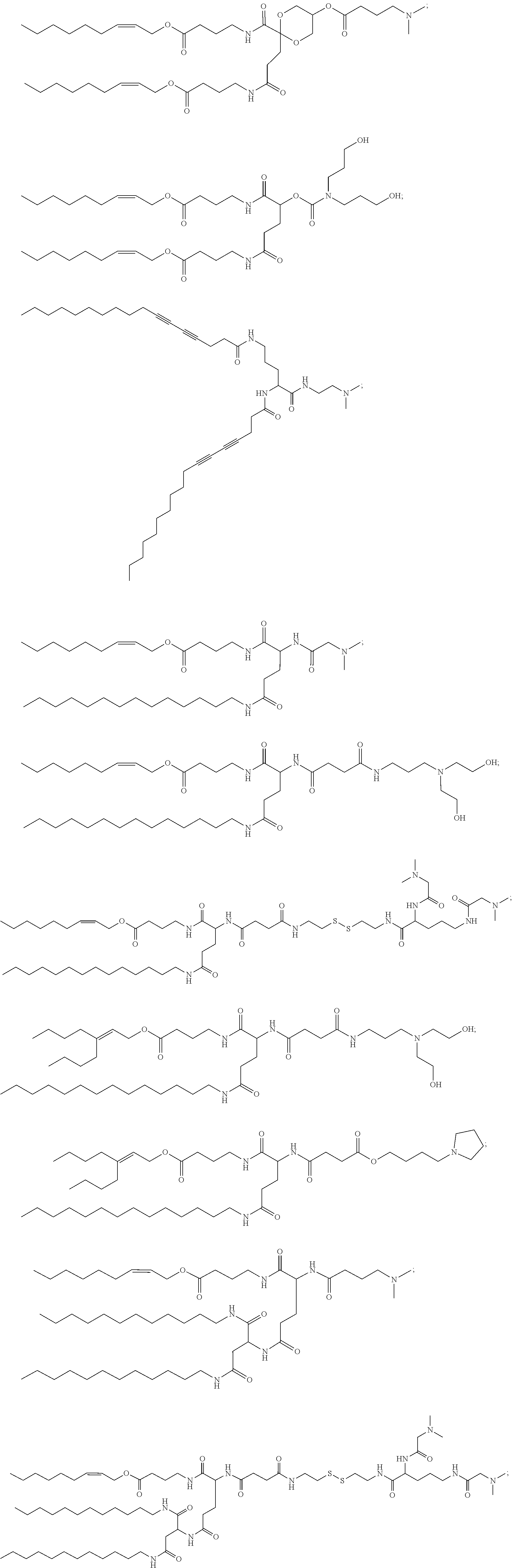

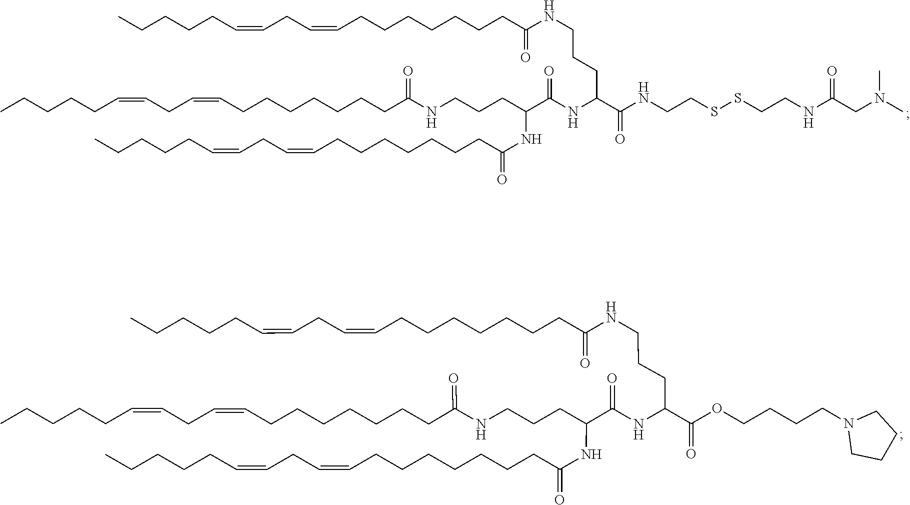

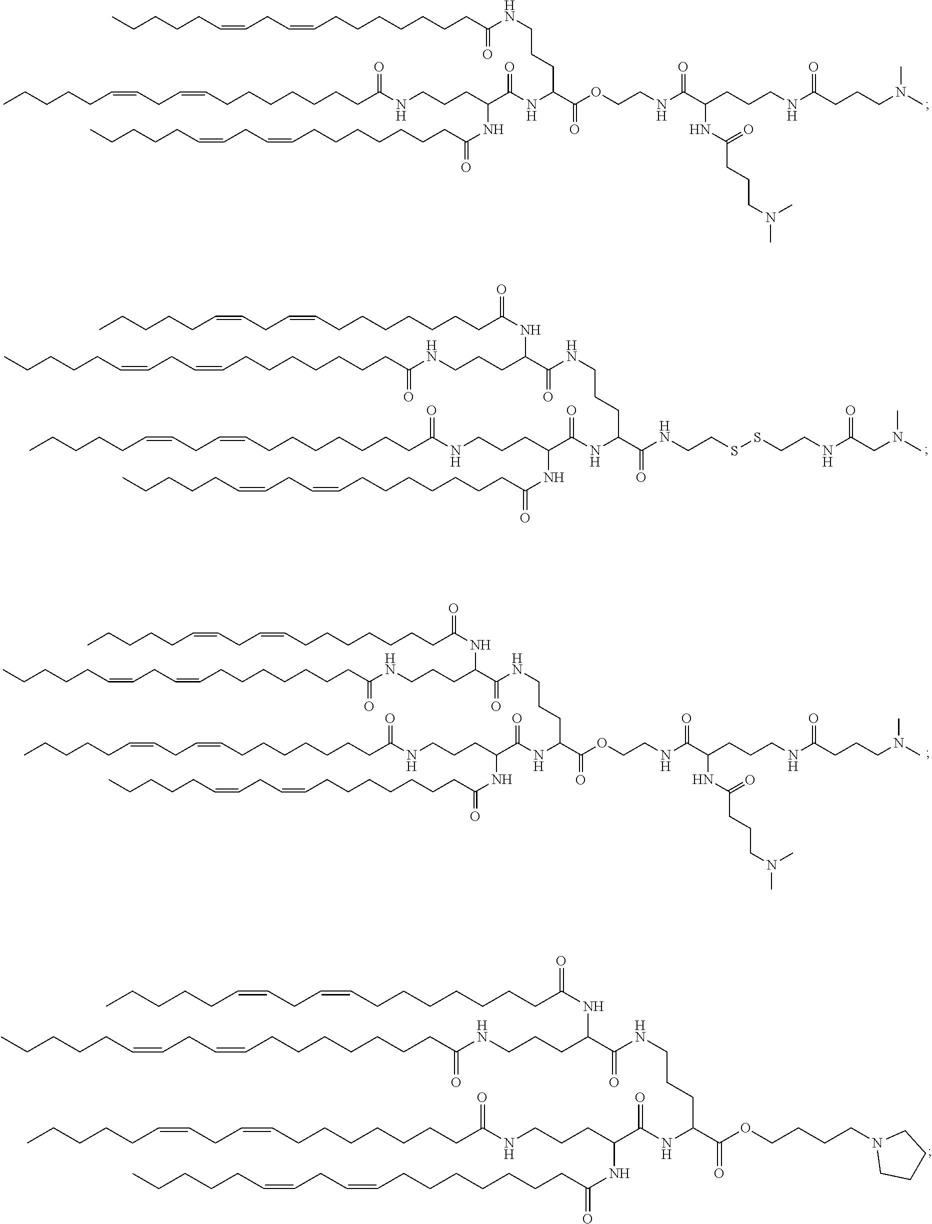

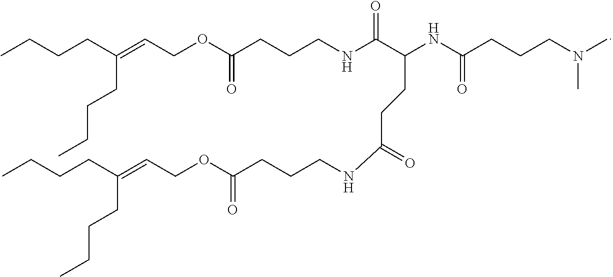 [SMILES: CCCCC(=CCOC(=O)CCCNC(=O)CCC(NC(=O)CCCN(C)C)C(=O)NCCCC(=O)OCC=C(CCCC)CCCC)CCCC]

Another object of the present invention relates to a mixture comprising one or more lipid(s) of formula (I) as previously defined.

The lipid of formula (I) as previously defined may be present under the form of a racemic mixture.

The mixture comprising the lipid of formula (I) as previously defined may be enriched in one diastereomer of the lipid of formula (I) as previously defined. In particular, the mixture of the lipid of formula (I) as previously defined may have at least 95%, at least 90%, at least 80% or at least 70% diastereomeric excess.

The mixture comprising the lipid of formula (I) as previously defined may be enriched in one enantiomer of the lipid of formula (I) as previously defined. In particular, the mixture of the lipid of formula (I) as previously defined may have at least 95%, at least 90%, at least 80% or at least 70% enantiomer excess.

The mixture comprising the lipid of formula (I) as previously defined may be chirally pure. In particular, the lipid of formula (I) as previously defined may be in the form of a single optical isomer.

The mixture comprising the lipid of formula (I) as previously defined may be enriched in one optical isomer of the lipid of formula (I) as previously defined.

Where a double bond is present (such as a carbon-carbon double bond or carbon-nitrogen double bond), there can be isomerism in the configuration of the double bond (such as cis/trans or E/Z isomerism). Where the configuration of a double bond is illustrated in a chemical structure, it is preferably understood that the corresponding isomer can also be present. The amount of isomer present can vary, depending on the relative stabilities of the isomers and the energy required to switch between the isomers. Accordingly, some double bonds may be, for practical purposes, present in only a single configuration, whereas others (for example where the relative stabilities are similar and the energy of conversion low) may be present as inseparable equilibrium mixture of configurations.

The lipid of formula (I) as previously defined may include one or more biodegradable groups. The biodegradable group(s) may include one or more bonds that may undergo bond breaking reactions in a biological environment (such as for example in an organism, organ, tissue, cell, or organelle). Examples of functional groups that contain a biodegradable bond include, but are not limited to, esters, dithiols and/or oximes.

The present invention also relates to a process for preparing the lipid of formula (I) as previously defined, and to a process for preparing the mixture comprising the lipid of formula (I) as previously defined

Thus, the present invention also relates to a process for preparing the lipid of formula (I) as previously defined or the mixture comprising the lipid of formula (I) as previously defined.

For example, the process for preparing the lipid of formula (I) as previously defined or the mixture comprising the lipid of formula (I) as previously defined may comprise the steps of:
Attaching one or two chemical functions of the chosen biocompatible template to an hydrophobic (or lipophilic) group of atoms as defined in formula (I) and/or (II);
Optionally introducing a spacer region using the remaining chemical functions of the biocompatible template that now also contains the hydrophobic (or lipophilic) area. This spacer region can be defined according formula (I) and/or (Ill), and may include one or more chemical bonds or entities sensitive to their environment, able to enhance the lipid degradability, preferably the tunable lipid degradability. This spacer can also include a chemical functions able to be attached to the polar region as previously defined in Formula (I);
finally introducing a polar region, preferably a tunable polar region, that will act as the hydrophilic part of the whole chemical entity. This polar region can be constructed according to the formula (IV) as previously defined. In the case of a spacer region previously attached to the biocompatible template, this polar region can be chemically attached to it. If the spacer region is absent, the polar region can be attached directly to the above-described biocompatible template, using one or two of its remaining chemical functions.

This process can be modified, with its steps switched, depending on the global synthetic process chosen to obtain the targeted lipid, preferably tunable lipid. It means that in some case for instance, the polar region might be first attached to the spacer region before this new entity might be attached to the biocompatible template. The person skilled in the art is able to select the appropriate process to obtain the lipid of formula (I) as previously defined or the mixture comprising the lipid of formula (I) as previously defined.

The lipid of formula (I) as previously defined or the mixture comprising the lipid of formula (I) as previously defined may also be prepared by well-known organic synthesis methods, including the methods described in the examples of the present invention.

Examples of synthesis methods that may be used to synthesize the lipid of formula (I) as previously defined or the mixture comprising the lipid of formula (I) as previously defined, include, but are not limited to, coupling methods involving carbodiimide such as diisopropylcarbodiimide or uronium salts-based coupling agents such as HBTU, or nitrogen-based protecting group chemistry (as described for example in Greene's Protective Groups in Organic Synthesis, T. W. Greene et. al., Wiley-Interscience New York City, 1999).

In particular, the lipid of formula (I) as previously defined may comprise at least one protonatable or deprotonatable group, such that the whole lipid may be positively charged at a first pH (such a pH ranging from 3.0 to 6.9, preferably from 4.5 to 6.9) below physiological pH (such a pH ranging from 7.0 to 7.4), and may be neutral at a second pH, higher than the first pH and preferably at or above physiological pH. It will be understood by one having ordinary skill in the art that the addition or removal of protons as a function of pH is an equilibrium process, and that the reference to a charged or a neutral lipid refers to the nature of the predominant species and does not require that all of the lipid be present in the charged or neutral form.

The lipid of formula (I) as previously defined may have more than one protonatable or deprotonatable group or may be zwitterionic.

The pKa's values of the lipid of formula (I) as previously defined may be lower than the pH observed in physiological medium (such as a pH ranging from 7.0 to 7.4). Preferably, the lipid of formula (I) as previously defined has a global pKa value ranging from 3.0 to 6.9, preferably from 4.5 to 6.90.

In the present invention, the pKa value preferably corresponds to a dimensionless number related to the acid dissociation constant of the corresponding acid specie and is thus a quantitative measure of the acidic strength in solution of a chemical specie.

Preferably, the pKa value of the lipid of formula (I) as previously defined is finely tuned to stay under the value of the observed in physiological medium (such as a pH ranging from 7.0 to 7.4). Thus, at this pH, all the lipid of formula (I) as previously defined can be present in solution mostly under their non-protonated form (such as basic form). It will be understood by one having ordinary skill in the art that the value of the pKa constant as defined above may cover the acidic strength of the lipid of formula (I) as previously defined. Thus, the present invention may also cover a lipid of formula (I) bearing several protonable moieties (protonatable lipid) and present at physiological pH mainly under its neutral form (such as non-protonated form).

Preferably, protonatable lipids according to the invention have a pKa for the protonatable group in the range of about 4 to about 7. Accordingly, such lipids will be cationic at a lower pH, whereas formulation including the tunable lipids, preferably the tunable lipids, will be largely (though not completely) surface neutralized at physiological pH of around pH 7.4. One of the benefits of choosing this value for pKa is that at least some nucleic acid associated with the outside surface will lose its electrostatic interaction at physiological pH and be removed by a simple dialysis, thus greatly reducing the particle's susceptibility to clearance.

Advantageously, the lipid of formula (I) as previously defined may incorporate a neutral nitrogen-centred chemical function ables to get protonated at acidic pH, preferably at highly acidic pH, while remaining neutral at physiological pH.

Thus, the lipids of formula (I) as previously defined may be under free form, as well as pharmaceutically acceptable salts and stereoisomers thereof. Thus, the lipid, preferably the tunable lipid, of the present invention can be under its protonated form, that is cationic form.

By “lipid under free form” it is preferably understood the lipid in a non-salt form. The free form may be regenerated by treating the lipid salt with a suitable dilute aqueous base solution such as aqueous NaOH, potassium carbonate, ammonia and/or sodium bicarbonate solution, preferably dilute aqueous NaOH, potassium carbonate, ammonia and/or sodium bicarbonate solution.

The pharmaceutically acceptable salts of the lipids of formula (I) as previously defined can be synthesized from the lipids of formula (I) as previously defined, which contain a basic or acidic moiety, by conventional chemical methods. Generally, the salts of the basic lipids may be prepared either by ion exchange chromatography or by reacting the free base with stoichiometric amounts or with an excess of the desired salt-forming inorganic or organic acid in a suitable solvent or various combinations of solvents. Similarly, the salts of the acidic lipids may be formed by reactions with the appropriate inorganic or organic base.

Examples of pharmaceutically acceptable salts of the lipids of formula (I) as previously defined include, but are not limited to, non-toxic salts of the lipids of formula (I) as previously defined as those formed by reacting a basic lipid with an inorganic or organic acid.

Examples of non-toxic salts include, but are not limited to, those derived from inorganic acids such as hydrochloric acid, hydrobromic acid, sulfuric acid, sulfamic acid, phosphoric acid and nitric acid, as well as salts prepared from organic acids such as formic acid, acetic acid, propionic acid, succinic acid, glycolic acid, stearic acid, lactic acid, malic acid, tartaric acid, citric acid, ascorbic acid, palmoic acid, maleic acid, hydroxymaleic acid, phenylacetic acid, glutamic acid, benzoic acid, salicylic acid, sulfanilic acid, 2-acetoxy-benzoic acid, fumaric acid, toluenesulfonic acid, methanesulfonic acid, ethane disulfonic acid, oxalic acid, isethionic acid, and/or trifluoroacetic (TFA) acid.

When the lipids of formula (I) as previously defined are acidic lipids, suitable “pharmaceutically acceptable salts” refers to salts prepared from pharmaceutically acceptable non-toxic bases including inorganic bases and organic bases. Salts prepared from pharmaceutically acceptable inorganic non-toxic bases include, but are not limited to, salts of aluminum, ammonium, calcium, copper, ferric, ferrous, lithium, magnesium, manganic salts, manganous, potassium, sodium, zinc and mixtures thereof. Preferably, the base is selected from ammonium, calcium, magnesium, potassium and/or sodium. Salts prepared from pharmaceutically acceptable organic non-toxic bases include, but are not limited to, salts of primary amines, secondary amines, tertiary amines, substituted amines including naturally occurring substituted amines, cyclic amines and basic ion exchange resins, such as arginine, betaine, caffeine, choline, N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and mixtures thereof.

The lipids of formula (I) as previously defined may potentially be internal salts or zwitterions, since under physiological conditions a deprotonated acidic moiety in the compound, such as a carboxyl group, may be anionic, and this electronic charge might then be balanced off internally against the cationic charge of a protonated or alkylated basic moiety.

Preferably, in the case of the lipids of formula (I) being zwitterions, the deprotonated acidic moiety should have a pKa ranging from 4.5 to 7.0, so that its electronic negative charge does not compensate the cationic charge of a protonated or alkylated basic moiety in a medium bearing a pH value ranging from 4.5 to 7.0.

The lipid of formula (I) as previously defined may be formulated, optionally with one or more active ingredients, into an active supra-molecular architecture called a “lipid particle”.

Examples of lipid particles include, but are not limited to, liposomes, lipoplexes, micelles and lipid picoparticles, microparticles and/or nanoparticles.

By “liposome” it is preferably understood a structure having lipid-containing membranes enclosing an aqueous interior.

By “lipoplexe” is preferably understood a system obtained by complexation between a nucleic acid and a lipidic composition containing one or several lipids. Those complexes are usually obtained by adding a nucleic acid solution to a lipid dispersion followed by gentle mixing and short incubation.

Thus, another object of the present invention relates to a lipid particle comprising one or more lipid of formula (I) as previously defined or to a composition comprising the lipid particle as previously defined.

Thus, the lipid particle and the composition comprising the lipid particle as previously defined may include one or more lipids of formula (I) as previously defined.

The present invention also relates to a composition comprising the lipid of formula (I) as previously defined.

The composition comprising the lipid of formula (I) as previously defined may further include one or more active ingredients, and optionally:
one or more neutral lipids, and/or
one or more components capable of reducing aggregation of said composition, and/or
one or more sterols or sterol derivatives, and/or
one or more lipids bearing a targeting ligand.

Neutral lipids can be, when present, any lipid species, which exists either in an uncharged or neutral zwitterionic form at physiological pH. Preferably, the neutral lipid contains saturated fatty acids with carbon chain, potentially mono or di-unsaturated, which carbon chain's lengths stand in the range of C to C22. More preferably, the neutral lipid includes mono or di-unsaturated fatty acids with carbon chain lengths in the range of C10 to C20. More preferably, examples of neutral lipids include, but are not limited to, phospholipids such as diacylphosphatidylethanolamine, diacylphosphatidylcholine, dihydrosphingomyelin, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoyloleoyl-phosphatidylethanolamine (POPE), palmitoyloleyolphosphatidylglycerol (POPG), dioleoylphosphatidylethanolamine,4-(N-maleimidomethyl)-cyclohexane-1-carboxylate(DOPE-mal), dipalmitoylphosphatidylethanolamine(DPPE), dimyristoyl phosphatidylethanolamine (DMPE), distearoylphosphatidylethanolamine(DSPE), monomethylphosphatidylethanolamine, dimethylphosphatidylethanolamine, dielaidoylphosphatidylethanolamine(DEPE), stearoyloleoylphosphatidylethanolamine(SOPE), lysophosphatidylcholine and dilinoleoylphosphatidylcholine, nonphosphorous containing lipids such as for example stearylamine, dodecylamine, hexadecylamine, acetyl palmitate, glycerolricinoleate, hexadecyl stereate, isopropyl myristate, amphoteric acrylic polymers, triethanolamine-lauryl sulfate, alkyl aryl sulfate polyethyloxylated fatty acid amides, dioctadecyldimethyl ammonium bromide, ceramide, sphingomyelin, cerebrosides, cephalin, and mixtures thereof.

The neutral lipid that may be present in the composition of the present invention may comprise or may consist of one or more phospholipids (such as a cholesterol-free lipid composition).

The sterol that may be present in the composition of the present invention may comprise or consist of cholesterol or a derivative thereof (such as a phospholipid-free lipid composition).

Examples of components capable of reducing aggregation of said composition include, but are not limited to, polyethylene glycol (PEG)-modified lipids (PEG-modified lipids), polyethylene glycol (PEG) optionally substituted, conjugated and/or coupled, hydrophilic polymers other than PEG, poloxamers, monosialoganglioside (Gml), polyamide oligomers (PAO), and mixtures thereof.

Examples of PEG-modified lipids include, but are not limited to, PEG-modified phosphatidylethanolamine and phosphatidic acid, PEG-ceramide conjugates (such as PEG-CerC14 or PEG-CerC20), PEG-modified dialkylamines, PEG-modified 1,2-diacyloxypropan-3-amines, PEG-modified diacylglycerols and dialkylglycerols, mPEG (MW=2000)-diastearoylphosphatidylethanolamine (PEG-DSPE), mPEG (MW=2000)-dimyristoylphosphatidylethanolamine (PEG-DMPE), mPEG (MW=2000)-dipalmitoylphosphatidylethanolamine (PEG-DPPE), mPEG (MW=2000)-dioleoyl-phosphatidylethanolamine (PEG-DOPE), 1-(monomethoxy-polyethyleneglycol)-2,3-dimyristoylglycerol (PEG-DMG), N-[(methoxypoly(ethylene glycol) 2000 carbamyl]-1,2-dimyristyloxlpropyl-3-amine (PEG-c-DMA), a pegylated phosphatidylethanoloamine (PEG-PE),4-O-(2′,3′-dictetradecanoyloxy)propyl-1-O-(w-methoxy(polyethoxy)ethyl)butanedioate(PEG-S-DMG), a pegylated ceramide (PEG-cer), or a PEG dialkoxypropyl carbamate such as ( )-methoxy(polyethoxy)ethyl-N-(2,3-di(tetradecanoxy)propyl)carbamate or 2,3-di(tetradecanoxy) propyl-N-(c)-methoxy(polyethoxy)ethyl)carbamate, and mixtures thereof.

By “polyethylene glycol (PEG)” it is preferably understood a linear, water-soluble polymer of ethylene glycol repeating units with two terminal hydroxyl groups. PEGs are classified by their molecular weights and include, but are not limited to, monomethoxypolyethylene glycol (MePEG-OH), monomethoxypolyethylene glycol-succinate (MePEG-S), monomethoxypolyethylene glycol-succinimidyl succinate (MePEG-SNHS), monomethoxypolyethylene glycolamine (MePEG-NH), monomethoxypolyethylene glycol tresylate (MePEG-TRES), monomethoxypolyethylene glycol-imidazolyl-carbonyl (MePEG-IM), as well as such compounds containing a terminal hydroxyl group instead of a terminal methoxy group (such as HO-PEG-S, HO-PEG-S NHS, HO-PEG-NH), and mixtures thereof.

The PEG moiety of the PEG-conjugated lipid of the present invention may have an average molecular weight ranging from 550 daltons to 10,000 daltons, preferably ranging from 750 daltons to 5,000 daltons (such as from 1,000 daltons to 5,000 daltons, from 1,500 daltons to 3,000 daltons, from 750 daltons to 3,000 daltons, from 750 daltons to 2,000 daltons), and even more preferably of 2,000 daltons or 750 daltons.

The PEG may be optionally substituted by an alkyl, alkoxy, acyl, or aryl group.

The PEG can be conjugated directly to the lipid or may be linked to the lipid via a linker moiety. Any linker moiety suitable for coupling the PEG to a lipid can be used including, but not limited to, non-ester containing linker moieties and ester-containing linker moieties. Preferably, the linker moiety is a non-ester-containing linker moiety.

Suitable non-ester-containing linker moieties include, but are not limited to, amido (—C(O)NH—), amino (—NR—), carbonyl (—C(O)—), carbamate (—NHC(O)O), urea (—NHC(O)NH), disulfide (—S—S—), ether (—O—), succinyl (—(O)CCHCHC(O—), succinamidyl (—NHC(O)CHCHC(O)NH—), as well as combinations thereof (such as a linker containing both a carbamate linker moiety and an amido linker moiety). Preferably, a carbamate linker is used to couple the PEG to the lipid.

Suitable ester-containing linker moieties include, but are not limited to, carbonate (—OC(O)O—), succinoyl phosphate esters (—O—(O)POHO—), sulfonate esters, and combinations thereof.

Phosphatidylethanolamines having a variety of acyl chain groups of varying chain lengths and degrees of saturation can be conjugated to PEG.

Such phosphatidylethanolamines are commercially available, or can be isolated or synthesized using conventional techniques known by one having ordinary skill in the art.

Phosphatidylethanolamines containing saturated or unsaturated fatty acids with carbon chain lengths in the range of C₁₀ to C20 or phosphatidylethanolamines with mono- or di-unsaturated fatty acids and mixtures of saturated and unsaturated fatty acids can be used. Preferably, phosphatidylethanolamines containing saturated or unsaturated fatty acids with carbon chain lengths in the range of C₁₀ to C20 are used. More preferably, suitable phosphatidylethanolamines include, but are not limited to, dimyristoyl-phosphatidylethanolamine (DMPE), dipalmitoyl-phosphatidylethanolamine (DPPE), dioleoyl-phosphatidylethanolamine (DOPE), distearoyl-phosphatidylethanolamine (DSPE), and mixtures thereof.

The PEG may be coupled together with a diacylglycerol (DAG) moiety. By “diacylglycerol” or “DAG” it is preferably understood a compound having 2 fatty acyl chains having both independently between 2 and 30 carbons bonded to the 1- and 2-position of glycerol by ester linkages. The acyl chains can be saturated or may have varying degrees of unsaturation.

Suitable acyl groups include, but are not limited to, lauroyl (C₁₂), myristoyl (C₁₄), palmitoyl (C₁₆), stearoyl (C₁₈), and/or icosoyl (C₂₀). Preferably, the two acyl chains are identical, more preferably the two acyl chains are both myristoyl (such as dimyristoyl), or both stearoyl (such as distearoyl).

The PEG may be coupled together with a dialkyloxypropyl (DAA) moiety. By “dialkyloxypropyl” or “DAA” it is preferably understood a compound having 2 alkyl chains which have independently between 2 and 30 carbons. The alkyl chains can be saturated or may have varying degrees of unsaturation. Preferably, the PEG-DAA conjugate is a PEG-didecyloxypropyl (C₁₀) conjugate, a PEG-dilauryloxypropyl (C₁₂) conjugate, a PEG-dimyristyloxypropyl (C₁₄) conjugate, a PEG-dipalmityloxypropyl (C₁₆) conjugate, or a PEG-distearyloxypropyl (C₁₈) conjugate.

When the PEG is coupled, it preferably has an average molecular weight of 750 or 2,000 daltons.

Preferably, the terminal hydroxyl group of the PEG is substituted with a methyl group, thereby creating a terminal methoxy end group.

The component capable of reducing aggregation of the composition may also be chosen between other hydrophilic polymers other than PEG. Examples of suitable hydrophilic polymers other than PEG include, but are not limited to, polyglycerol, polysarcosine, polyphosphotriesters, polyvinylpyrrolidone, polymethyloxazoline, polyethyloxazoline, polyhydroxypropylmethacrylamide, polymethacrylamide and polydimethylacrylamide, polylactic acid, polyglycolic acid, and derivatized celluloses such as hydroxymethylcellulose or hydroxyethylcellulose, and mixtures thereof, preferably polyglycerol, polyvinylpyrrolidone, polymethyloxazoline, polyethyloxazoline, polyhydroxypropylmethacrylamide, polymethacrylamide and polydimethylacrylamide, polylactic acid, polyglycolic acid, and derivatized celluloses such as hydroxymethylcellulose or hydroxyethylcellulose, and mixtures thereof.

The component capable of reducing aggregation of the composition may also be chosen in the poloxamer family. By “poloxamer” it is preferably understood well-known class of non-ionic triblock copolymers composed by a central hydrophobic chain of polyoxypropylene (PPO) surrounded by two hydrophilic chains belonging to the polyethylene oxide (PEO) family [PEO-PPO-PEO]. These poloxamers can be linear or X-shaped, those latest being known as poloxamines. Examples of poloxamers include, but are not limited to, the meroxapols, also known as “reverse poloxamers” in the literature, which present a hydrophilic block based on a polyethylene oxide polymer, surrounded by two hydrophobic blocks, based on polypropylene oxide polymers. Poloxamers can be named after their manufacturer's commercial name, such as “Pluronic” “Symperonic” or “Tetronic”. For example, poloxamers suitable to be used as com-ponent able to reduce aggregation of the composition might be chosen between F127, F108, F68, F87, P123, L61, L64, F88 or F98.

The poloxamer may be associated with another component capable of reducing aggregation such as polyethylene glycol (PEG)-modified lipids (PEG-modified lipids) and/or polyethylene glycol (PEG) optionally substituted, conjugated and/or coupled as previously defined.

Examples of monosialoganglioside (Gml) include, but are not limited to, ganglioside GD1b, GQ1b, GT1b, GD1a, GM4 and/or or GD3.

Examples of polyamide oligomers (PAO) include, but are not limited to, polyamide 6 dimer, polyamide 6 trimer, polyamide 6 tetramer, polyamide 6 cyclic dimer, polyamide 6 cyclic trimer, and/or polyamide 6 cyclic tetramer.

The composition may also include one or more sterols or sterol derivatives, such as cholesterol and/or derivatives thereof. Examples of cholesterol derivatives include, but are not limited to, polar analogues such as 5C-cholestanol, 5C-coprostanol, cholesteryl-(2-hydroxy)-ethyl ether, cholesteryl-(4′-hydroxy)-butyl ether, and 6-ketocholestanol, non-polar analogues such as 5C-cholestane, cholestenone, 5C-cholestanone, 5C-cholestanone, and cholesteryl decanoate, and mixtures thereof. Preferably, the cholesterol derivative is a polar analogue such as cholesteryl-(4′-hydroxy)-butyl ether.

The composition may further include one or more lipids bearing a targeting ligand.

Examples of targeting ligand conjugatable with lipids include, but are not limited to, any peptides, proteins, antibodies, sugar, aptamers, small organic and inorganic molecules for examples RGDF peptides, A54 peptides, cRGD, Lyp-1 peptide, hEGF ligands, Anti-CD44 antibody, HIV trans-activating transcriptional activator peptide, Ega1 nanobodies, hyaluronic acid, galactose, mannose, transferrin, folate, and mixtures thereof.

Examples of lipids able to conjugate with targeting ligands include but are not limited to phospholipids such as dimyristoyl-phosphatidylethanolamine (DMPE), dipalmitoyl-phosphatidylethanolamine (DPPE), dioleoyl-phosphatidylethanolamine (DOPE), distearoyl-phosphatidylethanolamine (DSPE), and mixtures thereof or sterols such as cholesterol, 5C-cholestanol, 5C-coprostanol, cholesteryl-(2-hydroxy)-ethyl ether, cholesteryl-(4′-hydroxy)-butyl ether, and 6-ketocholestanol, and mixtures thereof.

The composition may further include one or more active ingredients such as nucleic acid(s), polysaccharide(s), lipid(s), polynucleotide(s), morpholino oligonucleotide(s), aptamer(s), protein(s), peptide(s), polypeptide(s), peptoid(s), small organic or inorganic molecule(s), drug(s) or any compounds of pharmaceutical interest, antigen(s), or mixtures thereof, to form picoparticles, microparticles, nanoparticles, liposomes, lipoplexes or micelles with the lipid of formula (I) as previously defined.

More particularly, examples of drug(s) or any compounds of pharmaceutical interest include, but are not limited to, folate or sodium valproate, antibodies, antigens, lymphokines, interleukins, necrosis and apoptosis factors, interferons, growth factors, tissue plasminogen activators, factor VIlIc, erythropoietin, insulin, calcitocin, thymidine kinase, and combination thereof.

The composition may further include adjuvants capable of specifically targeting a determinant at the surface and/or inside the cells. These adjuvants can be covalently or non-covalently attached to the lipid of formula (I) or to any other component contained in the composition comprising the lipid of formula (I).

Examples of adjuvant capable of specifically targeting a determinant at the surface and/or inside the cells include, but are not limited to, ligands of receptors expressed at the surface of the target cells, for example a sugar, folate, transferrin, insulin, hormone, prostaglandins, peptide, antibody, nanobody, metabolite, vitamins or any other molecule which can recognize an extracellular receptor, or an element of intracellular vectorization for targeting specific compartments such as the mitochondria, nucleus or cytoplasm, such as for example a nuclear or cytoplasm or mitochondrial localization signal such as for example a sugar, a ligand or a ligand fragment, a hormone, a peptide or polypeptide, an antibody (such as polyclonal antibodies, monoclonal antibodies, antibody fragments, humanized antibodies, recombinant antibodies and recombinant human antibodies), a protein, cytokines, growth factors, apoptotic factors, a metabolite, vitamins, an aptamer, differentiation-inducing factors, cell-surface receptors and their ligands, hormones, or any other molecule which can recognize an extracellular receptor, and mixtures thereof. The adjuvant can also be a fluorophore such as rhodamine, fluorescein or biotin.

The composition may further include signalling molecules and molecules that may be involved in the signalling pathways inside of the target cells.

The lipids of formula (I) as previously defined may be combined with other lipid compounds, polymers (synthetic or natural), surfactants, cholesterol, carbohydrates, saccharides, glycerol, cyclodextrins, magnetic crystal or particles, particles based on organic or inorganic compounds, histones, deoxycholic acid, proteins or combination thereof, to form the lipid particles as previously defined. Thus, the composition as previously defined may also comprise other lipid compounds, polymers (synthetic or natural), surfactants, cholesterol, carbohydrates, saccharides, glycerol, cyclodextrins, magnetic crystal or particles, particles based on organic or inorganic compounds, histones, deoxycholic acid, proteins or combination thereof.

The present invention also relates to process for preparing the composition as previously defined.

For example, the process for preparing the composition as previously defined may comprise the steps of:
preparing an organic or aqueous phase by dissolving one or more lipids of the present invention, preferably tunable lipids of the present invention, one or more neutral neutral lipids as previously defined, one or more components capable of reducing aggregation of said composition as previously defined, and optionally one or more sterol derivatives as previously defined, and optionally one or more lipids bearing a targeting ligand as previously defined, and optionally additional components as previously defined. When an organic phase is prepared, the chosen organic solvent must be miscible with water and may include, but not limited to, ethanol, tetrahydrofuran and/or acetonitrile;
preparing an organic or aqueous phase comprising one or more active ingredients as previously defined. Preferably, the pH of this organic or aqueous phase is controlled to be inferior to the pKa of the lipid of formula (I) as previously defined, preferably of the tunable lipid of formula (I) as previously defined;
mixing both phases to form of the composition comprising lipid particles encapsulating one or more active ingredients to be delivered in a lipidic architecture.

Suitable physical methods enabling a correct mixture phases include, but are not limited to, microfluidic mixing, microinjection, extrusion, T-mixing flow focusing, pressure-driven flow focusing, microfluidization, sonication, and combination thereof.

Advantageously, the nitrogen-containing portion of the lipids of formula (I) as previously defined, preferably of the tunable lipids of the present invention, may be used to complex the active ingredient, thereby enhancing their delivery and preventing their degradation.

In particular, the lipids of formula (I) as previously defined, preferably the tunable lipids of formula (I) as previously defined, are particularly attractive for delivering active ingredients for several reasons: they contain nitrogen-based groups for interacting with the active ingredient to be delivered such as DNA, RNA, or other polynucleotides, and other negatively charged active ingredients, such as negatively-charged drugs of interest like folate or sodium valproate.

They are also extremely useful for buffering the pH, for causing endo-osmolysis, and for protecting the agent to be delivered. Furthermore, they can be synthesized from commercially available starting materials, and/or they are pH responsive and can be engineered with a desired pKa in the correct broad range (pKa varying from 4 to 6.9, preferably from 4.5 to 6.9).

Thus, the lipid of formula (I) and/or the lipid particles as previously defined may be used to protect and deliver an active ingredient to a target site of interest (such as cell, tissue and/or organ).

The active ingredient to be delivered by the picoparticles, microparticles, nanoparticles, liposomes, lipoplexes or micelles may be in the form of a gas, liquid, or solid.

Preferably, the lipids of formula (I) as previously defined are biocompatible and biodegradable, and the formed picoparticles, microparticles, nanoparticles, liposomes, lipoplexes or micelles as previously defined are also biodegradable and biocompatible and may be used to provide controlled, sustained release of the active ingredient to be delivered.

The composition as previously defined may optionally be combined with a pharmaceutical excipient to form a pharmaceutical composition.

Thus, another object of the present invention relates to a pharmaceutical composition comprising the composition as previously defined and a pharmaceutical excipient.

The present invention also relates to the first therapeutic use of the lipid of formula (I) as previously defined, of the lipid particles as previously defined and/or of the composition as previously defined.

In particular, the lipid of formula (I) as previously defined, the lipid particles as previously defined and/or the composition as previously defined can be used for treating and/or preventing and/or diagnosing a disease and/or a disorder.

Thus, the present invention also relates to the use of the lipid of formula (I) as previously defined, of the lipid particles as previously defined and/or of the composition as previously defined for the manufacture of a medicament.

The present invention also relates to the lipid of formula (I) as previously defined, the lipid particles as previously defined and/or the composition as previously defined for its use as a medicament.

The present invention also relates to a method for treating and/or preventing and/or diagnosing a disease and/or a disorder comprising the use of the lipid of formula (I) as previously defined, of the lipid particles as previously defined and/or of the composition as previously defined.

Examples of diseases and disorders include, but are not limited to, cancers, bacterial or viral infections, inflammatory and genetic disorders, and/or metabolic deficiencies such as diabetes.

The lipids of formula (I) as previously defined, the lipid particles as previously defined and/or the compositions as previously defined are particularly suitable for delivering an active ingredient such as nucleic acid(s), polysaccharide(s), lipid(s), polynucleotide(s), morpholino oligonucleotide(s), aptamer(s), protein(s), peptide(s), polypeptide(s), peptoid(s), small organic or inorganic molecule(s), drug(s) or any compounds of pharmaceutical interest, antigen(s), or mixtures thereof, to subject(s), organ(s), cell(s) and/or tissue(s).

The subject(s) may be human(s), animal(s) and/or organism(s) such as plant(s).

The tissues can be for example, but not limited to, muscle, connective, epithelial or nervous tissues. Methods to obtain samples from various tissues and methods to establish primary cell lines and immortalized cell lines are well known in the art (see for example Freshney, R. I. Culture of Animal Cells: A Manual of Basic Technique, Fifth Edition, 2005 Ed: John Wiley & Sons, Inc.).

Thus, another object of the present invention relates to the use of the lipids of formula (I) as previously defined, the lipid particles as previously defined and/or the compositions as previously defined as a vector for delivering an active ingredient such as nucleic acid(s), polysaccharide(s), lipid(s), polynucleotide(s), morpholino oligonucleotide(s), aptamer(s), protein(s), peptide(s), polypeptide(s), peptoid(s), small organic or inorganic molecule(s), drug(s) or any compounds of pharmaceutical interest, antigen(s), or mixtures thereof, to subject(s), organ(s), cell(s) and/or tissue(s).

The present invention relates to the lipids of formula (I) as previously defined, the lipid particles as previously defined and/or the compositions as previously defined for their use as a vector for delivering an active ingredient such as nucleic acid(s), polysaccharide(s), lipid(s), polynucleotide(s), morpholino oligonucleotide(s), aptamer(s), protein(s), peptide(s), polypeptide(s), peptoid(s), small organic or inorganic molecule(s), drug(s) or any compounds of pharmaceutical interest, antigen(s), or mixtures thereof, to subject(s), organ(s), cell(s) and/or tissue(s).

The lipids of formula (I) as previously defined, the lipid particles as previously defined and/or the compositions as previously defined can be used in vitro, ex vivo and in vivo.

The present invention also relate to a method for delivering an active ingredient such as nucleic acid(s), polysaccharide(s), lipid(s), polynucleotide(s), morpholino oligonucleotide(s), aptamer(s), protein(s), peptide(s), polypeptide(s), peptoid(s), small organic or inorganic molecule(s), drug(s) or any compounds of pharmaceutical interest, antigen(s), or mixtures thereof, to subject(s), organism(s), organ(s), cell(s) and/or tissue(s) comprising a step of contacting the lipids of formula (I) as previously defined, the lipid particles as previously defined and/or the compositions as previously defined with the active ingredient.

The method can be performed in vivo, ex vivo or in vitro.

The lipids of formula (I) as previously defined and/or the lipid particles as previously defined may be included in any pharmaceutical, cosmetic, veterinarian, diagnostic and/or research product compositions.

Thus, the composition as previously defined may be a pharmaceutical, cosmetic, veterinarian, diagnostic and/or research product composition.

Thus, the present invention also relates to the pharmaceutical, cosmetic, veterinarian, diagnostic and/or research product use of the composition as previously defined.

The lipids of formula (I) as previously defined and/or the lipid particles as previously defined may also be used for other purposes as well as, for example, for coating, additives, excipients and/or materials for bioengineering purposes.

Thus, another object of the present invention relates to the use of the lipids of formula (I) as previously defined and/or the lipid particles as previously defined as materials for coating, additives, excipients and/or materials for bioengineering purposes.

Examples of materials for bioengineering purposes include, but are not limited to, filtration membrane, synthetic biotissues, and/or hydrogels.

The present invention also relates to a method for transfecting cells with a nucleic acid and/or a protein and/or a peptide and/or a polysaccharide and/or a lipid and/or a small organic or inorganic molecule and/or any type of bioactive molecule using the lipid of formula (I) as previously defined, the lipid particle as previously defined and/or the composition as previously defined.

The method may comprise the step of contacting said cells with the lipid of formula (I) as previously defined, the lipid particle as previously defined and/or the composition as previously defined and a nucleic acid and/or a protein and/or a peptide and/or a polysaccharide and/or a lipid and/or a small organic or inorganic molecule and/or any type of bioactive molecule.

The present invention also relates to transfected cells obtained by the method.

The transfected cells may be used in cell therapy, preferably in cell gene therapy or CAR T-cell therapy.

The present invention also relates to the use of the lipid of formula (I) as previously defined, the lipid particle as previously defined and/or the composition as previously defined with a nucleic acid and/or a protein and/or a peptide and/or a polysaccharide and/or a lipid and/or a small organic or inorganic molecule and/or any type of bioactive molecule for transfecting cells.

The present invention also relates to the lipid of formula (I) as previously defined, the lipid particle as previously defined and/or the composition as previously defined, for transfecting cells with a nucleic acid and/or a protein and/or a peptide and/or a polysaccharide and/or a lipid and/or a small organic or inorganic molecule and/or any type of bioactive molecule

According to the present invention, any kind of cell culture material or cell culture methods can be used respectively in 2 dimensions (2D) or onto 3 dimensional (3D) supports to transfect said cells called “target cells”. Preferably, 2D devices are defined as support that allow cells to be cultured in an adherent way (cells adhere at the bottom of the well), in suspension way (cells remains floating into the culture medium), or in co-culture (2 or more different kinds of cell types separated or not with an insert) such as treated or non-treated cell culture plates and dishes (from 1536-wells to 4-well cell culture plates, 35 mm, 60 mm, 90 mm, 100 mm petri dishes) or cell flasks (such as 25 cm², 75 cm² or 150 cm²). The present invention may also be applied to 3D matrices such as hydrogel or solid 3D scaffolds of various compositions such as collagen, atelocollagen, glycosan, polystyrene, hyaluronic acid, polycaprolactone and/or polyethylene glycol used as cell culture material or in cell culture methods.

The lipids of formula (I) as previously defined, the lipid particles as previously defined and/or the compositions as previously defined can also be used in combination with molecules known to influence a transfection procedure. Examples of molecules known to influence a transfection procedure include, but are not limited to, polypeptides recombinant or not, liposomes, cationic lipids, cationic polymers such as polyethylenimine, protamine sulphate, polybrene, poly-L-lysine, DEAE Dextran, polylactic-co-glycolic acid, chitosans, amphiphilic polymers such as poloxamer, chemically modified or not, polyethylene glycol and derivatives, anionic polymers such as starch and hyaluronic acid, and combination thereof. These molecules known to influence a transfection procedure can be used before, during and/or after the transfection. Thus, these molecules known to influence a transfection procedure can be added to the cells from 1 week before to 4 weeks before and/or after the transfection, more preferentially from 1 day before to 7 days before and/or after the transfection.

The present invention also relates to the use of magnetic-based particles preferably magnetic-based nanoparticles such as iron-based nanoparticles, with the lipids of formula (I) as previously defined, the lipid particles as previously defined and/or the compositions as previously defined in in cellulo delivery of active ingredients as previously defined.

The present invention also relates to a method for in cellulo delivery of active ingredients as previously defined with magnetic-based particles, preferably magnetic-based nanoparticles such as iron-based nanoparticles, with the lipids of formula (I) as previously defined, the lipid particles as previously defined and/or the compositions as previously defined.

In case, where the lipids of formula (I) as previously defined, the lipid particles as previously defined and/or the compositions as previously defined are used in combination with magnetic-based particles, preferably magnetic-based nanoparticles, a permanent or oscillating or electro-mediated magnetic field can be applied to the experiment after the cells, the lipids of formula (I) as previously defined, the lipid particles as previously defined and/or the compositions as previously defined, and the magnetic-based nanoparticles have been put in contact. This step can last from 10 seconds to 96 h, preferentially from 1 minute to 48 h, and more preferentially from 5 min to 4 h.

Preferably, the composition as previously defined further includes a nucleic acid as active ingredient to form a nucleic acid-lipid particle, preferably nucleic acid-lipid nanoparticle, with the lipid of formula (I) as previously defined.

The nucleic acid-lipid nanoparticle can also comprise other active ingredient as previously defined such as drug(s) or any compounds of pharmaceutical interest as previously defined. The active ingredient of the nucleic acid-lipid particle can be used as a template for producing one or several proteins of interest.

The nucleic acid can encode in an effective manner for a polypeptide of pharmaceutical interest, which, during its expression in the host cell, makes it possible to remedy a malfunction of the receiving organism.

As a result, the composition of the present invention can be used in in vitro and in vivo research and development or in in vivo and ex vivo gene therapy and/or nucleic acid-based therapy and/or cell therapy.

The nucleic acid can also encode in an effective manner for a polypeptide capable of producing an immune response against it in humans or animals or of inducing an immune response.

As a result, the composition of the present invention finds a particular application in gene therapy and/or cell therapy, for example in the field of vaccines, and of immunotherapy, in particular for treating or preventing cancers or bacterial or viral infections.

Thus, the present invention also relates to the composition as previously defined for its use in gene therapy and/or nucleic acid-based therapy and/or cell therapy, for example in the field of vaccines and of immunotherapy.

Examples of cell therapy include, but are not limited to, cell gene therapy and/or CAR T-cell therapy.

In particular, the present invention relates to the composition as previously defined for its use for treating and/or preventing cancers, bacterial or viral infections, inflammatory and genetic disorders, and metabolic deficiencies such as diabetes.

The present invention also relates to the use of the composition as previously defined for treating or preventing cancers, bacterial or viral infections, inflammatory and genetic disorders, and metabolic deficiencies such as diabetes.

The present invention also relates to a method for treating or preventing cancers, bacterial or viral infections, inflammatory and genetic disorders, and metabolic deficiencies such as diabetes comprising administering the composition as previously defined.

The lipids of formula (I) as previously defined, the lipid particles as previously defined and/or the compositions as previously defined may be used for systemic applications, as they can exhibit extended circulation lifetimes following intravenous (i.v.) injection, they can accumulate at distal sites (including sites physically separated from the administration site).

The lipids of formula (I) as previously defined, the lipid particles as previously defined and/or the compositions as previously defined may be used for topical, cutaneous, oral, rectal, vaginal, parenteral, intranasal, intravenous, intramuscular, intracerebral, sub-cutaneous, intraocular, trans-dermal, intratracheal, intravitreal and/or intraperitoneal administration, preferably for topical, cutaneous, oral, rectal, vaginal, parenteral, intranasal, intravenous, intramuscular, sub-cutaneous, intraocular, trans-dermal, intratracheal and/or intraperitoneal administration.

The present invention also relates to a kit comprising the lipid of formula (I) as previously defined, the lipid particles as previously defined and/or the composition as previously defined further comprising a nucleic acid and/or a protein and/or a peptide and/or a polysaccharide and/or a lipid and/or a small organic or inorganic molecule and/or any type of bioactive molecule, and optionally instructions of use.

### BRIEF DESCRIPTION OF FIGURES

FIG. 1 Reaction scheme for the synthesis of molecule I: i) Oleylamine, DIC, HOBt, DMF/CHCl₃; ii) Piperidine, DMF; iii) Dimethylglycine (DMG), pyBOP, DIPEA, DMF/DCM.

FIG. 2: Reaction scheme for the synthesis of molecule II i) N,N-dimethylaminobutyric acid, TCFH, Methyl-imidazole, MeCN/DCM.

FIG. 3: Reaction scheme for the synthesis of molecule III: i) Oleylamine, DIC, HOBt, DMF/CHCl₃; ii) Piperidine, DMF; iii) Dimethylaminobutyric acid (DMABA), DIC, HOBt, DMF/CHCl₃.

FIG. 4: Reaction scheme for the synthesis of molecule IV: i) Diethylaminoethanol, Discuccinimidyl carbonate, Et₃N, MeCN; ii) 1b, Et₃N, DCM.

FIG. 5: Reaction scheme for the synthesis of molecule V: i) Boc₂O, NaOH, H₂O/t-BuOH; ii) 3-aminopropylimidazole, HBTU, DIPEA, DMF/DCM; iii) TFA, DCM iv) linoleic acid, HBTU, DIPEA, DMF/DCM.

FIG. 6: Reaction scheme for the synthesis of molecule VI: i) 3-aminopropylimidazole, COMU, DIPEA, DMF; ii) TFA, DCM; iii) 2-hexyldecanoic acid, COMU, DIPEA, DMF; iv) Piperidine, DMF; v) linoleic acid, HBTU, DIPEA, DMF.

FIG. 7: Reaction scheme for the synthesis of molecule VII: i) p-TsOH, Toluene reflux, Dean-Stark; ii) N,N-dimethylglycine, TCFH, Methyl-imidazole, MeCN/DCM.

FIG. 8: Reaction scheme for the synthesis of molecule VIII: i) N,N-dimethylaminobutyric acid, TCFH, Methyl-imidazole, MeCN/DCM.

FIG. 9: Reaction scheme for the synthesis of molecule 9a: i) Phtalimide, PPh₃, DIAD, THF; ii) NH₂NH₂, 70 °C; iii) HCl conc., 70 °C.

FIG. 10: Reaction scheme for the synthesis of molecule IX: i) 9a, DIC, HOBt, Et₃N, DMF/CHCl₃; ii) Piperidine; iii) DMABA, pyBOP, DIPEA, DMF/DCM.

FIG. 11: Reaction scheme for the synthesis of molecule X: i) Oleic acid, HBTU, DIPEA, HOBt, DCM/DMF.

FIG. 12: Reaction scheme for the synthesis of molecule XI: i) Lauric acid, HBTU, DIPEA, HOBt, DMF.

FIG. 13: Reaction scheme for the synthesis of molecule XII i) 9a, DIC, HOBt, Et₃N, DMF/CHCl₃; ii) Piperidine; iii) DMABA, pyBOP, DIPEA, DMF/DCM.

FIG. 14: Reaction scheme for the synthesis of molecule XIII: i) DMG, HBTU, DIPEA, HOBt, DCM/DMF; ii)TFA, DCM; iii) DIC, HOBt, Et₃N, DCM/DMF; iv) TFA, DCM, RT; v) Linoleic acid, HBTU, HOBt DIPEA, DMF/DCM.

FIG. 15: Reaction scheme for the synthesis of molecule 14d: i) DIC, HOBt, Et₃N, DCM/DMF; ii) TFA, DCM; iii) N,N-dimethylaminobutyric acid, TCFH, Methyl-imidazole, MeCN/DCM; iv) TBAF, THF 0 °C; v) DIC, HOBt, Et₃N, DCM/DMF. vi) TFA, DCM.

FIG. 16: Reaction scheme for the synthesis of molecule XIV: i) Linoleic acid, HBTU, DIPEA, HOBt, DMF/DCM.

FIG. 17: Reaction scheme for the synthesis of molecule 15d: i) Boc₂O, EtOH, 97%; ii) Lauric Acid, DIC, HOBt, Et₃N, DCM/DMF; iii) Trifluoroacetic acid (TFA), DCM; iv) Fmoc-Glu(OH), DIC, HOBt, Et₃N, DCM/DMF; Piperidine, RT.

FIG. 18: Reaction scheme for the synthesis of molecule XV: i) N,N-dimethylglycine, HBTU, DIPEA, DMF.

FIG. 19: Reaction scheme for the synthesis of molecule XVI: i) (Boc)-Orn(Boc)OH, DIC, HOBt, Et₃N, DCM/DMF; ii) TFA, DCM; iii) DMABA, HBTU, DIPEA, DMF.

FIG. 20: Reaction scheme for the synthesis of molecule XVII: i) PyBOP, DIPEA, DCM/DMF; ii) 3-(dimethylamino)propane-1,2-diol, ppTs, Toluene reflux.

FIG. 21: Reaction scheme for the synthesis of molecule XVIII: i) NaBH₄ EtOH; ii) N,N-dimethylaminobutyric acid, TCFH, Methyl-imidazole, MeCN/DCM.

FIG. 22: Reaction scheme for the synthesis of molecule XIX: i) 2-{[Dimethyl(2-methyl-2-propanyl)silyl]oxy}-1,3-propanediol, p-TsOH, Toluene/THF; ii) TBAF, THF, 0 °C; iii) Imidazole, MeCN; iv) TCFH, Methyl-imidazole, MeCN/DCM.

FIG. 23: Reaction scheme for the synthesis of molecule 20b: i) Boc₂O, NaOH, t-BuOH, H₂O; ii) (Z)-2-nonen-1-ol; DIC, HOBt, Et₃N, DCM/DMF; iii) TFA, DCM; iv) a-ketoglutaric acid, PyBOP, DIPEA.

FIG. 24: Reaction scheme for the synthesis of molecule XX: i) 2-{[Dimethyl(2-methyl-2-propanyl)silyl]oxy}-1,3-propanediol; ii) TBAF, THF, 0 °C; iii) N,N-dimethylaminobutyric acid, TCFH, Methyl-imidazole, MeCN/DCM.

FIG. 25: Reaction scheme for the synthesis of molecule XXI: i) NaBH₄ EtOH; ii) DSC, Et₃N, DCM; iii) TBDMSCI, Imidazole, DMF; iv) Et₃N, DCM; v) TBAF, THF.

FIG. 26:. Reaction scheme for the synthesis of molecule XXII: i)N,N-dimethylethylenediamine, TCFH, Methyl-imidazole, MeCN/DCM; ii) TFA, DCM; iii) 4,6-Heptadecadiynoic acid, HBTU, HOBt, DIPEA, DMF.

FIG. 27: Reaction scheme for the synthesis of molecule XXIII: i) 20a, COMU, DIPEA, DMF; ii) TFA, DCM; iii) Tetradecylamine, DIC, HOBt, Et₃N, DMF; iv) Piperidine, DMF; v) DMG, TCFH, 1-methylimidazole, MeCN/DCM.

FIG. 28: Reaction scheme for the synthesis of molecule XXIV: i) Dodecylamine, HOBt, DIC, DMF/CHCl₃. ii) Piperidine; iii) 20a, pyBOP, DIPEA, DMF; iv) Piperidine; v) DMABA, 1-methylimidazole, TCFH, MeCN.

FIG. 29: Reaction scheme for the synthesis of molecule XXV: i) Fmoc-Orn(Boc)-OH, HOBt, DIC, Et₃N, DMF/DCM; ii) Piperidine; iii) BocOrn(Boc)OH, HBTU, DIPEA, DMF; iv) TFA, DCM; v) Linoleic acid, pyBOP, DIPEA, DMF/DCM.

FIG. 30: Reaction scheme for the synthesis of molecule XXVI: i) 4-(1-pyrrolidinyl)-1-butanol, DCC, DMAP, DCM; ii) Piperidine, DMF; iii) BocOrn(Boc)OH, HBTU, DIPEA, DMF/DCM; iv) TFA, DCM; v) Linoleic acid, pyBOP, DIPEA, DMF.

FIG. 31: Reaction scheme for the synthesis of molecule XXVII: i) Ethanolamine, EDC HCl, HOBt, DMF; ii) TFA, DCM; iii) DMABA, HBTU, DIPEA, DMF; iv) Fmoc-Orn(Boc)-OH, DCC, DMAP, Et₃N; v) Piperidine, DMF; vi) Boc-Orn(Boc)-OH, HBTU, DIPEA, DMF; vii) TFA, DCM; viii) Linoleic acid, pyBop, DIPEA, DMF/DCM.

FIG. 32: Reaction scheme for the synthesis of molecule XXVIII; i) DIC, HOBt, Et₃N, DCM/DMF. ii) TFA, DCM; iii) Linoleic acid, HBTU, DIPEA, HOBt, DMF/DCM.

FIG. 33: Reaction scheme for the synthesis of molecule XXIX: i) DIC, HOBt, Et₃N, DCM/DMF; ii) TFA, DCM; iii) Linoleic acid, HBTU, DIPEA, HOBt DMF/DCM.

FIG. 34: Reaction scheme for the synthesis of molecule XXX: i) DIC, HOBt, Et₃N, DCM/DMF; ii) TFA, DCM; iii) BocOrn(Boc)OH, DIC, HOBt, Et₃N, DCM/DMF; iv) TFA, DCM; v) Linoleic acid, PyBOP, DIPEA, DMF.

FIG. 35: Reaction scheme for the synthesis of molecule 31a: i) 3-Butyl-2-hepten-1-ol, DCC, DMAP, DCM; ii) TFA, DCM.

FIG. 36: Reaction scheme for the synthesis of molecule XXXI; i) 31a, DIC, HOBt, DMF/CHCl₃; ii) Piperidine, DMF; iii) DMABA, HBTU, DIPEA, DMF.

FIG. 37: 1 Reaction scheme for the synthesis of molecule 32c i) Boc₂O, EtOH ii) 2-hexyldecanoic acid, DCC, DMAP, DCM iii) TFA, DCM iv) FmocGluOH, DIC, HOBt, DMF, CHC1₃ v) NaN₃, DMF, 50 °C.

FIG. 38: Reaction scheme for the synthesis of molecules XXXII, XXXIII, XXXIV i) DMABA, HBTU, DIPEA, DMF/DCM ii) Propylene Oxide, MeOH, 60 °C iii) DMG, HBTU, DIPEA, DMF/DCM.

FIG. 39:: Reaction scheme for the synthesis of molecules XXXV i) Hexadecylamine, DIC, HOBt, DMF/CHCl₃ ii) TFA, Et₃Si, DCM iii) 32b, COMU, DIPEA, DMF iv) Piperidine, DMF v) DMG, HBTU, DIPEA, DMF/DCM.

FIG. 40: Reaction scheme for the synthesis of molecules XXXVI, XXXVII i) Malic acid, EDC, Cl, HOBt, NEt₃, DMF/DCM ii) DMABA, EDC, DMAP, DCM iii) DMG, EDC, DMAP, DCM.

FIG. 41: Reaction scheme for the synthesis of molecule 38c i) Succinic anhydride, NEt₃, DCM/DMF ii) 2-hexyldecanol, DCC, DMAP, DCM iii) TFA, DCM iv) FmcoGluOH, DIC, HOBt, NEt₃, DMF/DCM v) NaN₃, DMF, 50 °C.

FIG. 42: Reaction scheme for the synthesis of molecules XXXVIII, XXXIX i) DMG, HBTU, DIPEA, DMF/DCM ii) Propylene Oxide, MeOH, 60 °C.

FIG. 43: Reaction scheme for the synthesis of molecules XL i) Succinic anhydride, DMAP, DCM/DMF ii) 32b, COMU, DIPEA, DMF iii) TFA, DCM iv) 40a, COMU, DIPEA, DMF v) Piperidine, DMF vi) DMG, HBTU, DIPEA, DMF.

FIG. 44: Reaction scheme for the synthesis of molecules XLI i) 3-(dimethylamino)-1,2-propanediol, 1₂, PPh₃, Imidazole, DCM ii) TFA, DCM iii) 32b, COMU, DIPEA, DMF iv) NaN₃, DMF v) Dodecanoic acid, PyBOP, DIPEA, DMF.

FIG. 45: A, B: Evaluation of LNPs mRNA formulation in a transfection procedure using a mRNA coding for GFP protein: HeLa cell line was transfected with a growing dose of mRNA (0.1-20 μg) formulated in lipid nanoparticles based of tunable lipids I and XIV, or complexed in the same range using a commercially available transfection reagent (Tr Reag). After 24 h incubation, % of GFP positive cells (A) and % alive and not permeabilized cells (B) were evaluated by flow cytometry.

FIG. 46: A,B: Evaluation of LNPs mRNA formulation in a transfection procedure using a mRNA coding for GFP protein: Jurkat T-cells cell line was transfected with a growing dose of mRNA (0.1-20 μg) formulated in lipid nanoparticles based of tunable lipids I and XIV, or complexed in the same range using a commercially available transfection reagent (Tr Reag). After 24 h incubation, % of GFP positive cells (A) and % alive and not permeabilized cells (C) were evaluated by flow cytometry.

FIG. 47: Evaluation of LNPs mRNA formulation in a transfection procedure using a mRNA coding for GFP protein: PANC-1 cancer cell line was transfected with a growing dose of mRNA (0.1-2 μg) formulated in lipid nanoparticles based of tunable lipids I, XXXIV, XXXV, XXXVIII, XXXVI. After 24 h incubation, % of GFP positive cells was evaluated by flow cytometry.

FIG. 48:: Evaluation of LNPs mRNA formulation in a transfection procedure using a mRNA coding for GFP protein: RAW264.7 macrophage cell line was transfected with a growing dose of mRNA (1-20 μg) formulated in lipid nanoparticles based of tunable lipids I, XXXIV, XXXV, XXXVIII. After 24 h incubation, % of GFP positive cells was evaluated by flow cytometry.

FIG. 49: Evaluation of LNPs mRNA formulation in a transfection procedure using a mRNA coding for GFP protein: THP-1 monocyte cell line was transfected with a growing dose of mRNA (1-20 μg) formulated in lipid nanoparticles based of tunable lipids I, XXXIV, XXXVI, XXXVIII. After 24 h incubation, % of GFP positive cells was evaluated by flow cytometry.

FIG. 50 A, B: Bioluminescence signal (A) 3 h after i.p. administration of 3 mRNA-LNPs formulation: F-Luc mRNA-LNP I, F-Luc mRNA LNP XIV and F-Luc mRNA LNP DOTAP in nude mice; Kinetics of bioluminescence (B) signal over 25 h after i.p. administration 2 mRNA-LNPs formulation:: F-Luc mRNA-LNP I and F-Luc mRNA LNP XIV in nude mice (dose equivalent to 10 μg mRNA).

FIG. 51 A, B, C, D: Relative gene expression from firefly luciferase in different organs using tunable lipids. A: relative gene expression in liver's tissues from mouse with F-Luc-mRNA I, F-Luc-mRNA VI and F-Luc-mRNA XIV IP 10 μg Luc-mRNA. B: relative gene expression in lungs' tissues from mouse with F-Luc-mRNA I, F-Luc-mRNA VI and F-Luc-mRNA XIV IP 10 μg Luc-mRNA. C: relative gene expression in kidneys' tissues from mouse with F-Luc-mRNA I, F-Luc-mRNA VI and F-Luc-mRNA XIV IP 10 μg Luc-mRNA. D: relative gene expression in spleen's tissues from mouse with F-Luc-mRNA I, F-Luc-mRNA VI and F-Luc-mRNA XIV IP 10 μg Luc-mRNA.

FIG. 52: Relative gene expression from firefly luciferase in different organs 6 hours after injection using tunable lipids: relative gene expression in lungs, liver's and spleen's tissues from mouse with F-Luc-mRNA I, F-Luc-mRNA XXXIII and F-Luc-mRNA XXXIV; 10 μg Luc-mRNA. after i.v. administration of 3 mRNA-LNPs formulations.

FIG. 53 A, B, C: Humoral response to OVA subunit vaccine (10 μg) in absence or presence of vaccine adjuvant (Alum 1×) and to mRNA(OVA)-LNPs (mRNA dose equals to 10 μg, based on tunable lipids I & XIV) upon sub-cutaneous prime boost injection (DO—14) in C57BL6J mice. A: monitoring of the total Anti-OVA IgG production over time; B: monitoring of the Anti-OVA IgG1 production over time; C monitoring of the Anti-OVA IgG2c production over time.

FIG. 54 A, B: Th1 Humoral biomarker (IgG2c/lgG1) and B: CD8+ T cell response to OVA subunit vaccine (10 μg) in absence or presence of vaccine adjuvant (Alum 1×) and to mRNA(OVA)-LNPs (mRNA dose equals to 10 μg, based on tunable lipids I & XIV) upon sub-cutaneous prime boost injection (DO—14) in C57BL6J mice.

FIG. 55: Evaluation of LNPs DNA formulation in a transfection procedure using a DNA coding for F-Luc protein: Jurkat T cell line was transfected with a growing dose of DNA (0.1-10 μg) formulated in lipid nanoparticles based on tunable lipids XI, or complexed in the same range using a commercially available transfection reagent (MTX reagent, OZ Biosciences). After 48 h incubation, luminescence of lysed transfected cells has been assessed using the Luciferase Assay Kit (OZ Biosciences).

FIG. 56: Confocal microscopy pictures of fluorescently labelled LNPs based on tunable lipid XI encapsulating Cy5-labelled NTsiRNA, after 4 h of contact with HEK-293 cells. Cell nucleus has been labelled using Hoechst dye, siRNA with cyanine 5, while LNPs have been labelled using DiO. Picture on the left displays internalization of the LNPs after 4 h exposure. Picture on the right displays siRNA-Cy5 uptake.

FIG. 57: Evaluation of LNPs siRNA formulation in a transfection procedure using a siRNA coding against GFP protein: HEK-293 cell line, modified to express stably GFP, was transfected with a growing dose of siRNA (5 nM-100 nM) formulated in lipid nanoparticles based of tunable lipids I and XI at two different N/P ratio (4 and 6), or complexed in the same range using a commercially available transfection reagent (Lullaby, OZ biosciences SAS). After 72 h incubation, Mean of GFP fluorescence was evaluated by flow cytometry.

FIG. 58: Evaluation of LNPs siRNA formulation in a transfection procedure using a siRNA coding against GFP protein: THP-1 cell line, modified to express stably GFP, was transfected with a growing dose of siRNA (5 nM-100 nM) formulated in lipid nanoparticles based of tunable lipids I and XI at two different N/P ratio (4 and 6), or complexed in the same range using a commercially available transfection reagent (Lullaby, OZ biosciences SAS). After 72 h incubation, Mean of GFP fluorescence was evaluated by flow cytometry.

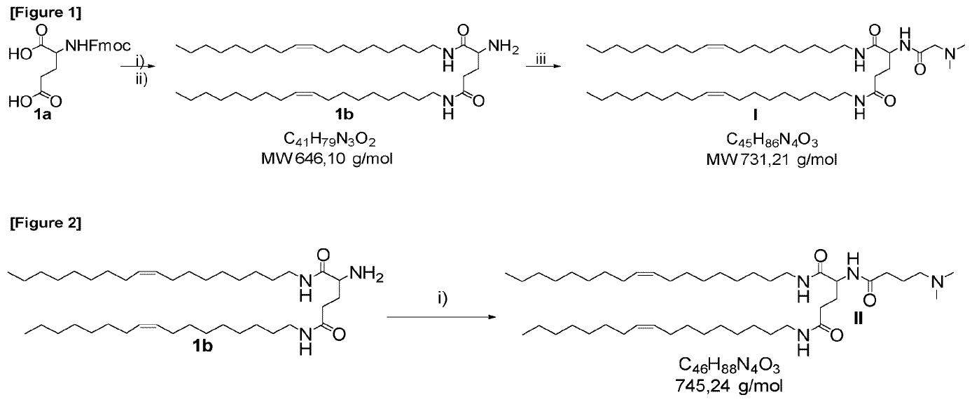

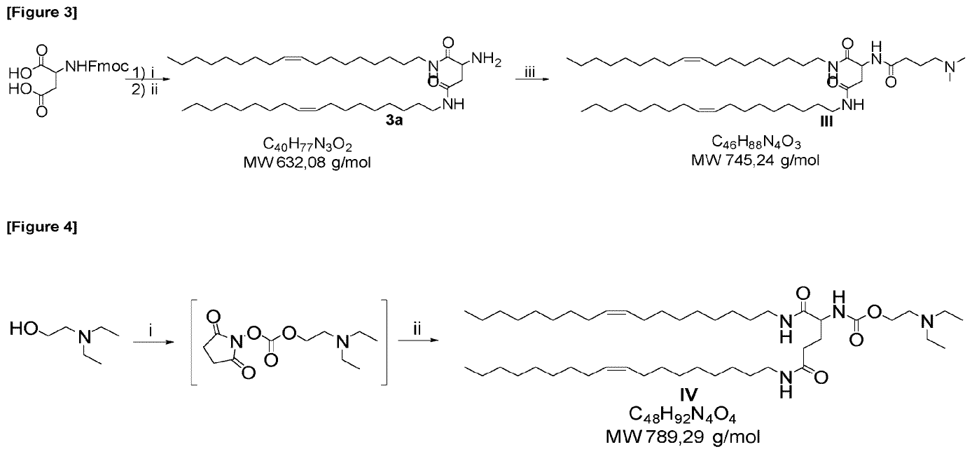

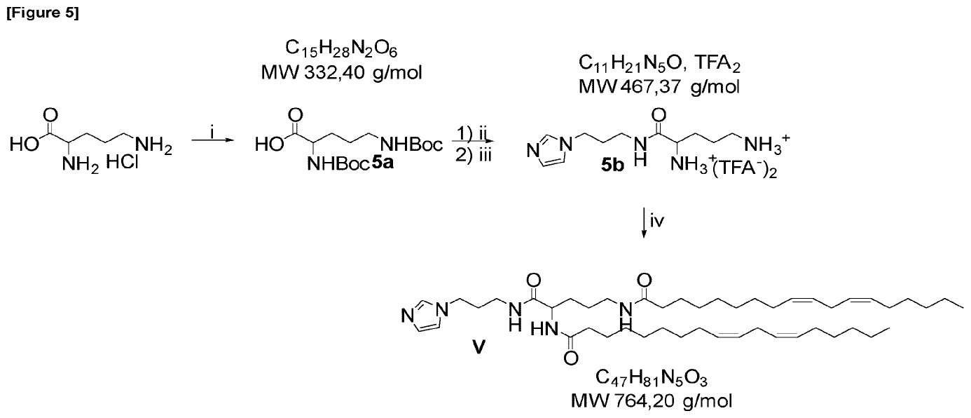

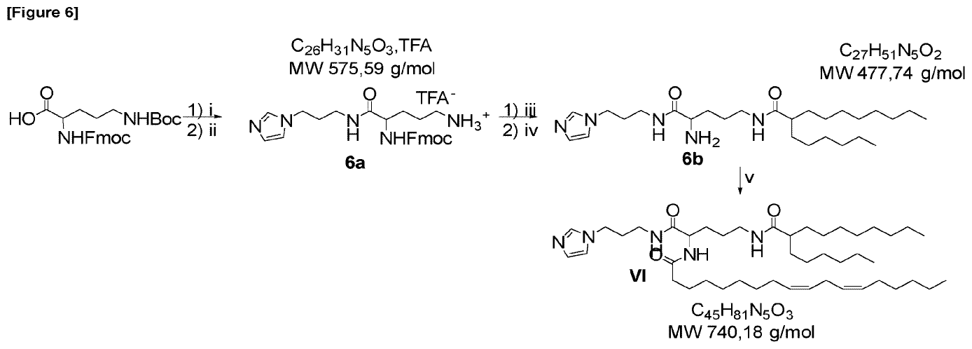

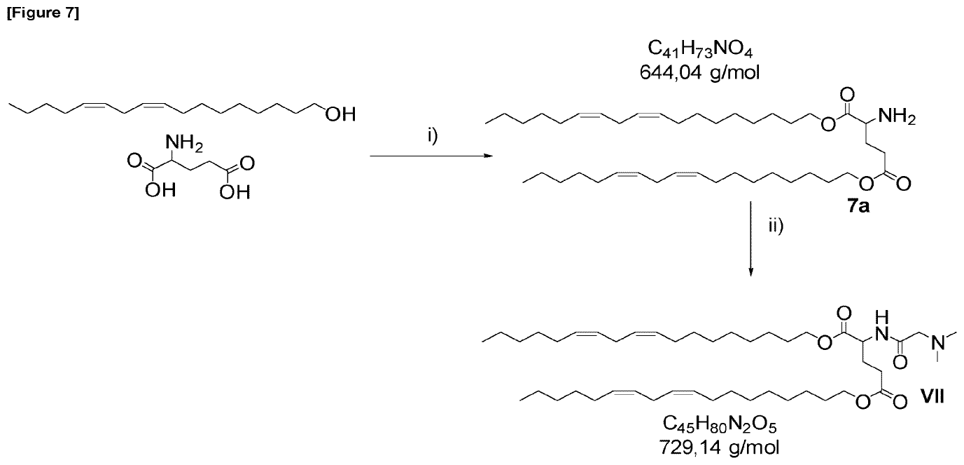

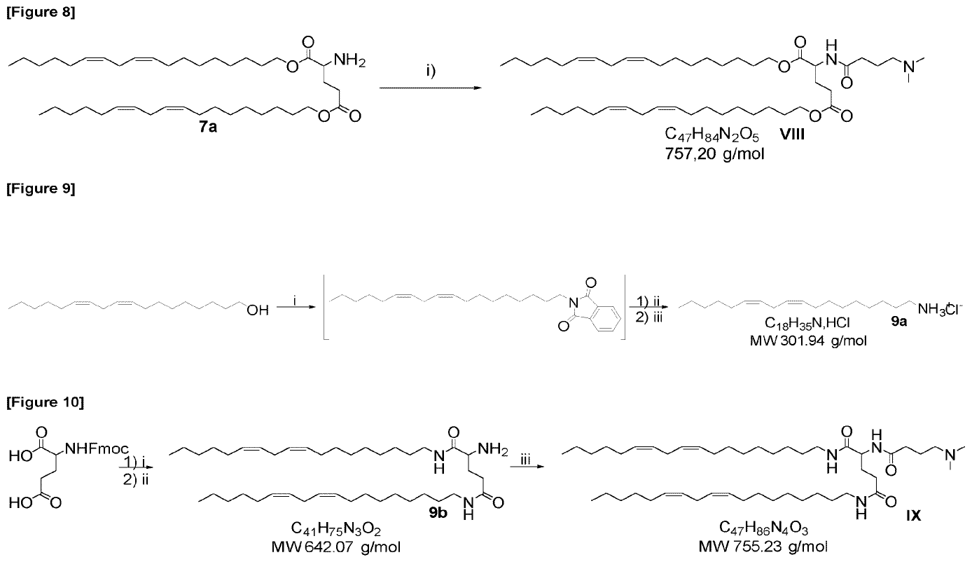

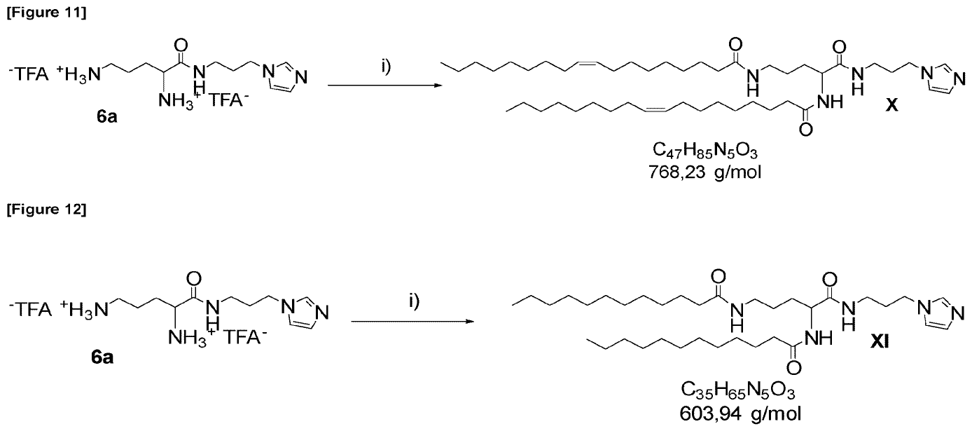

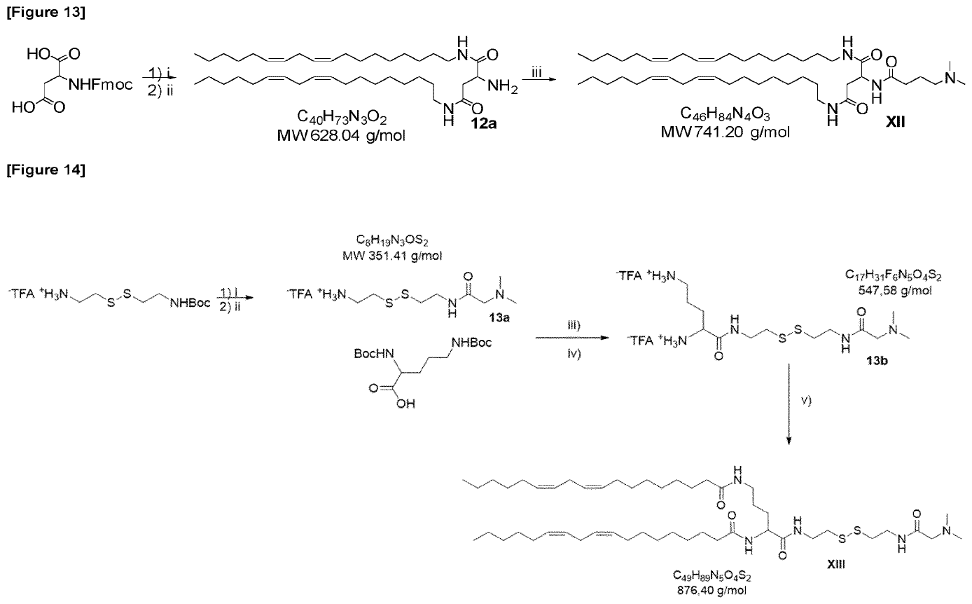

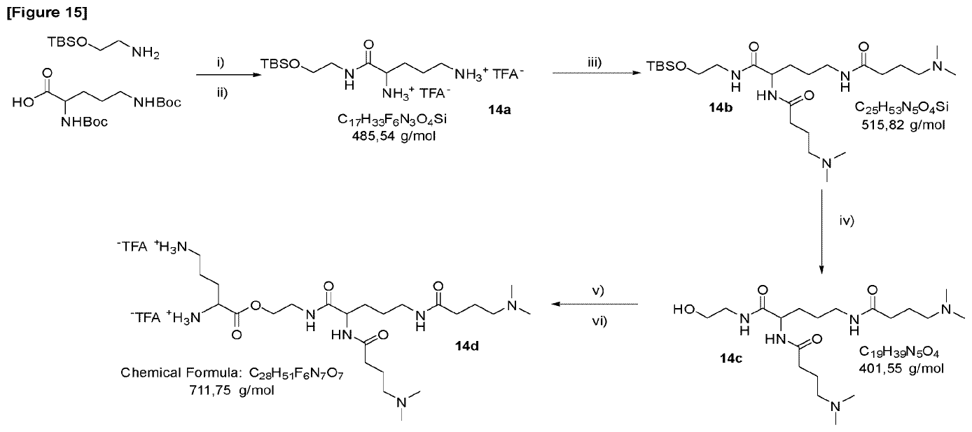

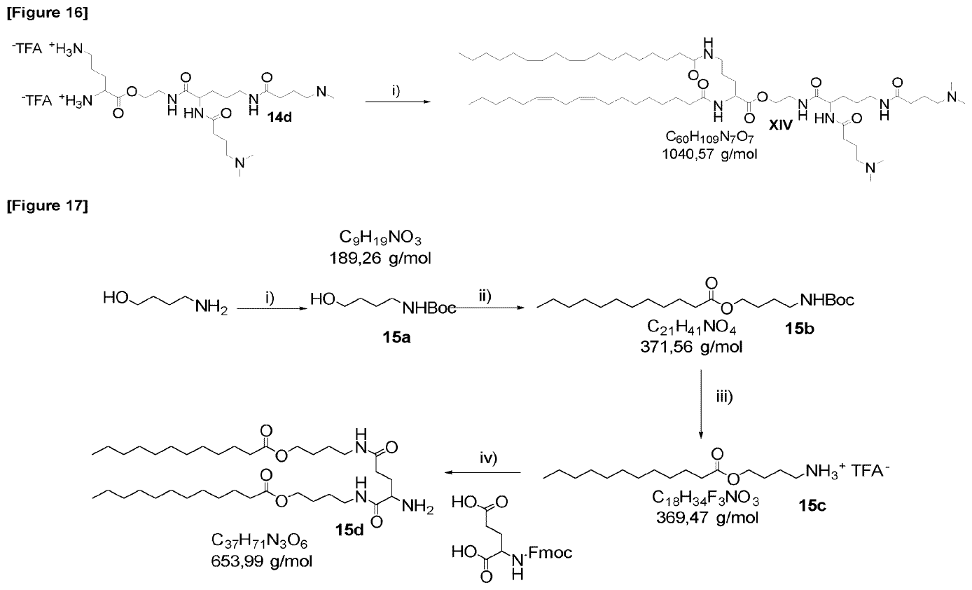

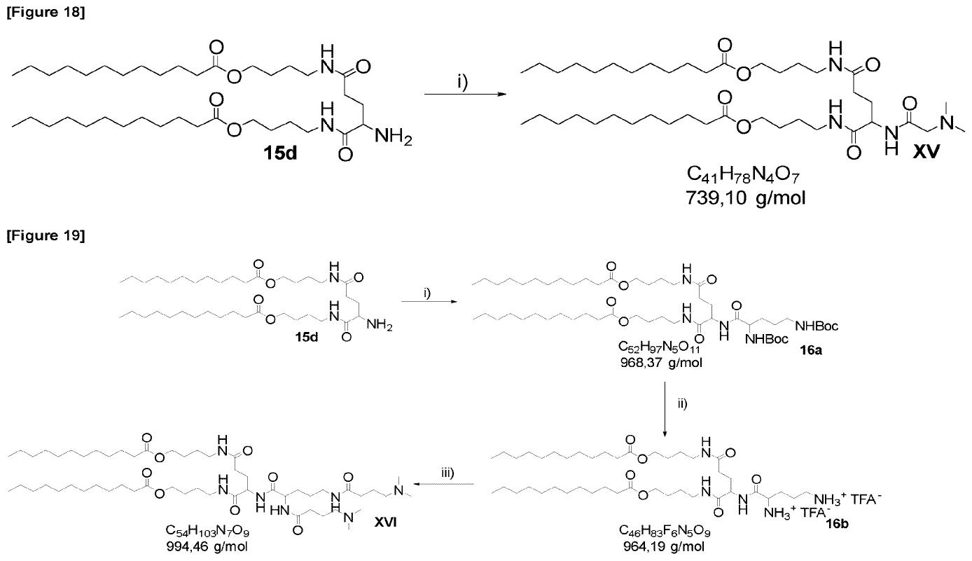

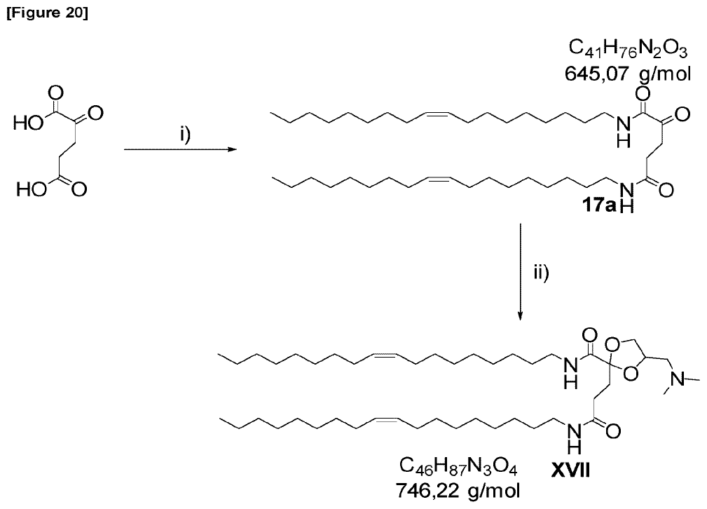

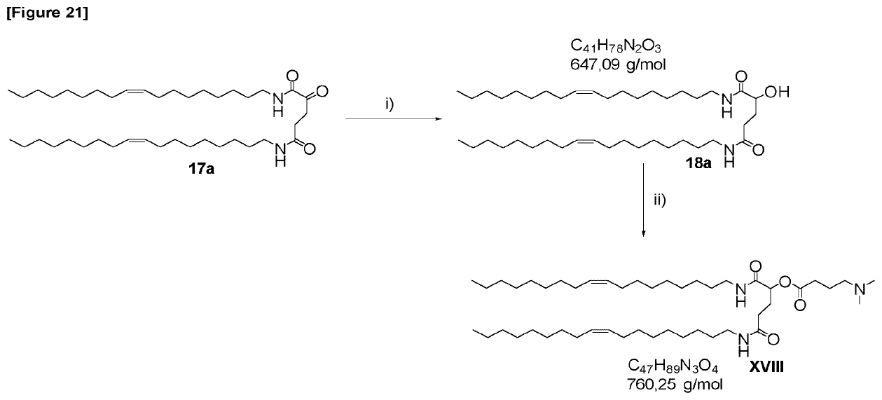

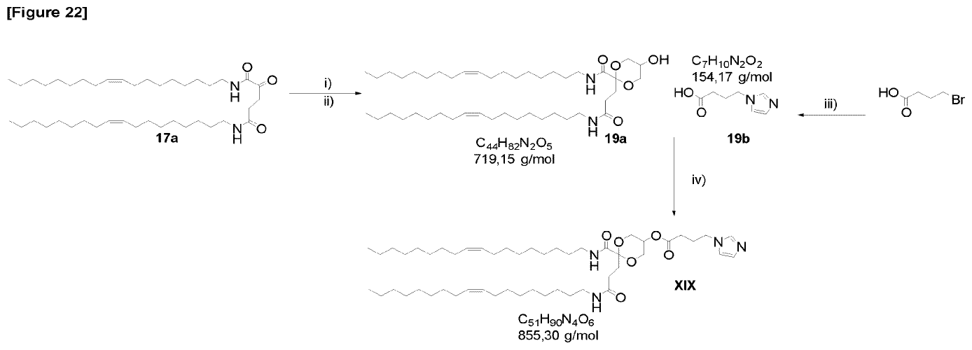

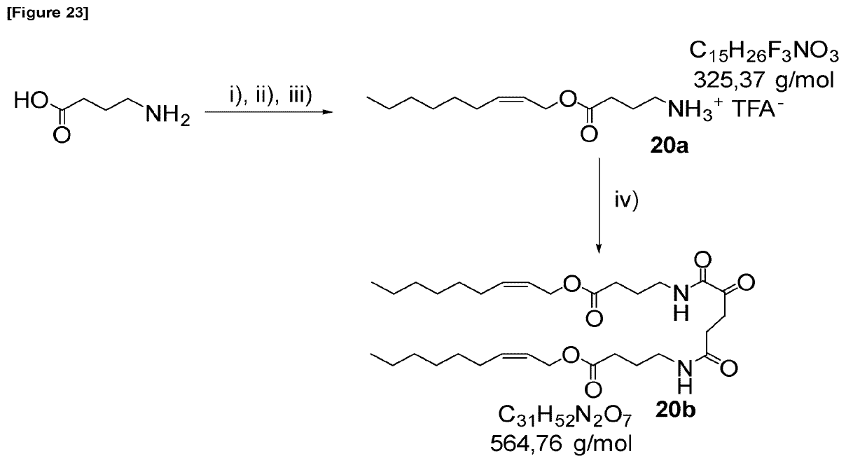

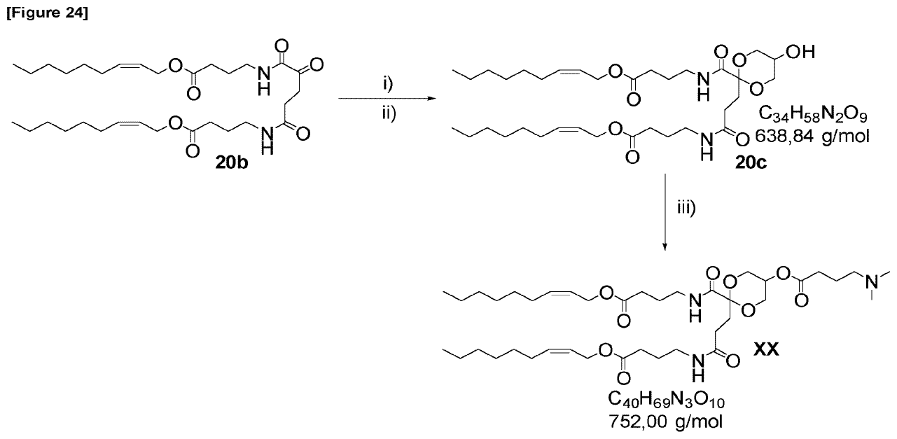

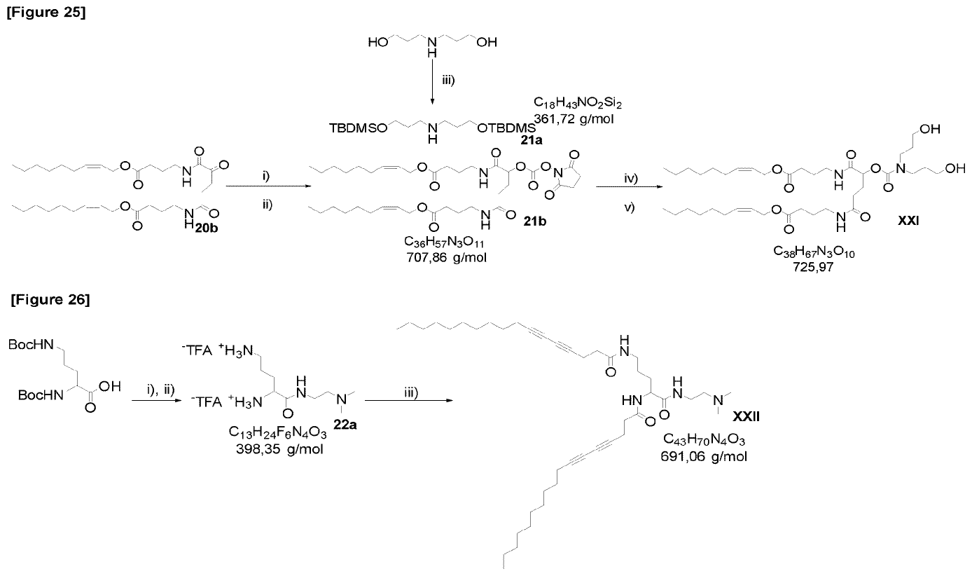

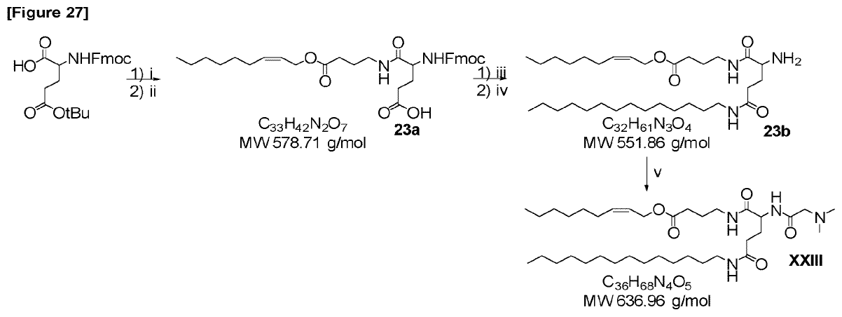

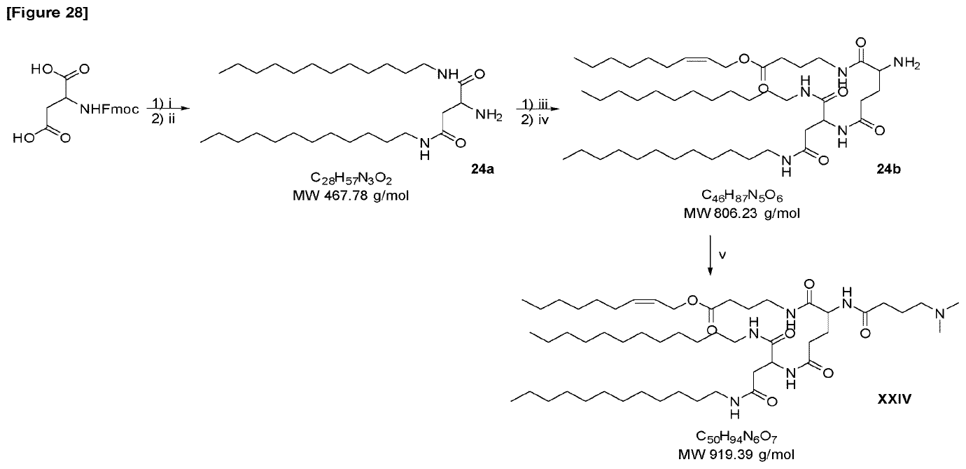

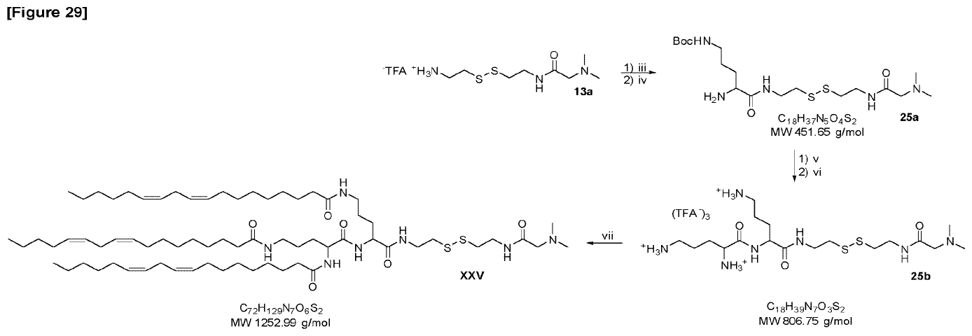

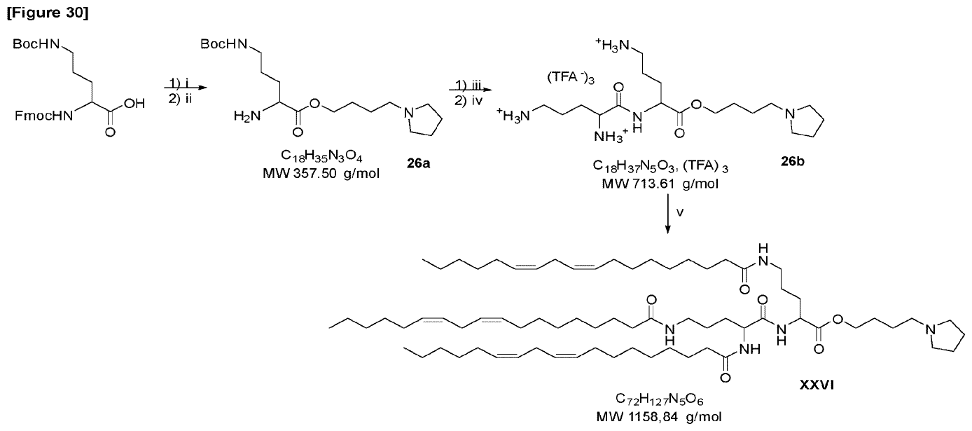

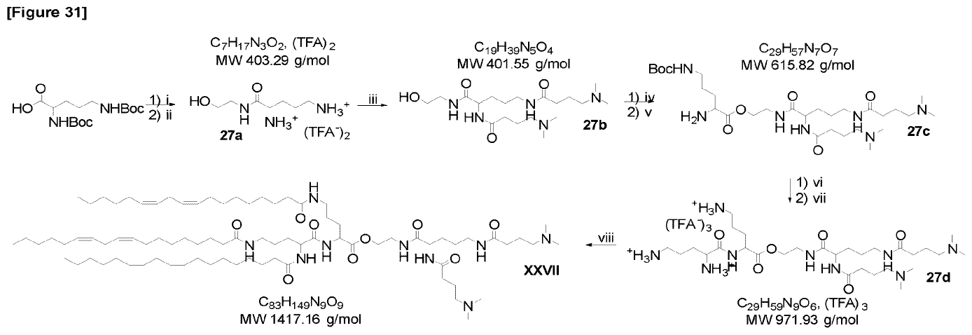

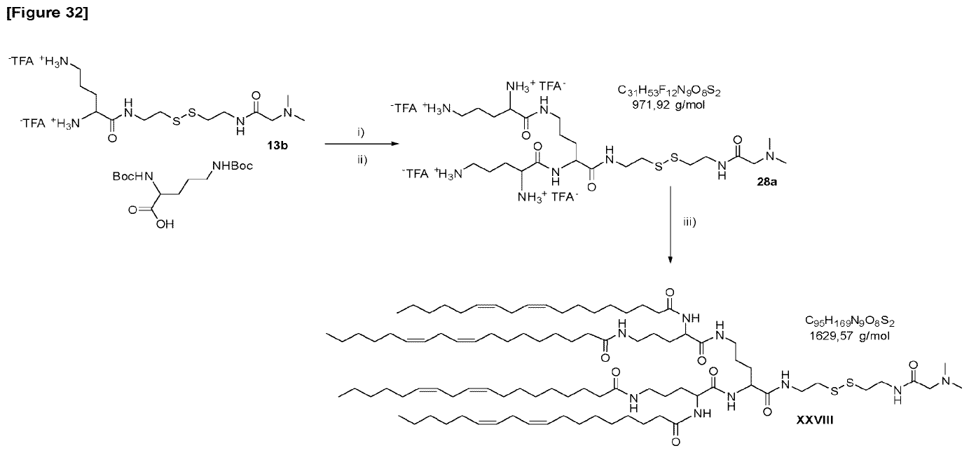

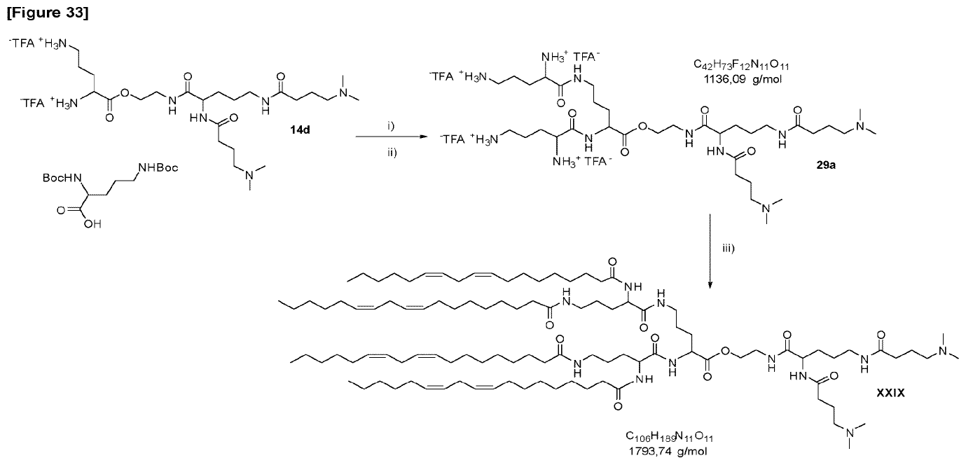

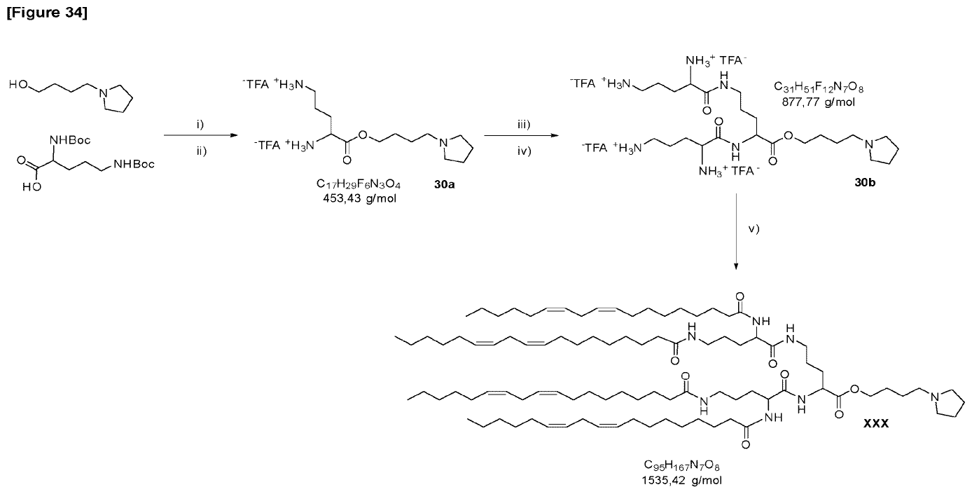

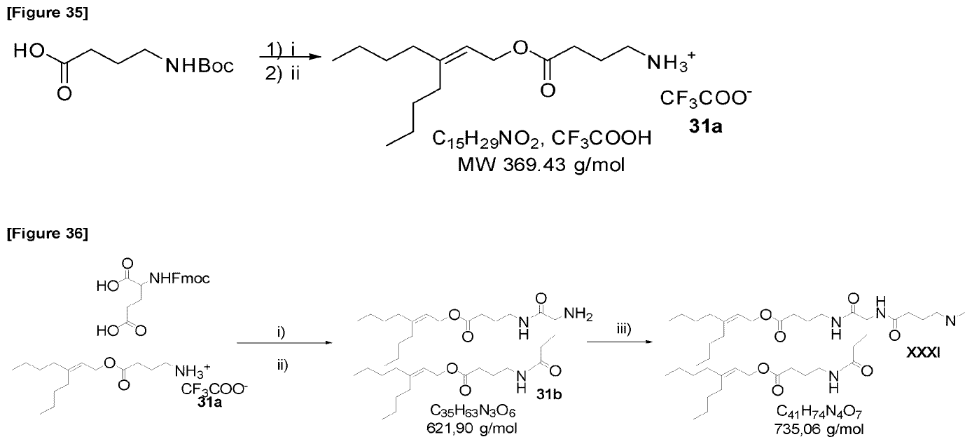

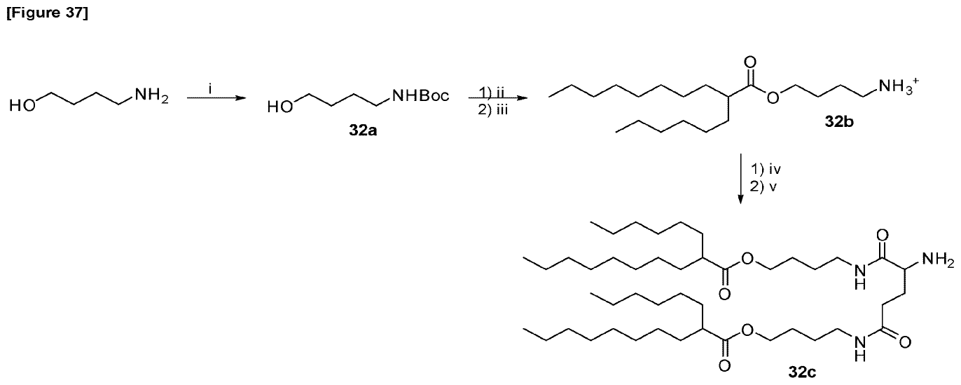

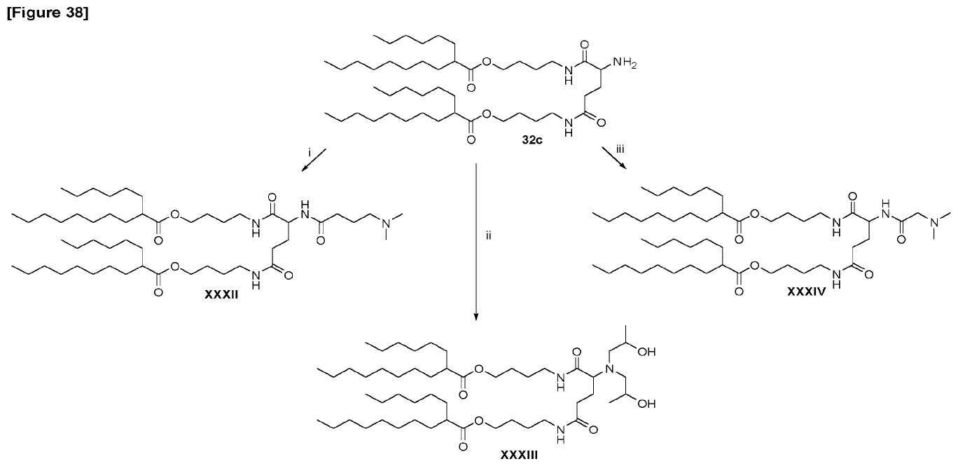

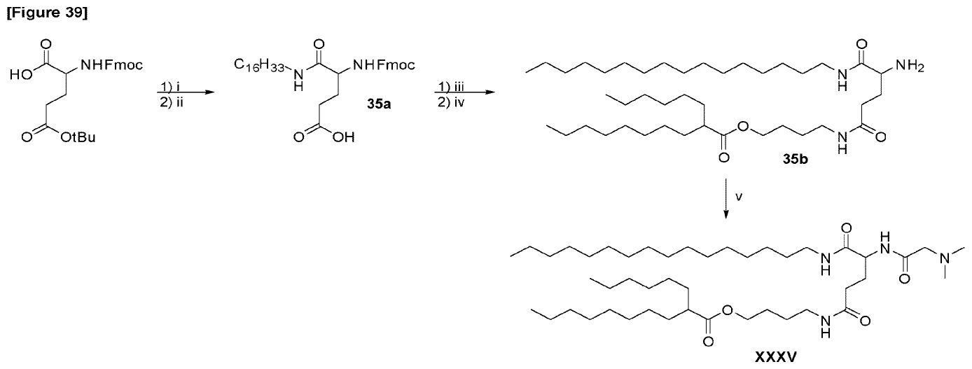

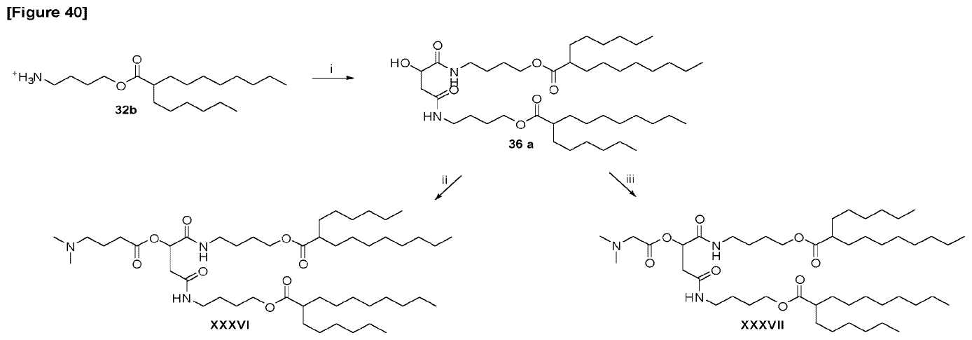

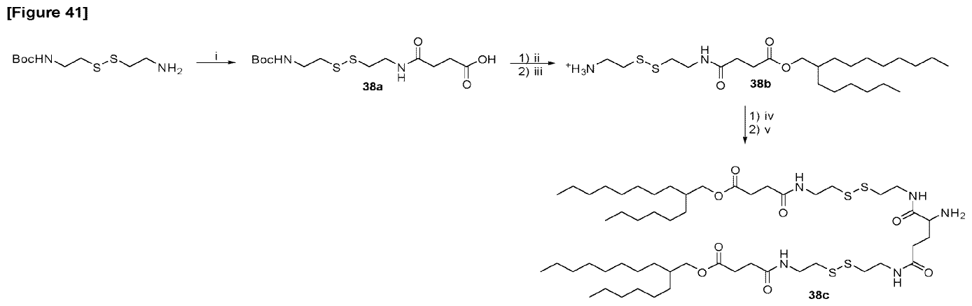

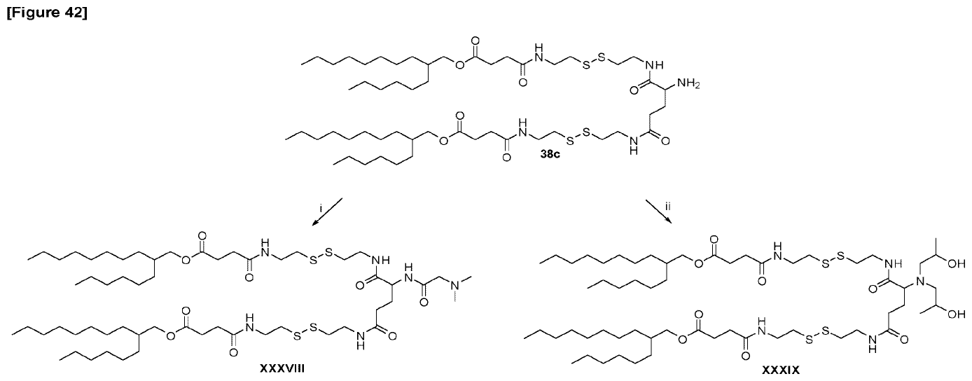

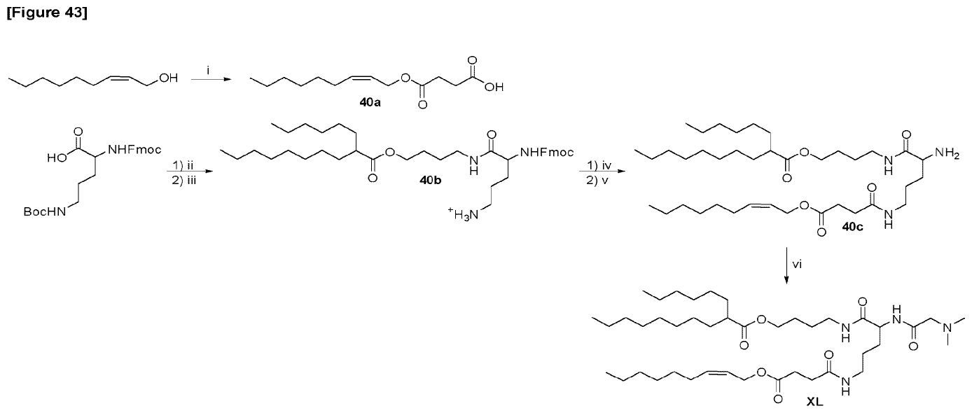

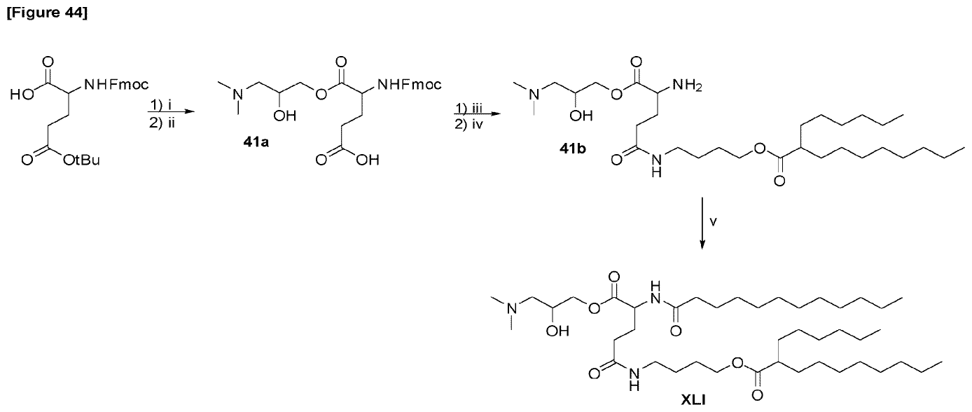

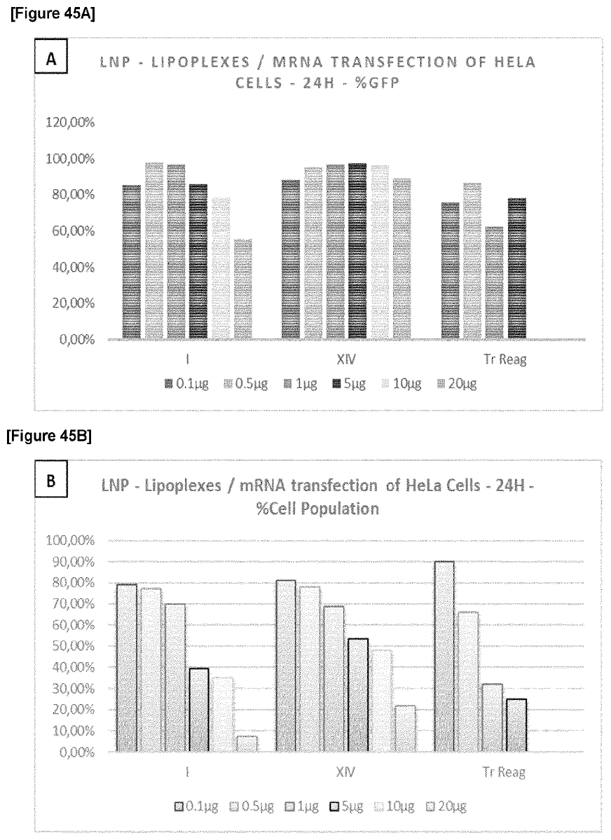

### EXAMPLES

### I—Material and Methods

### a) Materials

Most of the solvents and reagents have been obtained from VWR Prolabo (Briare, France), Sigma-Aldrich SA (St Quentin-Fallavier, France), TCl Europe N V (Zwijndrecht, Belgium), and Bachem Biochimie SARL (Voisin-le-Bretonneux, France). All reagents have been purchased with reagent grade have been used without any further purification procedure, unless otherwise stated. Amino-acids used in this work all derived from natural one (i.e., L-isomer), unless otherwise stated.

### b) Methods

Thin layer chromatographies (TLC) are carried out on aluminium plates covered with silica gel 60 Fasa (Merck). The compounds are developed under UV light (254 nm), with iodine, by immersion in a ninhydrin developer (0.2% in butanol) followed by a stage of heating at 150 °C in the case of the compounds possessing nitrogen-based functions, or by immersion in a cerium/concentrated molybdate developer: 90/10/15/1) followed by a stage of heating at 110 °C in the case of the Sulfur-containing compounds. The synthesized products are purified on chromatography columns on silica. The flash chromatography separations are carried out on silica gel 60 (230-400 mesh ASTM) (Merck). Particle size and zeta potential measurements: The mean hydrodynamic particle size and charge measurements were performed using Dynamic Light Scattering (DLS) and Laser Doppler Velocimetry (LDV), respectively, using Malvern Nano ZS instrument and DTS software (Malvern Instruments, UK) in a water of Grade 2. Various amounts of cationic poloxamers were diluted in 100 μl of water and mixed with an equal volume of the same water containing DNA. The measurements were carried out in automatic mode and the results are presented as mean+/−SEM, n=3. Each mean represents the average value of 30 measurements.

### Synthesis Molecule I

### 2-amino-N,N′-bis[(Z)-octadec-9-enyl]pentanediamide (1b)

Fmoc-glutamic acid (10 mmol; 3.69 g) is dissolved in 80 mL of anhydrous DMF under stirring and under an inert atmosphere. DIC (32 mmol; 4.96 mL), then HOBt (32 mmol; 4.32 g), in solution in 20 mL anhydrous DMF, are successively added to the reaction medium. The reaction is then stirred for 3 hours before oleylamine (30 mmol; 11.6 mL), in solution in 100 mL of anhydrous CHCl₃, is added dropwise to the reaction medium. After 36 hours, 30 mL of piperidine are added and the reaction medium is stirred for another 4 hours. The reaction is stopped and the reaction medium is evaporated to dryness under a rough vacuum. The residue is taken back in 100 mL of EtOAc and heated until dissolution. The solution is put in falcons at −20 °C. The formed precipitate is washed several times by centrifugation with cold EtOAc and Et2O and dried under high vacuum. A white solid is obtained with a yield of 93% (9.3 mmol; 6.0 g). TLC:Rf=0.4 (DCM:MeOH 95/5 (v/v)).

### 2-[[2-(dimethylamino)acetyl]amino]-N,N′-bis[(Z)-octadec-9-enyl]pentanediamide (1)

N,N′-dimethylglycine (0.28 mmol; 29 mg) is placed in a 25 mL flask under an inert atmosphere then dissolved in 2 mL of anhydrous DMF. DIPEA (0.63 mmol; 109 μL) and pyBOP (0.3 mmol; 156 mg) are added to the solution successively. The reaction medium is stirred under an inert atmosphere at ambient temperature for 15 minutes before 1b (0.25 mmol; 162 mg), in solution in 2 mL of anhydrous DMF and 2 mL of anhydrous DCM, is added to the reaction medium.

After 24 hours, the reaction is stopped and the reaction medium is evaporated to dryness under a high vacuum. The coupling product is then purified by flash chromatography on silica gel (elution gradient from 4 to 10% MeOH in DCM). In this way, the product is isolated in a yield of 34% (0.171 mmol; 125 mg). TLC:Rf=0.2 (DCM:MeOH 95/5 (v/v))

### Synthesis Molecule II

### 2-[4-(dimethylamino)butanoylamino]-N′—[(Z)-heptadec-9-enyl]-N—[(Z)-octadec-9-enyl]pentanediamide (II)

N,N-dimethylaminobutyric acid (DMABA, 40 mg, 0.23 mmoles) is dissolved using strong stirring under argon, in 4 mL of dry 2/1 (v/v) mixture solution of MeCN and DCM that already contains methylimidazole (74 μL, 0.92 mmoles). After complete dissolution, a solution of 2 mL of DCM containing compound 1b (0.25 mmoles, 161 mg) is added under agitation. The mixture is cooled down to zero degrees, and Chloro-N,N,N′,N′-tetramethylformamidinium hexafluorophosphate (TCFH) (98 mg, 0.35 mmoles) is then introduced in one portion. After addition, the reaction is let warmed to RT during 12 h before being concentrated. The obtained residue is then resuspended in 15 mL of ethyl acetate and washed twice with water and once with brine, dried on sodium sulphate, filtered and concentrated. The product is collected by flash chromatography on silica gel using a DCM/methanol mixture as eluent. A gradient has been used ranging from 98/2 to 90/10 (DCM/MeOH, v/v). 149 mg (0.2 mmoles, 85% yield) of a gluey solid are collected. TLC (DCM/MeOH: 95/5) rf: 0.3.

### Synthesis Molecule III

### 2-amino-N,N′-bis[(Z)-octadec-9-enyl]butanediamide (3a)

Compound 3a has been isolated from the coupling of Fmoc-Aspartic acid (2.8 mmol; 1 g) and oleylamine (8.4 mmol; 3.26 mL) using DIC (8.1 mmol; 1.26 mL), HOBt (9.25 mmol; 1.25 g) and then 8 mL of piperidine in DMF (30 mL) and CHCl₃ (30 mL) according to the procedure described for compound 1b. Reaction time: 96 h room temperature; Yield: 68% (1.9 mmol; 1.2 g, white solid; TLC:Rf=0.4 (DCM:MeOH 95/5 (v/v)).

### 2-[4-(dimethylamino)butanoylamino]-N,N′-bis[(Z)-octadec-9-enyl]butanediamide (III)

DMABA (0.25 mmol; 162 mg) is placed in a dry 25 mL flask under an inert atmosphere then dissolved in 2.5 mL of anhydrous DMF under stirring. DIC (0.75 mmol; 116 μL) and HOBt (0.75 mmol; 101 mg) are successively added to the reaction medium. The reaction medium is stirred under an inert atmosphere at ambient temperature for 3 hours before 3a (0.25 mmol; 158 mg), in solution in 2.5 mL of anhydrous DCM, is added to the reaction medium with triethylamine (0.88 mmol; 122 μL). After 48 hours, the reaction is stopped and the reaction medium is evaporated to dryness under a high vacuum. The coupling product is then purified by flash chromatography on silica gel (elution gradient from 4 to 10% MeOH in DCM). In this way the product is isolated in a yield of 12% (0.03 mmol; 22 mg). TLC:Rf=0.3 (DCM:MeOH 95/5 (v/v)).

### Synthesis Molecule IV

### 2-(diethylamino)ethyl-N-[4-[[(Z)-heptadec-9-enyl]amino]-1-[[(Z)-octadec-9-enyl]carbamoyl]-4-oxo-butyl]carbamate (IV)

N,N′-Disuccinimidyl carbonate (1.5 mmol; 384 mg) is added to a solution of diethylamino ethanol (1 mmol; 132 μL) in 5 mL of anhydrous MECN under stirring. After 5 minutes triethylamine (3 mmol; 420 μL) is added and the reaction medium is stirred under an inert atmosphere and at ambient temperature until total alcohol consumption. When the reaction is over, the solvent is evaporated and the product dried under high vacuum. The residue is taken back in 5 mL of anhydrous DCM while 1b (0.5 mmol; 323 mg) and triethylamine (2 mmol; 140 μL) are added to the reaction medium. After stirring at ambient temperature under an inert atmosphere for 12 hours, 10 mL of DCM are added and the organic layer is washed with an aqueous solution of NH₂Cl and brine. The organic layer is dried on MgSO₄ and evaporated to dryness under a high vacuum. The residue is purified by flash chromatography on silica gel (elution gradient from 2 to 8% MeOH in DCM). In this way, the product is isolated in a yield of 15% (0.08 mmol; 59 mg). TLC:Rf=0.3 (DCM:MeOH 95/5 (v/v)).

### Synthesis Molecule V

### 5-amino-2-(tert-butoxycarbonylamino)pentanoic acid/(Boc-Orn(Boc)OH (5a))

NaOH (24.7 mmoles; 987 mg) is dissolved in 25 mL of water in a 100 mL flask. A solution of ornithine hydrochloride (11 mmol; 1.9 g) in 8 mL t-BuOH is added to the reaction medium followed by Boc₂O (23.7 mmol; 5.2 g) also dissolved in 8 mL of t-BuOH. Boc₂O is added dropwise to the previous solution. After stirring at ambient temperature for 16 hours, 50 mL of petroleum ether is added and the aqueous layer is extracted 3 times with 50 mL ether. The organic layer is washed two times with NaHCO₃. The pH of the aqueous phases is acidified to 2 with concentrated HCl and extracted again 5 times with 100 mL of Et₂O. The organic extracts are washed with water, brine, dried on MgSO₄ and evaporated to dryness under high vacuum. The obtained oil is crystallized with petroleum ether and filtered, the filtrate is evaporated and crystallized also to give a white powder in a yield of 85% (20.1 mmol; 6.7 g).

5-((3-(1H-imidazol-1-yl)propyl)amino)-5-oxopentane-1,4-diaminium (5b) 3-aminopropylimidazole (2 mmol; 238 μL)) is placed in a dry 25 mL flask under an inert atmosphere then dissolved in 10 mL of anhydrous DMF under stirring. 2-(1H-benzotriazol-1-yl)-1,1,3,3-tetramethyluronium hexafluorophosphate (HBTU) (1.5 mmol; 569 mg) and DIPEA (2 mmol; 340 μL) dissolved in 20 mL DCM are successively added to the reaction medium. The reaction is then maintained under an inert atmosphere at ambient temperature for 15 minutes before compound 8a (1.5 mmol, 500 mg) in solution in 1 mL of anhydrous DCM and 3 mL of DMF, is added to the reaction medium. Reaction time: 48 h room temperature. The crude is taken back in 50 mL of EtOAc and washed 3 times with water and brine. The organic layer is dried on MgSO₄ and evaporated. The product is crystallized in petroleum ether, dried and is then dissolved in 60 mL DCM and 3 mL of trifluoroacetic acid is added to the reaction medium. The reaction is then maintained for one night under stirring at ambient temperature. The reaction medium is evaporated to dryness under a rough vacuum. The final residue is taken back four times, in succession, in DCM and four times in Et₂O then evaporated to dryness. The reaction is quantitative (1.5 mmoles, 701 mg).

### (9Z,12Z)—N-[5-(3-imidazol-1-yipropylamino)-4-[[(9Z,12Z)-octadeca-9,12-dienoyl]amino]-5-oxo-pentyl]octadeca-9,12-dienamide (V)

Molecule V has been isolated from the coupling of Linoleic acid (1.5 mmol, 468 μL) and compound 5b (0.6 mmol; 280 mg) using HBTU (1.5 mmol; 569 mg) and DIPEA (3 mmol; 509 μL) dissolved in 6 mL DCM and 4 mL of DMF according to the procedure described for the molecule 5b. Reaction time: 48 h room temperature; Purification: Silica gel chromatography gradient 98/2 to 94/6 (DCM/MeOH, v/v). Yield: 45% (206 mg, 0.27 mmol, TLC (DCM/MeOH: 90/10) Rf: 0.3).

### Synthesis Molecule VI

### 5-((3-(1H-imidazol-1-yl)propyl)amino)-4-((((9H-fluoren-9-yl)methoxy)carbonyl)amino)-5-oxopentan-1-aminium (6a)

Fmoc-Ornithine(Boc)-OH (1 mmol, 454 mg) is placed beforehand in a dry 50 mL flask under an inert atmosphere then dissolved in 10 mL of anhydrous DMF under stirring. 1-Cyano-2-ethoxy-2-oxoethylidenaminooxy)dimethylamino-morpholino-carbenium hexafluorophosphate (COMU 1.1 mmol; 471 mg) and DIPEA (2 mmol; 346 μL) are successively added to the reaction medium. The reaction is then maintained under an inert atmosphere and 0 °C for 15 minutes before 3-aminopropylimidazole (1.1 mmol; 131 μL) is added to the reaction medium under stirring. The solution is allowed to warm to ambient temperature under an inert atmosphere overnight. The reaction medium is evaporated to dryness under a rough vacuum. The crude is taken back in 50 mL of EtOAc and washed with 1M HCl, NaHCO₃ and brine. The organic layer is dried on MgSO₄ and evaporated. The product is used without further purification. TLC:Rf=0.4 (DCM:MeOH 90/10 (v/v)).

The product is taken back in 50 mL DCM and 2 mL of TFA are added. The reaction is then maintained for one night under stirring at ambient temperature. The reaction medium is evaporated to dryness under a rough vacuum. The final residue is taken back four times, in succession, in DCM and four times in Et₂O then evaporated to dryness. The reaction is quantitative; Yield 66% (2steps, 380 mg, 0.66 mmoles).

### N-[4-amino-5-(3-imidazol-1-ylpropylamino)-5-oxo-pentyl]-2-hexyl-decanamide (6b)

2-hexyldecanoic acid (1.5 mmol, 440 μL) is placed beforehand in a dry 50 mL flask under an inert atmosphere then dissolved in 15 mL of anhydrous DMF under stirring. COMU (1.5 mmol 642 mg) and DIPEA (3 mmol; 519 μL) are successively added to the reaction medium. The reaction is then maintained under an inert atmosphere and 0 °C for 15 minutes before 6a (570 mg), dissolved in 5 mL of anhydrous DMF, is added to the reaction medium under stirring. The solution is allowed to warm to ambient temperature under an inert atmosphere overnight. The reaction medium is evaporated to dryness under a rough vacuum. The crude is taken back in 50 mL of EtOAc and washed with 1M HCl, NaHCO₃ and brine. The organic layer is dried on MgSO₄ and purified by flash chromatography on silica gel (elution gradient from 2 to 5% MeOH in DCM). In this way, the intermediary product is isolated, in a yield of 80% (0.8 mmol; 560 mg). TLC:Rf=0.5 (DCM:MeOH 90/10 (v/v)).

The product is taken back in 20 mL DMF and 5 mL of piperidine are added. The reaction is then maintained for one night under stirring at ambient temperature. The reaction medium is evaporated to dryness under a rough vacuum. The residue is triturated several times in ether, filtrated and used as it is.

### (9Z,12Z)—N-[4-(2-hexyldecanoylamino)-1-(3-imidazol-1-ylpropylcarbamoyl)butyl]octadeca-9,12-dienamide (VI)

Molecule VI has been isolated from the coupling of Linoleic acid (4 mmol, 1.1 g) and crude compound 6b (0.8 mmol; 382 mg) using HBTU (4 mmol; 1.5 mg) and DIPEA (6 mmol; 1 mL) dissolved in 20 mL of DMF according to the procedure described for the molecule 5b. Reaction time: 48 h room temperature; Purification: Silica gel chromatography gradient 96/4 to 90/10 (DCM/MeOH, v/v). Yield: 22% (130 mg, 0.18 mmol, TLC (DCM/MeOH: 95/5) Rf: 0.4.).

### Synthesis Molecule VII

### Bis[(9Z,12Z)-octadeca-9,12-dienyl]2-aminopentanedioate (7a)

L-Glutamic acid (220 mg, 1.5 mmoles), Linoleyl alcohol (1.16 mL, 3.75 mmoles) and para-toluenesulfonic acid hydrate (715 mg, 3.75 mmoles) are dissolved under stirring in dry toluene (30 mL). The flask is equipped with a Dean-Stark trap and a condenser. The reaction is then stirred under reflux for 18 h before being cooled-down and concentrated. The residue is then taken back in DCM, washed twice with a saturated sodium bicarbonate solution, twice with water, and once with brine. Organic extracts are the dried-on magnesium sulfate, filtered and concentrated under vacuum. The product is collected by flash chromatography on silica gel using a gradient of a petrol spirit/ethyl acetate (from 100/0 v/v to 70/30 v/v) mixture as eluent. 322 mg (0.5 mmol, 30% yield) of a colourless oil is collected. TLC (PS/EA: 80/20) rf: 0.4.

### Bis[(9Z,12Z)-octadeca-9,12-dienyl]-2-[[2-(dimethylamino)acetyl]amino]pentanedioate (VII)

Molecule VII has been isolated from the coupling of DMG (24 mg, 0.23 mmoles) and compound 7a (0.25 mmoles, 161 mg) using methylimidazole (74 μL, 0.92 mmoles) and TCFH (98 mg, 0.35 mmoles) dissolved in 4 mL of 2/1 (v/v) solution of MeCN and DCM, according to the procedure described for the molecule II. Reaction time: 12 h 0 °C to RT; Purification: Silica gel chromatography gradient 98/2 to 90/10 (DCM/MeOH, v/v). Yield: 72% (124 mg of gluey solid, 0.17 mmol, TLC (DCM/MeOH: 95/5) Rf: 0.2.).

### Synthesis Molecule VIII

### bis[(9Z,12Z)-octadeca-9,12-dienyl]2-[4-(dimethylamino)butanoylamino]pentanedioate (VIII)

Molecule VIII has been isolated from the coupling of DMABA (40 mg, 0.23 mmoles) and compound 7a (0.25 mmoles, 161 mg) using methylimidazole (74 μL, 0.92 mmoles) and TCFH (98 mg, 0.35 mmoles) dissolved in 4 mL of 2/1 (v/v) solution of MeCN and DCM, according to the procedure described for the molecule II. Reaction time: 12 h 0 °C to RT; Purification: Silica gel chromatography gradient 98/2 to 90/10 (DCM/MeOH, v/v). Yield: 72% (136 mg of gluey solid, 0.180 mmol, TLC (DCM/MeOH: 95/5) Rf: 0.2.).

### Synthesis Molecule IX

### [(9Z,12Z)-octadeca-9,12-dienyl]ammonium, chloride (9a)

A solution of linoleyl alcohol (7.6 mmol; 2.02 g) and diisopropyl azodicarboxylate (12 mmol; 6.4 mL) in 14 mL of THF was added at 0 °C to a solution of PPh₃ (9 mmol, 2.36 g) and phthalimide (9 mmol; 1.32 g) in 10 mL of THF over 20 min. The reaction medium is allowed to warm to ambient temperature overnight. After the total conversion of the alcohol, hydrazine hydrate (2 mL, 50 wt %) is added, and the resulting solution is heated at reflux for 6 h. The solution is allowed to cool to ambient temperature, and 10 mL concentrated HCl was slowly added. The reaction medium is then refluxed for 2 h, then allowed to cool to ambient temperature, and stirred overnight. The precipitate was removed by filtration, and the solvent was removed in vacuo. The residual solid is dissolved in water and extracted 3 times with 40 mL DCM. The aqueous layer was concentrated to provide the ammonium in a yield of 53% (4.02 mmol; 1.21 g).

### 2-amino-N,N′-bis[(9Z,12Z)-octadeca-9,12-dienyl]pentanediamide (9b)

Following the procedure described for compound 1b, compound 9b is obtained in two steps by first reacting Fmoc-glutamic acid (0.6 mmol; 222 mg) and compound 9a (1.5 mmol; 453 mg) in presence of DIC (1.92 mmol; 297 μL) HOBt (1.92 mmol; 259 mg), and Et₃N (3 mmol; 408 μL) dissolved in 6 mL DMF and 6 mL CHCl₃. Piperidine (3 mL) is then added after 36 h. Reaction time: 40 h RT; Yield: 84% (white solid, 0.5 mmol; 380 mg), TLC (DCM/MeOH: 95/5) Rf: 0.2.).

### 2-[4-(dimethylamino)butanoylamino]-N,N′-bis[(9Z,12Z)-octadeca-9,12-dienyl]pentanediamide (IX)

Following the procedure described for compound I, molecule IX is obtained by coupling DMABA (0.3 mmol; 50 mg) and compound 9b (0.2 mmol; 129 mg) using DIPEA (0.6 mmol; 100 μL) and pyBOP (0.3 mmol; 156 mg) dissolved in 4 mL of DMF. Reaction time: 24 h RT; Purification: Silica gel chromatography gradient 98/2 to 92/8 (DCM/MeOH, v/v). Yield: 45% (0.09 mmol; 68 mg), TLC (DCM/MeOH: 95/5) Rf: 0.3.).

### Synthesis Molecule X

### (Z)—N-[5-(3-imidazol-1-ylpropylamino)-4-[[(Z)-octadec-9-enoyl]amino]-5-oxo-pentyl]octadec-9-enamide (X)

Oleic acid (251 μL, 0.8 mmoles) is dissolved under argon in 4 mL of a DMF solution that already contains DIPEA (175 μL, 1 mmoles) and HOBt (122 mg, 0.9 mmoles). After dissolution, a solution of 4 mL of DCM containing compound 5b (0.25 mmoles, 117 mg) is added under agitation. The mixture is cooled down to zero degrees, and HBTU (341 mg, 0.9 mmoles) is then introduced portion wise over 30 minutes. After addition, the reaction is let warmed to RT during 12 h, and stirred for 36 additional hours before being concentrated. The obtained residue is then resuspended in 30 mL of ethyl acetate and put 3 h at −20 °C. The resulting liquid and solid are then separated by centrifugation at 3000 rpm, and this procedure is repeated two times. All the liquid are collected and concentrated under vacuum while the solids are discarded. The product is collected by flash chromatography on silica gel using a DCM/methanol mixture as eluent. A gradient has been used ranging from 99/1 to 90/10 (DCM/MeOH, v/v). 102 mg (0.133 mmol, 53% yield) of a white creamy solid are collected. TLC (DCM/MeOH: 95/5) rf: 0.2.

### Synthesis Molecule XI

### N-[4-(dodecanoylamino)-5-(3-imidazol-1-ylpropylamino)-5-oxo-pentyl]dodecanamide (XVI)

Following a similar protocol than for compound X but using Lauric acid (0.8 mmoles, 160 mg) lead to the obtention of compound XI with 68% yield (0.17 mmoles, 103 mg) as a white powder. TLC (DCM/MeOH: 95/5) rf: 0.25.

### Synthesis Molecule XII

### 2-amino-N,N′-bis[(9Z,12Z)-octadeca-9,12-dienyl]butanediamide (12a)

Following the procedure described for compound 1b, compound 12a is obtained in two steps by first reacting Fmoc-aspartic acid (0.6 mmol; 213 mg) and compound 9a (2 mmol; 604 mg) in presence of DIC (1.92 mmol; 297 μL) HOBt (1.92 mmol; 259 mg), and Et₃N (3 mmol; 408 μL) dissolved in 6 mL DMF and 6 mL CHCl₃. Piperidine (3 mL) is then added after 36 h. Reaction time: 40 h RT; Yield: 69% (white solid, 0.41 mmol; 260 mg), TLC (DCM/MeOH: 90/10) Rf: 0.4).

### 2-[4-(dimethylamino)butanoylamino]-N,N′-bis[(9Z,12Z)-octadeca-9,12-dienyl]butanediamide (XII)

Following the procedure described for compound I, molecule XII is obtained by coupling DMABA (0.6 mmol; 101 mg) and compound 12a (0.4 mmol; 260 mg) using DIPEA (1.2 mmol; 207 μL) and pyBOP (0.6 mmol; 312 mg) dissolved in 8 mL of DMF. Reaction time: 24 h RT; Purification: Silica gel chromatography gradient 98/2 to 92/8 (DCM/MeOH, v/v). Yield: 33% (0.13 mmol; 97 mg), TLC (DCM/MeOH: 95/5) Rf: 0.3.).

### Synthesis Molecule XIII

### 2-[2-[[2-(dimethylamino)acetyl]amino]ethyldisulfanyl]ethylammonium (13a)

DMG (1 mmol, 103 mg) is placed in a 50 mL flask under an argon then dissolved in 10 mL of DMF under stirring. HBTU (1 mmol; 379 mg) and DIPEA (2 mmol; 346 μL) are successively added to the reaction medium. The reaction is then maintained under an inert atmosphere at RT for 15 minutes before Boc-Cystamine (0.83 mmol; 186 mg), in solution in 4 mL of DCM and 2 mL of DMF, is added to the reaction medium. After stirring at RT under argon for 16 hours, the reaction is stopped and the reaction medium is evaporated to dryness under a rough vacuum. The crude is taken back in 50 mL of EtOAc and washed 3 times with water and brine. The organic layer is dried on MgSO₄ and evaporated. The product, used without further purification (TLC:Rf=0.2 (DCM:MeOH 90/10 (v/v)).

Purified product is then dissolved in 40 mL of DCM, before 2 mL of trifluoroacetic acid are introduced into the reaction medium. The reaction is then maintained for 12 hours under stirring at RT. The reaction medium is evaporated to dryness under a vacuum. The final residue is taken back four times, in succession, in DCM and four times in Et₂O then evaporated to dryness. Yield 83% (2 steps, 0.83 mmoles, 291 mg).

### [5-[2-[2-[[2-(dimethylamino)acetyl]amino]ethyldisulfanyl]ethylamino]-5-oxo-4-[(2,2,2-trifluoroacetyl)ammonio]pentyl]-(2,2,2-trifluoroacetyl)ammonium (13b)

Boc-L-Orn(Boc)-OH, 365 mg, 1.1 mmoles) is dissolved under Argon in 10 mL of a 9/1 v/v mixture of dry DCM/DMF, in a three-necks round-bottom flask equipped with a dropping funnel. HOBt (162 mg, 1.2 mmoles) and DIC (201 μL, 1.3 mmoles) are subsequently added and the reaction is stirred at RT for 3 h, increasing the turbidity of the medium. Then compound 13a (351 mg, 1 mmoles) and Et₃N (210 μL, 1.5 mmoles) dissolved in 10 mL of a 9/1 v/v mixture of dry DCM/DMF are added over 30 min dropwise via the dropping funnel. After the completion of the addition, the reaction is stirred 36 h at room temperature and concentrated to dryness. The crude mixture is then resuspended in 20 mL of ethyl acetate and put 3 h at −20 °C. The resulting liquid and solid are then separated by centrifugation at 3000 rpm, and this procedure is repeated three times. The solid residues are collected and washed subsequently three times using diethyl ether, before being dried overnight under vacuum.

The crude compound is dissolved under argon in 25 mL of dry DCM. 190 μL (2.5 mmoles) of trifluoracetic acid (TFA) are then added slowly and the reaction is pursued until complete conversion evidenced by TLC (2 h). The mixture is then concentrated, co-evaporated thrice with 20 mL of DCM, thrice with 20 mL of diethyl ether (Et₂O), and dried under high vacuum overnight. The product is then used as it without further purification (0.63 mmoles, 345 mg, 63%).

### (9Z,12Z)—N-[5-[2-[2-[[2-(dimethylamino)acetyl]amino]ethyldisulfanyl]ethylamino]-4-[[(9Z,12Z)-octadeca-9,12-dienoyl]amino]-5-oxo-pentyl]octadeca-9,12-dienamide (XIII)

Following the procedure described for compound X, molecule XIII is obtained by coupling Linoleic acid (404 μL, 1.3 mmoles) with compound 13b (0.63 mmoles, 345 mg) with DIPEA (263 μL, 1.5 mmoles), HOBt (183 mg, 1.35 mmoles) and HBTU (530 mg, 1.4 mmoles) dissolved in 6 mL DMF and 6 mL DCM. Reaction time: 18 h 0 °C to RT; Purification: Silica gel chromatography gradient 99/1 to 90/10 (DCM/MeOH, v/v). Yield: 78% (429 mg of white solid, 0.49 mmol, TLC (DCM/MeOH: 90/10) Rf: 0.4.).

### Synthesis Molecule XIV

### a) Synthesis Molecule 14d

### 2,5-diamino-N-[2-[tert-butyl(dimethyl)silyl]oxyethyl]pentanamidebis-trifluoroacetate. (14a)

Following the procedure described for compound 13b, molecule 14a is obtained in two steps by coupling first Boc-L-Orn(Boc)-OH, (3.65 g, 11 mmoles) with 2-[[tert-Butyl(dimethyl)silyl]oxy]ethylamine (2.04 mL, 10 mmoles) with HOBt (1.62 g, 12 mmoles), DIC (2.01 mL, 13 mmoles) Et₃N (2.10 mL, 15 mmoles) dissolved in 10 mL DMF and 90 mL DCM. Deprotection is carried out using 1.9 mL (25 mmoles) TFA in 250 mL DCM. Reaction time 72 h RT then 6 h RT. Yield: 71% (3.45 g of white solid, 7.1 mmoles).

### N-[2-[tert-butyl(dimethyl)silyl]oxyethyl]-2,5-bis[4-(dimethylamino)butanoylamino]pentanamide (14b)

Molecule 14b has been isolated from the coupling of DMABA (400 mg, 2.4 mmoles) and compound 14a (485 mg, 1 mmole) using methylimidazole (650 μL, 8 mmoles) and TCFH (1.13 g, 4 mmoles) dissolved in 50 mL of 2/1 (v/v) solution of MeCN and DCM, according to the procedure described for the molecule II. Reaction time: 18 h 0 °C to RT; Purification: Silica gel chromatography gradient 98/2 to 90/10 (DCM/MeOH, v/v). Yield: 82% (423 mg of white solid, 0.82 mmol, TLC (DCM/MeOH: 95/5) Rf: 0.5.).

### 2,5-bis[4-(dimethylamino)butanoylamino]-N-(2-hydroxyethyl)pentanamide (14c)

Compound 14b (258 mg, 0.5 mmoles) is dissolved in 12 mL of dry THF. Tetrabuylammonium fluoride (TBAF, 1M in THF, 2 mmoles, 2 mL) is then introduced and the reaction is stirred for 2 h at 0 °C. After concentration of the medium, the obtained residue is resuspended in 15 mL of ethyl acetate and washed twice with water and once with brine, dried on sodium sulphate, filtered and concentrate, yielding the pure compound with 95% yield (0.48 mmoles, 193 mg).

### [1-[2-[2,5-bis[4-(dimethylamino)butanoylamino]pentanoylamino]ethoxycarbonyl]-4-[(2,2,2-trifluoroacetyl)ammonio]butyl]-(2,2,2-trifluoroacetyl)ammonium (14d)

Following the procedure described for compound 13b, molecule 14d is obtained in a two steps sequence by coupling first Boc-L-Orn(Boc)-OH, (92 mg, 0.275 mmoles) with compound 14c (100 mg, 0.25 mmoles) with HOBt (41 mg, 0.3 mmoles), DIC (50.1 μL, 0.33 mmoles) and Et₃N (53 μL, 0.375 mmoles) dissolved in 3 mL of a 9/1 v/v mixture of dry DCM/DMF. Deprotection is carried out after intermediary purification, by using 76 μL (1 mmoles) TFA in 2.5 mL DCM. Reaction time 36 h RT then 2 h RT. Yield: 79% (140 mg of colourless syrup, 0.197 mmoles).

### b) Synthesis Molecule XIV

### 2-[2,5-bis[4-(dimethylamino)butanoylamino]pentanoylamino]ethyl-2,5-bis[[(9Z,12Z)-octadeca-9,12-dienoyl]amino]pentanoate (XIV)

Following the procedure described for compound X, molecule XIV is obtained by coupling linoleic acid (81 μL, 0.33 mmoles) with compound 14d (0.125 mmoles, 88.8 mg) with DIPEA (70 μL, 0.3 mmoles), HOBt (45 mg, 0.33 mmoles) and HBTU (106 mg, 0.28 mmoles) dissolved in 2 mL DMF and 2 mL DCM. Reaction time: 36 h 0 °C to RT; Purification: Silica gel chromatography gradient 98/2 to 90/10 (DCM/MeOH, v/v). Yield: 83% (108 mg of white solid, 0.104 mmol, TLC (DCM/MeOH: 95/5) Rf: 0.2).

### Synthesis Molecule XV

### a) Synthesis of Molecule 15d

### tert-butyl N-(4-hydroxybutyl)carbamate (15a)

4-aminobutan-1-ol (10 mmoles, 922 μL) is dissolved in 20 mL of dry absolute ethanol. The mixture is then cooled down to zero degree. Then 2.3 mL of Boc₂O (10 mmoles) are added dropwise over 30 minutes while stirring at 0 °C. The reaction is then pursued for 48 h before being concentrated under vacuum. The oily residue is the redissolved in 20 mL of a 1/1 (v/v) mixture of DCM and water. The phase is decanted in a funnel, and the aqueous layer is extracted thrice with 5 mL of DCM. Combined organic extracts are then washed with brine, dried on magnesium sulfate, filtered and concentrated to dryness. The oil is used without additional purification. (97% yield, 1.84 g, 9.7 mmoles). TLC (DCM/MeOH: 95/5) rf: 0.7.

### 4-(tert-butoxycarbonylamino)butyl dodecanoate (15b)

Compound 15b is obtained according to the procedure describing molecule III coupling Lauric acid (3.3 mmoles, 661 mg) and compound 15a (3 mmoles, 567 mg) using 557 μL of DIC (3.6 mmoles), 486 mg of HOBt (3.6 mmoles) and triethylamine (546 μL, 3.9 mmoles) in solution of 56 mL DCM and 4 mL DMF. The crude mixture is concentrated then resuspended in 40 mL of ethyl acetate and put 3 h at −20 °C. The resulting liquid and solid are then separated by centrifugation at 3000 rpm, and this procedure is repeated three times. All the liquid are collected and concentrated under vacuum. The product is collected by flash chromatography on silica gel using a petrol spirit/ethyl acetate (80/20 v/v) mixture as eluent. 781 mg (2.1 mmol, 71% yield) of a colourless oil is collected. TLC (PS/EA: 80/20) rf: 0.5.

### 4-dodecanoyloxybutylammonium trifluoroacetate (15c)

Compound 15b (781 mg, 2.1 mmoles) is dissolved under argon in 40 mL of dry DCM. 2 equivalents (4.2 mmoles, 321 μL) of trifluoracetic acid (TFA) are then added slowly and the reaction is pursued until complete conversion evidenced by TLC (2 h). The mixture is then concentrated, co-evaporated thrice with 20 mL of DCM, thrice with 20 mL of diethyl ether (Et₂O), and dried under high vacuum overnight. The product is then used as it without further purification (2.08 mmoles, 768 mg, 99%).

### 4-[[4-amino-5-(4-dodecanoyloxybutylamino)-5-oxo-pentanoyl]amino]butyl dodecanoate (15d)

Compound 15d is obtained following the 2-steps procedure described for molecule 1b, by first coupling N-[(9H-Fluoren-9-ylmethoxy)carbonyl]-L-glutamic Acid (Fmoc-Glu-OH, 259 mg, 0.7 mmoles) with compound 15c (768 mg, 2.08 mmoles) using HOBt (331 mg, 2.45 mmoles), DIC (325 μL, 2.1 mmoles) and Et₃N (394 μL, 2.8 mmoles) dissolved in chloroform (7 mL) and DMF (7 mL), then by removing the Fmoc group using piperidine (2 mL). The crude mixture is then concentrated then resuspended in 40 mL of ethyl acetate and placed 3 h at −20 °C. The resulting liquid and solid are then separated by centrifugation at 3000 rpm, and this procedure is repeated three times. All the liquid are collected and concentrated under vacuum while the solids are discarded. The product is collected by flash chromatography on silica gel using a DCM/methanol mixture as eluent. A gradient has been used ranging from 98/2 to 94/6 (DCM/MeOH, v/v). 163 mg (0.25 mmol, 36% yield) of a white viscous solid are collected. TLC (DCM/MeOH: 95/5) rf: 0.2.

### b) Synthesis of Molecule XV

### 4-[[4-[[2-(dimethylamino)acetyl]amino]-5-(4-dodecanoyloxybutylamino)-5-oxo-pentanoyl]amino]butyl dodecanoate (XV)

Following the procedure described for compound X, molecule XV is obtained by coupling DMG (34 mg, 0.325 mmoles) with compound 15d (0.25 mmoles, 163 mg) with DIPEA (90 μL, 0.5 mmoles), HOBt (50 mg, 0.375 mmoles) and HBTU (142 mg, 0.375 mmoles) dissolved in 2 mL DMF and 2 mL DCM. Reaction time: 36 h 0 °C to RT; Purification: Silica gel chromatography gradient 99/1 to 92/8 (DCM/MeOH, v/v). Yield: 48% (89 mg of white solid, 0.12 mmol, TLC (DCM/MeOH: 95/5) Rf: 0.4).

### Synthesis Molecule XVI

### 4-[[4-[2,5-bis(tert-butoxycarbonylamino)pentanoylamino]-5-(4-dodecanoyloxybutylamino)-5-oxo-pentanoyl]amino]butyl dodecanoate (16a)

Boc-L-Orn(Boc)-OH Boc-L-Orn(Boc)-OH, (365 mg, 1.1 mmoles) is dissolved under Argon in 10 mL of a 9/1 v/v mixture of dry DCM/DMF, in a three-necks round-bottom flask equipped with a dropping funnel. HOBt (162 mg, 1.2 mmoles) and DIC (201 μL, 1.3 mmoles) are subsequently added and the reaction is stirred at RT for 3 h. Then compound 15d (654 mg, 1 mmoles) and Et₃N (210 μL, 1.5 mmoles) dissolved in 10 mL of a 9/1 v/v mixture of dry DCM/DMF are added over 30 min dropwise via the dropping funnel. After the completion of the addition, the reaction is stirred 36 h at room temperature and concentrated to dryness. The crude mixture is then resuspended in 20 mL of ethyl acetate and put 3 h at −20 °C. The resulting liquid and solid are then separated by centrifugation at 3000 rpm, and this procedure is repeated three times. The solid residues are collected and washed subsequently three times using diethyl ether, before being dried overnight under vacuum, yielding the desired compound pure as white solid with 85% yield (823 mg, 0.85 mmoles). TLC (DCM/MeOH: 95/5) rf: 0.4.

### 4-[[5-(4-dodecanoyloxybutylamino)-4-(2-methylhexanoylamino)-5-oxo-pentanoyl]amino]butyl dodecanoate bis-trifluoroacetate (16b)

Boc protecting groups have been removed following the procedure described for compound 15c using compound 16a (823 mg, 0.85 mmoles) and 2 equivalents (1.7 mmoles, 130 μL of TFA dissolved under argon in 18 mL of DCM for 3 h at RT. The product is isolated with 98% yield (0.833 mmoles, 803 mg).

### 4-[[4-[2,5-bis[4-(dimethylamino)butanoylamino]pentanoylamino]-5-(4-dodecanoyloxybutylamino)-5-oxo-pentanoyl]amino]butyl dodecanoate (XVI)

Molecule XVI has been isolated following the procedure described for molecule 5b, by coupling DMABA (109 mg, 0.65 mmoles) with compound 22b (0.5 mmoles, 482 mg) using DIPEA (305 μL, 1.75 mmoles) and HBTU (663 mg, 1.75 mmoles) in 8 mL DMF. Reaction time: 48 h 0 °C to RT; Purification: Silica gel chromatography gradient 99/1 to 90/10 (DCM/MeOH, v/v). Yield: 56% (278 mg of white solid, 0.28 mmol, TLC (DCM/MeOH: 95/5) Rf: 0.25).

### Synthesis Molecule XVII

### N,N′-bis[(Z)-octadec-9-enyl]-2-oxo-pentanediamide (17a)

α-ketoglutaric acid (146 mg, 1 mmole) is dissolved in a 4/1 (v/v) mixture of DCM/DMF under argon. DIPEA (558 μL, 3.2 mmoles) is then added and the reaction is stirred for 5 minutes. PyBOP (1.56 g, 3 mmoles) is introduced and the mixture is stirred for another 5 minutes, turning orange in colour. Oleylamine (1.16 mL 3 mmoles) is finally added dropwise over 15 minutes, and the reaction is maintained at room temperature for 48 h, before being concentrated. The oily residue is taken back in 30 mL of EtOAc, placed 3 h at −20 °C, and the resulting solid is eliminated by centrifugation at 3000 rpm. The remaining liquid is concentrated, and the product is obtained by purification via by flash chromatography on silica gel using a DCM/methanol mixture as eluent. A gradient has been used ranging from 99/1 to 95/5 (DCM/MeOH, v/v). 310 mg (0.48 mmol, 48% yield) of a light-yellow solid is collected. TLC (DCM/MeOH: 95/5) rf: 0.45.

### 4-[(dimethylamino)methyl]-N—[(Z)-octadec-9-enyl]-2-[3-[[(Z)-octadec-9-enyl]amino]-3-oxo-propyl]-1,3-dioxolane-2-carboxamide (XVII)

Compound 17a (161 mg, 0.25 mmoles), 3-(dimethylamino)propane-1,2-diol (59 μL, 0.5 mmoles) and pyridinium-para-toluenesulfonate (PPTS, 11 mg, 0.05 mmoles) are dissolved under stirring in dry toluene (20 mL). The round-bottom flask is equipped with a Dean-Stark trap and a condenser. The reaction is then stirred under reflux for 18 h before being cooled-down and concentrated. The residue is then taken back in DCM, washed twice with a saturated sodium bicarbonate solution, twice with water, and once with brine. Organic extracts are the dried on magnesium sulfate, filtered and concentrated under vacuum. The product is collected by flash chromatography on silica gel using a gradient of a DCM/methanol (from 100/0 v/v to 95/5 v/v) mixture as eluent. 63 mg (0.085 mmol, 34% yield) of a colourless solid is collected. TLC (DCM/MeOH: 97/3) rf: 0.3.

### Synthesis Molecule XVIII

### 2-hydroxy-N,N′-bis[(Z)-octadec-9-enyl]pentanediamide (18a)

Compound 17a (0.25 mmoles, 161 mg) is dissolved in 2.5 mL of absolute ethanol. The reaction is then cooled down to 0 °C and sodium borohydride (19 mg, 0.5 mmoles) is introduced in 2 equal portions. The reaction then takes place during 3 h at 0 °C, before being quenched at 0 °C with 5 mL of saturated aqueous ammonium chloride. The aqueous phase is then extracted with ethyl acetate (3*10 mL). Combined organic extracts are washed with water a brine, dried on magnesium sulfate, filtered and concentrated. Pure product (149 mg, 0.23 mmoles) is collected as a colourless oil and used as it in the next step (91% yield).

### [4-[[(Z)-octadec-9-enyl]amino]-1-[[(Z)-octadec-9-enyl]carbamoyl]-4-oxo-butyl]4-(dimethylamino)butanoate (XVIII)

Esterification leading to molecule XVIII has been accomplished adapting the procedure described for compound II, linking DMABA (40 mg, 0.24 mmoles) with compound 18a (129 mg, 0.2 mmoles) using methylimidazole (65 μL, 0.8 mmoles) and TCFH (113 mg, 0.4 mmoles dissolved in 3 5 mL of dry 2/1 (v/v) mixture solution of MeCN and DCM). Reaction time: 18 h 0 °C to RT; Purification: Silica gel chromatography gradient 98/2 to 90/10 (DCM/MeOH, v/v). Yield: 61% (93 mg of white solid, 0.122 mmol, TLC (DCM/MeOH: 90/10) Rf: 0.4).

### Synthesis Molecule XIX

### 5-hydroxy-N—[(Z)-octadec-9-enyl]-2-[3-[[(Z)-octadec-9-enyl]amino]-3-oxo-propyl]-1,3-dioxane-2-carboxamide (19a)

Ketalization of molecule 19a (161 mg, 0.25 mmoles) using 2-{[Dimethyl(2-methyl-2-propanyl)silyl]oxy}-1,3-propanediol (103 mg, 0.5 mmoles) and PPTS (11 mg, 0.05 mmoles) dissolved toluene (20 mL) has been accomplished following the procedure described for compound XVII during 18 h.

Hydroxyl functions are then released following the procedure describing 14c, using TBAF (1M in THF, 1 mmoles, 1 mL) in 5 mL of dry THF. After concentration of the medium, the targeted product is recovered by flash chromatography on silica gel using a DCM/methanol mixture as eluent. A gradient has been used ranging from 98/2 to 90/10 (DCM/MeOH, v/v). 49 mg (0.068 mmoles, 27% yield, two steps) of a white solid are collected. TLC (DCM/MeOH: 90/10) rf: 0.5.

### 4-imidazol-1-ylbutanoic acid (19b)

4-bromobutyric acid (10 mmoles, 1.67 g) is dissolved in dry MeCN (100 mL). Imidazole (10.5 mmoles, 714 mg) is then introduced and the reaction is stirred overnight at 60 °C before being concentrated. The resulting solid is then precipitated in diethyl ether, filtered and washed thrice with cold diethyl ether. Product (9.6 mmoles, 1.48 g, 96%) is used as it without further purification.

### [2-[3-[[(Z)-octadec-9-enyl]amino]-3-oxo-propyl]-2-[[(Z)-octadec-9-enyl]carbamoyl]-1,3-dioxan-5-yl]4-imidazol-1-ylbutanoate (XIX)

Compound XIX has been isolated adapting the procedure described for compound II, linking compound 19b (15.4 mg, 0.1 mmoles) with compound 19a (72 mg, 0.1 mmoles) using methylimidazole (33 μL, 0.4 mmoles) and TCFH (113 mg, 0.4 mmoles) dissolved in 2 mL of a 2/1 (v/v) mixture solution of MeCN and DCM. Reaction time: 18 h 0 °C to RT; Purification: Silica gel chromatography gradient 98/2 to 90/10 (DCM/MeOH, v/v). Yield: 52% (44 mg of white solid, 0.122 mmol, TLC (DCM/MeOH: 90/10) Rf: 0.3).

### Synthesis Molecule XX

### a) Synthesis Molecule 20b

### [(Z)-non-2-enyl]4-aminobutanoate trifluoroacetate (20a)

Boc protecting groups have first been introduced according to the procedure described for compound 5a, using 4-aminobutyric acid (1.03 g, 10 mmoles), NaOH (440 mg, 11 mmoles), and Boc₂O (10.5 mmoles 2.41 mL) in a 1/1 mixture of water and tert-BuOH. Reaction time is 12 h.

Following the procedure described for compound 13b, the vacuum-dried crystallized compound (2.03 g), is then coupled with (Z)-2-nonen-1-ol (1.77 mL, 10.5 mmoles), using DIC (1.7 mL, 11 mmoles), HOBt (1.49 g, 11 mmoles), and Et₃N (2.1 mL, 15 mmoles) dissolved in 100 mL of a 9/1 v/v mixture of dry DCM/DMF during 36 h. Accordingly after initial workup, the amine functions are then released using 1.3 mL of TFA in 60 mL of DCM for 6 h. The product is then used pure as it after workup without further purification (8.8 mmoles, 4.97 g, 88% 3 steps).

### [(Z)-non-2-enyl]4-[[5-[[4-[(Z)-non-2-enoxy]-4-oxo-butyl]amino]-4,5-dioxo-pentanoyl]amino]butanoate (20b)

Molecule 20b has been isolated following the procedure describing compound 17a, in which α-ketoglutaric acid (146 mg, 1 mmole) and Compound 20a (813 mg, 2.5 mmoles) are linked using DIPEA (558 μL, 3.2 mmoles) and PyBOP (1.56 g, 3 mmoles) dissolved in 20 mL of a 4/1 (v/v) mixture of DCM/DMF under argon. Reaction time: 48 h RT; Purification: Silica gel chromatography gradient 98/2 to 92/8 (DCM/MeOH, v/v). Yield: 42% (237 mg of light-yellow solid, 0.42 mmol, TLC (DCM/MeOH: 95/5) Rf: 0.25).

### b) Synthesis Molecule XX

### [(Z)-non-2-enyl]4-[3-[5-hydroxy-2-[[4-[(Z)-non-2-enoxy]-4-oxo-butyl]carbamoyl]-1,3-dioxan-2-yl]propanoylamino]butanoate (20c)

Ketalization of molecule 20b (565 mg, 1 mmoles) using 2-{[Dimethyl(2-methyl-2-propanyl)silyl]oxy}-1,3-propanediol (412 mg, 2 mmoles) and PPTS (44 mg, 0.2 mmoles) dissolved toluene (100 mL) has been accomplished following the procedure described for compound XVII during 18 h.

Hydroxyl functions are then released following the procedure describing 14c, using TBAF (1M in THF, 4 mmoles, 4 mL) in 20 mL of dry THF, 2 h at 0 °C. After concentration of the medium, the targeted product is recovered by flash chromatography on silica gel using a DCM/methanol mixture as eluent. A gradient has been used ranging from 98/2 to 90/10 (DCM/MeOH, v/v). 83 mg (0.13 mmoles, 13%, two steps) of a white solid are collected. TLC (DCM/MeOH: 95/5) rf: 0.35.

### [(Z)-non-2-enyl]4-[3-[5-[4-(dimethylamino)butanoyloxy]-2-[[4-[(Z)-non-2-enoxy]-4-oxo-butyl]carbamoyl]-1,3-dioxan-2-yl]propanoylamino]butanoate (XX)

Compound XX has been isolated adapting the procedure described for compound II, by linking compound DMABA (40 mg, 0.24 mmoles) with compound 20c (83 mg, 0.13 mmoles) using methylimidazole ((65 μL, 0.8 mmoles) and TCFH (113 mg, 0.4 mmoles) dissolved in 5 mL of a 2/1 (v/v) mixture solution of MeCN and DCM. Reaction time: 18 h 0 °C to RT; Purification: Silica gel chromatography gradient 98/2 to 90/10 (DCM/MeOH, v/v). Yield: 64% (63 mg of white solid, 0.083 mmol, TLC (DCM/MeOH: 90/10) Rf: 0.25).

### Synthesis Molecule XXI

### 3-[tert-butyl(dimethyl)silyl]oxy-N-[3-[tert-butyl(dimethyl)silyl]oxypropyl]propan-1-amine (21a)

3-(3-Hydroxy-propylamino)-propan-1-ol (10 mmoles, 1.33 g) is dissolved in dry DMF (100 mL). The solution is cooled down to 0 °C before Imidazole (21 mmoles, 1.43 g) and tert-Butyldimethylsilyl Chloride (25 mmoles, 3.75 g) are then introduced over 1 h in 4 equal portions. The reaction is stirred overnight at room temperature before being concentrated. The obtained residue is then redissolved in 100 mL of DCM and washed twice saturated sodium carbonate solution, twice with water and once with brine, dried on sodium sulphate, filtered and concentrated. The product is collected by flash chromatography on silica gel using a DCM/Eₜ₂O (50/50 v/v) mixture as eluent). 3.4 g (9.4 mmoles, 94% yield) of an oily solid are collected. TLC (DCM/Et₂O (50/50 v/v)) rf: 0.5.

### [(Z)-non-2-enyl]4-[[4-(2,5-dioxopyrrolidin-1-yl)oxycarbonyloxy-5-[[4-[(Z)-non-2-enoxy]-4-oxo-butyl]amino]-5-oxo-pentanoyl]amino]butanoate (21 b)

Compound 20b (565 mg, 1 mmoles) is first reduced as described for molecule 18a by using sodium borohydride (76 mg, 2 mmoles) reacted in 10 mL of absolute ethanol at 0 °C for 3 h. The crude compound is then dissolved in 10 mL of dry DCM. Triethylamine (1.3 mmoles, 182 μL) is added, followed 5 minutes after by N,N-disuccinimidyl carbonate (307 mg, 1.2 mmoles). The solution is stirred at room temperature for 12 h before washing the organics with saturated ammonium chloride solution, water and brine. The organic phase is then dried on magnesium sulfate, filtered and concentrated. The product is collected by flash chromatography on silica gel using a 95/5 v/v DCM/methanol mixture as eluent. 580 mg (0.82 mmoles, 82% yield 2 steps) of a white solid are collected. TLC (DCM/MeOH: 95/5) rf: 0.4.

### [(Z)-non-2-enyl]4-[[4-[bis(3-hydroxypropyl)carbamoyloxy]-5-[[4-[(Z)-non-2-enoxy]-4-oxo-butyl]amino]-5-oxo-pentanoyl]amino]butanoate (XXI)

Compound 21a (181 mg, 0.5 mmoles) is dissolved under argon in 5 mL of a 1/1 (v/v) mixture of dry DCM and DMF. 2 equivalents (1 mmoles, 140 μL) of t Et₃N are then added slowly and the reaction is stirred for 5 minutes. Then, compound 21b (0.5 mmoles, 353 mg) is added and the mixture is stirred for 24 h at room temperature. The mixture is then concentrated, and redissolved in 80 mL of ethyl acetate. The organic phase is then washed with a saturated aqueous ammonium chloride solution, twice with MilliQ water, once with brine, dried on MgSO₄, filtered and concentrated. The obtained crude product is then redissolved in 25 mL of dry THF. TBAF (1M in THF, 4 mmoles, 4 mL) is then introduced and the reaction is stirred for 4 h at 0 °C. After concentration of the medium, the targeted product is recovered by flash chromatography on silica gel using a DCM/methanol mixture as eluent. A gradient has been used ranging from 98/2 to 94/6 (DCM/MeOH, v/v). 400 mg (0.55 mmoles, 55% yield, two steps) of a colourless oil is collected. TLC (DCM/MeOH: 95/5) rf: 0.4.

### Synthesis Molecule XXII

### [5-[2-(dimethylamino)ethylamino]-5-oxo-4-[(2,2,2-trifluoroacetyl)ammonio]pentyl]-(2,2,2-trifluoroacetyl)ammonium (22a)

Compound 22a has been isolated in two steps by first adapting the procedure described for compound II, by linking compound Boc-L-Orn(Boc)-OH (365 mg, 1.1 mmoles) with N,N-dimethylethylenediamine (66 μL, 0.6 mmoles) using methylimidazole (299 μL, 3.7 mmoles) and TCFH (520 mg, 1.85 mmoles) dissolved in 28 mL of a 2/1 (v/v) mixture solution of MeCN and DCM. Reaction time: 18 h 0 °C to RT; Purification: Silica gel chromatography gradient 98/2 to 95/5 (DCM/MeOH, v/v). Yield: 77% (185 mg 0.46 mmoles, MW=402.5 g/mol, 77%). Hydroxyl functions are then released adapting the procedure described for compound 15c using 76 μL (1 mmoles) of TFA in 10 mL of DCM until completion (2 h). Usual workup provides compound as colourless solid (0.245 mmoles, 97 mg, 98%).

### N-[5-[2-(dimethylamino)ethylamino]-4-(heptadeca-4,6-diynoylamino)-5-oxo-pentyl]heptadeca-4,6-diynamide (XXII)

Molecule XXII has been isolated following the procedure described for molecule X, by coupling heptadeca-4,6-diynoic acid (193 mg, 0.74 mmoles) with compound 22b (0.245 mmoles, 97 mg) using DIPEA (175 μL, 1 mmoles) and HBTU (341 mg, 0.9 mmoles) in 4 mL DMF and 4 mL DCM. Reaction time: 36 h 0 °C to RT; Purification: Silica gel chromatography gradient 95/5 to 90/10 (DCM/MeOH, v/v). Yield: 49% (83 mg of white solid, 0.120 mmol, TLC (DCM/MeOH: 95/5) Rf: 0.15).

### Synthesis Molecule XXIII

### 4-(9H-fluoren-9-ylmethoxycarbonylamino)-5-[[4-[(Z)-non-2-enoxy]-4-oxo-butyl]amino]-5-oxo-pentanoic acid (23a)

Fmoc-Glu(OtBu)-OH (2.5 mmol; 1.06 g) is placed in a 100 mL flask under an argon then dissolved in 40 mL of anhydrous DMF under stirring. COMU (2.5 mmol; 1.07 g) and DIPEA (5 mmol; 865 μL) are successively added to the reaction medium. The reaction is then maintained under an argon and 0 °C for 15 minutes before 20a (4.9 mmol; 1.67 g), dissolved in 10 mL of anhydrous DMF, is added to the reaction medium under stirring. The solution is allowed to warm to RT overnight. before being stopped evaporated to dryness under vacuum. The crude is taken back in 50 mL of EtOAc and washed with 1M HCl, NaHCO₃ and brine. The organic layer is dried on MgSO₄ and purified by flash chromatography on silica gel (elution gradient from 30 to 60% EtOAc in petroleum ether). In this way, the intermediate is isolated, in a yield of 74% (1.85 mmol; 1.2 g). TLC:Rf=0.3 (EtOAc:Petroleum ether 40/60 (v/v)) and then dissolved in 90 mL of DCM containing 10 mL of trifluoroacetic acid. The reaction is then stirred for one night at RT. The reaction medium is evaporated to dryness. The mixture is then concentrated, co-evaporated thrice with 50 mL of DCM, thrice with 50 mL of diethyl ether (Et2O), and dried under high vacuum overnight (74% 2 steps, 1.85 mmoles 1.07 g) [(Z)-non-2-enyl]4-[[2-amino-5-oxo-5-(tetradecylamino)pentanoyl]amino]butanoate (23b) Molecule 23b has been isolated in two consecutive steps following the procedure described for molecule 1b, by coupling tetradecylamine (1.5 mmol; 320 mg) with compound 23a (1 mmol; 579 mg) using DIC (2 mmol; 313 μL), HOBt (2 mmol; 270 mg) and Et₃N (2 mmol; 272 μL) in 18 mL DMF and 30 mL CHCl₃. Piperidine (10 mL) is added after 30 h and stirred 6 h more. Reaction time: 36 h RT; Yield: 61% (0.61 mmol; 337 mg) TLC:Rf=0.4 (DCM:MeOH 90/10 (v/v)).

### [(Z)-non-2-enyl]4-[[2-[[2-(dimethylamino)acetyl]amino]-5-oxo-5-(tetradecylamino)pentanoyl]amino]butanoate (XXIII)

Compound XXIII has been isolated adapting the procedure described for compound II, by linking compound DMG (0.23 mmol; 24 mg) with compound 23b (0.3 mmol; 170 mg) using methylimidazole (0.81 mmol; 65 μL) and TCFH (0.28 mmol; 79 mg) dissolved in 5 mL of a 2/1 (v/v) mixture solution of MeCN and DCM. Reaction time: 12 h 0 °C to RT; Purification: Silica gel chromatography gradient 98/2 to 95/5 (DCM/MeOH, v/v). Yield: 83% (121 mg of white solid, 0.19 mmol, TLC (DCM/MeOH: 90/10) Rf: 0.4).

### Synthesis Molecule XXIV

### 2-amino-N,N′-didodecyl-butanediamide (24a)

Following the procedure described for compound 1b, molecule 24a is obtained in two steps by coupling Fmoc-aspartic acid (3 mmol; 1.07 g) and Dodecylamine (9 mmol; 2.07 mL) using DIC (10.5 mmol; 1.66 mL) and HOBt (10.5 mmol; 1.42 g) dissolved in 40 mL of DMF and 40 mL of CHCl₃. After 24 h stirring at RT, Fmoc deprotection is carried out by introducing 9 mL of piperidine and stirring 4 h at RT. Reaction time: 24 h+4 h RT; Yield: 82% (2.46 mmol; 1.12 g); TLC:Rf=0.4 (DCM:MeOH 95/5 (v/v)).).

### [(Z)-non-2-enyl]-4-[[2-amino-5-[[3-(dodecylamino)-1-(dodecylcarbamoyl)-3-oxo-propyl]amino]-5-oxo-pentanoyl]amino]butanoate (24b)

20a (1.89 mmol; 1.09 g) is placed in a dry 100 mL flask under argon then dissolved in 20 mL of anhydrous DMF under stirring. DIPEA (5.7 mmol; 987 μL) and pyBOP (2.46 mmol; 1.28 g) are added to the solution successively. The reaction medium is stirred under argon at RT for 15 minutes before 24a (2.46 mmol; 1.12 g), in solution in 10 mL of anhydrous DMF and 5 mL of anhydrous DCM, is added to the reaction medium. After stirring at RT for 36 hours, 7 mL of piperidine are added and the reaction medium is stirred for another 4 hours. The reaction is stopped and the reaction medium is evaporated to dryness. The coupling product is then purified by flash chromatography on silica gel (elution gradient from 0 to 5% MeOH in DCM). In this way, the product is isolated in a yield of 42% (0.79 mmol; 636 mg). TLC:Rf=0.5 (DCM:MeOH 90/10 (v/v)).

### [(Z)-non-2-enyl]-4-[[2-[4-(dimethylamino)butanoylamino]-5-[[3-(dodecylamino)-1-(dodecylcarbamoyl)-3-oxo-propyl]amino]-5-oxo-pentanoyl]amino]butanoate (XXIV)

Compound XXIV has been isolated adapting the procedure described for compound II, by linking compound 24b (0.26 mmol; 210 mg) with DMABA (0.2 mmol; 34 mg) using methylimidazole (0.7 mmol; 120 μL) and TCFH (0.6 mmol; 170 mg) dissolved in 5 mL of a 2/1 (v/v) mixture solution of MeCN and DCM. Reaction time: 12 h 0 °C to RT; Purification: Silica gel chromatography gradient 98/2 to 90/10 (DCM/MeOH, v/v). Yield: 61% (147 mg of white solid, 0.16 mmol, TLC (DCM/MeOH: 90/10) Rf: 0.3).

### Synthesis Molecule XXV

### tert-butyl-N-[4-amino-5-[2-[2-[[2-(dimethylamino)acetyl]amino]ethyldisulfanyl]ethylamino]-5-oxo-pentyl]carbamate (25a)

Fmoc-Orn(Boc)-OH (1 mmol; 1.07 g) is placed beforehand in a dry 50 mL flask under an inert atmosphere then dissolved in 10 mL of anhydrous DMF under stirring. DIC (1.2 mmol; 188 μL), then HOBt (1.2 mmol; 162 mg), in solution in 1 mL anhydrous DMF, are successively added to the reaction medium. The reaction is then maintained under an inert atmosphere at ambient temperature for 2 and a half hours. 13a (0.83 mmol; 292 mg) and triethylamine (2 mmol; 281 μL) in solution in 6 mL of anhydrous DCM is added dropwise to the reaction medium. After stirring at ambient temperature under an inert atmosphere for 24 hours, 3 mL of piperidine are added and the reaction medium is stirred for another 4 hours. The reaction is stopped and the reaction medium is evaporated to dryness under a rough vacuum. The residue is taken back in 20 mL of EtOAc and washed 3 times with water and brine. The organic layer is dried on MgSO₄ and purified by flash chromatography on silica gel (elution gradient from 2 to 6% MeOH in DCM). In this way, the product is isolated, in a yield of 87% (0.72 mmol; 485 mg). TLC:Rf=0.3 (DCM:MeOH 90/10 (v/v))

The product is taken back in 20 mL of DMF and 2 mL of piperidine is added and the reaction medium is stirred for 4 hours. The reaction is stopped and the reaction medium is evaporated to dryness under a rough vacuum. The residue is triturated several times in petroleum ether, filtrated and used as it is.

### [4-azaniumyl-5-[[4-azaniumyl-1-[2-[2-[[2-(dimethylamino)acetyl]amino]ethyldisulfanyl]ethylcarbamoyl]butyl]amino]-5-oxo-pentyl]ammonium (25b)

First, Boc-L-Orn(Boc)-OH (1.4 mmol; 465 mg) is placed beforehand in a dry 25 mL flask under an inert atmosphere then dissolved in 10 mL of anhydrous DMF under stirring. HBTU (1.4 mmol; 531 mg) and DIPEA (2.8 mmol; 485 μL) are added to the solution successively. The reaction medium is stirred under an inert atmosphere at ambient temperature for 15 minutes before 25a (0.72 mmol; 325 mg), in solution in 10 mL of anhydrous DMF and 1 mL of anhydrous DCM, is added to the reaction medium. After 12 hours, the reaction is stopped and the reaction medium is evaporated to dryness under a high vacuum. The crude is taken back in 100 mL of EtOAc and washed with 1M HCl, NaHCO₃ and brine. The organic layer is dried on MgSO₄ and purified by flash chromatography on silica gel (elution gradient from 2 to 5% MeOH in DCM). In this way, the product is isolated, in a yield 63% (2 steps, 392 mg, 0.51 mmol). The obtained compound (0.1 mmol; 79 mg) is dissolved in 5 mL of DCM, then 250 μL of trifluoroacetic acid is added to the reaction medium. The reaction is then maintained for four hours under stirring at ambient temperature. The reaction medium is evaporated to dryness under a rough vacuum. The final residue is taken back four times, in succession, in DCM and four times in Et₂O then evaporated to dryness. The reaction is quantitative.

### (9Z,12Z)—N-[5-[[1-[2-[2-[[2-(dimethylamino)acetyl]amino]ethyldisulfanyl]ethylcarbamoyl]-4-[[(9Z,12Z)-octadeca-9,12-dienoyl]amino]butyl]amino]-4-[[(9Z,12Z)-octadeca-9,12-dienoyl]amino]-5-oxo-pentyl]octadeca-9,12-dienamide (XXV)

Compound XXV has been isolated adapting the procedure described for compound 1b, by linking compound Linoleic acid (1.8 mmol; 505 mg) with compound 25b (0.51 mmol; 411 mg) using DIPEA (2 mmol; 353 μL) and PyBOP (1.8 mmol; 936 mg) dissolved in 15 mL of DMF. Reaction time: 24 h RT; Purification: Silica gel chromatography gradient 98/2 to 94/6 (DCM/MeOH, v/v). Yield: 26% (166 mg of white solid, 0.13 mmol, TLC (DCM/MeOH: 95/5) Rf: 0.4).

### Synthesis Molecule XXVI

### 4-pyrrolidin-1-ylbutyl 2-amino-5-(tert-butoxycarbonylamino)pentanoate (26a)

Fmoc-Orn(Boc)-OH (0.5 mmol; 227 mg) is placed in a 50 mL flask under an argon and is then dissolved in 20 mL of DCM. DCC (0.6 mmol; 124 mg) and DMAP (0.1 mmol; 11 mg) are added to the reaction medium followed by 4-(1-pyrrolidinyl)-1-butanol (0.7 mmol; 100 mg).

After stirring at RT under argon, for 15 hours, the reaction is stopped and the reaction medium is evaporated to dryness. The crude is taken back in 20 mL of DCM and washed with 1M HCl, NaHCO₃ and brine. The organic layer is dried on MgSO₄ and purified by flash chromatography on silica gel (elution gradient from 4 to 10% MeOH in DCM). In this way, the intermediate product is isolated, and then taken back in 20 mL of DMF and 2 mL of piperidine is added and the reaction medium is stirred for 4 hours. The reaction is stopped and the reaction medium is evaporated to dryness under a rough vacuum. The residue is triturated several times in petroleum ether, filtrated and used as it is (82% yield, two steps, 0.41 mmoles, 147 mg).

### [4-azaniumyl-5-[[4-azaniumyl-1-(4-pyrrolidin-1-ylbutoxycarbonyl)butyl]amino]-5-oxo-pentyl]ammonium (26b)

Compound 26b is obtained following the two steps procedure experienced for the synthesis of compound 25b. First, Boc-L-Orn(Boc)-OH (0.7 mmol; 233 mg) and compound 26a (0.41 mmol; 147 mg) are linked together using HBTU (0.7 mmol; 265 mg) and DIPEA (1.4 mmol; 243 μL) dissolved in 9 mL of DMF; the reaction is then stirred for 16 h at RT. After isolation and purification of the intermediate (flash chromatography on silica gel, elution gradient from 2 to 5% MeOH in DCM), amine functions are released by reacting TFA (1 mL) in 10 mL DCM during 12 h. Yield: 51% (2 steps, 149 mg, 0.21 mmol).

### 4-pyrrolidin-1-ylbutyl-2-[2,5-bis[[(9Z,12Z)-octadeca-9,12-dienoyl]amino]pentanoylamino]-5-[[(9Z,12Z)-octadeca-9,12-dienoyl]amino]pentanoate (XXVI)

Compound XXVI has been isolated adapting the procedure described for compound 1b, by linking Linoleic acid (0.74 mmol; 206 mg) with compound 26b (0.21 mmol; 149 mg) using DIPEA (1.4 mmol; 247 μL) and PyBOP (0.74 mmol; 385 mg) dissolved in 8 mL of DMF. Reaction time: 24 h RT; Purification: Silica gel chromatography gradient 98/2 to 94/6 (DCM/MeOH, v/v). Yield: 32% (78 mg of white solid, 0.07 mmol, TLC (DCM/MeOH: 95/5) Rf: 0.3).

### Synthesis Molecule XXVII

### [4-azaniumyl-5-(2-hydroxyethylamino)-5-oxo-pentyl]ammonium (27a)

A solution of, Boc-L-Orn(Boc)-OH (2 mmol; 665 mg) in DMF is stirred at room temperature and EDC-HCl (2.4 mmol; 460 mg), HOBt (2.4 mmol; 324 mg) are successively added to the reaction medium. The reaction is stirred under Argon at RT for one hour before ethanolamine (10 mmol; 604 μL) is added. After stirring at RT for 24 hours more, the reaction is stopped and the reaction medium is evaporated to dryness. The residue is taken back in 50 mL of water and the amide is extracted 3 times with CHCl₃, the organic layers are dried on MgSO₄ and purified by flash chromatography on silica gel (elution gradient from 2 to 6% MeOH in DCM). The isolated intermediary product (1.82 mmol; 683 mg) is then dissolved in 90 mL of DCM, then 5 mL of trifluoroacetic acid are added to the reaction medium. The reaction is then maintained for 16 hours under stirring at ambient temperature. The mixture is then concentrated, co-evaporated thrice with 50 mL of DCM, thrice with 50 mL of diethyl ether (Et₂O), and dried under high vacuum overnight Yield: 91% (2 steps, 734 mg, 1.82 mmol).

### 2,5-bis[4-(dimethylamino)butanoylamino]-N-(2-hydroxyethyl)pentanamide (27b)

Compound 27b has been isolated adapting the procedure described for compound 5b, by coupling DMABA (4.55 mmol, 763 mg) with compound 27a (1.82 mmol; 734 mg) using DIPEA (9 mmol; 1.55 mL) and HBTU (4.55 mmol; 1.72 g) dissolved in 30 mL of DCM and 5 mL DMF. Reaction time: 48 h RT; Purification: Silica gel chromatography gradient 98/2 to 95/5 (DCM/MeOH, v/v). Yield: 51% (373 mg of white solid, 0.93 mmol, TLC (DCM/MeOH: 95/5) Rf: 0.4).

### 2-[2,5-bis[4-(dimethylamino)butanoylamino]pentanoylamino]ethyl2-amino-5-(tert-butoxycarbonylamino)pentanoate (27c)

Compound 27c is obtained following the two steps procedure experienced for the synthesis of compound 26a. First, Fmoc-L-Orn(Boc)-OH (1 mmol; 454 mg) and compound 27b (0.93 mmol; 373 mg) are coupled together using DCC (1.2 mmol; 248 mg) and DMAP (0.2 mmol; 22 mg) dissolved in 40 mL of DCM; the reaction is then stirred for 16 h at RT. After isolation and purification of the intermediate (flash chromatography on silica gel, elution gradient from 2 to 5% MeOH in DCM), Fmoc group is released by reacting piperidine (2 mL) in 20 mL DMF during 4 h. Yield: 76% (2 steps, 437 mg, 0.71 mmol).

### [4-azaniumyl-5-[[4-azaniumyl-1-[2-[2,5-bis[4-(dimethylamino)butanoylamino]pentanoylamino]ethoxycarbonyl]butyl]amino]-5-oxo-pentyl]ammonium (27d)

Compound 27d is obtained following the two steps procedure experienced for the synthesis of compound 25b. First, Boc-L-Orn(Boc)-OH (1.4 mmol; 465 mg) and compound 27c ((0.71 mmol; 437 mg) are linked together using HBTU (1.4 mmol; 531 mg) and DIPEA (2.8 mmol; 485 μL) dissolved in 15 mL of DMF; the reaction is then stirred for 16 h at RT. After isolation and purification of the intermediate (flash chromatography on silica gel, elution gradient from 2 to 5% MeOH in DCM), amine functions are released by reacting TFA (2 mL) in 30 mL DCM during 12 h. Yield: 59% (2 steps, 408 mg, 0.42 mmol).

### 2-[2,5-bis[4-(dimethylamino)butanoylamino]pentanoylamino]ethyl 2-[2,5-bis[[(9Z,12Z)-octadeca-9,12-dienoyl]amino]pentanoylamino]-5-[[(9Z,12Z)-octadeca-9,12-dienoyl]amino]pentanoate (XXVII)

Compound XXVII has been isolated adapting the procedure described for compound 1b, by linking Linoleic acid (1.5 mmol; 421 mg) with compound 27d (0.42 mmol; 408 mg) using DIPEA (2 mmol; 353 μL) and PyBOP (1.5 mmol; 780 mg) dissolved in 16 mL of DMF and 2 mL DCM. Reaction time: 24 h RT; Purification: Silica gel chromatography gradient 98/2 to 94/6 (DCM/MeOH, v/v). Yield: 26% (156 mg of white solid, 0.11 mmol, TLC (DCM/MeOH: 95/5) Rf: 0.3).

### Synthesis Molecule XXVIII

### [4-methyl-5-[[4-[[2-methyl-5-[(2,2,2-trifluoroacetyl)ammonio]pentanoyl]amino]-5-oxo-5-(4-pyrrolidin-1-ylbutoxy)pentyl]amino]-5-oxo-pentyl]-(2,2,2-trifluoroacetyl)ammonium-bis-trifluoroacetate (28a)

Compound 28a is obtained following the two steps procedure experienced for the synthesis of compound 13b. First, Boc-L-Orn(Boc)-OH (365 mg, 1.1 mmoles) and compound 13a (274 mg, 0.5 mmoles) are linked together using HOBt (162 mg, 1.2 mmoles) and DIC (201 μL, 1.3 mmoles) dissolved in 20 mL of a 9/1 v/v mixture of dry DCM/DMF that contains also Et₃N (210 μL, 1.5 mmoles); the reaction is then stirred for 72 h at RT. After isolation and purification of the intermediate compound, amine functions are released by reacting TFA (190 μL) in 25 mL DCM during 2 h. Yield: 48% (2 steps, 0.24 mmoles, 234 mg).

### (9Z,12Z)—N-[5-[[4-[2,5-bis[[(9Z,12Z)-octadeca-9,12-dienoyl]amino]pentanoylamino]-5-[2-[2-[[2-(dimethylamino)acetyl]amino]ethyldisulfanyl]ethylamino]-5-oxo-pentyl]amino]-4-[[(9Z,12Z)-octadeca-9,12-dienoyl]amino]-5-oxo-pentyl]octadeca-9,12-dienamide (XXXX)

Compound XXVIII has been isolated adapting the procedure described for compound X, by linking Linoleic acid (404 μL, 1.3 mmoles) with compound 28a (0.21 mmoles, 204 mg) using DIPEA (263 μL, 1.5 mmoles), HBTU (530 mg, 1.4 mmoles) and HOBt (183 mg, 1.35 mmoles) dissolved in 6 mL of DMF and 6 mL DCM. Reaction time: 36 h 0 °C to RT; Purification: Silica gel chromatography gradient 95/5 to 90/10 (DCM/MeOH, v/v). Yield: 54% (179 mg of white solid, 0.11 mmol, TLC (DCM/MeOH: 95/5) Rf: 0.3).

### Synthesis Molecule XXIX

### [5-[[1-[2-[2,5-bis[4-(dimethylamino)butanoylamino]pentanoylamino]ethoxycarbonyl]-4-[[2-methyl-5-[(2,2,2-trifluoroacetyl)ammonio]pentanoyl]amino]butyl]amino]-4-methyl-5-oxo-pentyl]-(2,2,2-trifluoroacetyl)ammonium (29a)

Compound 29a is obtained following the two steps procedure experienced for the synthesis of compound 13b. First, Boc-L-Orn(Boc)-OH (183 mg, 0.55 mmoles) and compound 14d (178 mg, 0.25 mmoles) are linked together using HOBt (81 mg, 0.6 mmoles) and DIC (100 μL, 0.65 mmoles) dissolved in 10 mL of a 9/1 v/v mixture of dry DCM/DMF that contains also Et₃N 105 μL, 0.75 mmoles); the reaction is then stirred for 72 h at RT. After isolation and purification of the intermediate compound, amine functions are released by reacting TFA (95 μL) in 15 mL DCM during 2 h. Yield: 51% (2 steps; 0.127 mmoles, 145 mg).

### 2-[2,5-bis[4-(dimethylamino)butanoylamino]pentanoylamino]ethyl2,5-bis[2,5-bis[[(9Z,12Z)-octadeca-9,12-dienoyl]amino]pentanoylamino]pentanoate (XXXIX)

Compound XXXIX has been isolated adapting the procedure described for compound X, by linking Linoleic acid ((202 μL, 0.65 mmoles) with compound 29a (0.1 mmoles, 113 mg) using DIPEA (132 μL, 0.75 mmoles), HBTU (265 mg, 0.7 mmoles) and HOBt (92 mg, 0.68 mmoles) dissolved in 6 mL of DMF and 6 mL DCM. Reaction time: 36 h 0 °C to RT; Purification: Silica gel chromatography gradient 93/7 to 90/10 (DCM/MeOH, v/v). Yield: 54% (179 mg of white solid, 0.11 mmol, TLC (DCM/MeOH: 95/5) Rf: 0.2).

### Synthesis Molecule XXX

### [5-oxo-5-(4-pyrrolidin-1-ylbutoxy)-4-[(2,2,2-trifluoroacetyl)ammonio]pentyl]-(2,2,2-trifluoroacetyl)ammonium (30a)

Compound 30a is obtained following the two steps procedure experienced for the synthesis of compound 13b. First, Boc-L-Orn(Boc)-OH (10 mmoles, 3.32 g) and 4-(1-Pyrrolidinyl)-1-butanol (1.43 g, 10 mmoles) are linked together using HOBt (1.49 g, 11 mmoles) and DIC (1.7 mL, 11 mmoles) dissolved in 100 mL of a 9/1 v/v mixture of dry DCM/DMF that contains also Et₃N (2.1 mL, 15 mmoles); the reaction is then stirred for 36 h at RT. After isolation and purification of the intermediate compound, amine functions are released by reacting TFA (1.3 mL) in 60 mL DCM during 2 h. Yield: 83% (2 steps; 8.3 mmoles, 3.76 g).

### [4-methyl-5-[[4-[[2-methyl-5-[(2,2,2-trifluoroacetyl)ammonio]pentanoyl]amino]-5-oxo-5-(4-pyrrolidin-1-ylbutoxy)pentyl]amino]-5-oxo-pentyl]-(2,2,2-trifluoroacetyl)ammonium (30b)

Compound 30b is obtained following the two steps procedure experienced for the synthesis of compound 13b. First, Boc-L-Orn(Boc)-OH (1.46 g, 4.4 mmoles) and 30a (906 mg, 2 mmoles) are linked together using HOBt (648 mg, 4.8 mmoles) and DIC (800 μL, 5.2 mmoles) dissolved in 40 mL of a 9/1 v/v mixture of dry DCM/DMF that contains also Et₃N (2.1 mL, 15 mmoles); the reaction is then stirred for 72 h at RT. After isolation and purification of the intermediate compound, amine functions are released by reacting TFA (760 μL) in 30 mL DCM during 6 h. Yield: 44% (2 steps; 8.3 mmoles, 772 mg).

### 4-pyrrolidin-1-ylbutyl-2,5-bis[2,5-bis[[(9Z,12Z)-octadeca-9,12-dienoyl]amino]pentanoylamino]pentanoate (XXX)

Compound XXX has been isolated adapting the procedure described for compound X, by linking Linoleic acid (404 μL, 1.3 mmoles) with compound 30b (0.25 mmoles, 219 mg) using DIPEA (263 μL, 1.5 mmoles), HBTU (530 mg, 1.4 mmoles) and HOBt (183 mg, 1.35 mmoles) dissolved in 6 mL of DMF and 6 mL DCM. Reaction time: 36 h 0 °C to RT; Purification: Silica gel chromatography gradient 95/5 to 90/10 (DCM/MeOH, v/v). Yield: 59% (227 mg of white solid, 0.148 mmol, TLC (DCM/MeOH: 95/5) Rf: 0.25).

### Synthesis Molecule XXXI

### [4-(3-butylhept-2-enoxy)-4-oxo-butyl]ammonium (31a)

3-Butyl-2-hepten-1-ol (5.5 mmol; 1.11 mL) is added under stirring to a solution of N-BOC-gamma-Aminobutyric acid (5 mmol, 1.02 g), DCC (5.5 mmol; 1.13 g) and DMAP (1 mmol; 122 mg) dissolved in 50 mL of anhydrous DCM. The reaction is then maintained under an inert atmosphere at ambient temperature for 16 hours. After total consumption of the acid, the reaction medium is evaporated to dryness under a rough vacuum. The crude is taken back in 50 mL of DCM and washed with 1M HCl, NaHCO₃ and brine. The organic layer is dried on MgSO₄ et then purified by flash chromatography on silica gel (elution gradient from 0 to 50% EtOAc in petroleum ether). In this way, the product is isolated in a yield of 79% (3.95 mmol; 1.4 g). TLC:Rf=0.4 (EtOAc:Petroleum ether 30/70 (v/v))

Residue (3.95 mmol; 1.4 g) is then dissolved in 100 mL of DCM, then 5 mL of trifluoroacetic acid is added to the reaction medium. The reaction is then maintained for one night under stirring at ambient temperature. The reaction medium is evaporated to dryness under a rough vacuum. The final residue is taken back four times, in succession, in DCM and four times in Et₂O then evaporated to dryness. The reaction is quantitative.

### 3-butylhept-2-enyl 4-[[4-amino-5-[[4-(3-butylhept-2-enoxy)-4-oxo-butyl]amino]-5-oxo-pentanoyl]amino]butanoate (31b))

Following the procedure described for compound 15d, molecule 31b is obtained in two steps by coupling (Fmoc-Glu-OH, 259 mg, 0.7 mmoles) and compound 31a (1.82 mmol; 672 mg) using HOBt (331 mg, 2.45 mmoles), DIC (325 μL, 2.1 mmoles) and Et₃N (394 μL, 2.8 mmoles) dissolved in chloroform (7 mL) and DMF (7 mL), and stirring 48 h. Then, the Fmoc group is removed by using piperidine (2 mL) and stirring 4 h RT. Reaction time: 48 h+4 h RT; Purification: Silica gel chromatography gradient 95/5 to 90/10 (DCM/MeOH, v/v).Yield: 41% (0.287 mmol; 179 mg); TLC:Rf=0.4 (DCM:MeOH 90/10 (v/v)).).

### 3-butylhept-2-enyl-4-[[5-[[4-(3-butylhept-2-enoxy)-4-oxo-butyl]amino]-4-[4-(dimethylamino)butanoylamino]-5-oxo-pentanoyl]amino]butanoate (XXXI)

Compound XXXI has been isolated adapting the procedure described for compound 5b, by coupling DMABA (0.72 mmol, 121 mg) with compound 31b (0.287 mmol; 179 mg) using DIPEA (1.42 mmol; 245 μL) and HBTU (0.72 mmol; 271 mg) dissolved in 2.5 mL of DCM and 2.5 mL DMF. Reaction time: 24 h RT; Purification: Silica gel chromatography gradient 98/2 to 90/10 (DCM/MeOH, v/v). Yield: 38% (81 mg of white solid, 0.11 mmol, TLC (DCM/MeOH: 95/5) Rf: 0.2).

### Synthesis Molecule XXXII

### a) Synthesis Molecule 32c

### tert-butyl N-(4-hydroxybutyl)carbamate (32a)

4-aminobutan-1-ol (10 mmoles, 922 μL) is dissolved in 20 mL of dry absolute ethanol. The mixture is then cooled down to zero degree. Then 2.3 mL of Boc₂O (10 mmoles) are added dropwise over 30 minutes while stirring at 0 °C. The reaction is then pursued for 48 h before being concentrated under vacuum. The oily residue is the redissolved in 20 mL of a 1/1 (v/v) mixture of DCM and water. The phase is decanted in a funnel, and the aqueous layer is extracted thrice with 5 mL of DCM. Combined organic extracts are then washed with brine, dried on magnesium sulfate, filtered and concentrated to dryness. The oil is used without additional purification. (97% yield, 1.84 g, 9.7 mmoles). TLC (DCM/MeOH: 95/5) rf: 0.7.

### 4-((2-hexyldecanoyl)oxy)butan-1-aminium (32b)

2-hexyldecanoic acid (8.8 mmol; 2.3 g) is placed beforehand in a dry 100 mL flask under an inert atmosphere then dissolved in 45 mL of DCM under stirring. DCC (10.6 mmol; 1.34 g) and DMAP (2 mmol; 244 mg) are added to the reaction medium followed by 4-boc-amino-1-butanol (10.6 mmol; 2 g). After stirring at ambient temperature under an inert atmosphere for 15 hours, the medium is filtered, to remove DCU, and washed with NH₄Cl, NaHCO₃ and brine. The organic layer is dried on MgSO₄ and purified by flash chromatography on silica gel (elution gradient from 5 to 15% EtOAC in petroleum ether). In this way, the product is isolated, in a yield of 88% (7.8 mmol; 3.34 g). TLC:Rf=0.5 (EtOAC:EP 10/90 (v/v)) The oily compound is dissolved under argon in 100 mL of dry DCM. 2 mL of trifluoracetic acid (TFA) are then added and the reaction is pursued until complete conversion evidenced by TLC (5 h). The mixture is then concentrated, co-evaporated thrice with 100 mL of DCM, thrice with 100 mL of diethyl ether (Et₂O), and dried under high vacuum overnight. The product is then used as it without further purification (7.9 mmoles, 3.4 g, quantitative yield).

### 4-[[4-amino-5-[4-(2-hexyldecanoyloxy)butylamino]-5-oxo-pentanoyl]amino]butyl-2-hexyldecanoate (32c)

Fmoc-glutamic acid (2.6 mmol; 959 mg) is dissolved in 20 mL of anhydrous DMF under stirring and under an inert atmosphere. DIC (7.9 mmol; 1.22 mL), then HOBt (7.9 mmol; 1.07 g) are successively added to the reaction medium. The reaction is then stirred for 3 hours before 32b (7.9 mmol; 3.4 g), in solution in 30 mL of anhydrous CHCl₃, is added dropwise to the reaction medium. After 48 hours, the reaction is stopped and the reaction medium is evaporated to dryness under a rough vacuum. The residue is taken back in 50 mL of EtOAc and filtered to remove DIU. The organic layer is washed with water and brine, dried over MgSO₄ and evaporated. The crude is dissolved in 50 mL DMF and NaN₃ (5.2 mmol; 338 mg) is added. The reaction medium is heated for 12 hours at 50 °C. After the complete deprotection of the Fmoc (TLC Rf=0.5 DCM:MeOH 95/5 (v/v)), the medium is evaporated and the crude purified by flash chromatography on silica gel (elution gradient from 2 to 5% MeOH in DCM). In this way, the product is isolated, in a yield of 62% (1.61 mmol; 1.23 g).

### Synthesis Molecule XXXII; XXXIII; XXXIV

### 4-[[4-[4-(dimethylamino)butanoylamino]-5-[4-(2-hexyldecanoyloxy)butylamino]-5-oxo-pentanoyl]amino]butyl 2-hexyldecanoate (XXXII)

DMABA (0.26 mmol, 44 mg) is placed in a dry 25 mL flask under an inert atmosphere then dissolved in 2 mL of anhydrous DMF under stirring. HBTU (0.26 mmol; 99 mg) and DIPEA (0.3 mmol; 51 μL)) are successively added to the reaction medium. The reaction is then maintained under an inert atmosphere at ambient temperature for 15 minutes before 32c (0.09 mmol; 69 mg), in solution in 1 mL of anhydrous DCM, is added to the reaction medium. After stirring at ambient temperature under an inert atmosphere for 48 hours, the reaction is stopped and the reaction medium is evaporated to dryness under a rough vacuum. The product is purified by flash chromatography on silica gel (elution gradient from 4 to 10% MeOH in DCM). In this way the product is isolated, in a yield of 65% (0.05 mmol; 52 mg). TLC:Rf=0.3 (DCM:MeOH 90/10 (v/v))

### 4-[[4-[bis(2-hydroxypropyl)amino]-5-[4-(2-hexyldecanoyloxy)butylamino]-5-oxo-pentanoyl]amino]butyl 2-hexyldecanoate (XXXIII)

32c (0.25 mmol; 190 mg) is dissolved in 5 mL of MeOH in a dry 50 mL flask under an inert atmosphere. A large excess of propylene oxide (1 mL) is added dropwise and the reaction medium is heated to 60 °C for 16 hours. The methanol is then evaporated and the crude is purified by flash chromatography on silica gel (elution gradient from 2 to 5% MeOH in DCM). In this way the product is isolated, in a yield of 56% (0.14 mmol; 123 mg). TLC:Rf=0.6 (DCM:MeOH 90/10 (v/v))

### 4-[[4-[[2-(dimethylamino)acetyl]amino]-5-[4-(2-hexyldecanoyloxy)butylamino]-5-oxo-pentanoyl]amino]butyl 2-hexyldecanoate (XXXIV)

Following the procedure described for compound XXXII, molecule XXXIV is obtained by coupling DMG (0.25 mmol, 26 mg) with 32c (0.25 mmol, 190 mg) with DIPEA (1 mmol; 170 μL) and HBTU (0.5 mmol; 190 mg) dissolved in 4 mL DMF and 1 mL DCM. Reaction time: 36 h RT; Purification: Silica gel chromatography gradient 96/4 to 90/10 (DCM/MeOH, v/v). Yield: 60% (125 mg, 0.15 mmol, TLC (DCM/MeOH: 90/10) Rf: 0.3).

### Synthesis Molecule XXXV

### 4-(9H-fluoren-9-ylmethoxycarbonylamino)-5-(hexadecylamino)-5-oxo-pentanoic acid (35a)

Fmoc-Glu(OtBu)-OH (1 mmol; 426 mg) is dissolved in 10 mL of anhydrous DMF under stirring and under an inert atmosphere. DIC (1.6 mmol; 247 μL), then HOBt (1.6 mmol; 216 mg) are successively added to the reaction medium. The reaction is then stirred for 3 hours before hexadecylamine (1.6 mmol; 387 mg), in solution in 100 mL of anhydrous CHCl₃, is added dropwise to the reaction medium. After 48 hours, the reaction is stopped and the reaction medium is evaporated to dryness under a rough vacuum.

The residue is taken back in 50 mL of EtOAc and filtered to remove DIU. The organic layer is washed with water and brine, dried over MgSO₄ and evaporated. The white product is washed several times with ether and then dissolved in 15 mL DCM. The medium is stirred under argon before 4 mL of TFA and 500 μL of triethylsilane are added. After 15 hours, the tert-Butyl group is completely deprotected (TLC Rf=0.4 EtOAc/EP 40/60 (v/v)). The mixture is then concentrated, co-evaporated thrice with 50 mL of DCM, thrice with 50 mL of diethyl ether (Et₂O), and dried under high vacuum overnight. The product is then used as it without further purification (0.71 mmol, 421 mg). Yield of 71% over two steps.

### 4-[[4-amino-5-(hexadecylamino)-5-oxo-pentanoyl]amino]butyl 2-hexyldecanoate (35b)

35a (0.48 mmol, 289 mg) is placed beforehand in a dry 50 mL flask under an inert atmosphere then dissolved in 10 mL of anhydrous DMF under stirring. COMU (0.5 mmol; 214 mg) and DIPEA (1 mmol; 180 μL) are successively added to the reaction medium. The reaction is then maintained under an inert atmosphere and 0 °C for 15 minutes before 32b (0.5 mmol; 214 mg) is added to the reaction medium under stirring. The solution is allowed to warm to ambient temperature under an inert atmosphere overnight. The mixture is evaporated to dryness under a rough vacuum. The residue is taken back in 50 mL of EtOAc and heated until dissolution. The solution is put in falcons at −20 °C. The formed precipitate is washed several times by centrifugation with cold EtOAc dried under high vacuum. A white solid is obtained. TLC:Rf=0.4 (DCM:MeOH 95/5 (v/v)).

The product is taken back in 30 mL DMF and 2 mL of piperidine are added. The reaction is then maintained for one night under stirring at ambient temperature. The reaction medium is evaporated to dryness under a rough vacuum. The product is then purified by flash chromatography on silica gel (elution gradient from 2 to 5% MeOH in DCM). In this way the pure compound is isolated, in a yield of 50% (0.24 mmol; 160 mg). TLC:Rf=0.6 (DCM:MeOH 90/10 (v/v))

### 4-[[4-[[2-(dimethylamino)acetyl]amino]-5-(hexadecylamino)-5-oxo-pentanoyl]amino]butyl 2-hexyldecanoate (XXXV)

Following the procedure described for compound XXXIV, molecule XXXV is obtained by coupling DMG (0.2 mmol, 21 mg) with 35a (0.1 mmol, 68 mg) with DIPEA (0.4 mmol; 68 μL) and HBTU (0.2 mmol; 76 mg) dissolved in 2 mL DMF and 1 mL DCM. Reaction time: 36 h RT; Purification: Silica gel chromatography gradient 96/4 to 90/10 (DCM/MeOH, v/v). Yield: 67% (51 mg, 0.067 mmol, TLC (DCM/MeOH: 90/10) Rf: 0.3).

### Synthesis of molecule XXXVI & XXXVII

### 4-[[4-[4-(2-hexyldecanoyloxy)butylamino]-3-hydroxy-4-oxo-butanoyl]amino]butyl 2-hexyldecanoate (36a)

Malic acid (1.57 mmol; 209 mg) is placed in a dry 50 mL flask under an inert atmosphere then dissolved in 25 mL of anhydrous DCM under stirring. HOBt (4 mmol; 540 mg), in 2 mL DMF, and 32b (4.67 mmol; 2 g) are successively added to the reaction medium followed by NEt₃ (0.5 mmol; 51 μL)). The reaction is then maintained under stirring before EDC, Cl (4.7 mmol; 902 mg) is added to the reaction medium. After stirring at ambient temperature under an inert atmosphere for 20 hours, the reaction is stopped and the reaction medium is evaporated to dryness under a rough vacuum. The product is purified by flash chromatography on silica gel (elution gradient from 40 to 80% EtOAC in EP). In this way the product is isolated, in a yield of 55% (0.86 mmol; 650 mg). TLC:Rf=0.5 (EtOAc:EP 50/50 (v/v))

### 4-[[4-[4-(2-hexyldecanoyloxy)butylamino]-3-hydroxy-4-oxo-butanoyl]amino]butyl 2-hexyldecanoate (XXXVI)

DMABA (0.2 mmol; 34 mg) and DMAP (0.05 mmol; 6.1 mg) are dissolved in 2 mL of anhydrous DCM with molecular sieve under stirring and under an inert atmosphere. 36a (0.13 mmol; 100 mg), then EDC (0.2 mmol; 38 mg) are successively added to the reaction medium. After 48 hours, the reaction is stopped and the reaction medium is evaporated to dryness under a rough vacuum. The residue is taken back in 20 mL of DCM, washed with water and brine. The organic layer is dried over MgSO₄ and evaporated. The crude purified by flash chromatography on silica gel (elution gradient from 4 to 10% MeOH in DCM). In this way, the product is isolated, in a yield of 66% (0.09 mmol; 74 mg).

### Synthesis 4-[[3-[2-(dimethylamino)acetyl]oxy-4-[4-(2-hexyldecanoyloxy)butylamino]-4-oxo-butanoyl]amino]butyl 2-hexyldecanoate (XXXVII)

Following the procedure described for compound XXXVI, molecule XXXVII is obtained by coupling DMG (0.2 mmol, 21 mg) with 36a (0.1 mmol, 75 mg) with DMAP (0.04 mmol; 5 mg) and EDC (0.3 mmol; 58 mg) dissolved in 2 mL DCM. Reaction time: 36 h RT; Purification: Silica gel chromatography gradient 100/0 to 96/4 (DCM/MeOH, v/v). Yield: 65% (55 mg, 0.065 mmol, TLC (DCM/MeOH: 95/5) Rf: 0.7).

### Synthesis of molecule 38c

### 4-[2-[2-(tert-butoxycarbonylamino)ethyldisulfanyl]ethylamino]-4-oxo-butanoic acid (38a)

Boc-cystamine (5.3 mmol; 1.34 g)) is dissolved under argon in 20 mL of a 1/1 (v/v) mixture of dry DCM and DMF. Triethylamine (7.9 mmol: 1.1 mL) is then added slowly and the reaction is stirred for 5 minutes. Then, succinic anhydride (10.6 mmoles, 1.06 g) is added and the mixture is stirred for 12 h at room temperature. The reaction is then concentrated and the crude is purified by flash chromatography on silica gel (elution gradient from 2 to 8% MeOH in DCM). In this way, the product is isolated, in a yield of 93% (4.9 mmol; 1.6 g).

### 2-[2-[[4-(2-hexyldecoxy)-4-oxo-butanoyl]amino]ethyldisulfanyl]ethylammonium (38b)

38a (0.6 mmol, 194 mg) is placed in a dry 25 mL flask under an argon then dissolved in 6 mL of DCM under stirring. DCC (1 mmol; 206 mg) and DMAP (0.1 mmol; 12 mg) are added to the reaction medium. The reaction is then stirred for 10 minutes before 2-hexyldecanol (0.7 mmol; 170 mg) is introduced. After stirring at RT under argon for 16 hours, the reaction is stopped and the medium is evaporated under vacuum. The crude is taken back in 20 mL of DCM and washed with 1M HCl, NaHCO₃ and brine. The organic extract is dried on MgSO₄ and purified by flash chromatography on silica gel (elution gradient from 20 to 50% EtOAc in EP, TLC:Rf=0.5 (EtOAc/EP 50/50 (v/v)) before being dissolved in 20 mL DCM while 1 mL of trifluoroacetic acid is added. The reaction is then maintained for one night under stirring at RT. The reaction medium is evaporated and is co-evaporated, four times in DCM and four times in Et₂O before drying. Yield 84% (2 steps, 0.51 mmoles, 299 mg).

### 2-hexyldecyl4-[2-[2-[[4-amino-5-[2-[2-[[4-(2-hexyldecoxy)-4-oxo-butanoyl]amino]ethyldisulfanyl]ethylamino]-5-oxo-pentanoyl]amino]ethyldisulfanyl]ethylamino]-4-oxo-butanoate (38c)

Fmoc-glutamic acid (0.2 mmol; 74 mg) is dissolved in 2 mL of anhydrous DMF under stirring and under an inert atmosphere. DIC (0.5 mmol; 77 μL), then HOBt (0.5 mmol; 68 mg) are successively added to the reaction medium. The reaction is then stirred for 3 hours before 38a (0.5 mmol; 300 mg) with NEt₃ (0.6 mmol; 84 μL), in solution in 2 mL of anhydrous CHCl₃, is added dropwise to the reaction medium. After 48 hours, the reaction is stopped and the reaction medium is evaporated to dryness under a rough vacuum. The residue is taken back in 50 mL of EtOAc and filtered to remove DIU. The organic layer is washed with water and brine, dried over MgSO₄ and evaporated. The crude is dissolved in 10 mL DMF and NaN₃ (20 mg) is added. The reaction medium is heated for 16 hours at 50 °C. After the complete deprotection of the Fmoc (TLC Rf=0.4 DCM:MeOH 95/5 (v/v)), the medium is evaporated and the crude purified by flash chromatography on silica gel (elution gradient from 2 to 6% MeOH in DCM). In this way, the product is isolated, in a yield of 46% (0.09 mmol; 100 mg).

### Synthesis of molecule XXXVIII & XXXIX

### 2-hexyldecyl 4-[2-[2-[[4-[[2-(dimethylamino)acetyl]amino]-5-[2-[2-[[4-(2-hexyldecoxy)-4-oxo-butanoyl]amino]ethyldisulfanyl]ethylamino]-5-oxo-pentanoyl]amino]ethyldisulfanyl]ethylamino]-4-oxo-butanoate (XXXVIII)

Following the procedure described for compound XXXIV, molecule XXXVIII is obtained by coupling DMG (0.2 mmol, 21 mg) with 38c (0.09 mmol, 100 mg) with DIPEA (0.4 mmol; 68 μL) and HBTU (0.2 mmol; 76 mg) dissolved in 2 mL DMF. Reaction time: 36 h RT; Purification: Silica gel chromatography gradient 98/2 to 92/8 (DCM/MeOH, v/v). Yield: 42% (51 mg, 0.04 mmol, TLC (DCM/MeOH: 90/10) Rf: 0.3).

### 2-hexyldecyl 4-[2-[2-[[4-[bis(2-hydroxypropyl)amino]-5-[2-[2-[[4-(2-hexyldecoxy)-4-oxo-butanoyl]amino]ethyldisulfanyl]ethylamino]-5-oxo-pentanoyl]amino]ethyldisulfanyl]ethylamino]-4-oxo-butanoate (XXXIX)

Following the procedure described for compound XXXIII, molecule XXXIX is obtained by alkylation of 38c (0.07 mmol, 75 mg) with a large excess of propylene oxide (500 μL) in 2 mL of MeOH. Reaction time: 16 h RT; Purification: Silica gel chromatography gradient 98/2 to 90/10 (DCM/MeOH, v/v). Yield: 13% (10 mg, 0.009 mmol, TLC (DCM/MeOH: 90/10) Rf: 0.3).

### Synthesis of Molecule XL

### 4-[(Z)-non-2-enoxy]-4-oxo-butanoic acid (40a)

Cis-2-noneol (35 mmol; 5.92 mL) is dissolved under argon in 70 mL of a 2/1 (v/v) mixture of dry DCM and DMF. DMAP (35 mmol; 4 g) is added slowly and the reaction is stirred for 5 minutes. Then, succinic anhydride (53 mmol, 5.3 g) is added and the mixture is stirred for 48 h at room temperature. The reaction is then concentrated and the crude is taken back in 50 mL of EtOAc and washed with HCl 1M, water and brine. The organic layer is dried over MgSO₄ and evaporated. A clear oil is obtained in a yield of 97% (34 mmol; 8.3 g).

### [4-(9H-fluoren-9-ylmethoxycarbonylamino)-5-[4-(2-hexyldecanoyloxy)butylamino]-5-oxo-pentyl]ammonium (40b)

FmocOrn(Boc)OH (1 mmol, 454 mg) is placed beforehand in a dry 50 mL flask under an inert atmosphere then dissolved in 10 mL of anhydrous DMF under stirring. COMU (1.2 mmol; 514 mg) and DIPEA (3 mmol; 509 μL) are successively added to the reaction medium. The reaction is then maintained under an inert atmosphere and 0 °C for 15 minutes before 32b (1.3 mmol; 560 mg) is added to the reaction medium under stirring. The solution is allowed to warm to ambient temperature under an inert atmosphere overnight. The mixture is evaporated to dryness under a rough vacuum. The residue is taken back in 50 mL of EtOAc and washed with HCl 1M, water and brine. The organic layer is dried over MgSO₄ and evaporated. The product is then dissolved in 50 mL DCM with 2.5 mL of TFA. After 15 hours, the mixture is concentrated, co-evaporated thrice with 50 mL of DCM, thrice with 50 mL of diethyl ether (Et₂O), and dried under high vacuum overnight. The product is then used as it without further purification (850 mg).

### 4-[[2-amino-5-[[4-[(Z)-non-2-enoxy]-4-oxo-butanoyl]amino]pentanoyl]amino]butyl 2-hexyldecanoate (40c)

40a (0.8 mmol; 194 mg) is placed beforehand in a dry 50 mL flask under an inert atmosphere then dissolved in 10 mL of anhydrous DMF under stirring. COMU (0.9 mmol; 385 mg) and DIPEA (2 mmol; 340 μL) are successively added to the reaction medium. The reaction is then maintained under an inert atmosphere and 0 °C for 15 minutes before 40b (1 mmol, 850 mg), dissolved in 5 ml DMF, is added to the reaction medium under stirring. The solution is allowed to warm to ambient temperature under an inert atmosphere overnight. The mixture is evaporated to dryness under a rough vacuum. The residue is taken back in 50 mL of EtOAc and washed with HCl 1M, water and brine. The organic layer is dried over MgSO₄ and evaporated. The product is then dissolved in 20 mL DMF with 4 mL of piperidine. After 15 hours, the mixture is concentrated and the crude purified by flash chromatography on silica gel (elution gradient from 2 to 8% MeOH in DCM). In this way, the product is isolated, in a yield of 42% (0.42 mmol; 280 mg).

### 4-[[2-[[2-(dimethylamino)acetyl]amino]-5-[[4-[(Z)-non-2-enoxy]-4-oxo-butanoyl]amino]pentanoyl]amino]butyl 2-hexyldecanoate (XL)

Following the procedure described for compound XXXIV, molecule XL is obtained by coupling DMG (0.2 mmol, 21 mg) with 40c (0.15 mmol, 100 mg) with DIPEA (0.4 mmol; 68 μL) and HBTU (0.3 mmol; 114 mg) dissolved in 5 mL DMF. Reaction time: 36 h RT; Purification: Silica gel chromatography gradient 98/2 to 92/8 (DCM/MeOH, v/v). Yield: 42% (47 mg, 0.06 mmol, TLC (DCM/MeOH: 90/10) Rf: 0.3).

### Synthesis of Molecule XLI

### 5-[3-(dimethylamino)-2-hydroxy-propoxy]-4-(9H-fluoren-9-ylmethoxycarbonylamino)-5-oxo-pentanoic acid (41a)

Iodine (1.5 mmol; 171 mg) is dissolved in 20 mL of anhydrous DCM under stirring and under an inert atmosphere with molecular sieve. Triphenylphosphine (1.5 mmol; 393 mg), imidazole (3.3 mmol; 224 mg), then Fmoc-Glu(OtBu)-OH (1 mmol; 426 mg) are successively added to the reaction medium. The reaction is then stirred for 10 minutes before 3-(dimethylamino)-1,2-propanediol (1.5 mmol; 178 μL) is added dropwise to the reaction medium. After 20 hours, 30 mL of DCM is added and the organic layer is washed twice with HCl 1M and brine, dried over MgSO₄ and evaporated. The crude is purified by flash chromatography on silica gel (elution gradient from 40 to 80% EtOAC in EP). In this way the product is isolated, in a yield of 90% (0.9 mmol; 500 mg). TLC:Rf=0.5 (EtOAc:EP 50/50 (v/v)) The product is then dissolved in 50 mL DCM with 2.5 mL of TFA. After 15 hours, the mixture is concentrated, co-evaporated thrice with 50 mL of DCM, thrice with 50 mL of diethyl ether (Et₂O), and dried under high vacuum overnight. The product is then used as it without further purification.

### 4-[[4-amino-5-[3-(dimethylamino)-2-hydroxy-propoxy]-5-oxo-pentanoyl]amino]butyl 2-hexyldecanoate (41b)

41a (0.5 mmol, 242 mg) is placed beforehand in a dry 50 mL flask under an inert atmosphere then dissolved in 5 mL of anhydrous DMF under stirring. COMU (0.6 mmol; 257 mg) and DIPEA (1.5 mmol; 255 μL) are successively added to the reaction medium. The reaction is then maintained under an inert atmosphere and 0 °C for 15 minutes before 32b (0.6 mmol; 257 mg), dissolved in 4 mL DCM/DMF, is added to the reaction medium under stirring. The solution is allowed to warm to ambient temperature under an inert atmosphere overnight. The mixture is evaporated to dryness under a rough vacuum. The residue is taken back in 50 mL of EtOAc and washed with HCl 1M, water and brine. The organic layer is dried over MgSO₄ and evaporated. The product is then dissolved in 10 mL DMF with 50 mg of NaN₃, and the reaction medium is heated at 50 °C. After 15 hours, the mixture is concentrated and the crude is purified by flash chromatography on silica gel (elution gradient from 2 to 5% MeOH in DCM). In this way the product is isolated, in a yield of 34% (0.17 mmol; 96 mg). TLC:Rf=0.5 (DCM/MeOH 95/5 (v/v))

### 4-[[5-[3-(dimethylamino)-2-hydroxy-propoxy]-4-(dodecanoylamino)-5-oxo-pentanoyl]amino]butyl 2-hexyldecanoate (XLI)

41b (0.17 mmol, 96 mg) is placed beforehand in a dry 25 mL flask under an inert atmosphere then dissolved in 5 mL of anhydrous DMF under stirring. PyBOP (0.22 mmol; 116 mg) and DIPEA (0.4 mmol; 70 μL) are successively added to the reaction medium. The reaction is then maintained under an inert atmosphere 5 minutes before dodecanoic acid (0.2 mmol; 54 mg) is added to the reaction medium under stirring. After 20 hours, the mixture is evaporated to dryness under a rough vacuum. The crude is purified by flash chromatography on silica gel (elution gradient from 1 to 5% MeOH in DCM). In this way the product is isolated, in a yield of 45% (0.076 mmol; 56 mg). TLC:Rf=0.6 (DCM/MeOH 95/5 (v/v)).

### Other Synthesis Methods

### Synthesis Molecule N,N′-bis[(Z)-octadec-9-enyl]-2-[[4-oxo-4-(2-pyrrolidin-1-ylethylamino)butanoyl]amino]pentanediamide

### 4-oxo-4-(2-pyrrolidin-1-ylethylamino)butanoic acid

2-pyrrolidin-1-ylethanamine (251 μL, 2 mmoles) is dissolved under argon in 20 mL of a 1/1 (v/v) mixture of dry DCM and DMF. 2 equivalents (4 mmoles, 560 μL) of triethylamine (Et₃N) are then added slowly and the reaction is stirred for 5 minutes. Then, succinic anhydride (4 mmoles, 400 mg) is added and the mixture is stirred for 12 h at room temperature. The reaction is then concentrated, and redissolved in 80 mL of ethyl acetate. The organic phase is then washed with a saturated aqueous ammonium chloride solution, twice with MilliQ water, once with brine, dried on MgSO₄, filtered and concentrated. This work-up lead to the clean expected product with 79% yield (339 mg, 1.58 mmoles), after high vacuum drying TLC (DCM/MeOH: 95/5) rf: 0.7.

### N,N′-bis[(Z)-octadec-9-enyl]-2-[[4-oxo-4-(2-pyrrolidin-1-ylethylamino)butanoyl]amino]pentanediamide

Molecule N,N′-bis[(Z)-octadec-9-enyl]-2-[[4-oxo-4-(2-pyrrolidin-1-ylethylamino)butanoyl]amino]pentanediamide has been isolated from the coupling of compound-oxo-4-(2-pyrrolidin-1-ylethylamino)butanoic acid (54 mg, 0.25 mmoles) and compound 2-amino-N,N′-bis[(Z)-octadec-9-enyl]pentanediamide (0.25 mmoles, 161 mg) using methylimidazole (74 μL, 0.92 mmoles), and TCFH (98 mg, 0.35 mmoles) in 4 mL of 2/1 (v/v) mixture solution of MeCN and DCM according to the procedure described for the compound 2-[4-(dimethylamino)butanoylamino]-N′—[(Z)-heptadec-9-enyl]-N—[(Z)-octadec-9-enyl]pentanediamide. Reaction time: 12 h room temperature; Purification: Silica gel chromatography gradient 98/2 to 90/10 (DCM/MeOH, v/v). Yield: 65% (135 mg, 0.16 mmoles, white solid TLC (DCM/MeOH: 90/10) rf: 0.4.).

### Synthesis Molecule 2-[[4-[2-[2-[[2-(dimethylamino)acetyl]amino]ethyldisulfanyl]ethylamino]-4-oxo-butanoyl]amino]-N,N′-bis[(Z)-octadec-9-enyl]pentanediamide

### 4-[2-[2-(tert-butoxycarbonylamino)ethyldisulfanyl]ethylamino]-4-oxo-butanoic acid

Compound 4-[2-[2-(tert-butoxycarbonylamino)ethyldisulfanyl]ethylamino]-4-oxo-butanoic acid is obtained by reacting Boc-Cystamine (5.3 mmol; 1.34 g), succinic anhydride (10.6 mmol; 1.06 g) and triethylamine (7.95 mmol; 1.1 mL) 20 mL of DCM and 5 mL of DMF according to the method described for compound 4-oxo-4-(2-pyrrolidin-1-ylethylamino)butanoic acid. Reaction time: 16 h room temperature; Purification: Silica gel chromatography gradient 98/2 to 95/5 (DCM/MeOH, v/v). Yield: 93% (1.6 g, 4.92 mmol, TLC (DCM/MeOH: 90/10) Rf: 0.6.)

### 2-[2-[[4-[[4-[[(Z)-octadec-9-enyl]amino]-1-[[(Z)-octadec-9-enyl]carbamoyl]-4-oxo-butyl]amino]-4-oxo-butanoyl]amino]ethyldisulfanyl]ethylammonium trifluoroacetate

4-[2-[2-(tert-butoxycarbonylamino)ethyldisulfanyl]ethylamino]-4-oxo-butanoic acid (1.6 mmol; 500 mg) is placed beforehand in a dry 100 mL flask under an inert atmosphere then dissolved in 40 mL of DCM under stirring. DIC (2.3 mmol; 360 μL) and HOBt (2.3 mmol; 314 mg), dissolved in 5 mL of anhydrous DMF, are successively added to the reaction medium. The reaction is then maintained under an inert atmosphere at ambient temperature for 2 and a half hours. 2-amino-N,N′-bis[(Z)-octadec-9-enyl]pentanediamide (2.2 mmol; 1.4 g) in solution in 10 mL of anhydrous DCM and 5 mL of anhydrous DMF with triethylamine (2.9 mmol; 400 μL) is added to the reaction medium under stirring. After 48 hours, the reaction is stopped and the reaction medium is evaporated to dryness under a rough vacuum. The intermediate product is first purified by flash chromatography on silica gel (elution gradient from 2 to 8% MeOH in DCM), then dissolved in 33 mL of DCM in a 50 mL flask, before 2.5 mL of trifluoroacetic acid is added to the reaction medium. The reaction is then maintained for one night under stirring at ambient temperature. The reaction medium is evaporated to dryness under a rough vacuum. In order to drive off the trifluoroacetic acid excess, the final residue is taken back four times, in succession, in DCM and four times in Et₂O then evaporated to dryness. In this way, the pure product is isolated, in a yield of 42% (0.68 mmol; 657 mg, 2 steps).

### Synthesis Molecule 2-[[4-[2-[2-[[2-(dimethylamino)acetyl]amino]ethyldisulfanyl]ethylamino]-4-oxo-butanoyl]amino]-N,N′-bis[(Z)-octadec-9-enyl]pentanediamide & 2-[[4-[2-[2-[4-(dimethylamino)butanoylamino]ethyldisulfanyl]ethylamino]-4-oxo-butanoyl]amino]-N,N′-bis[(Z)-octadec-9-enyl]pentanediamide

### 2-[[4-[2-[2-[[2-(dimethylamino)acetyl]amino]ethyldisulfanyl]ethylamino]-4-oxo-butanoyl]amino]-N,N′-bis[(Z)-octadec-9-enyl]pentanediamide

DMG (0.4 mmol, 41 mg) is placed in a dry 25 mL flask under an inert atmosphere then dissolved in 2 mL of anhydrous DMF under stirring. 2-(1H-benzotriazol-1-yl)-1,1,3,3-tetramethyluronium hexafluorophosphate (HBTU, 0.4 mmol; 152 mg) and DIPEA (0.8 mmol; 136 μL)) are successively added to the reaction medium. The reaction is then maintained under an inert atmosphere at ambient temperature for 15 minutes before 2-[2-[[4-[[4-[[(Z)-octadec-9-enyl]amino]-1-[[(Z)-octadec-9-enyl]carbamoyl]-4-oxo-butyl]amino]-4-oxo-butanoyl]amino]ethyldisulfanyl]ethylammonium trifluoroacetate (0.2 mmol; 198 mg), in solution in 1 mL of anhydrous DCM and 3 mL of DMF, is added to the reaction medium. After stirring at ambient temperature under an inert atmosphere for 48 hours, the reaction is stopped and the reaction medium is evaporated to dryness under a rough vacuum. The product is purified by flash chromatography on silica gel (elution gradient from 2 to 8% MeOH in DCM). In this way the product is isolated, in a yield of 32% (0.064 mmol; 60 mg). TLC:Rf=0.4 (DCM:MeOH 95/5 (v/v)).

### 2-[[4-[2-[2-[4-(dimethylamino)butanoylamino]ethyldisulfanyl]ethylamino]-4-oxo-butanoyl]amino]-N,N′-bis[(Z)-octadec-9-enyl]pentanediamide

Molecule 2-[[4-[2-[2-[4-(dimethylamino)butanoylamino]ethyldisulfanyl]ethylamino]-4-oxo-butanoyl]amino]-N,N′-bis[(Z)-octadec-9-enyl]pentanediamide has been isolated from the coupling of DMABA (0.6 mmol, 101 mg) and 2-[2-[[4-[[4-[[(Z)-octadec-9-enyl]amino]-1-[[(Z)-octadec-9-enyl]carbamoyl]-4-oxo-butyl]amino]-4-oxo-butanoyl]amino]ethyldisulfanyl]ethylammonium trifluoroacetate (0.3 mmol; 298 mg) using HBTU (0.6 mmol; 227 mg) and DIPEA (1.2 mmol; 204 μL) in 6 mL DMF and 2 mL DCM, according to the procedure described for the molecule 2-[[4-[2-[2-[[2-(dimethylamino)acetyl]amino]ethyldisulfanyl]ethylamino]-4-oxo-butanoyl]amino]-N,N′-bis[(Z)-octadec-9-enyl]pentanediamide. Reaction time: 48 h room temperature; Purification: Silica gel chromatography gradient 98/2 to 92/8 (DCM/MeOH, v/v). Yield: 35% (110 mg, 0.10 mmol, TLC (DCM/MeOH: 95/5) Rf: 0.4.).

### Synthesis Molecule bis[(9Z,12Z)-octadeca-9,12-dienyl]2-[[4-oxo-4-(4-pyrrolidin-1-ylbutoxy)butanoyl]amino]pentanedioate

### 4-[[4-[(9Z,12Z)-octadeca-9,12-dienoxy]-1-[(9Z,12Z)-octadeca-9,12-dienoxy]carbonyl-4-oxo-butyl]amino]-4-oxo-butanoic acid

Compound Bis[(9Z,12Z)-octadeca-9,12-dienyl]2-aminopentanedioate (1 mmol; 644 mg) is placed beforehand in a dry 50 mL flask under an inert atmosphere then dissolved in 15 mL of DCM and 5 mL of anhydrous DMF under stirring. Succinic anhydride (1.5 mmol; 150 mg) and triethylamine (2 mmol; 280 μL) are added to the reaction medium. After stirring at ambient temperature under an inert atmosphere for 16 hours, the reaction is stopped and the reaction medium is evaporated to dryness under a high vacuum. The crude is purified by flash chromatography on silica gel (elution gradient from 0 to 6% MeOH in DCM). The product is obtained in a yield of 91% (0.91 mmol; 677 mg). TLC:Rf=0.5 (DCM:MeOH 95/5 (v/v)).

### bis[(9Z,12Z)-octadeca-9,12-dienyl]2-[[4-oxo-4-(4-pyrrolidin-1-ylbutoxy)butanoyl]amino]pentanedioate

4-(1-pyrrolidinyl)-1-butanol (0.7 mmol; 100 mg) is added under stirring to a solution of 4-[[4-[(9Z,12Z)-octadeca-9,12-dienoxy]-1-[(9Z,12Z)-octadeca-9,12-dienoxy]carbonyl-4-oxo-butyl]amino]-4-oxo-butanoic acid (0.47 mmol; 350 mg), N,N′-Dicyclohexylcarbodiimide (DCC, 0.7 mmol; 145 mg) and 4-(Dimethylamino)pyridine (DMAP, 0.094 mmol; 11 mg) in 18 mL of DCM. After stirring at ambient temperature under an inert atmosphere for 15 hours, the reaction is stopped and the reaction medium is evaporated to dryness under high vacuum. The crude is taken back in 20 mL of DCM and washed with 1M HCl, NaHCO₃ and brine. The organic layer is dried on MgSO₄ and purified by flash chromatography on silica gel (elution gradient from 2 to 10% MeOH in DCM). In this way, the product is isolated, in a yield of 73% (0.34 mmol; 298 mg). TLC:Rf=0.3 (DCM:MeOH 95/5 (v/v)).

### Synthesis Molecule bis[(9Z,12Z)-octadeca-9,12-dienyl]2-[[4-[3-[bis(2-hydroxyethyl)amino]propylamino]-4-oxo-butanoyl]amino]pentanedioate

### bis[(9Z,12Z)-octadeca-9,12-dienyl]2-[[4-[3-[bis(2-hydroxyethyl)amino]propylamino]-4-oxo-butanoyl]amino]pentanedioate

Molecule bis[(9Z,12Z)-octadeca-9,12-dienyl]2-[[4-[3-[bis(2-hydroxyethyl)amino]propylamino]-4-oxo-butanoyl]amino]pentanedioate has been isolated from the coupling of compound 4-[[4-[(9Z,12Z)-octadeca-9,12-dienoxy]-1-[(9Z,12Z)-octadeca-9,12-dienoxy]carbonyl-4-oxo-butyl]amino]-4-oxo-butanoic acid (0.4 mmol, 298 mg) and N-(3-aminopropyl)diethanolamine (0.5 mmol; 76 μL) using HBTU (0.4 mmol; 152 mg) and DIPEA (0.8 mmol; 136 μL) dissolved in 8 mL of DMF according to the procedure described for the molecule 2-[[4-[2-[2-[[2-(dimethylamino)acetyl]amino]ethyldisulfanyl]ethylamino]-4-oxo-butanoyl]amino]-N,N′-bis[(Z)-octadec-9-enyl]pentanediamide Reaction time: 16 h RT; Purification: Silica gel chromatography gradient 98/2 to 90/10 (DCM/MeOH, v/v). Yield: 66% (0.26 mmol; 234 mg), TLC (DCM/MeOH: 95/5) Rf: 0.2.).

### Synthesis Molecule 2-[[2-[2,5-bis[4-(dimethylamino)butanoylamino]pentanoylamino]acetyl]amino]ethyl 4-[[4-[[(9Z,12Z)-octadeca-9,12-dienyl]amino]-1-[[(9Z,12Z)-octadeca-9,12-dienyl]carbamoyl]-4-oxo-butyl]amino]-4-oxo-butanoate

### tert-butyl-N-[4-(tert-butoxycarbonylamino)-5-[[2-(2-hydroxyethylamino)-2-oxo-ethyl]amino]-5-oxo-pentyl]carbamate

Molecule tert-butyl-N-[4-(tert-butoxycarbonylamino)-5-[[2-(2-hydroxyethylamino)-2-oxo-ethyl]amino]-5-oxo-pentyl]carbamate has been isolated from the coupling of Boc-L-Orn(Boc)-OH (1.5 mmol, 500 mg) and 2-amino-N-(2-hydroxyethyl)acetamide (2 mmol; 236 mg) using HBTU (1.5 mmol; 569 mg) and DIPEA (2 mmol; 340 μL) dissolved in 20 mL of DMF according to the procedure described for the molecule 2-[[4-[2-[2-[[2-(dimethylamino)acetyl]amino]ethyldisulfanyl]ethylamino]-4-oxo-butanoyl]amino]-N,N′-bis[(Z)-octadec-9-enyl]pentanediamide. Reaction time: 48 h room temperature; Purification: Silica gel chromatography gradient 96/4 to 90/10 (DCM/MeOH, v/v). Yield: 62% (402 mg, 0.93 mmol, TLC (DCM/MeOH: 95/5) Rf: 0.3.).

### 4-[[4-[[(9Z,12Z)-octadeca-9,12-dienyl]amino]-1-[[(9Z,12Z)-octadeca-9,12-dienyl]carbamoyl]-4-oxo-butyl]amino]-4-oxo-butanoic acid

Molecule 4-[[4-[[(9Z,12Z)-octadeca-9,12-dienyl]amino]-1-[[(9Z,12Z)-octadeca-9,12-dienyl]carbamoyl]-4-oxo-butyl]amino]-4-oxo-butanoic acid is obtained following the procedure described for the synthesis of molecule 4-oxo-4-(2-pyrrolidin-1-ylethylamino)butanoic acid using 2-amino-N,N′-bis[(9Z,12Z)-octadeca-9,12-dienyl]pentanediamide (1.28 g, 2 mmoles), Et₃N (4 mmoles, 560 μL), succinic anhydride (4 mmoles, 400 mg) dissolved under argon in 20 mL of a 1/1 (v/v) mixture of dry DCM and DMF. Reaction time: 18 h, 78% yield (1.16 g, 1.56 mmoles), TLC (DCM/MeOH: 95/5) rf: 0.5.

### [4-azaniumyl-5-[[2-[2-[4-[[4-[[(9Z,12Z)-octadeca-9,12-dienyl]amino]-1-[[(9Z,12Z)-octadeca-9,12-dienyl]carbamoyl]-4-oxo-butyl]amino]-4-oxo-butanoyl]oxyethylamino]-2-oxo-ethyl]amino]-5-oxo-pentyl]ammonium

Compound 4-[[4-[[(9Z,12Z)-octadeca-9,12-dienyl]amino]-1-[[(9Z,12Z)-octadeca-9,12-dienyl]carbamoyl]-4-oxo-butyl]amino]-4-oxo-butanoic acid (0.3 mmol, 224 mg) is placed in a dry 25 mL flask under an argon then dissolved in 5 mL of DCM under stirring. DCC (0.6 mmol; 124 mg) and DMAP (0.1 mmol; 12 mg) are added to the reaction medium. The reaction is then stirred for 10 minutes before 4-[[4-[[(9Z,12Z)-octadeca-9,12-dienyl]amino]-1-[[(9Z,12Z)-octadeca-9,12-dienyl]carbamoyl]-4-oxo-butyl]amino]-4-oxo-butanoic acid (0.4 mmol; 173 mg) is introduced. After stirring at RT under argon for 16 hours, the reaction is stopped and the medium is evaporated under vacuum. The crude is taken back in 20 mL of DCM and washed with 1M HCl, NaHCO₃ and brine. The organic extract is dried on MgSO₄ and purified by flash chromatography on silica gel (elution gradient from 2 to 5% MeOH in DCM, TLC:Rf=0.5 (DCM:MeOH 90/10 (v/v)) before being dissolved in 20 mL DCM while 1 mL of trifluoroacetic acid is added. The reaction is then maintained for one night under stirring at RT. The reaction medium is evaporated and is coevaporated, four times in DCM and four times in Et₂O before drying. Yield 84% (2 steps, 0.252 mmoles, 299 mg).

### 2-[[2-[2,5-bis[4-(dimethylamino)butanoylamino]pentanoylamino]acetyl]amino]ethyl 4-[[4-[[(9Z,12Z)-octadeca-9,12-dienyl]amino]-1-[[(9Z,12Z)-octadeca-9,12-dienyl]carbamoyl]-4-oxo-butyl]amino]-4-oxo-butanoate

Molecule 2-[[2-[2,5-bis[4-(dimethylamino)butanoylamino]pentanoylamino]acetyl]amino]ethyl 4-[[4-[[(9Z,12Z)-octadeca-9,12-dienyl]amino]-1-[[(9Z,12Z)-octadeca-9,12-dienyl]carbamoyl]-4-oxo-butyl]amino]-4-oxo-butanoate has been isolated from the coupling of compound [4-azaniumyl-5-[[2-[2-[4-[[4-[[(9Z,12Z)-octadeca-9,12-dienyl]amino]-1-[[(9Z,12Z)-octadeca-9,12-dienyl]carbamoyl]-4-oxo-butyl]amino]-4-oxo-butanoyl]oxyethylamino]-2-oxo-ethyl]amino]-5-oxo-pentyl]ammonium (0.25 mmol; 290 mg) and 2 equivalents of DMABA (0.6 mmol; 168 mg) using HBTU (0.6 mmol; 227 mg) and DIPEA (1.2 mmol; 204 μL) dissolved in 6 mL of DMF and 2 mL of DCM according to the procedure described for the molecule 2-[[4-[2-[2-[[2-(dimethylamino)acetyl]amino]ethyldisulfanyl]ethylamino]-4-oxo-butanoyl]amino]-N,N′-bis[(Z)-octadec-9-enyl]pentanediamide. Reaction time: 12 h RT; Purification: Silica gel chromatography gradient 98/2 to 90/10 (DCM/MeOH, v/v). Yield: 58% (0.15 mmol; 117 mg), TLC (DCM/MeOH: 95/5) Rf: 0.2).

### Synthesis Molecule 4-[[4-[[4-[4-[4-[(dimethylamino)methyl]triazol-1-yl]butylamino]-4-oxo-butanoyl]amino]-5-(4-dodecanoyloxybutylamino)-5-oxo-pentanoyl]amino]butyl dodecanoate

### 4-[[4-(4-dodecanoyloxybutylamino)-1-(4-dodecanoyloxybutylcarbamoyl)-4-oxo-butyl]amino]-4-oxo-butanoic acid

Molecule 4-[[4-(4-dodecanoyloxybutylamino)-1-(4-dodecanoyloxybutylcarbamoyl)-4-oxo-butyl]amino]-4-oxo-butanoic acid has been isolated following the procedure described for compound 4-oxo-4-(2-pyrrolidin-1-ylethylamino)butanoic acid, using compound 4-[[4-amino-5-(4-dodecanoyloxybutylamino)-5-oxo-pentanoyl]amino]butyl dodecanoate (491 mg, 0.75 mmoles), Et₃N (1.5 mmoles, 210 μL) and succinic anhydride (1.5 mmoles, 150 mg) dissolved under argon in 4 mL of a 1/1 (v/v) mixture of DCM and DMF. Reaction time: 12 h RT. Yield 82% (463 mg, 0.62 mmoles TLC (DCM/MeOH: 95/5) rf: 0.6.

### 4-[[4-[[4-(4-azidobutylamino)-4-oxo-butanoyl]amino]-5-(4-dodecanoyloxybutylamino)-5-oxo-pentanoyl]amino]butyl dodecanoate

Compound 4-[[4-(4-dodecanoyloxybutylamino)-1-(4-dodecanoyloxybutylcarbamoyl)-4-oxo-butyl]amino]-4-oxo-butanoic acid, (231 mg, 0.31 mmoles) is dissolved under Argon in 3 mL of a 9/1 v/v mixture of dry DCM/DMF, in a three-necks round-bottom flask equipped with a dropping funnel. HOBt (55 mg, 0.4 mmoles) and DIC (62 μL, 0.4 mmoles) are subsequently added and the reaction is stirred at RT for 2 h30, increasing the turbidity of the medium. Then 4-Azidobutylamine (1M in DCM, 470 μL, 0.47 mmoles) and Et₃N (87 μL, 0.62 mmoles) dissolved in 3 mL of a 9/1 v/v mixture of dry DCM/DMF are added over 30 min dropwise via the dropping funnel. After the completion of the addition, the reaction is stirred 33 h at room temperature and concentrated to dryness. The crude mixture is then resuspended in 20 mL of ethyl acetate and put 3 h at −20 °C. The resulting liquid and solid are then separated by centrifugation at 3000 rpm, and this procedure is repeated three times. All the liquid are collected and concentrated under vacuum while the solids are discarded. The remaining oil is then dried overnight under vacuum, yielding the desired compound (182 mg, 0.21 mmoles). TLC (DCM/MeOH: 98/2) rf: 0.5.

### 4-[[4-[[4-[4-[4-[(dimethylamino)methyl]triazol-1-yl]butylamino]-4-oxo-butanoyl]amino]-5-(4-dodecanoyloxybutylamino)-5-oxo-pentanoyl]amino]butyl dodecanoate

Copper sulphate heptahydrate (0.012 mmoles, 3 mg), ascorbic acid (Sigma, 0.06 mmoles, 12 mg) and triphenylphosphine (0.012 mmoles, 3 mg) are mixed together under stirring in 1 mL of dry DMSO. While this solution is stirring (15 minutes), compound 4-[[4-[[4-(4-azidobutylamino)-4-oxo-butanoyl]amino]-5-(4-dodecanoyloxybutylamino)-5-oxo-pentanoyl]amino]butyl dodecanoate (87 mg 0.1 mmoles) and N,N-Dimethylpropargylamine (108 μL, 1 mmoles) are dissolved in 4 mL of a THF. After complete dissolution of the starting materials, the copper-based yellowish solution is added to the reaction flask and stirred with the starting material for 12 h at room temperature. The reaction is then diluted in 10 mL of water and extracted with 4×20 mL of DCM. The organic extracts are then combined, dried on magnesium sulphate, filtered and concentrated under vacuum. The product is collected by flash chromatography on silica gel using a DCM/methanol mixture as eluent. A gradient has been used ranging from 99/1 to 90/10 (DCM/MeOH, v/v). 135 mg (0.145 mmol, 49% yield) of a white solid are collected. TLC (DCM/MeOH: 95/5) rf: 0.3.

### Synthesis Molecule [(Z)-non-2-enyl]-4-[[2-[[4-[3-[bis(2-hydroxyethyl)amino]propylamino]-4-oxo-butanoyl]amino]-5-oxo-5-(tetradecylamino)pentanoyl]amino]butanoate

### 4-[[1-[[4-[(Z)-non-2-enoxy]-4-oxo-butyl]carbamoyl]-4-oxo-4-(tetradecylamino)butyl]amino]-4-oxo-butanoic acid (31a)

Molecule 4-[[1-[[4-[(Z)-non-2-enoxy]-4-oxo-butyl]carbamoyl]-4-oxo-4-(tetradecylamino)butyl]amino]-4-oxo-butanoic acid has been isolated following the procedure described for compound 4-oxo-4-(2-pyrrolidin-1-ylethylamino)butanoic acid, using compound [(Z)-non-2-enyl]4-[[2-amino-5-oxo-5-(tetradecylamino)pentanoyl]amino]butanoate (83 mg, 0.15 mmoles), Et₃N (0.4 mmoles, 56 μL) and succinic anhydride (0.3 mmoles, 30 mg) dissolved under argon in 3 mL of DCM. Reaction time: 16 h RT. Purification: Silica gel chromatography gradient 100/0 to 95/5 (DCM/MeOH, v/v). Yield: 91% (89 mg of white solid, 0.14 mmol, TLC (DCM/MeOH: 90/10) Rf: 0.4).

### [(Z)-non-2-enyl]-4-[[2-[[4-[3-[bis(2-hydroxyethyl)amino]propylamino]-4-oxo-butanoyl]amino]-5-oxo-5-(tetradecylamino)pentanoyl]amino]butanoate (XXXI)

Compound [(Z)-non-2-enyl]-4-[[2-[[4-[3-[bis(2-hydroxyethyl)amino]propylamino]-4-oxo-butanoyl]amino]-5-oxo-5-(tetradecylamino)pentanoyl]amino]butanoate has been isolated adapting the procedure described for compound 2-[4-(dimethylamino)butanoylamino]-N′—[(Z)-heptadec-9-enyl]-N—[(Z)-octadec-9-enyl]pentanediamide, by linking compound 4-[[1-[[4-[(Z)-non-2-enoxy]-4-oxo-butyl]carbamoyl]-4-oxo-4-(tetradecylamino)butyl]amino]-4-oxo-butanoic acid 0.14 mmol; 89 mg) with N-(3-Aminopropyl)diethanolamine (0.12 mmol; 18 μL) using methylimidazole (0.4 mmol; 32 μL) and TCFH (0.14 mmol; 40 mg) dissolved in 4 mL of a 2/1 (v/v) mixture solution of MeCN and DCM. Reaction time: 12 h 0 °C to RT; Purification: Silica gel chromatography gradient 98/2 to 90/10 (DCM/MeOH, v/v). Yield: 64% (61 mg of white solid, 0.08 mmol, TLC (DCM/MeOH: 95/5) Rf: 0.3).

### Synthesis Molecule [(Z)-non-2-enyl]4-[[2-[[4-[2-[2-[2,5-bis[[2-(dimethylamino)acetyl]amino]pentanoylamino]ethyldisulfanyl]ethylamino]-4-oxo-butanoyl]amino]-5-oxo-5-(tetradecylamino)pentanoyl]amino]butanoate

### 2-[2-[[4-[[1-[[4-[(Z)-non-2-enoxy]-4-oxo-butyl]carbamoyl]-4-oxo-4-(tetradecylamino)butyl]amino]-4-oxo-butanoyl]amino]ethyldisulfanyl]ethylammonium

Boc-cystamine (0.28 mmol; 91 mg) is placed in a 25 mL flask under argon then dissolved in 2 mL of anhydrous DMF under stirring. HBTU (0.28 mmol; 106 mg) and DIPEA (0.56 mmol; 97 μL) are added to the solution successively. The reaction medium is stirred under argon at RT for 15 minutes before compound [(Z)-non-2-enyl]4-[[2-amino-5-oxo-5-(tetradecylamino)pentanoyl]amino]butanoate (0.14 mmol; 89 mg), in solution in 1 mL of anhydrous DMF and 1 mL of anhydrous DCM, is added to the reaction medium. After 12 hours, the reaction is stopped and the reaction medium is evaporated to dryness under a high vacuum. The crude is taken back in 10 mL of EtOAc and washed with 1M HCl, NaHCO₃ and brine. The organic layer is dried on MgSO₄ and purified by flash chromatography on silica gel (elution gradient from 2 to 5% MeOH in DCM). The isolated intermediate (0.1 mmol; 79 mg) is dissolved in 5 mL of DCM, then 250 μL of trifluoroacetic acid is added to the reaction medium. The reaction is then maintained for 4 hours under stirring at RT. The mixture is then concentrated, co-evaporated thrice with 50 mL of DCM, thrice with 50 mL of diethyl ether (Et₂O), and dried under high vacuum overnight Yield: 67% (2 steps, 84 mg, 0.094 mmol).

### [4-azaniumyl-5-[2-[2-[[4-[[1-[[4-[(Z)-non-2-enoxy]-4-oxo-butyl]carbamoyl]-4-oxo-4-(tetradecylamino)butyl]amino]-4-oxo-butanoyl]amino]ethyldisulfanyl]ethylamino]-5-oxo-pentyl]ammonium

Compound [4-azaniumyl-5-[2-[2-[[4-[[1-[[4-[(Z)-non-2-enoxy]-4-oxo-butyl]carbamoyl]-4-oxo-4-(tetradecylamino)butyl]amino]-4-oxo-butanoyl]amino]ethyldisulfanyl]ethylamino]-5-oxo-pentyl]ammonium is obtained following the two steps procedure experienced for the synthesis of compound 2-[2-[[4-[[1-[[4-[(Z)-non-2-enoxy]-4-oxo-butyl]carbamoyl]-4-oxo-4-(tetradecylamino)butyl]amino]-4-oxo-butanoyl]amino]ethyldisulfanyl]ethylammonium. First Boc-L-Orn(Boc)-OH (0.2 mmol; 66 mg) and compound 32a (0.1 mmol; 90 mg) are linked together using HBTU (0.2 mmol; 76 mg) and DIPEA (0.4 mmol; 69 μL) dissolved in 3 mL of DMF for 16 h at RT. After isolation and purification of the intermediate, amine functions are released by reacting TFA (250 μL) in 5 mL DCM during 1 h. Yield: 61% (2 steps, 69 mg, 0.061 mmol).

### [(Z)-non-2-enyl]4-[[2-[[4-[2-[2-[2,5-bis[[2-(dimethylamino)acetyl]amino]pentanoylamino]ethyldisulfanyl]ethylamino]-4-oxo-butanoyl]amino]-5-oxo-5-(tetradecylamino)pentanoyl]amino]butanoate

Following the procedure described for compound 2-[[2-(dimethylamino)acetyl]amino]-N,N′-bis[(Z)-octadec-9-enyl]pentanediamide, molecule [(Z)-non-2-enyl]4-[[2-[[4-[2-[2-[2,5-bis[[2-(dimethylamino)acetyl]amino]pentanoylamino]ethyldisulfanyl]ethylamino]-4-oxo-butanoyl]amino]-5-oxo-5-(tetradecylamino)pentanoyl]amino]butanoate is obtained by coupling DMG (0.18 mmol; 19 mg) and compound [4-azaniumyl-5-[2-[2-[[4-[[1-[[4-[(Z)-non-2-enoxy]-4-oxo-butyl]carbamoyl]-4-oxo-4-(tetradecylamino)butyl]amino]-4-oxo-butanoyl]amino]ethyldisulfanyl]ethylamino]-5-oxo-pentyl]ammonium (0.06 mmol; 68 mg) using DIPEA (0.4 mmol; 69 μL) and pyBOP (0.2 mmol; 104 mg) dissolved in 3 mL of DMF. Reaction time: 48 h RT; Purification: Silica gel chromatography gradient 98/2 to 90/10 (DCM/MeOH, v/v). Yield: 32% (0.02 mmol; 21 mg), TLC (DCM/MeOH: 95/5) Rf: 0.2.).

### Synthesis Molecule 3-butylhept-2-enyl-4-[[2-[[4-[3-[bis(2-hydroxyethyl)amino]propylamino]-4-oxo-butanoyl]amino]-5-oxo-5-(tetradecylamino)pentanoyl]amino]butanoate

### a) Synthesis of Molecule [4-(3-butylhept-2-enoxy)-4-oxo-butyl]ammonium

### [4-(3-butylhept-2-enoxy)-4-oxo-butyl]ammonium

3-Butyl-2-hepten-1-ol (5.5 mmol; 1.11 mL) is added under stirring to a solution of N-Boc-gamma-aminobutyric acid (5 mmol, 1.02 g), DCC (5.5 mmol; 1.13 g) and DMAP (1 mmol; 122 mg) dissolved in 50 mL of anhydrous DCM. The reaction is then maintained under argon at RT for 16 hours. After total consumption of the acid the reaction medium is evaporated to dryness under vacuum. The crude is taken back in 50 mL of DCM and washed with 1M HCl, NaHCO₃ and brine. The organic layer is dried on MgSO₄ et then purified by flash chromatography on silica gel (elution gradient from 0 to 50% EtOAc in petroleum ether Leading to an intermediate (3.95 mmol; 1.4 g) that is dissolved in 100 mL of DCM in presence of 5 mL of TFA. The reaction is then maintained for one night under stirring at ambient temperature. The mixture is then concentrated, co-evaporated thrice with 50 mL of DCM, thrice with 50 mL of diethyl ether (Et₂O), and dried under high vacuum overnight Yield: 79% (2 steps, 1.46 g, 3.95 mmol).

### b) Synthesis of molecules 3-butylhept-2-enyl-4-[[2-[[4-[3-[bis(2-hydroxyethyl)amino]propylamino]-4-oxo-butanoyl]amino]-5-oxo-5-(tetradecylamino)pentanoyl]amino]butanoate & 4-pyrrolidin-1-ylbutyl-4-[[1-[[4-(3-butylhept-2-enoxy)-4-oxo-butyl]carbamoyl]-4-oxo-4-(tetradecylamino)butyl]amino]-4-oxo-butanoate

### 5-[[4-(3-butylhept-2-enoxy)-4-oxo-butyl]amino]-4-(9H-fluoren-9-ylmethoxycarbonylamino)-5-oxo-pentanoic acid

Molecule 5-[[4-(3-butylhept-2-enoxy)-4-oxo-butyl]amino]-4-(9H-fluoren-9-ylmethoxycarbonylamino)-5-oxo-pentanoic acid is obtained by adapting a two steps method described for the synthesis of molecule 4-(9H-fluoren-9-ylmethoxycarbonylamino)-5-[[4-[(Z)-non-2-enoxy]-4-oxo-butyl]amino]-5-oxo-pentanoic acid, in which Fmoc-Glu(OtBu)-OH (2.5 mmol; 1.06 g) and compound [4-(3-butylhept-2-enoxy)-4-oxo-butyl]ammonium 4.9 mmol; 1.67 g are coupled in presence of COMU (2.5 mmol; 1.07 g) and DIPEA (5 mmol; 865 μL) dissolved in 40 mL of DMF under stirring. Reaction time: 18 h 0 °C to RT; Purification: Silica gel chromatography gradient 70/30 to 40/60 (EP/EA, v/v). The ester function is then successfully cleaved using TFA (20 mL) in 20 mL DCM while stirring 24 h at RT. The mixture is then concentrated, co-evaporated thrice with 50 mL of DCM, thrice with 50 mL of diethyl ether (Et₂O), and dried under high vacuum overnight Yield: 74% (2 steps, 1.12 g, 1.85 mmol).

### 3-butylhept-2-enyl-4-[[2-amino-5-oxo-5-(tetradecylamino)pentanoyl]amino]butanoate

Following the procedure described for compound 2-amino-N,N′-bis[(Z)-octadec-9-enyl]pentanediamide, molecule 3-butylhept-2-enyl-4-[[2-amino-5-oxo-5-(tetradecylamino)pentanoyl]amino]butanoate is obtained in two steps by coupling Tetradecylamine (2.8 mmol; 592 mg) and compound 5-[[4-(3-butylhept-2-enoxy)-4-oxo-butyl]amino]-4-(9H-fluoren-9-ylmethoxycarbonylamino)-5-oxo-pentanoic acid (0.06 mmol; 68 mg) using DIC (3.7 mmol; 579 μL) and HOBt (3.7 mmol; 500 mg) dissolved in 30 mL of DMF and 30 mL of CHCl₃ containing Et₃N (3 mmol; 408 μL). After 36 h stirring at RT, Fmoc deprotection is carried out by introducing 10 mL of piperidine and stirring 4 h at RT. Reaction time: 36 h+4 h RT; Yield: 77% (1.42 mmol; 826 mg); TLC:Rf=0.4 (DCM:MeOH 90/10 (v/v)).).

### 4-[[1-[[4-(3-butylhept-2-enoxy)-4-oxo-butyl]carbamoyl]-4-oxo-4-(tetradecylamino)butyl]amino]-4-oxo-butanoic acid

Molecule 4-[[1-[[4-(3-butylhept-2-enoxy)-4-oxo-butyl]carbamoyl]-4-oxo-4-(tetradecylamino)butyl]amino]-4-oxo-butanoic acid has been isolated following the procedure described for compound 4-oxo-4-(2-pyrrolidin-1-ylethylamino)butanoic acid, using compound 3-butylhept-2-enyl-4-[[2-amino-5-oxo-5-(tetradecylamino)pentanoyl]amino]butanoate (1.42 mmol; 826 mg), Et₃N (2.1 mmol; 295 μL) and succinic anhydride (2.1 mmol; 210 mg) dissolved under argon in 20 mL of DCM and 10 mL of DMF. Reaction time: 16 h RT. Purification: Silica gel chromatography gradient 98/2 to 92/8 (DCM/MeOH, v/v). Yield: 88% (850 mg of white solid, 1.25 mmol, TLC (DCM/MeOH: 95/5) Rf: 0.4).

### 3-butylhept-2-enyl-4-[[2-[[4-[3-[bis(2-hydroxyethyl)amino]propylamino]-4-oxo-butanoyl]amino]-5-oxo-5-(tetradecylamino)pentanoyl]amino]butanoate

Compound 3-butylhept-2-enyl-4-[[2-[[4-[3-[bis(2-hydroxyethyl)amino]propylamino]-4-oxo-butanoyl]amino]-5-oxo-5-(tetradecylamino)pentanoyl]amino]butanoate has been isolated adapting the procedure described for compound 2-[4-(dimethylamino)butanoylamino]-N′—[(Z)-heptadec-9-enyl]-N—[(Z)-octadec-9-enyl]pentanediamide, by linking compound 4-[[1-[[4-(3-butylhept-2-enoxy)-4-oxo-butyl]carbamoyl]-4-oxo-4-(tetradecylamino)butyl]amino]-4-oxo-butanoic acid (0.5 mmol; 340 mg) with N-(3-Aminopropyl)diethanolamine (0.6 mmol; 91 μL) using methylimidazole (1.5 mmol; 120 μL) and TCFH (0.6 mmol; 170 mg) dissolved in 15 mL of a 2/1 (v/v) mixture solution of MeCN and DCM. Reaction time: 12 h 0 °C to RT; Purification: Silica gel chromatography gradient 98/2 to 94/6 (DCM/MeOH, v/v). Yield: 61% (256 mg of white solid, 0.31 mmol, TLC (DCM/MeOH: 90/10) Rf: 0.3).

### 4-pyrrolidin-1-ylbutyl-4-[[1-[[4-(3-butylhept-2-enoxy)-4-oxo-butyl]carbamoyl]-4-oxo-4-(tetradecylamino)butyl]amino]-4-oxo-butanoate

Compound 4-pyrrolidin-1-ylbutyl-4-[[1-[[4-(3-butylhept-2-enoxy)-4-oxo-butyl]carbamoyl]-4-oxo-4-(tetradecylamino)butyl]amino]-4-oxo-butanoate has been isolated adapting the procedure described for compound 20c, by coupling compound 4-[[1-[[4-(3-butylhept-2-enoxy)-4-oxo-butyl]carbamoyl]-4-oxo-4-(tetradecylamino)butyl]amino]-4-oxo-butanoic acid (0.5 mmol; 340 mg) with 4-(1-pyrrolidinyl)-1-butanol (0.7 mmol; 100 mg) using DCC (0.7 mmol; 145 mg) and DMAP (0.1 mmol; 12 mg) dissolved in 20 mL of a DCM. Reaction time: 15 h RT; Purification: Silica gel chromatography gradient 98/2 to 90/10 (DCM/MeOH, v/v). Yield: 58% (234 mg of white solid, 0.29 mmol, TLC (DCM/MeOH: 95/5) Rf: 0.3).

### Synthesis Molecule [(Z)-non-2-enyl]-4-[[2-[[4-[2-[2-[2,5-bis[[2-(dimethylamino)acetyl]amino]pentanoylamino]ethyldisulfanyl]ethylamino]-4-oxo-butanoyl]amino]-5-[[3-(dodecylamino)-1-(dodecylcarbamoyl)-3-oxo-propyl]amino]-5-oxo-pentanoyl]amino]butanoate

### 2-[2-[[4-[[4-[[3-(dodecylamino)-1-(dodecylcarbamoyl)-3-oxo-propyl]amino]-1-[[4-[(Z)-non-2-enoxy]-4-oxo-butyl]carbamoyl]-4-oxo-butyl]amino]-4-oxo-butanoyl]amino]ethyldisulfanyl]ethylammonium

Compound 2-[2-[[4-[[4-[[3-(dodecylamino)-1-(dodecylcarbamoyl)-3-oxo-propyl]amino]-1-[[4-[(Z)-non-2-enoxy]-4-oxo-butyl]carbamoyl]-4-oxo-butyl]amino]-4-oxo-butanoyl]amino]ethyldisulfanyl]ethylammonium is obtained following the two steps procedure experienced for the synthesis of compound 2-[2-[[4-[[4-[[(Z)-octadec-9-enyl]amino]-1-[[(Z)-octadec-9-enyl]carbamoyl]-4-oxo-butyl]amino]-4-oxo-butanoyl]amino]ethyldisulfanyl]ethylammonium trifluoroacetate. First, compound 4-[2-[2-(tert-butoxycarbonylamino)ethyldisulfanyl]ethylamino]-4-oxo-butanoic acid (0.9 mmol; 292 mg) and compound [(Z)-non-2-enyl]-4-[[2-amino-5-[[3-(dodecylamino)-1-(dodecylcarbamoyl)-3-oxo-propyl]amino]-5-oxo-pentanoyl]amino]butanoate (0.6 mmol; 484 mg) are linked together using DIC (0.9 mmol; 141 μL) and HOBt (0.9 mmol; 122 mg) dissolved in 12 mL of DMF and 4 mL of DCM containing Et₃N (0.8 mmol; 112 μL); the reaction is then stirred for 48 h at RT. After isolation and purification of the intermediate (flash chromatography on silica gel, elution gradient from 0 to 5% MeOH in DCM), amine functions are released by reacting TFA (550 μL) in 11 mL DCM during 12 h. Yield: 38% (2 steps, 265 mg, 0.23 mmol).

### [4-azaniumyl-5-[2-[2-[[4-[[4-[[3-(dodecylamino)-1-(dodecylcarbamoyl)-3-oxo-propyl]amino]-1-[[4-[(Z)-non-2-enoxy]-4-oxo-butyl]carbamoyl]-4-oxo-butyl]amino]-4-oxo-butanoyl]amino]ethyldisulfanyl]ethylamino]-5-oxo-pentyl]ammonium

Compound 2-[2-[[4-[[4-[[3-(dodecylamino)-1-(dodecylcarbamoyl)-3-oxo-propyl]amino]-1-[[4-[(Z)-non-2-enoxy]-4-oxo-butyl]carbamoyl]-4-oxo-butyl]amino]-4-oxo-butanoyl]amino]ethyldisulfanyl]ethylammonium is obtained following the two steps procedure experienced for the synthesis of compound 2-[2-[[4-[[1-[[4-[(Z)-non-2-enoxy]-4-oxo-butyl]carbamoyl]-4-oxo-4-(tetradecylamino)butyl]amino]-4-oxo-butanoyl]amino]ethyldisulfanyl]ethylammonium. First, Boc-L-Orn(Boc)-OH (0.4 mmol; 132 mg) and compound 2-[2-[[4-[[4-[[3-(dodecylamino)-1-(dodecylcarbamoyl)-3-oxo-propyl]amino]-1-[[4-[(Z)-non-2-enoxy]-4-oxo-butyl]carbamoyl]-4-oxo-butyl]amino]-4-oxo-butanoyl]amino]ethyldisulfanyl]ethylammonium (0.22 mmol; 254) are linked together using HBTU (0.4 mmol; 152 mg) and DIPEA (0.8 mmol; 138 μL) dissolved in 6 mL of DMF; the reaction is then stirred for 16 h at RT. After isolation and purification of the intermediate (flash chromatography on silica gel, elution gradient from 2 to 10% MeOH in DCM), amine functions are released by reacting TFA (350 μL) in 5 mL DCM during 12 h. Yield: 63% (2 steps, 176 mg, 0.14 mmol).

### [(Z)-non-2-enyl]-4-[[2-[[4-[2-[2-[2,5-bis[[2-(dimethylamino)acetyl]amino]pentanoylamino]ethyldisulfanyl]ethylamino]-4-oxo-butanoyl]amino]-5-[[3-(dodecylamino)-1-(dodecylcarbamoyl)-3-oxo-propyl]amino]-5-oxo-pentanoyl]amino]butanoate

Compound [(Z)-non-2-enyl]-4-[[2-[[4-[2-[2-[2,5-bis[[2-(dimethylamino)acetyl]amino]pentanoylamino]ethyldisulfanyl]ethylamino]-4-oxo-butanoyl]amino]-5-[[3-(dodecylamino)-1-(dodecylcarbamoyl)-3-oxo-propyl]amino]-5-oxo-pentanoyl]amino]butanoate has been isolated adapting the procedure described for compound II, by linking compound [4-azaniumyl-5-[2-[2-[[4-[[4-[[3-(dodecylamino)-1-(dodecylcarbamoyl)-3-oxo-propyl]amino]-1-[[4-[(Z)-non-2-enoxy]-4-oxo-butyl]carbamoyl]-4-oxo-butyl]amino]-4-oxo-butanoyl]amino]ethyldisulfanyl]ethylamino]-5-oxo-pentyl]ammonium (0.14 mmol; 178 mg) with DMG (0.35 mmol; 36 mg) using methylimidazole (1.05 mmol; 84 μL) and TCFH 0.42 mmol; 119 mg) dissolved in 15 mL of a 2/1 (v/v) mixture solution of MeCN and DCM. Reaction time: 12 h 0 °C to RT; Purification: Silica gel chromatography gradient 98/2 to 90/10 (DCM/MeOH, v/v). Yield: 44% (82 mg of white solid, 0.06 mmol, TLC (DCM/MeOH: 95/5) Rf: 0.2).

### II—Results

Encapsulation of mRNA in Lipid Nanoparticles Using Microfluidic Formulation

The lipids described in the present invention were then used to encapsulate mRNA via microfluidic formulation of lipid nanoparticles. First all lipidic components (i.e., tunable lipid, helper neutral lipid, sterol derivative and PEG-lipid) were dissolved separately in glass vial at 25 mg/mL in a 99/1 (v/v) solution of chloroform in methanol as different molar ratios (Table 1). Then, the necessary amount of each component is introduced in a flask, that is evaporated 2 h at 45 °C under gentle vacuum. This step leads to the obtention of dry lipidic film that is then dried under high vacuum for one night. The lipidic film is then dissolved in sterile-filtered EtOH, and sonicated during 10 minutes. The volume of ethanol is chosen according the desired final formulation volume needed. Meanwhile mRNA is dissolved in a 1 mM citrate buffer at pH=4, so that the final concentration of mRNA solution is 0.125 mg/mL.

Formulation into lipid nanoparticles is realized using the μ-encapsulator System from Dolomite Microfluidics into the MicroMixer chip. Briefly the ethanolic lipid solution and mRNA solution are injected into the system and formed LNPs are collected at the output. The pump parameters have been optimized for each formulation depending on the lipid used, the targeted final concentration of lipids required and the size of chosen nucleic acid. As a basis, a Flow Rate Ratio (FRR) equals to 3 and a total flow rate of 1000 μL/min could be set up. Collected LNPs are subsequently dialyzed against PBS+5% w/v sucrose overnight, filtered on a sterile 0.45 μm filter and stored at −80 °C.

TABLE 1
Molar compositions used for the formation
of LNPs based on tunable lipids

| Lipid components | mol (%) N = 1 | mol (%) N = 2 |
|---|---|---|
| Tunable Lipid | 50 | 25 |
| Helper neutral lipid | 10 | 15 |
| Sterol derivative | 38.5 | 57.75 |
| PEG-lipid (stealth lipid) | 1.5 | 2.25 |
| Global | 100 | 100 |

Characterization of mRNA LNPs

The size and charge of the collected LNPs are determined using DLS (Malvern Instruments NanoZS) by dissolving 50 μL of the collected solution in 950 μL of MilliQ water. Then, formed LNPs are destabilized by adding 1% v/v of Triton X-100 and incubate at 30 °C for 1 h and vortexing. After disruption of the formulation, a 1/100 solution of SyBr™Gold in water is prepared, and 0.5 μL of it added to 49.5 μL of the resulting mixture that includes Triton X-100. The SyBr™Gold is also added the same way to 49.5 μL of a solution that contains non-disrupted LNPs, and to 49.5 μL of the mRNA mother solution. Fluorescence of each sample is then read after 30 min incubation at RT using a Victor (Perkin Elmer) fluorimeter (excitation: 495 nm; emission: 537 nm;). The encapsulation efficiency (ee(%)) is easily obtained from these fluorescence measurements using the formula below:

$$ee ( \% ) = \frac{( {Fluo\_LNP}_{1 v \% triton} - Fluo\_LNP )}{{Fluo\_RNA}_{stock solution}} x 100$$

This methodology has been applied to several tunable lipids depicted in this invention (Table 2). For this first step of screening, 5-methoxyuridine-modified mRNA encoding for GFP (OZ Biosciences, Cat: MRNA15-1000) has been encapsulated.

TABLE 2
Main physical properties of mRNA(GFP) LNPs based on tunable lipids I, II, V, VI, XIV, XV

| LNPs | | | | | | | |
|---|---|---|---|---|---|---|---|
| Characterization | I | II | V | VIII | X | XIV | XV |
| Total Lipid | 3.75 | 3.75 | 3.75 | 3.75 | 3.75 | 3.75 | 3.75 |
| concentration (mM) | | | | | | | |
| Size (PDI) | 83 nm (0.06) | 113 nm (0.2) | 95 nm (0.113) | 117 nm (0.344) | 141 nm (0.109) | 115 nm (0.186) | 79 nm (0.11) |
| Zeta | −9.3 mV | 5.57 mV | −17 mV | +0.3 mV | +11.6 mV | +15.6 mV | −11.4 mV |
| Loaded RNA (ng/μL) | 94.6 | 98.8 | 89 | 84 | 83.6 | 91 | 78.3 |
| N/P Ratio | 6 | 6 | 6 | 6 | 6 | 6 | 6 |
| Encapsulation | 94.6% | 98.8% | 89% | 84% | 83.6% | 91% | 78.3% |
| Efficiency | | | | | | | |

All the tested lipids proved to be able to form nanoparticles when associated with a neutral phospholipid, a sterol derivative and a PEG-lipid under classical microfluidic handling and high encapsulation efficiency was achieved. Regarding physico-chemical properties, a majority of the lipid allowed the obtention of small size LNPs, with DLS measurements showing values around 100 nm. Especially in the case of lipids 1, V and XV, LNPs with size ranging below 100 nm could be obtained with polydispersity index around 0.1. Interestingly, value of Zeta potential varied from one lipid to another, even if the N/P ratio was maintained constant. Indeed, the surface charge of the LNP can vary from a quite negative value if lipid V is issued to a positive one, such in the case of lipid XIV. This implies that the geometry of the tunable lipid used has a clear influence on the future physical properties of the corresponding LNPs and probably on its biological behaviour. Consequently, a skilled scientist would have to test several lipids corresponding to the formula depicted in this invention and select the most suitable one depending on the properties he would want to reach for his LNPs.

### Cells

Human cervical carcinoma (HeLa) and human pancreatic cancer (PANC-1) and murine macrophage (RAW 264-7) cell lines were cultured in Dulbecco's Modified Eagle Medium (DMEM, Lonza, Walkersville, MD, USA) supplemented with 10% Foetal Bovine Serum (FBD, St. Louis, MO, USA), 2 mM final L-Glutamine, 100 units/ml penicillin and 100 μ/ml streptomycin (Lonza, Walkersville, MD, USA). Human immortalized Jurkat T-cells and human leukiemous monocytes (THP-1) were cultured in Roswell Park Memorial Institute medium (RPMI, Lonza, Walkersville, MD, USA) supplemented with 10% Foetal Bovine Serum (FBD, St. Louis, MO, USA), 2 mM final L-Glutamine, 100 units/ml penicillin and 100 μ/ml streptomycin (Lonza, Walkersville, MD, USA). All cell lines were cultured in 75 cm² flasks (Sarstedt AG & Co., Germany) at 37 °C in a humidified incubator (Sanyo, Tokyo, Japan) with 5% CO₂ atmosphere, and trypsinized at 80% confluency in order to maintain an exponential division rate before transduction assays. Transfection experiments were performed in 24 well-plates (Sarstedt AG & Co., Germany).

In Vitro Transfection of GFP mRNA Using LNPs

Two LNPs formulations based respectively on lipids I and XIV have then been tested in an in vitro transfection experiment to demonstrate their potency to efficiently deliver mRNA, while enabling mRNA gene expression. Transfection efficiency has been monitored by flow cytometry by evaluating the percent of transfected cells, and the percent of living cells using flow cytometry. A quantity of mRNA ranging from 0.1 μg to 20 μg has been tested and RmesFect, a commercially available transfection reagent (a cationic lipid with permanent positive charges) devoted to mRNA delivery, has been used as a positive control.

This result (FIG. 45) displays the potency of LNPs based on tunable lipids to transfect a cell line using a wide range of mRNA quantities, with a maximum of 90% of positive GFP cells Reached values proved to be better than those observed with a more classical transfection reagent, while maintaining cell viability. Indeed, regarding % of living cells, tunable lipids formulated as LNPs remained harmless in comparison to transfection reagent which led to a rapid decline in cell survival, with only 30% of cells still alive when 1 μg of mRNA is used (FIG. 45).

These results clearly show the potency of LNPs based on tunable lipids to be efficient in an in vitro transfection context, at level equivalent to standard transfection reagent, without compromising cell survival especially at high nucleic acid doses.

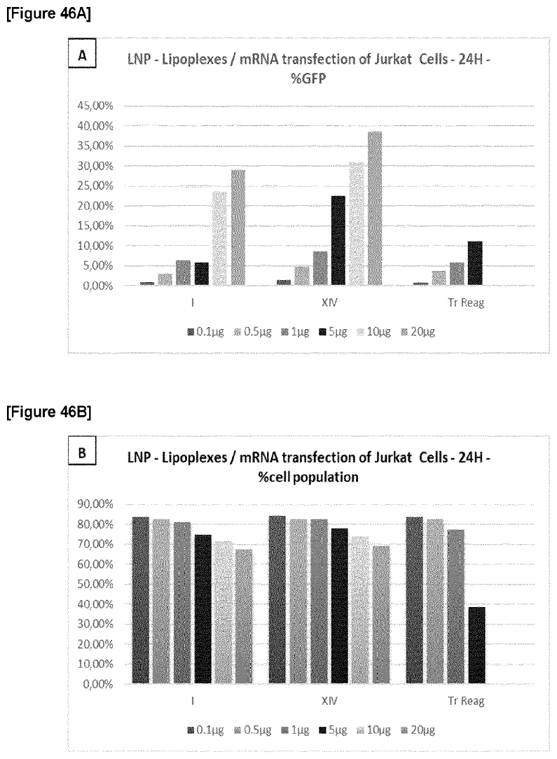

These results were confirmed in another cellular model known for being difficult to transfect in a laboratory setup using non-viral transfection reagent (FIG. 46). Jurkat T-cells, a suspension cell line was transfected as described above and. results using LNPs based on tunable lipids I and XIV are depicted in FIG. 46. Observed results are in accordance with those observed in HeLa cells. The transfection efficiency was able to reach 40% while in the same time the commercially available reagent provides only 10% of positive cells at the maximum mRNA quantity used. Furthermore, regarding % of living cells after transfection, tunable lipids formulated as LNPs remained the safest in the whole range of mRNA used quantities, whereas the transfection reagent used as control lead to a decline in cell survival with increasing mRNA quantities, with only 40% of cells still alive when 5 μg of mRNA is used.

In Vitro Transfection of GFP mRNA in Cancer Cell Lines Using LNPs

Several LNPs formulations based respectively on lipids I, XXXIV, XXXV, XXXVIII, XXXVI and XXXIII have then been tested in an in vitro transfection experiment to demonstrate their potency to efficiently deliver mRNA, while enabling mRNA gene expression. We decided this time to evaluate the efficacy of the compounds of the present invention in cancer cell lines, since LNPs represents a promising outlook by their capacity to efficiently deliver genetic material into cancer cells. We choose as models PANC-1 cells, a human pancreatic cancer cell line isolated from a pancreatic carcinoma of ductal cell origin. monitored by flow cytometry by evaluating the percent of transfected cells, and the percent of living cells using flow cytometry.

TABLE 3
Main physical properties of mRNA(GFP) LNPs based on tunable lipids I, XXXIV, XXXV, XXXVIII, XXXVI and XXXIII

| LNPs | | | | | | |
|---|---|---|---|---|---|---|
| Characterization | I | XXXIV | XXXV | XXXVIII | XXXVI | XXXIII |
| Total Lipid | 3.75 | 3.75 | 3.75 | 3.75 | 3.75 | 3.75 |
| concentration (mM) | | | | | | |
| Size (PDI) | 83 nm (0.06) | 136 nm (0.12) | 118.3 nm (0.108) | 106.2 nm (0.344) | 123.7 nm (0.093) | 131.5 nm (0.08) |
| Zeta | −9.3 mV | −5.9 mV | 8.23 mV | −15.5 mV | −2.07 mV | +7.56 mV |
| Loaded RNA (ng/μL) | 94.6 | 91.1 | 81.1 | 89.0 | 81.6 | 73 |
| N/P Ratio | 6 | 6 | 6 | 6 | 6 | 6 |
| Encapsulation | 94.6% | 91.1% | 81.1% | 89% | 81.6% | 73% |
| Efficiency | | | | | | |

Here again, all the tested lipids proved to be able to form nanoparticles when associated with a neutral phospholipid, a sterol derivative and a PEG-lipid under classical microfluidic conditions and high encapsulation efficiency was achieved. Regarding physico-chemical properties, a majority of the lipid allowed the obtention of small size LNPs, with DLS measurements showing values around 100 nm. Especially in the case of lipids I, and XXXV, LNPs with size ranging below or close than 100 nm could be obtained with polydispersity index around 0.1. Interestingly, value of Zeta potential varied from one lipid to another, even if the N/P ratio was maintained constant. Indeed, the surface charge of the LNP can vary from a quite negative value if lipid XXXVIII is issued to a positive one, such in the case of lipid XXXV. This implies that the geometry of the tunable lipid used has a clear influence on the future physical properties of the corresponding LNPs and probably on its biological behaviour.

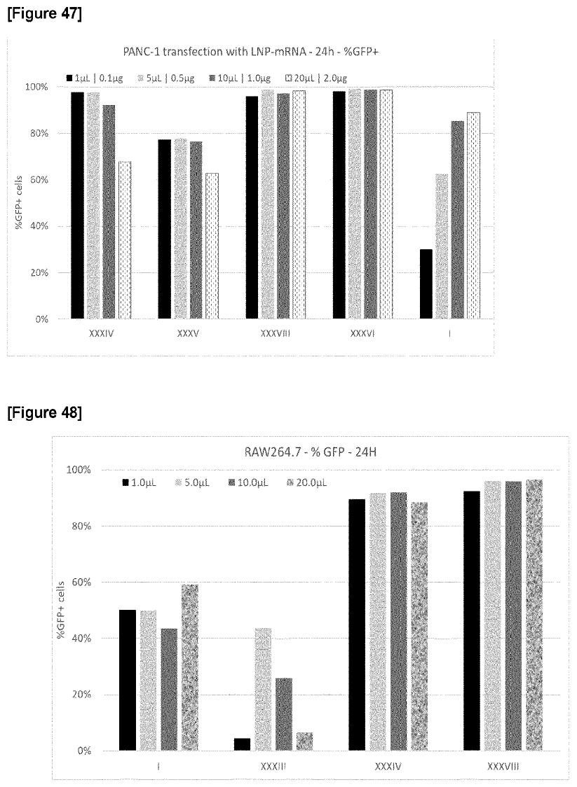

Five LNPs formulations based respectively on lipids I, XXXIV, XXXV, XXXVIII, XXXVI and XXXIII have then been tested in an in vitro transfection experiment in cancer cell lines, to demonstrate their potency to efficiently deliver mRNA, while enabling its gene expression. Transfection efficiency has been monitored by flow cytometry by evaluating the percent of transfected cells. A quantity of mRNA ranging from 0.1 μg to 20 μg has been tested (FIG. 47).

This result (FIG. 47) displays the potency of LNPs based on tunable lipids to transfect a cancer cell line using a wide range of mRNA quantities, with transfection values higher 90% of positive GFP cells for the best lipids candidates. Reached values proved to be better than those observed with a more classical transfection reagent, while maintaining cell viability.

These results clearly show the potency of LNPs based on tunable lipids to be efficient in an in vitro transfection context, at level equivalent to standard transfection reagent, without compromising cell survival especially at high nucleic acid doses.

In Vitro Transfection of GFP mRNA in Macrophages and Monocytes Cell Lines Using LNPs

Macrophages, the multifunctional phagocytes, play crucial roles in many physiological activities, such as immune regulation, cleaning up exogenous antigens, clearance of endogenous cell debris, tissue repair, regeneration, and fibrosis. In addition, macrophages are long-lived cells distributed all over the human body with a lifespan of months to years. Thus, these cells play critical roles in innate immunity and in the pathogeneses of several chronic and inflammatory diseases through the induction or resolution of inflammation and tissue repair. On the other hand, monocytes are a critical component of the innate immune system. They are the source of many other vital elements of the immune system, such as macrophages and dendritic cells. Monocytes play a role in both the inflammatory and anti-inflammatory processes that take place during an immune response. Knowing this, we evaluated the potency of the lipids of the present invention to efficiently transfect one macrophage cell line and one monocyte cell line using mRNA encoding for eGFP. We choose to evaluate several lipidic formulation displayed in table 3 on: RAW 264-7 a murine macrophage cell line and THP-1, a monocyte isolated from peripheral blood from an acute monocytic leukemia patient.

Lipids nanoparticles based on lipids I, XXXIII, XXXIV and XXXVIII were first evaluated on adherent RAW 264-7 cell lines, with tested quantities of mRNA ranging from 0.1 μg to 20 μg (FIG. 48). We could observed after 24 h that the different LNPs have different transfection behaviours, a fact that might be related to their different geometry. Thus while LNPs based on lipid XXXIII gave only about 40% of GFP positive cells at its optimal RNA quantity, lipids XXXIV and XXXVIII, gave consistent higher rate of transfection in a broader range of experimental conditions. All in all the four formulations provided consistent rates on transfection on this RAW 264-7 cell lines, broadening the field of applications of the molecules and method displayed within the present invention.

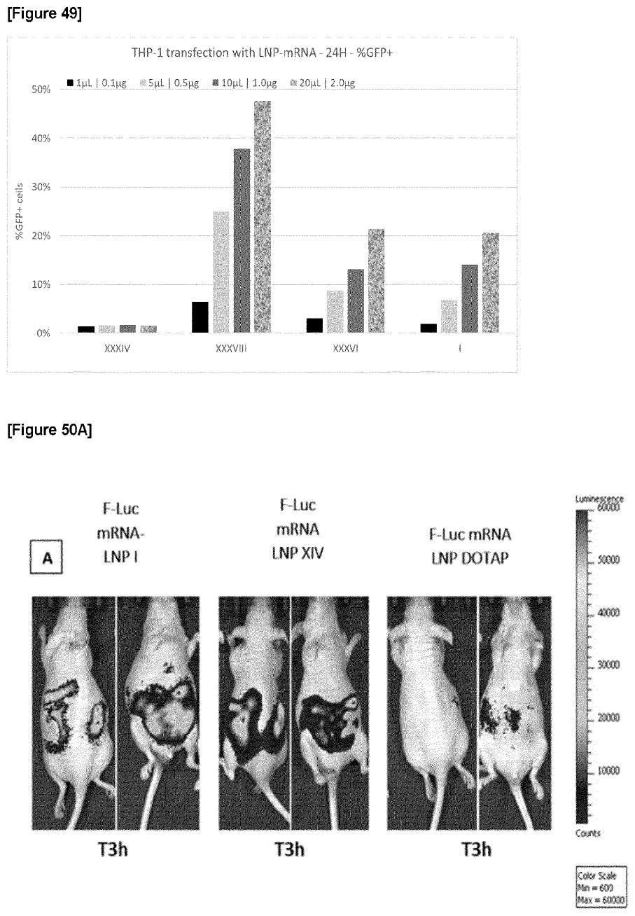

Results of transfection (FIG. 49) on the THP-1 cell line proved to be more difficult, as the best condition overall lead to about 50% of transfected cells using lipid XXXVIII. On the other hand, lipid XXXIV that proved to be efficient on other cell lines, didn't reached the same values of transfected cells THP-1. Lipid I and XXXVI demonstrated similar dose-dependant transfection profiles, peaking around 20% of transfected cells with the highest mRNA employed quantity.

This new set of experiment is a clear demonstration that the lipid of this invention could be used efficiently to transfect cell lines derived from immune cells such as macrophages or monocytes. Interestingly, we could also notice that LNPs based the same lipids presented different transfection patterns depending on the cell line employed. It is perfectly visible in the case of lipid XXXIV, which proved to be a really valuable LNP component to transfect macrophage cell line, whereas it was not as efficient on macrophages. This observation is an additional proof that people eager to use transfection driven by LNPs, should rely on a broad library of LNPs components, in particular regarding ionizable tunable lipids. This variety is a guarantee to have a reliable and efficient solution for mRNA transfection in a wide variety of cell lines, even on hard to transfect one.

In Vivo Transfection of F-Luc mRNA Using LNPs; Efficiency and Biodistribution

LNPs have been described as a really powerful gene delivery vehicle for in vivo application, due to their harmless nature and their potency to efficiently protect and deliver a wide array of nucleic acid. The tunable lipids described in this invention pushed the safe character even further as their whole design and synthesis has been developed to meet these important criteria, and to spread even more their use in vivo.

Lipids I, VI and XIV have been formulated into LNPs following the molar composition depicted before (Table 1). 5-methoxyuridine-modified mRNA encoding for Firefly Luciferase (OZ Biosciences, Cat: MRNA16-1000) has been entrapped into the LNP. To demonstrate the innovative nature of the tunable lipids, an additional LNP that includes a well-known cationic lipid has been chosen for comparison purposes. DOTAP has been selected for its widely documented in vivo behaviour and its permanent positive charge at physiological pH The main physical properties of the resulting LNPs are displayed in Table 4.

TABLE 4
Main physical properties of mRNA(F-Luc) LNPs based on
tunable lipids I, VI, XIV, and cationic lipid DOTAP

| LNPs Characterization | I | | VI | | XIV | | DOTAP | |
|---|---|---|---|---|---|---|---|---|
| Total Lipid concentration | 5 | mM | 5 | mM | 5 | mM | 5 | mM |
| Size (PDI) | 88 nm (0.09) | | 115 nm (0.13) | | 99 nm (0.13) | | 96 nm (0.23) | |
| Zeta | −7.3 | mV | −2.25 | mV | +4.85 | mV | +17 | mV |

| Loaded RNA (ng/μL) | 195 | 188 | 175 | 183 |
|---|---|---|---|---|
| N/P Ratio | 6 | 6 | 6 | 6 |
| Encapsulation Efficiency | 97.5% | 94% | 87.5% | 91.5% |

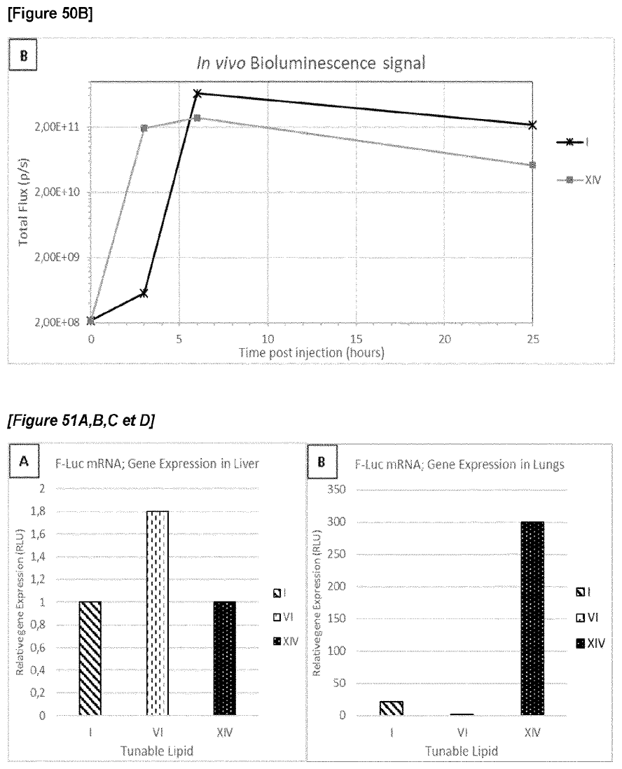

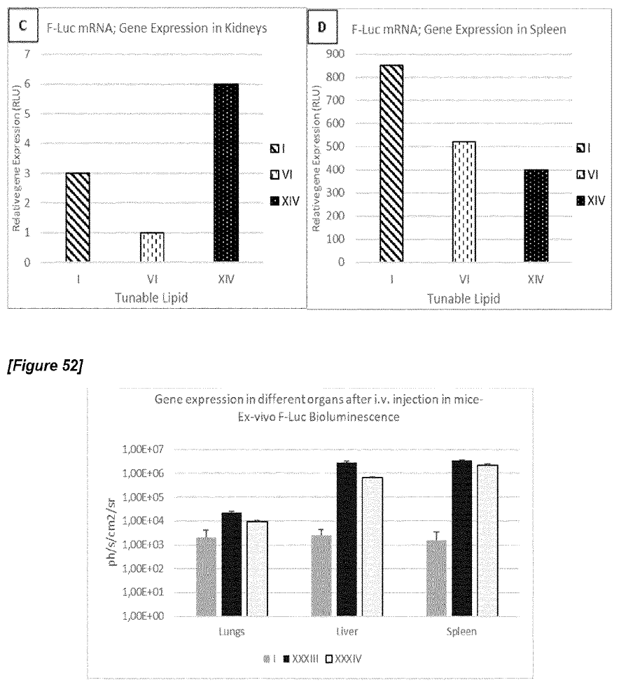

Overall, all LNPs formulations display consistent physico-chemical properties, controlled and reproducible size, charge, polydispersity and encapsulation rate to be used in small animals. A dose equivalent to 10 μg of mRNA has been injected i.p. (intra-peritoneally) in nude mice using three different LNPs. Tunable lipids I and XIV and DOTAP-based LNPs have been selected. Bioluminescence has been monitored over time using IVIS imaging system. Bioluminescence evaluation demonstrates that all three LNPs succeeded in delivering F-Luc mRNA into mice. Indeed 3 h post injection, luminescence could be observed in all animals at different proportions (FIGS. 50A&B). Specifically, F-Luc mRNA expression was higher in mice that received LNPs based on tunable lipids, whereas expression was much lower when DOTAP is used. Interestingly, the FIG. 50B that monitors the kinetics of the bioluminescence signal over time presents meaningful differences between the two LNP formulations. Indeed, LNPs based on tunable lipid I gave the highest overall luminescence signal, with a maximum after 6 hours post injection whereas LNPs based on lipid XIV allowed the maximum bioluminescence signal only after 3 h, earlier than with the other formulation. This result demonstrates the efficiency in vivo of the tunable lipids claimed in this invention, but also the complementary behaviour that can results from their structural diversity, providing the skilled scientist with formulations able to deliver efficiently nucleic acids with different kinetics in this case.

Biodistribution study involving tunable lipids-based LNPs (lipids I, VI, and XIV) has also been conducted in nude mice that were injected i.p. with Luciferase mRNA formulated into 3 different LNPs formulations at 10 μg RNA dose to evaluate the nucleic acid delivery into various organs. The liver, lung, kidney and spleen were collected and analysed through RT-PCR 6 hours after injection. As observed previously, tunable lipids-based LNPs are appropriately efficient to allow the observation and quantitation of Luciferase protein in quantity 6 hours after i.p. injection. LNPs based on lipid I provides high protein expression rate, observed in lung and spleen and to lower extend also in the kidney. LNPs based on lipid VI showed a high delivery in the spleen after 6 hours. Interestingly this formulation is also able to deliver mRNA in the liver at the chosen N/P ratio, with a rate comparable to those observed in the lungs. Meanwhile LNPs based on lipid XIV offers the highest rate of gene expression in lung, with a rate as high as those observed in spleen. It is also the most efficient between the three tested formulation in expressing the Luciferase in kidneys. This biodistribution study, displayed the efficiency of the LNPs based on tunable lipids to transport nucleic acid in different organs in vivo. Furthermore, the complementary nature of the results, allowing different expression rate in organs depending on the chosen tunable lipid, is a clear demonstration of the importance of the structural variety offered by this invention, which permits organ targeting through the choice of the tunable lipid's structure.

In Vivo Intra-Veinous (i.v.) Injection of F-Luc mRNA Using LNPs; Efficiency and Biodistribution

To dig further the potency of the molecules displayed in the present invention, we decided to test it in vivo in a different experimental setup. We decided to incorporate once more mRNA inside LNPs, and to inject it i.v. in wild-type mice. Lipids I, XXXIII and XXXIV have been formulated into LNPs following the molar composition depicted before (Table 5). 5-methoxyuridine-modified mRNA encoding for Firefly Luciferase (OZ Biosciences, Cat: MRNA16-1000) has been entrapped into the LNP. The main physical properties of the resulting LNPs are displayed in Table 5.

TABLE 5
Main physical properties of mRNA(F-Luc) LNPs
based on tunable lipids I, XXXIII, XXXIV

| LNPs Characterization | I | XXXIII | XXXIV |
|---|---|---|---|
| Total Lipid | 3.75 | 3.75 | 3.75 |
| concentration (mM) | | | |
| Size (PDI) | 89 nm (0.1) | 139 nm (0.14) | 121.2 nm (0.12) |
| Zeta | 0.3 mV | −1.2 mV | 3.35 mV |
| Loaded RNA (ng/μL) | 89.1 | 78.2 | 84.4 |
| N/P Ratio | 6 | 6 | 6 |
| Encapsulation | 89.1% | 78.2% | 84.4% |
| Efficiency | | | |

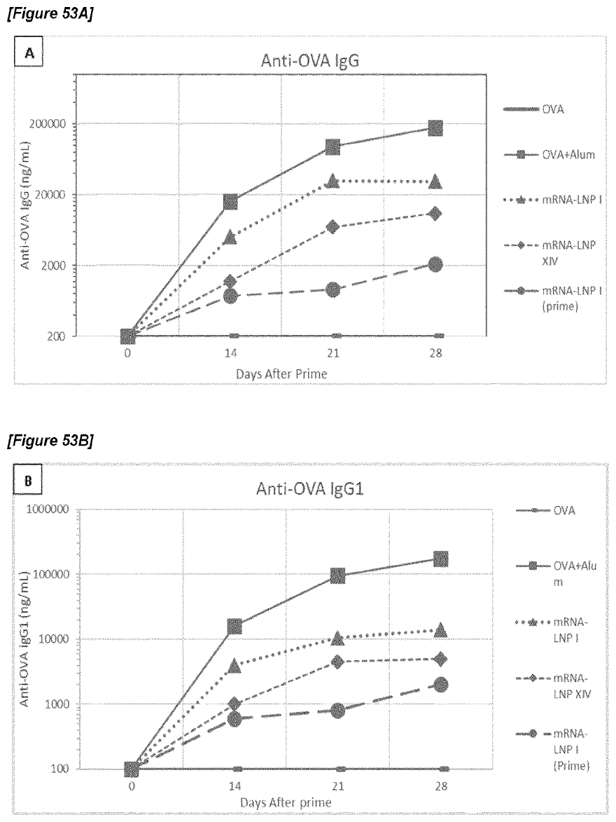

All LNPs formulations display here again consistent physico-chemical properties, controlled and reproducible size, charge, polydispersity and encapsulation rate to be used in small animals. A dose equivalent to 10 μg of mRNA has been injected i.v. in nude mice using the three different LNPs. Biodistribution studies (displayed in FIG. 52) have been conducted to evaluate the nucleic acid delivery into various organs. The liver, lungs, kidney and spleen were collected and analysed through RT-PCR 24 hours after injection. As observed previously, tunable lipids-based LNPs are appropriately efficient to allow the observation and quantitation of Luciferase protein in quantity 6 hours after i.v. injection. LNPs based on lipid XXXIII and XXXIV provides high protein expression rate, especially in liver and spleen and to lower extend also in the lungs. LNPs based on lipid XXXIV showed a high delivery in the spleen after 6 hours. Interestingly this formulation is also able to deliver mRNA in the liver at the chosen N/P ratio. Meanwhile LNPs based on lipid XXXIII offers the highest rate of gene expression in spleen, with almost rate as high as those observed in liver. It is also the most efficient between the three tested formulation in expressing the Luciferase in lungs. On the other hand, LNPs based on lipids I, that were highly efficient when injected i.p., provide more modest results when used i.v. with only average Luciferase expression rates spread almost equally between lungs, liver and spleen. This biodistribution study, displayed the efficiency of the LNPs based on tunable lipids to transport nucleic acid in different organs in vivo. In this case again, the different expression rates in organs depending on the chosen tunable lipid, is a clear demonstration of the importance of the structural variety offered by this invention, which permits organ targeting through the choice of the tunable lipid's structure.

In Vivo Immunization Experiment; Delivery of Antigen OVA mRNA Using LNPs

Recent studies clearly demonstrated the need for the community to develop new generation of vaccines, easy to handle, safe to the patient while remaining efficient in immunization. In this context, the emergence of vaccine-based LNPs represents a clear opportunity to reach pathologies impossible to fight with more classical vaccine approach. The tunable lipid presented in this document could envisioned as potent pillar for future nucleic acid-based vaccine, due to their ground-breaking features in term of efficiency and safe character. Thus, LNPs based on tunable lipids I and XIV have been formulated to encapsulate unmodified mRNA coding for the ovalbumin (OVA) antigen (OZ Biosciences, Cat: MRNA42-1000) and their physico-chemical properties determined (Table 6).

TABLE 6
Main physical properties of mRNA(OVA)
LNPs based on tunable lipids I and XIV.

| | | |
|---|---|---|
| LNPs Characterization | I | XIV |

| | | | | |
|---|---|---|---|---|
| Total Lipid concentration | 5 | mM | 5 | mM |

| | | |
|---|---|---|
| Size (PDI) | 111.4 nm (0.153) | 131.7 nm (0.149) |

| | | | | |
|---|---|---|---|---|
| Zeta | −9.01 | mV | −7.9 | mV |

| Loaded RNA (ng/μL) | 98.1 | 94 |
|---|---|---|
| N/P Ratio | 6 | 6 |
| Encapsulation Efficiency | 98.1% | 94% |

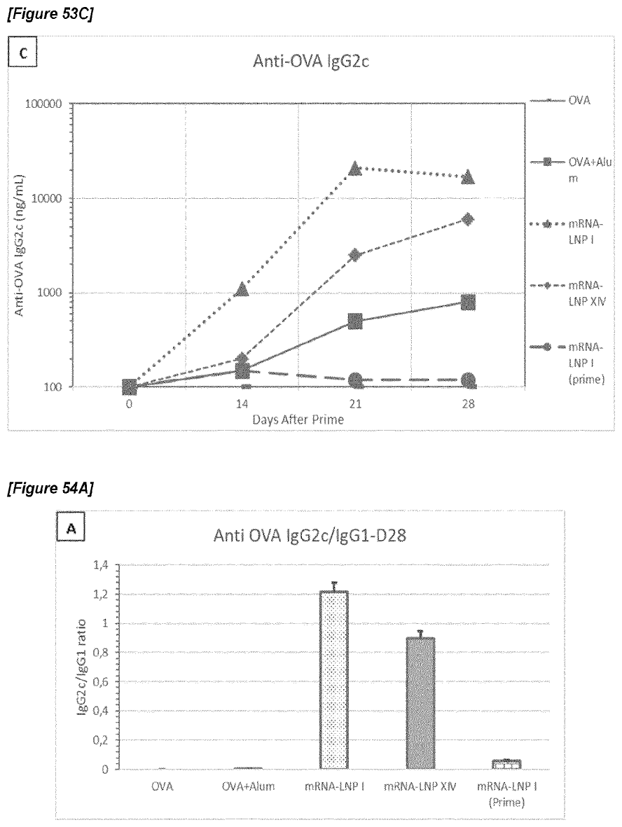

The two LNPs formulations showed consistent, and reproducible size, polydispersity charge and encapsulation efficiency. The two LNPs have been used in an immunization study to demonstrate their efficacy and potential in comparison to a classical recombinant subunit vaccine. C57BL6J mice received a prime boost (D0-14) immunizations of OVA antigen delivered by mRNA-based LNPs or by recombinant protein (alone or adjuvanted with aluminium-based adjuvant). To characterize the immuno-phenotyping of the mice, the sera and splenocytes were collected at different time (D0, D14, D21, D28) and analysed by ELISA and Elispot. The mice were immunized by s.c (sub-cutaneous) route with 100 μL per injection at 10 μg OVA subunit and 10 μg mRNA doses. Results are depicted in FIGS. 53 and 54, mRNA-OVA LNP formulations are efficient at priming a strong humoral response upon sub-cutaneous prime boost injection (D0-14). Indeed, a strong antibody response was measured after prime boost immunization. In addition, no significative signal was observed when the dose of 10 μg of OVA antigen is directly administrated alone. It is noteworthy, the importance of the boost (D₁₄) to obtain a long-lasting immune response. Both mRNA-OVA LNP based on lipid I and lipid XIV are efficient in generating an immune response following the administration of mRNA s.c., especially regarding the initial response of IgG2c. mRNA-OVA LNP formulations are efficient at priming a strong TH1 humoral response equivalent to the best TH1 adjuvants upon sub-cutaneous prime boost injection (D0-14). mRNA-OVA LNP formulations are also efficient at priming a strong anti-OVA CD8 T cell response equivalent to one of the most efficient adjuvants upon sub-cutaneous prime boost injection (D0-14) as shown by IFN□ Elispot results and also IL-18 production (not shown).

### Encapsulation of DNA in Lipid Nanoparticles Using Microfluidic Formulation;

The potency of the tunable lipids was further assessed for DNA delivery, several lipids belonging to this invention have been selected to encapsulate plasmid DNA into LNPs. After a initial screening, lipids I and XI have been selected as basis tunable compound for the formulation. The same microfluidic procedure used for encapsulation of mRNA has been translated to DNA applications. Ratio between all lipidic components (i.e., tunable lipid, helper neutral lipid, sterol derivative and PEG-lipid) used with mRNA has been kept (see table 1) with DNA, and the microfluidic formulation procedure remained unchanged. As a basis, a Flow Rate Ratio (FRR) equals to 3 and a total flow rate of 1000 μL/min has been set up. Collected LNPs are subsequently dialyzed against PBS+5% w/v sucrose overnight, filtered on a sterile 0.45 μm filter and stored at −20 °C. Plasmids DNA (pVectOZ) were designed and produced by OZ Biosciences. Three plasmids coding for Ppt, eGFP and F-Luc have been tested. Thus, LNPs based on tunable lipids I and XI have been formulated physico-chemical properties determined accordingly (Table 7).

TABLE 7
Main physical properties of pVectOZ plasmids-
based LNPs based on tunable lipids I and XI.

| Tunable Lipid | I | | I | | XI | |
|---|---|---|---|---|---|---|
| Total Lipid concentration | 4 | mM | 3.91 | mM | 4.6 | mM |
| DNA | pVectOZ(Ppt) | | pVectOZ | | pVectOZ | |
| | | | (eGFP) | | (F-Luc) | |
| Size (PDI) | 173 nm | | 158.9 nm | | 103 nm | |
| | (0.079) | | (0.044) | | (0.079) | |
| Zeta | +14.3 | mV | +4.61 | mV | +16.5 | mV |

| Loaded DNA (ng/μL) | 0.125 | 0.125 | 94 |
|---|---|---|---|
| N/P Ratio | 10 | 6.6 | 12 |
| Encapsulation Efficiency | 82% | 83% | 92% |

Results displayed on the table 7 clearly highlights the propensity of tunable lipids I and XI to efficiently encapsulate different DNA plasmids into LNPs. Due to higher size of DNA versus mRNA, N/P ratio have been adjusted, so that the size of the resulting nanostructures is maintained below 200 nm with polydispersity indexes having values lower than 0.1, that qualifies these structures for further testing and gives good insights about their mid-term stability.

### In Vitro Transfection of F-Luc DNA Using LNPs

The LNP formulation based on lipids XI has then been tested in an in vitro transfection experiment to demonstrate their potency to efficiently deliver DNA, while enabling DNA gene expression. Jurkat T-cells were used. Transfection efficiency has been assessed after 48 h incubation using the Luciferase Assay Kit (OZ Biosciences) by evaluating the luminescence intensity (Perkin Elmer Victor NOVO luminometer) obtained after lysis of the transfected cells, in presence of luciferin, magnesium and ATP. A quantity of DNA ranging from 0.1 μg to 10 μg has been tested while MTX reagent (OZ Biosciences SAS), a commercially available transfection reagent (a cationic lipid with permanent positive charges) devoted to DNA delivery, has been used as a positive control.

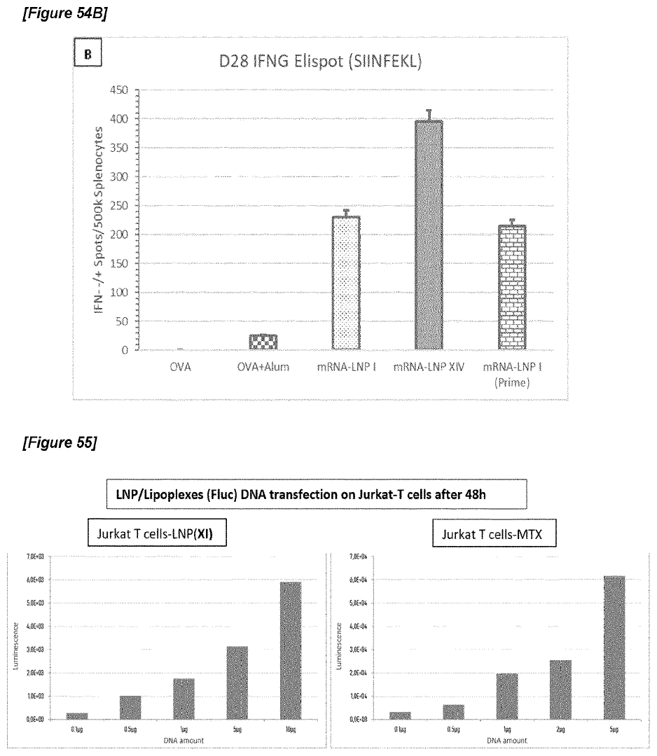

This result (FIG. 55) displays again the potency of LNPs based on tunable lipids to transfect a hard to transfect suspension cell line using a wide range of DNA quantities, with a growing of quantity of measured emitted light directly correlated with the amount of introduced DNA.

Reached luminescence values proved to be similar than those observed with a more classical transfection reagent, while maintaining cell viability. These results clearly show the potency of LNPs based on tunable lipids to be efficient in an in vitro DNA transfection, at level equivalent to standard transfection reagent, without compromising cell survival especially at high nucleic acid doses.

Encapsulation of siRNA in Lipid Nanoparticles Using Microfluidic Formulation;

Since tunable lipids proved to encapsulate efficiently large polynucleotides such as mRNA or DNA, their potency to efficiently protect and deliver smaller polynucleotides such as siRNA has also been investigated. Thus, several tunable lipids of to this invention have been selected to encapsulate NTsiRNA into lipid nanoparticles. After a selection round, lipids XI and XIV have been chosen as basis compound for the formulation. The same microfluidic used for encapsulation of mRNA has been translated to siRNA applications.

Chosen NTsiRNA have been labelled using Cyanine 5 (Cy5), to be able to monitor their uptake after LNPs exposure onto cells. Similarly, selected formulated LNPs have been labelled using DiO (Benzoxazolium, 3-octadecyl-2-[3-(3-octadecyl-2(3H)-benzoxazolylidene)-1-propenyl]-, perchlorate), a lipophilic green fluorescent dye widely used to label lipophilic regions. Ratio between all lipidic components (i.e., tunable lipid, helper neutral lipid, sterol derivative and PEG-lipid) used with siRNA have been kept (see table 1) with siRNA, the microfluidic formulation procedure remained unchanged. As a basis, a Flow Rate Ratio (FRR) equals to 3 and a total flow rate of 1000 μL/min has been set up. Collected LNPs are subsequently dialyzed against PBS+5% w/v sucrose overnight, filtered on a sterile 0.45 μm filter and stored at −20 °C.

Thus, LNPs based on tunable lipids I, XI and XIV have been formulated physico-chemical properties determined accordingly (Table 8).

TABLE 8
Main physical properties of siRNA-based LNPs based on tunable lipids XI and XIV

| Tunable Lipid | I | | I | | XI | | XI | | XI | | XI | | XIV | | XIV | |
|---|---|---|---|---|---|---|---|---|---|---|---|---|---|---|---|---|
| Total Lipid concentration | 4.74 | mM | 4.74 | mM | 4.74 | mM | 4.74 | mM | 4.74 | mM | 4.74 | mM | 4.74 | mM | 4.74 | mM |
| siRNA | eGFP | | eGFP | | eGFP | | eGFP | | NTsiRNA- | | NTsiRNA- | | NTsiRNA- | | NTsiRNA- | |
| | | | | | | | | | Cy5 | | Cy5 | | Cy5 | | Cy5 | |
| Size (PDI) | 108 nm | | 82 nm | | 94 nm | | 60 nm | | 116 nm | | 99 nm | | 114 nm | | 106 nm | |
| | (0.176) | | (0.102) | | (0.251) | | (0.153) | | (0.091) | | (0.073) | | (0.222) | | (0.198) | |
| Zeta | +3.9 | mV | +5.4 | mV | +6.1 | mV | +3.6 | mV | +18.2 | mV | +19.9 | mV | +27 | mV | −2.46 | mV |

| Loaded siRNA (ng/μL) | 0.125 | 0.125 | 0.125 | 0.125 | 0.125 | 0.125 | 0.125 | 0.125 |
|---|---|---|---|---|---|---|---|---|
| DiO Label Yes/No (Y/N) | N | N | N | N | N | Y | N | Y |
| N/P Ratio | 4 | 6 | 4 | 6 | 6 | 6 | 6 | 6 |
| Encapsulation | >90% | >90% | >90% | >90% | >90% | >90% | >90% | >90% |
| Efficiency | | | | | | | | |

Results displayed on the table 8 clearly highlights the propensity of tunable lipids XI and XIV to efficiently encapsulate siRNA into LNPs. Encapsulation rates of siRNA proved to be higher than 90% whatever the lipid used, whereas the size of the LNP is in all cases is maintained around 100 nm. However, polydispersity indexes observed when using lipid XI are better than with lipid XIV, with impressive values stable below 0.1. Insertion of DiO dye does not affect the morphology neither the physical properties nor integrity of the LNPs.

In-Vitro Uptake of Fluorescently Labelled siRNA Formulated in Lipid Nanoparticles.

The siRNA-based LNPs formulated above, have been used in an uptake study using confocal fluorescence microscopy. As already described before, the nanoparticles benefit from a double fluorescent-labelling on both the lipids used in the LNPs, and on the siRNA; that allow us to monitor uptake of the polynucleotide after exposure onto cells, as well as the potential internalization of the particles. Indeed, internalization is a crucial factor to control when envisioning extending the use of LNPs into a pre-clinical or clinical setting.

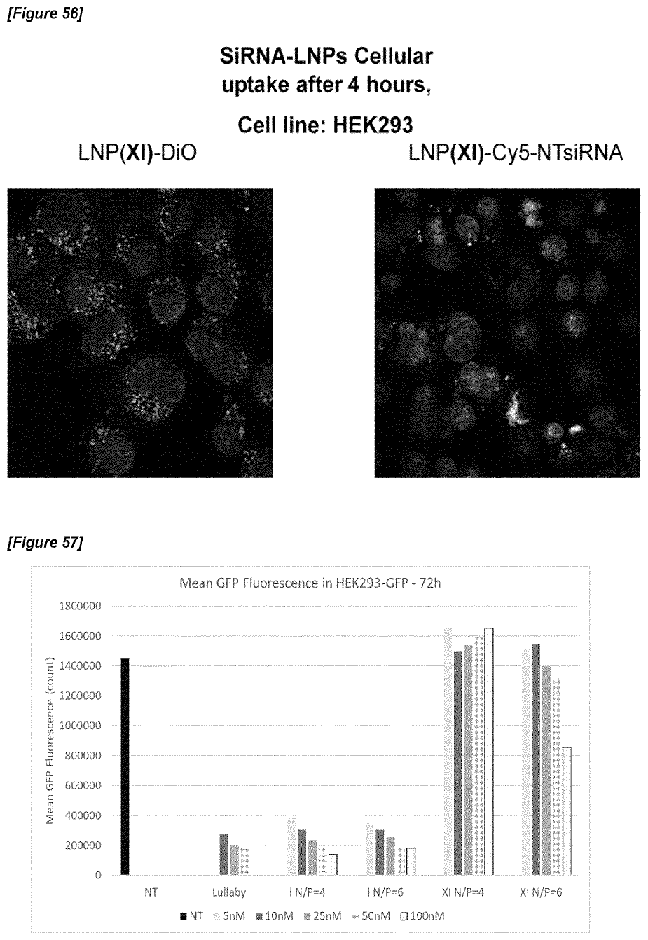

According, to the results displayed on table 8, LNPs based on tunable lipid XI have been selected for their improved physical properties. Five microliters of LNP have been put in contact of HEK-293 cells, before 4 hours of incubation. Pictures have then taken using a confocal microscope after nucleus coloration using Hoechst dye. These pictures are presented in FIG. 56.

On FIG. 56 (picture left), DiO-labelled particles are easily observed inside of the cytoplasm of cells, proving efficient internalization of the LNPs. The FIG. 56 (picture right) on the other hand, reveals the presence of Cy5-siRNA inside the cytoplasm that demonstrates efficient siRNA uptake after 4 h exposure of the formulated LNPs onto cells. These observations univocally prove the potency of tunable-based LNPs to act as efficient siRNA delivery platform able to protect and deliver efficiently siRNA inside of the cell's cytoplasm.

In Vitro Transfection of GFP siRNA Using LNPs

After having proved the propensity of LNPs to efficiently encapsulate and deliver siRNA, we decided to test those LNPs in an in vitro transfection setup. We used for this purpose stably transfected cell lines that stably expressed the eGFP fluorescent protein. Using flow cytometry, we wanted to measure the remaining mean of fluorescence after treatments with LNPs encapsulating siRNA against eGFP. A decrease in this value would mean that the LNPs are able to deliver safely inside the cells and that the siRNA targeting eGFP could be translated accordingly.

Two cells lines that stably express eGFP were selected: HEK-293 and THP-1. A growing concentration of siRNA ranging from 5 to 100 nM was applied on cells, encapsulated in LNPs.

We chose vectors based on lipids I and XI as lipidic platforms. LNPs were formulated at two different N/P ratio of 4 and 6. After administration of the particles, cells were cultured further 72 h before cytometry readout. As positive control, we used a well-known transfection reagent, employed for years to efficiently deliver siRNA inside cells (Lullaby, OZ Biosciences SAS, France).

Results on HEK-293 cells expressing eGFP are particularly self-speaking (FIG. 57). Application of LNPs based on lipid I induced a clear silencing of the secretion of the eGFP protein after 72 h, where we could observed that almost 90% of the fluorescence has been quenched by silencing of the gene in the best conditions. These results are similar than those observed with the positive control. Furthermore, no clear difference could be observed in this case between N/P=4 and N/P equal 6, the two conditions being as efficient in this experimental setup.

On the other hand, LNPs based on lipid XI failed to provide any eGFP fluorescence quenching before really high quantity were used, whatever the selected N/P ratio. It proves univocally that the selection of the right tunable component is crucial for the success of a LNP-mediated transfection setup.

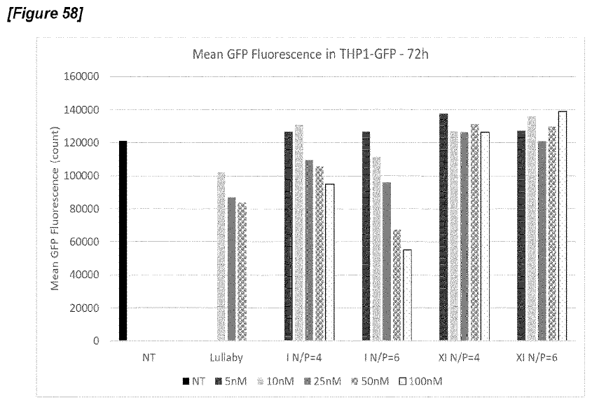

Similar results (FIG. 58) were observed when those same siRNA-LNPs were used to transfect THP-1 cell line stably expressing eGFP. Even on those cells known for being more difficult to transfect, LNPs based on lipid I were highly efficient allowing to reach around 50 percent of silencing using the optimal siRNA quantity. Furthermore in this case, the N/P ratio choice proved to have more influence, as LNPs formulated at N/P ratio of 6 were more efficient than at the ratio N/P of 4. As with HEK cells, formulation based on lipid XI was not usable in this experimental setup, highlighting the fact that the choice of the right tunable lipid is fundamental to reach optimum results. Those results also prove that lipids of the present invention could be employed at different N/P ratio to modulate the efficiency of the whole process. It offers thus an increased of flexibility to skilled scientists eager to use lipids of the current invention in their gene deliveries activities.

### Encapsulation of Oligonucleotides in Lipid Nanoparticles Using Microfluidic Formulation;

Oligonucleotides (ODN) are short DNA or RNA molecules, oligomers that have a wide range of applications in genetic testing, research, and forensics. For instance, aptamers, and antisense oligonucleotides are at the basis of many innovative therapeutic strategies due to their easy availability and their ability to target and modify very specific sequences of nucleic acid with high selectivity. A straightforward uptake, like for most small-molecule drugs, is hindered by the polyanionic backbone and the molecular size of ONs. Conversely, LNPs represent an interesting possibility as delivery platform able to protect and deliver efficiently single or double stranded oligonucleotides.

Thus, several lipids of to this invention have been selected to encapsulate a pool of four RNA ODNs into lipid nanoparticles. After a screening, lipid XI has been selected as basis tunable compound for formulations. The same microfluidic used for encapsulation of mRNA has been translated to ODN applications. 4 different RNA-ODN have been used for this encapsulation study, each formulated with lipid XI. The microfluidic formulation procedure remained unchanged. As a basis, a Flow Rate Ratio (FRR) equals to 3 and a total flow rate of 1000 μL/min has been set up. Collected LNPs are subsequently dialyzed against PBS+5% w/v sucrose overnight, filtered on a sterile 0.45 μm filter and stored at −80 °C. Results of physico-chemical properties of the different LNPs are displayed on the table 9 below.

TABLE 9
Main physical properties of ODNs-based LNPs based on tunable lipids XI

| | ODN1- | | ODN2- | | ODN3- | | ODN4- | |
|---|---|---|---|---|---|---|---|---|
| LNPs Characterization | LNP-XI | | LNP-XI | | LNP-XI | | LNP-XI | |
| Total Lipid concentration | 3 | mM | 3 | mM | 3 | mM | 3 | mM |
| Size (PDI) | 87.8 nm (0.043) | | 89.7 nm (0.054) | | 96.2 nm (0.135) | | 106.7 nm (0.119) | |
| Zeta | −1.39 | mV | −5.67 | mV | 3.56 | mV | −3.74 | mV |

| Loaded RNA (ng/μL) | 0.125 | 0.125 | 0.125 | 0.125 |
|---|---|---|---|---|
| N/P Ratio | 4 | 4 | 4 | 4 |
| Encapsulation Efficiency | 95% | 97% | 96% | 94% |

Results presented in table 9 highlight the potency of lipid XI to encapsulate various ODNs with superior efficiency. At a N/P of 4, all LNPs present a average size around or below 100 nm with controlled polydispersity close form 0.1 in all cases. Meanwhile, encapsulation rates are clearly in the upper range, with efficiencies in encapsulation close in all cases from 95%. Taken together these results validated the choice of lipid XI as basis for LNPs able to encapsulate efficiently ODNs. This invention offers thus to researchers to use an efficient encapsulation of ODNs of therapeutic interest.

1. Lipid of formula (I), or a stereoisomer thereof, or a pharmaceutically acceptable salt thereof:
R-Sp-Zₛ (I)
characterized in that:
the lipid does not include any positive charge at pH ranging from 7.0 to 7.4 whereas it displays one or several positive charges when the pH is below 7.0, preferably ranges from 2.0 and 7.0, more preferably ranges from 4.0 and 7.0 and even more preferably ranges from 4.5 and 7.0;
R is selected from: 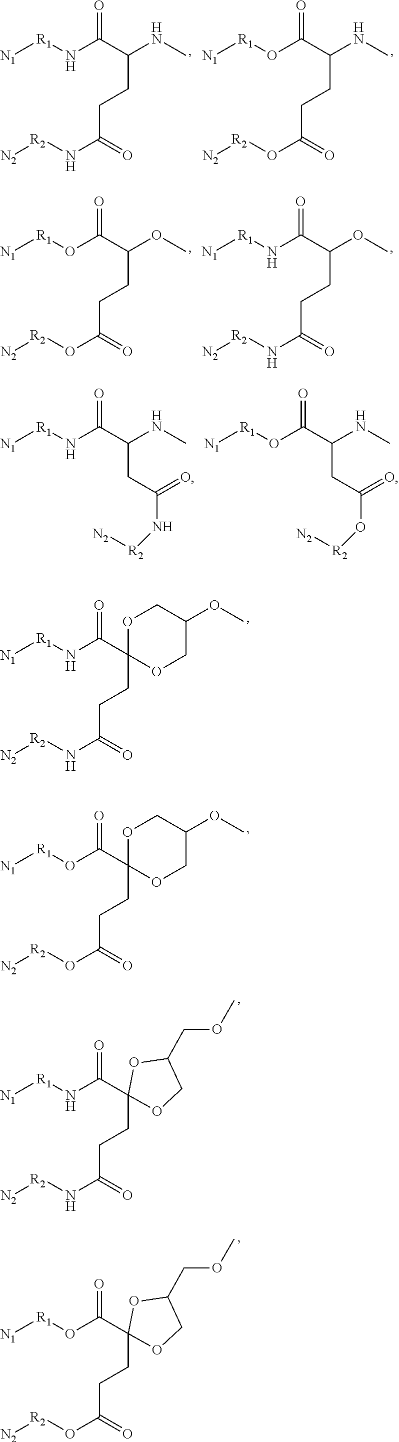 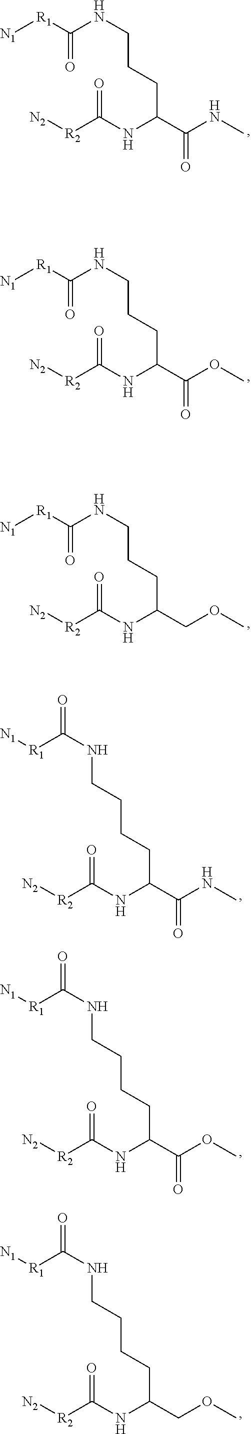 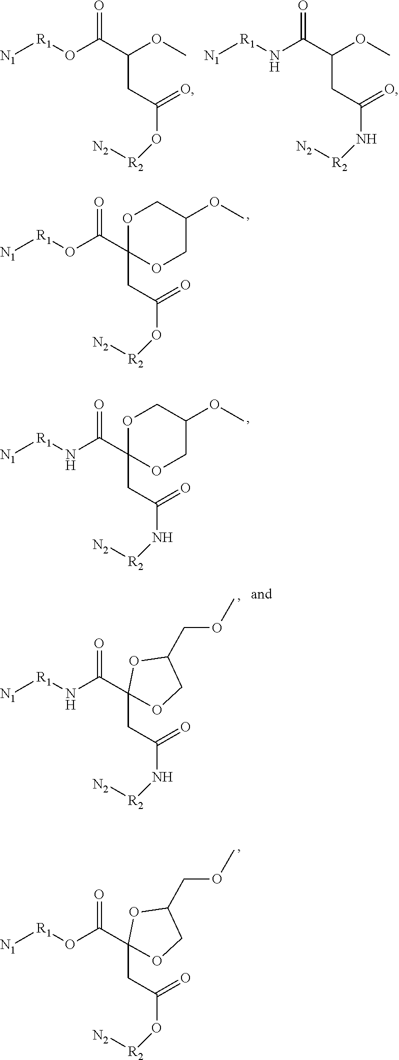
wherein:
N₁ and N₂, identical or different represent a linear, branched saturated or unsaturated, hydrocarbon group comprising from 1 to 8 carbon atoms, preferably from 1 to 4, used to terminate carbon chains, preferably N₁ and N2, identical or different, are either a methyl, ethyl, n-propyl, i-propyl, n-butyl, s-butyl, i-butyl or t-butyl group;
R₁ and R₂ are different and their structures are represented by formulae (VI a) and (VI b) respectively: 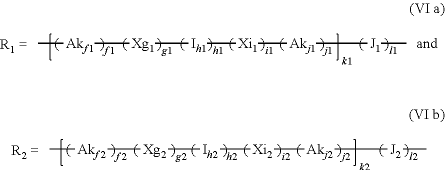
where:
Ak_{f1}, Akⱼ₁, Ak_{f2}, Akⱼ₂ identical or different, represent a linear or branched hydrocarbon group comprising from 1 to 22 carbon atoms, preferably from 1 to 12 carbon atoms, optionally comprising one or more heteroatoms;
f1 and f2 are integers equal to 0 or 1:
i1 and j2 are integers equal to 0 or 1:
Xg₁, Xi₁, Xg₂ and Xi₂, identical or different are selected from O, S, N(R₇), C(O), C(O)O, OC(O), C(O)N(R₇), N(R₇)C(O), OC(O)N(R₇), N(R₇)C(O)O, C(O)S, C(S)O, S(O), S(O)(O) and C(S), wherein R₇ is either an hydrogen (H) or a C₁-C₁₀ alkyl, C₂-C₁₀ alkenyl, or C₂-C₁₀ alkynyl, optionally substituted by one or more methyl, ethyl, n-propyl, i-propyl, n-butyl, s-butyl, i-butyl or t-butyl group(s); more preferably R₇ is selected from the group consisting of hydrogen (H), a C₁ alkyl (methyl) group, and a C₂, C₃, C₄, C₅, C₆, C₇, Cs, C₉, and C₁₀ alkyl, alkenyl, and alkynyl group, optionally comprising one or more heteroatoms, such as —O—, NH—, S and mixtures of thereof; preferably, it is likely understood that if Xg₁, Xi₁, Xg₂ and Xi₂happened to be different in nature while being independently represented by N(R₇), C(O)N(R₇), N(R₇)C(O), OC(O)N(R₇), N(R₇)C(O)O, then R₇ might be of several different nature among the possibilities depicted above within the same molecule:
g1 and g2 are integers equal to 0 or 1:
i1 and i2 are integers equal to 0 or 1:
Iₕ₁ and Iₕ₂, identical or different, both represent a linear or branched non-saturated hydrocarbon chains comprising from 2 to 16 carbon atoms, preferably from 2 to 8 carbon atoms and at least one unsaturation, more preferably Iₕ₁ and Iₕ₂. identical or different are independently chosen from: 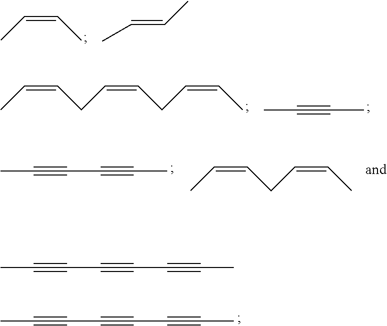
h1 and h2 are integers equal to 0 or 1:
J₁ and J₂ are branched hydrocarbon groups comprising from 1 to 20 carbon atoms, preferably from 1 to 12, and/or one or more heteroatoms;
l1 and l2 are integers equal to 0 or 1; and
k1 and k2 are integers comprised between 1 or 3, it being preferably understood that if l1 is equal to 1 then k1 is at least equal to 2; similarly, if l2 is equal to 1 then k2 is at least equal to 2; or
R₁ and R₂ are identical and their structure is represented by formula (VII): 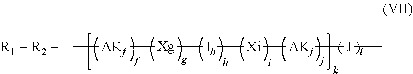
where:
Ak_{f} and Akⱼ, identical or different, represent a linear or branched hydrocarbon group comprising from 1 to 22 carbon atoms, preferably from 1 to 12 carbon atoms, optionally comprising one or more heteroatoms;
f is an integer equal to 0 or 1;
i is an integer equal to 0 or 1;
Xg and Xi, identical or different are selected from O, S, N(R₇), C(O), C(O)O, OC(O), C(O)N®, N(R₇)C(O), OC(O)N(R₇), N(R₇)C(O)O, C(O)S, C(S)O, S(O), S(O)(O) and C(S), wherein R₇ is either an hydrogen (H) or a C₁-C₁₀ alkyl, C₂-C₁₀ alkenyl, or C₂-C₁₀ alkynyl, optionally substituted by one or more methyl, ethyl, n-propyl, i-propyl, n-butyl, s-butyl, i-butyl or t-butyl group(s); more preferably R₇ is selected from the group consisting of hydrogen (H), a C₁ alkyl (methyl) group, and a C₂, C₃, C₄, C₅, C₆, C₇, Cs, C₉, and C₁₀ alkyl, alkenyl, and alkynyl group, optionally comprising one or more heteroatoms, such as —O—, NH—, S and mixtures of thereof;
g is an integer equal to 0 or 1;
i is an integer equal to 0 or 1;
Iₕ represent a linear or branched non-saturated hydrocarbon chain comprising from 2 to 16 carbon atoms, preferably from 2 to 8 carbon atoms and at least one unsaturation, more preferably Iₕ is selected from: 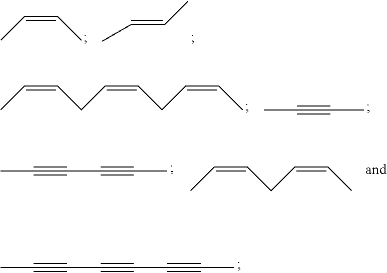
h is an integer equal to 0 or 1:
J is a branched hydrocarbon groups comprising from 1 to 20 carbon atoms, preferably from 1 to 12, and/or one or more heteroatoms;
l is an integer equal to 0 or 1; and
k is an integer comprised between 1 or 3; it being preferably understood that if 1 is equal to 1 then k is at least equal to 2,
Sp corresponds to the general formula (III): 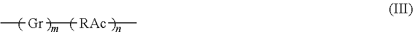
where:
Gr represents a linear or branched hydrocarbon group, comprising from 1 to 15 carbon atoms, preferably from 1 to 8, and optionally comprising one or more heteroatoms;
m is an integer equal to 0 or 1;
RAc represents an amino acid radical; and
n is an integer equal to 0 or 1; and
Zs corresponds to formula (IV): 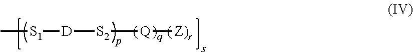
where:
S₁ and S₂, identical or different, represent a linear or branched hydrocarbon group comprising from 1 to 15 carbon atoms, preferably from 1 to 6 carbon atoms, optionally comprising one or more heteroatoms preferably chosen from nitrogen, oxygen, sulfur, bromine, iodine, chlorine and fluorine, more preferably from nitrogen, sulphur, bromine, iodine, chlorine and fluorine;
D represents one or more vinyl ether groups, one or more acylhydrazone groups, one or more carbonate bond, a disulfide bond, an ester bond or one or more photosensitive groups;
p is an integer equal to 0 or 1;
Q is a branched hydrocarbon group comprising from 1 to 20 carbon atoms, preferably from 1 to 12 carbon atoms, and one or more heteroatoms, and which can optionally be covalently coupled with the S₂ or RAc or Gr or R group on the one hand, and to at least two Q and/or Z groups on the other hand;
q is an integer comprised between 0 and 8, preferably between 0 and 3;
Z is a nitrogen (N) centered terminal ionizable moiety corresponding to formula (V): 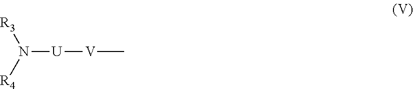
wherein:
R₃ and R₄ are either the same or different and selected from C₁-C₆ alkyl, C₂-C₆ alkenyl, or C₂-C₆ alkynyl, optionally substituted by one or more methyl, ethyl, n-propyl, i-propyl, n-butyl, s-butyl, i-butyl or t-butyl group(s), or R₃ and R₄ may join to form an optionally substituted by one or more methyl, ethyl, n-propyl, i-propyl, s-butyl, i-butyl or t-butyl group(s) heterocyclic ring such as pyrrol, pyrrolidine, pyridine, piperazine, morpholine, piperidine or indoline optionally substituted by one or more methyl, ethyl, n-propyl, i-propyl, s-butyl, i-butyl or t-butyl group(s);
N is a Nitrogen;
V is O, S, N(R₆), C(O), C(O)O, OC(O), C(O)N(R₆), N(R₆)C(O), OC(O)N(R₆), N(R₆)C(O)O, C(O)S, C(S)O, S(O), S(O)(O), C(S), or an optionally substituted heterocyclic ring such as a pyrrole, pyrrolidine, pyridine, piperazine, morpholine, piperidine or indoline, optionally substituted by one or more methyl, ethyl, n-propyl, i-propyl, n-butyl, s-butyl, i-butyl or t-butyl group(s), wherein R₆ is either an hydrogen (H) or a C₁-C₁₀ alkyl, C₂-C₁₀ alkenyl, or C₂-C₁₀ alkynyl, optionally substituted by one or more methyl, ethyl, n-propyl, i-propyl, n-butyl, s-butyl, i-butyl or t-butyl group(s); preferably R₆ is selected from the group consisting of hydrogen (H), a C₁ alkyl (methyl) group, and a C₂, C₃, C₄, C₅, C₆, C₇, C₈, C₉, and Clo alkyl, alkenyl, and alkynyl group, incorporating or not one or more heteroatoms, such as —O, —NH, —S and mixtures of thereof; and
U is either absent or is a C₁-C₁₂ alkyl, C₂-C₁₂ alkenyl, or C₂-C₁₂ alkynyl, optionally substituted by one or more methyl, ethyl, n-propyl, i-propyl, n-butyl, s-butyl, i-butyl or t-butyl group(s) and optionally substituted by one or more heteroatoms or group of heteroatoms preferably chosen from nitrogen, oxygen, sulfur, bromine, iodine, chlorine, fluorine or phosphorous;
where, Z does not include any positive charge at pH ranging from 7.0 to 7.4, whereas it displays one or several positive charges when the pH ranges from 2.0 and 7.0, preferably from 4.0 and 7.0 and more preferably from 4.5 and 7.0-1
r is an integer comprised between 1 and 16, preferably between 1 and 8, it being preferably understood that if q is equal to 1 then r is at least equal to 2 and that if r is greater than 1, then the Z groups can be identical or different; and
s is an integer equal to 1 or 2,
the following compounds being excluded from general formula (I): 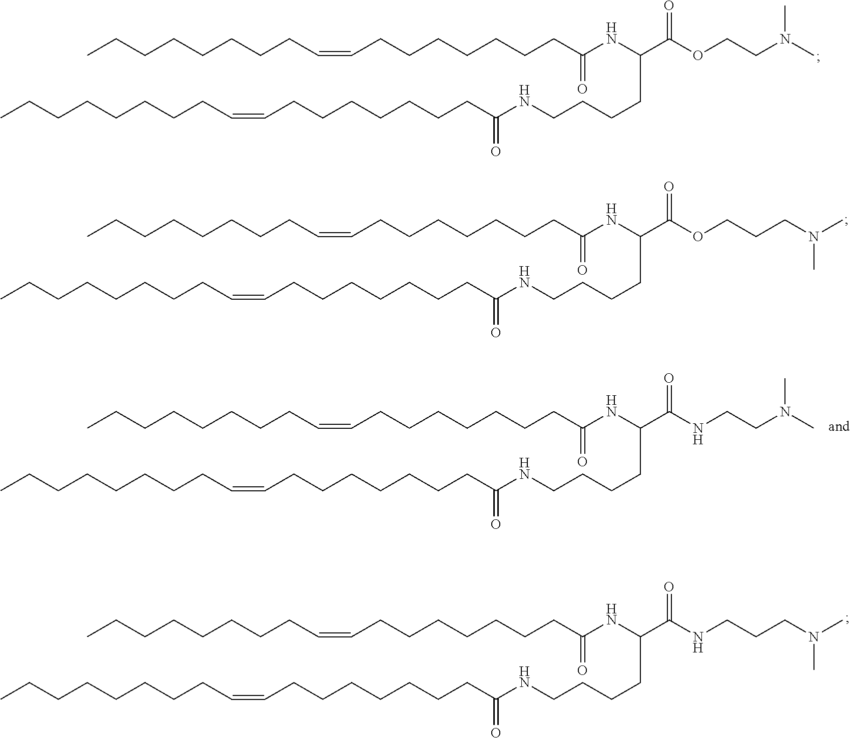
and
in formula (III):
Gr is absent or Gr serves as a spacer arm and corresponds to the molecular pattern noted as —W₄—Y₄—W₄—,
where Y₄ represents a bridging alkylene group comprising from 1 to 8 carbon atoms, preferentially from 1 to 4, whilst W₄ represents a
C(O)—O, C(O)—NH, C(O)—S, S(O), S(O)—O, C(S)—O, —O— or —NH— group;
or
Gr corresponds to the formula —W₄—Y₄—W₄—,
where Y₄ has the same meaning as previously defined and is connected by a carbonate or carbamate bond to the lipophilic region R on the one hand, and either to the RAc radical, or directly to the Si or Q or Z group on the other hand; and
in formula (III) the amino acid radical RAc is chosen from aspartic acid, glutamic acid, isoleucine, leucine, lysine, ornithine, glycine and phenylalanine, and more preferably from aspartic acid, glutamic acid, glycine, ornithine and lysine.

2. (canceled)

3. (canceled)

4. (canceled)

5. A lipid, or a stereoisomer thereof, or a pharmaceutically acceptable salt thereof, selected from:

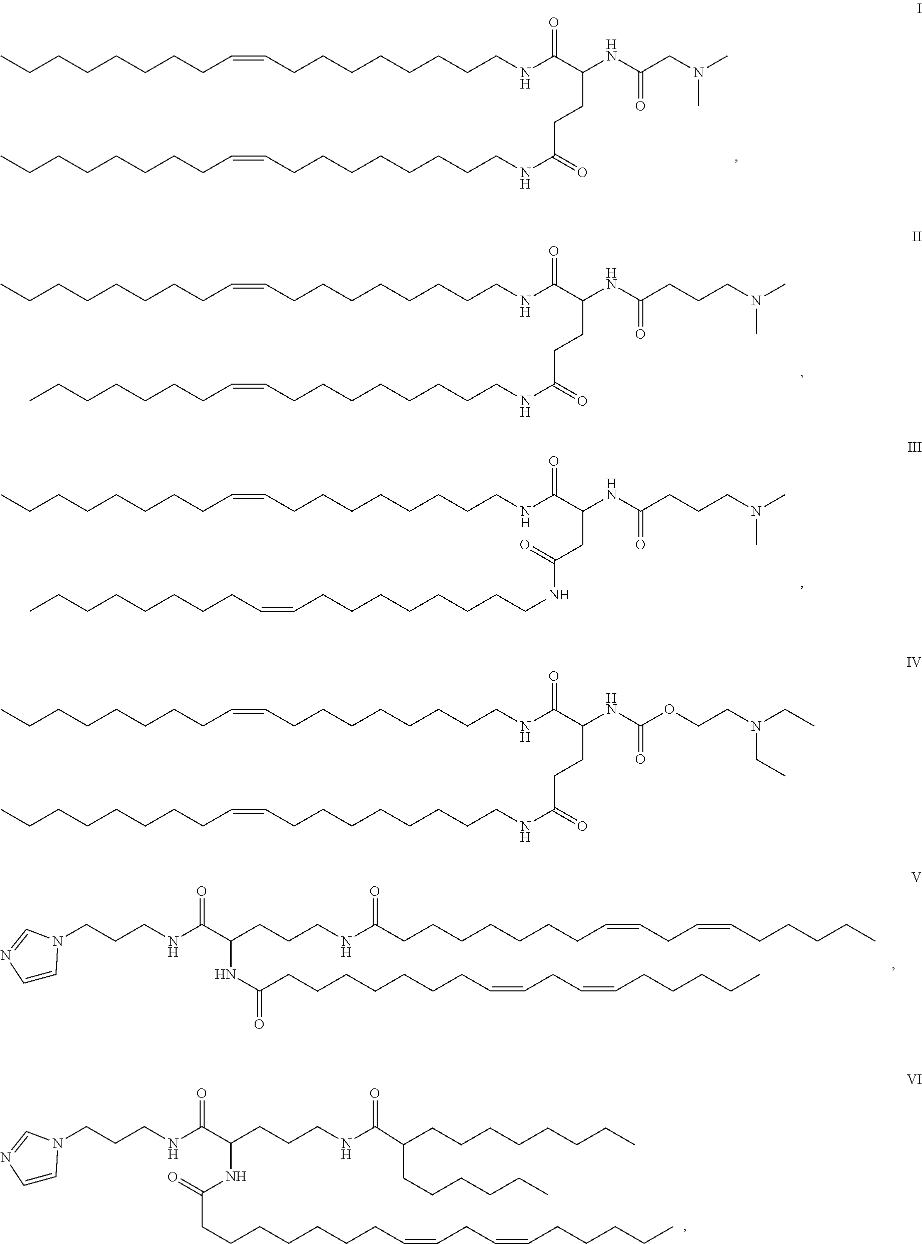

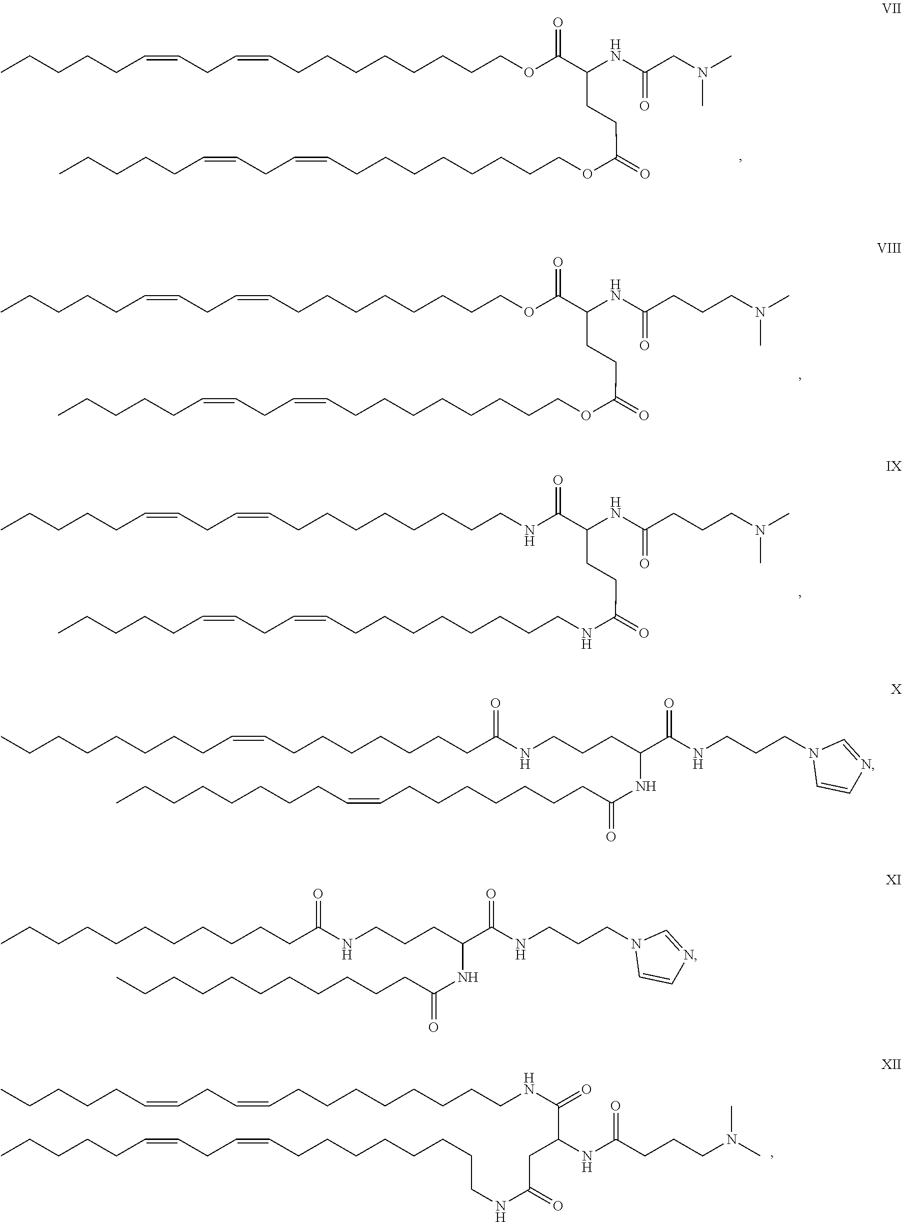

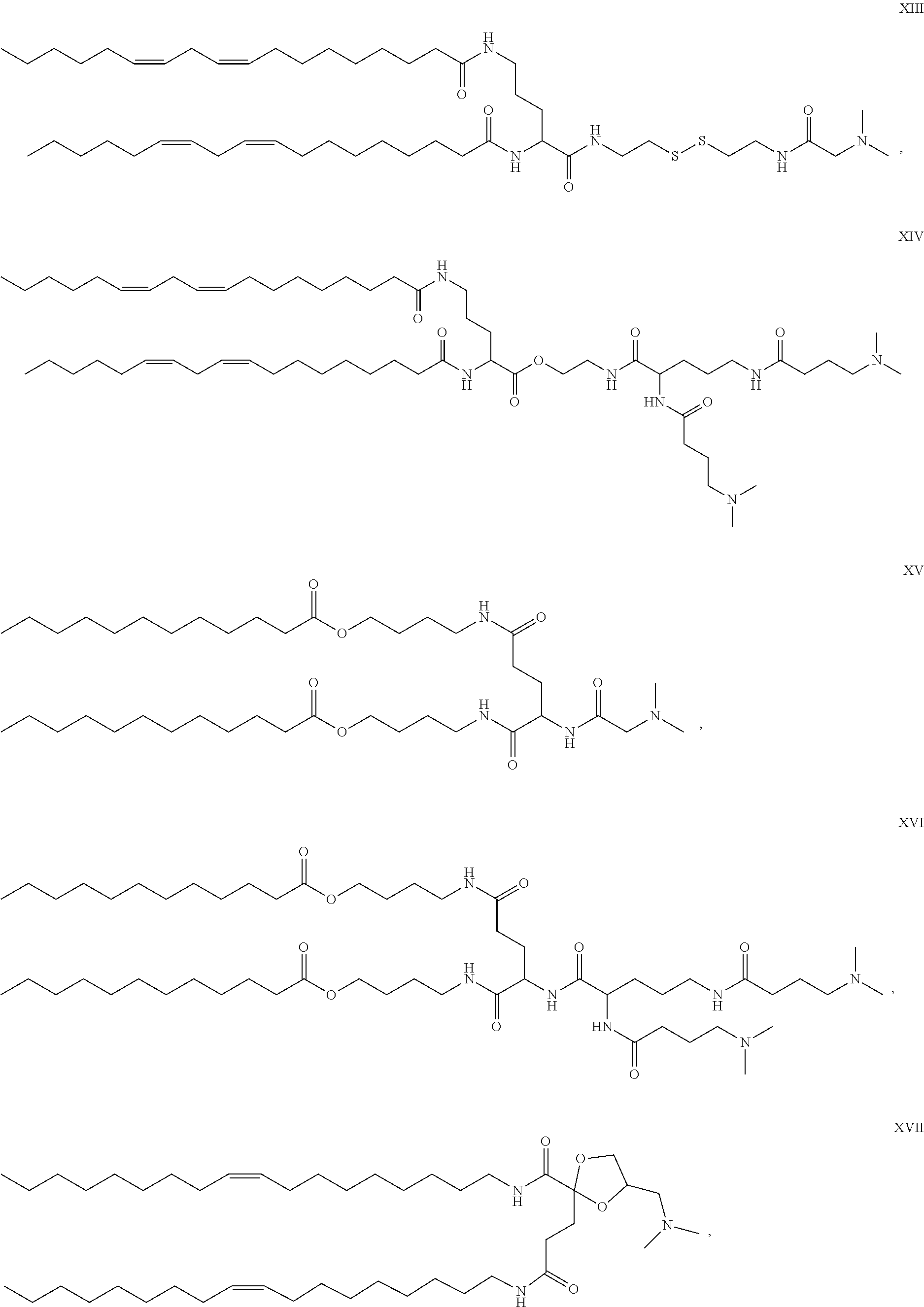

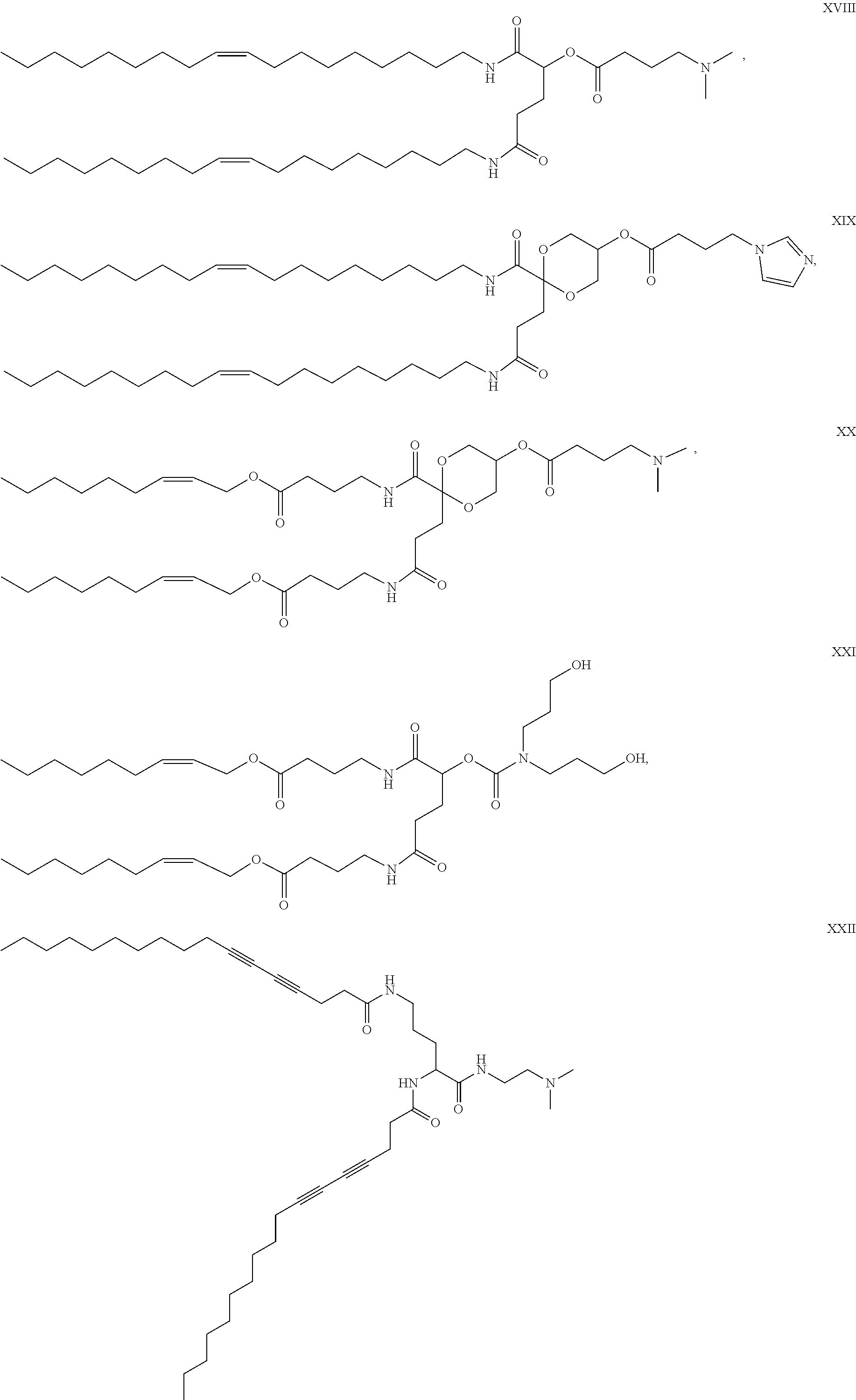

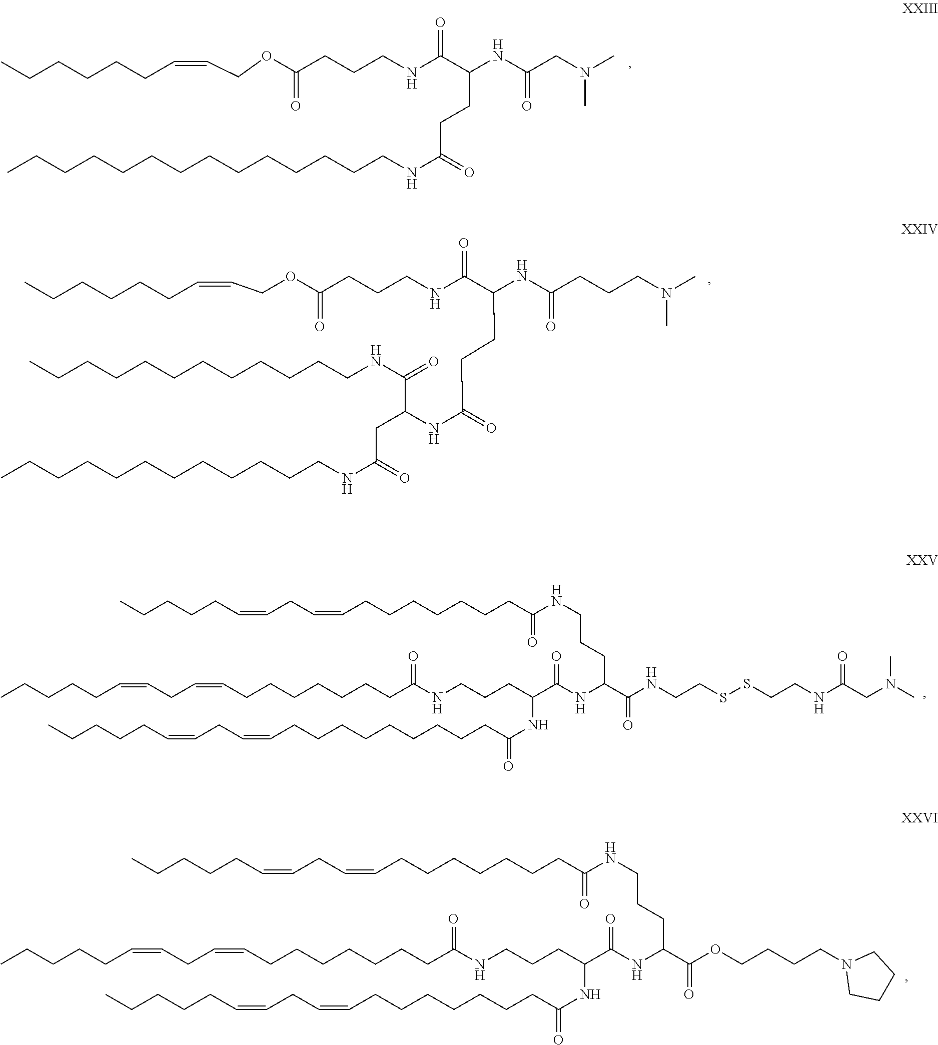

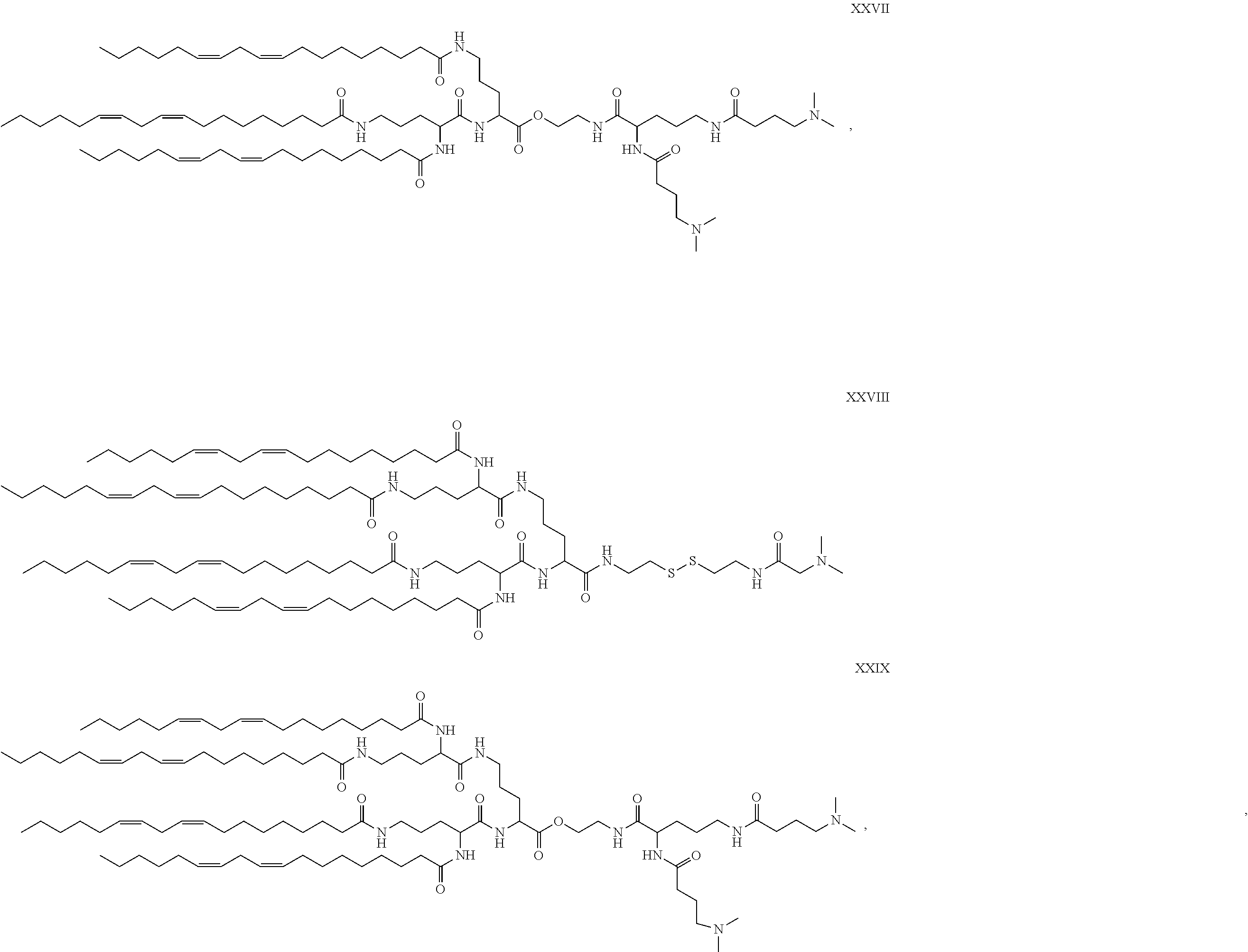

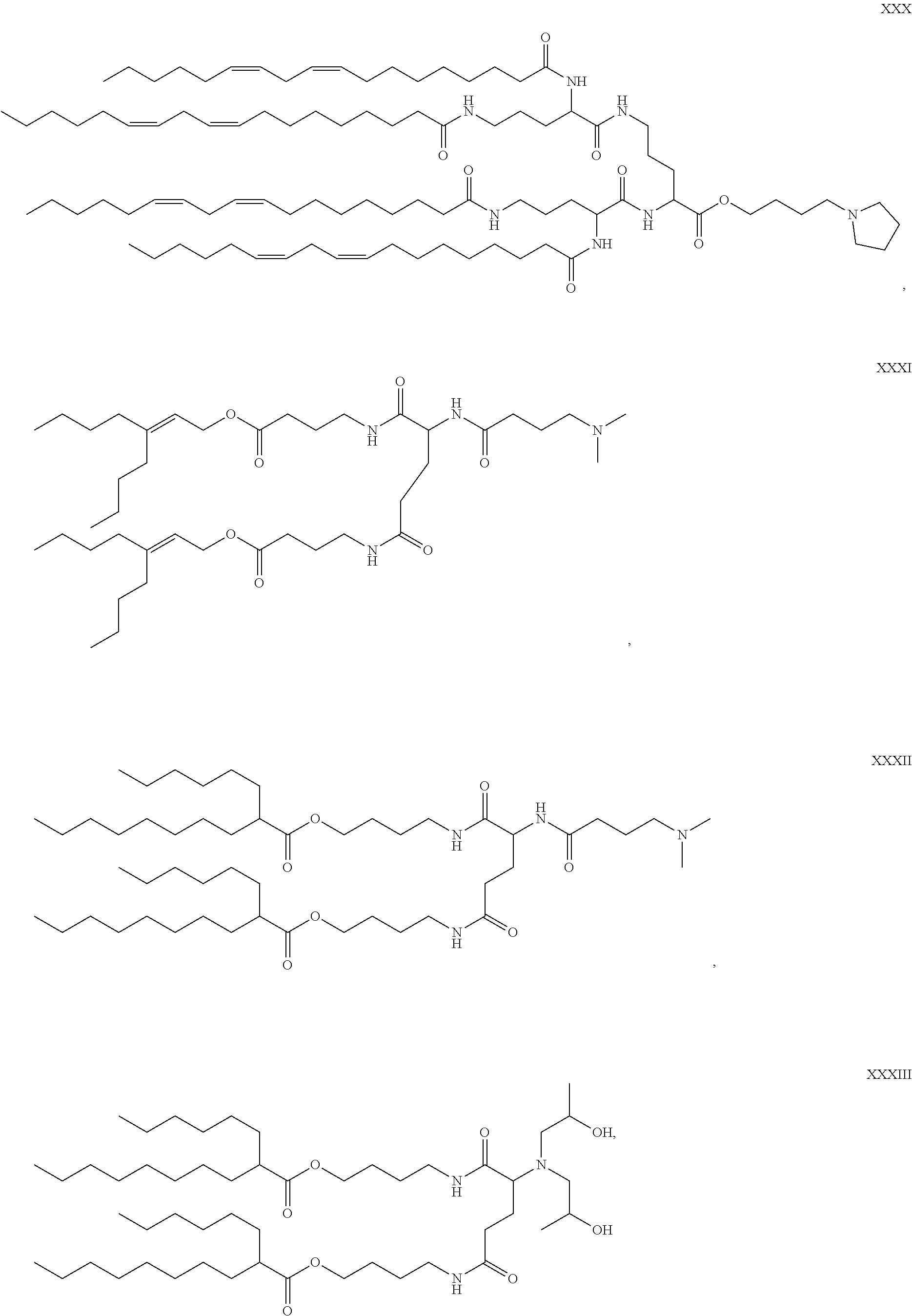

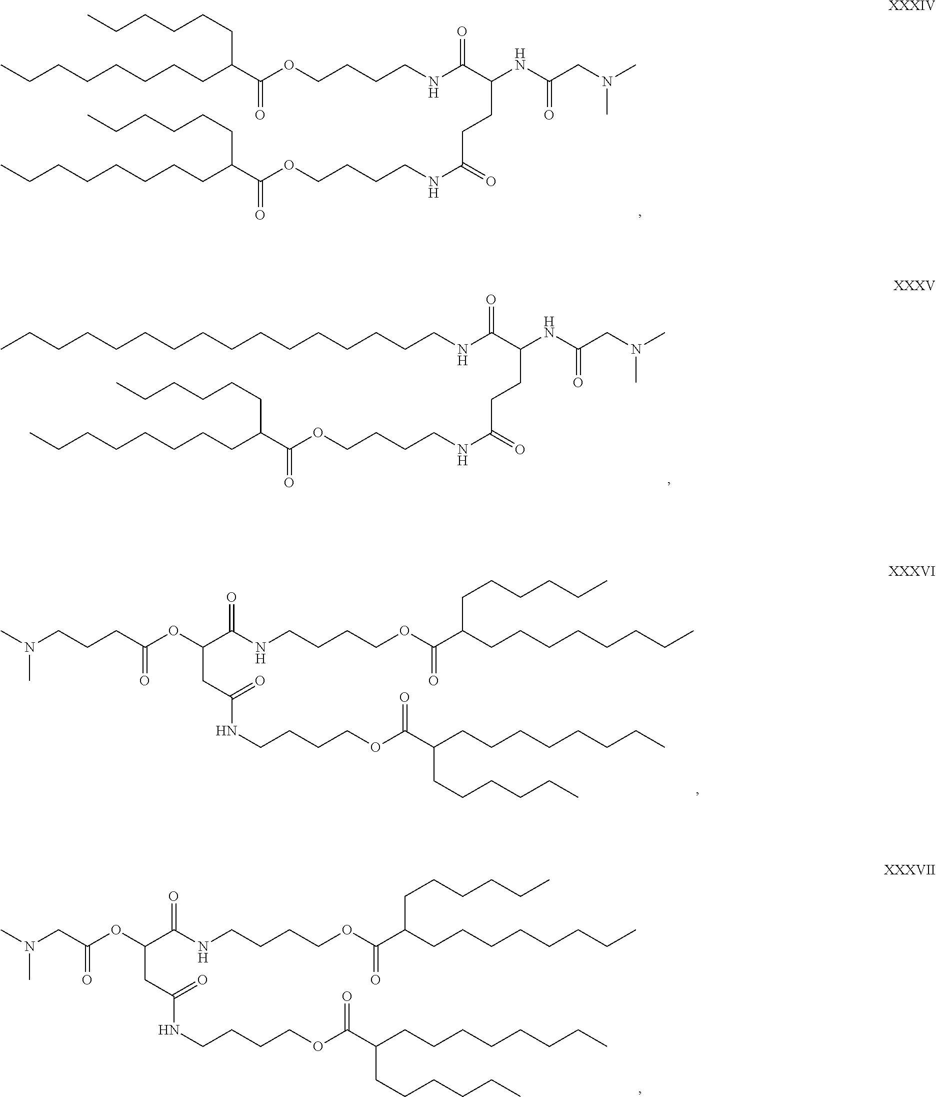

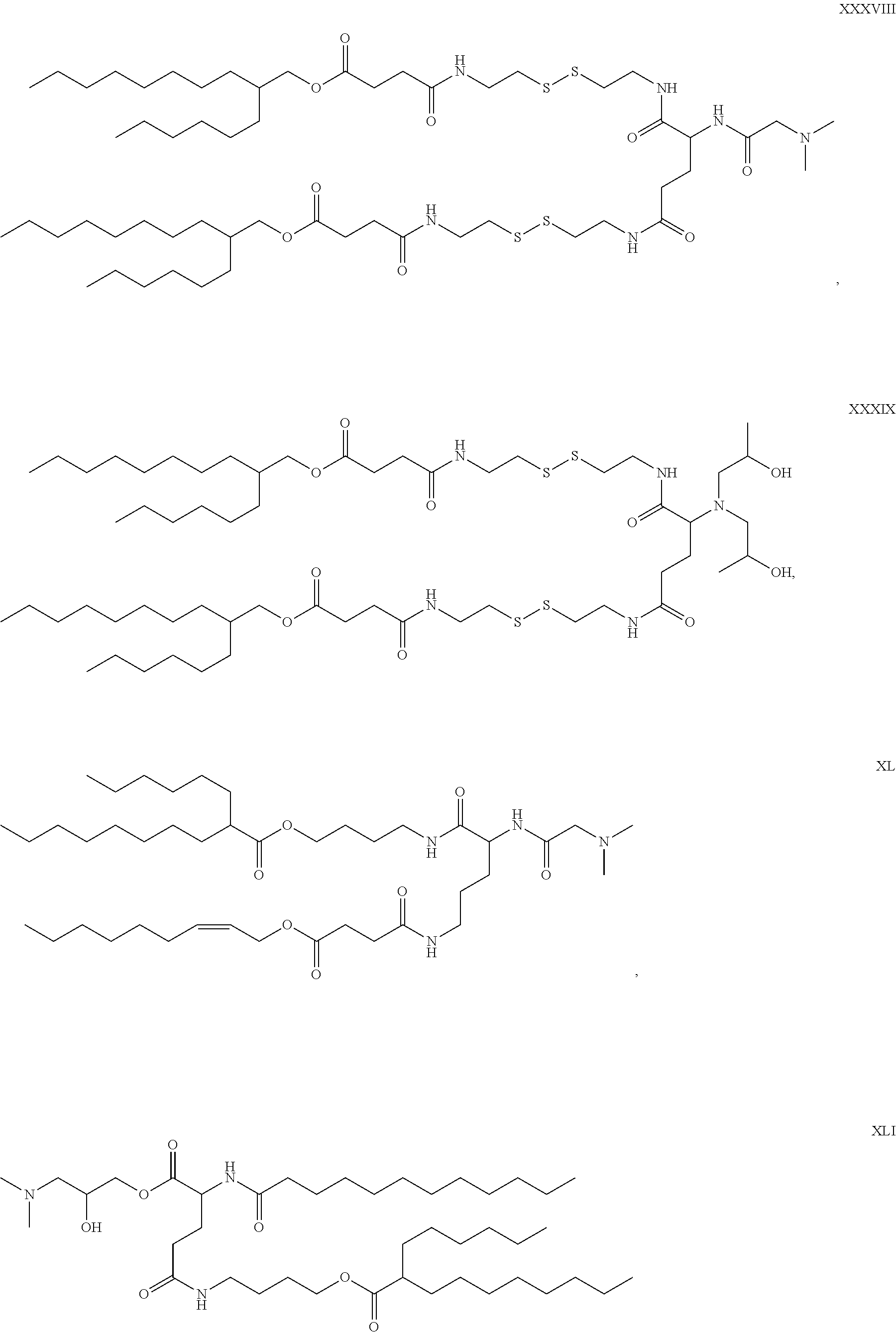

6. Composition comprising the lipid of formula (I) as defined in claim 1.

7. Composition according to claim 6, wherein said composition further comprises one or more active ingredients and optionally:
one or more neutral lipids, and/or
one or more components capable of reducing aggregation of said composition, and/or
one or more sterols or sterol derivatives, and/or
one or more lipids bearing a targeting ligand.

8. Process for manufacturing the lipid of formula (I) as defined in claim 1.

9. Lipid of formula (I) as defined in claim 1 for its use as a medicament.

10. Use of the lipid of formula (I) as defined in claim 1 as a vector for delivering an active ingredient to subject(s), organ(s), cell(s) and/or tissue(s).

11. Method for transfecting cells with a nucleic acid and/or a protein and/or a peptide and/or a polysaccharide and/or a lipid and/or a small organic or inorganic molecule and/or any type of bioactive molecule using the lipid of formula (I) as defined in claim 1.

12. Transfected cells obtained by the method as defined in claim 11.

13. Kit comprising the lipid of formula (I) as defined in claim 1 further comprising a nucleic acid and/or a protein and/or a peptide and/or a polysaccharide and/or a lipid and/or a small organic or inorganic molecule and/or any type of bioactive molecule, and optionally instructions of use.
